(19) United States
(12) Patent Application Publication
(10) Pub. No.: US 2025/0243186 A1
(43) Pub. Date: Jul. 31, 2025

(54) PIPERIDINYL INDOLE DERIVATIVES, PREPARATION METHODS AND MEDICINAL USES THEREOF

(71) Applicants: HANSOH BIO LLC, Rockville, MD (US); SHANGHAI HANSOH BIOMEDICAL CO., LTD., Shanghai (CN); JIANGSU HANSOH PHARMACEUTICAL GROUP CO., LTD., Lianyungang (CN)

(72) Inventors: Hugh Y. Zhu, Rockville, MD (US); Avinash Khanna, Rockville, MD (US); Matthew Kier, Rockville, MD (US); Lisa A. De Meese, Rockville, MD (US); Wei Zhou, Rockville, MD (US)

(21) Appl. No.: 18/705,084

(22) PCT Filed: Oct. 27, 2022
(86) PCT No.: PCT/CN2022/127975

§ 371 Date: Apr. 26, 2024

Related U.S. Application Data
(60) Provisional application No. 63/263,108, filed on Oct. 27, 2021.
(60) Provisional application No. 63/268,655, filed on Feb. 28, 2022.
(60) Provisional application No. 63/362,916, filed on Apr. 13, 2022.
(60) Provisional application No. 63/365,029, filed on May 20, 2022.
(60) Provisional application No. 63/366,101, filed on Jun. 9, 2022.

(51) Int. Cl. C07D 403/10; A61K 31/4045; A61K 31/438; A61K 31/444; C07D 401/10; C07D 413/14

(52) U.S. Cl. CPC C07D 403/10; A61K 31/4045; A61K 31/438; A61K 31/444; C07D 401/10; C07D 413/14; C07B 2200/05

(57) ABSTRACT

Compounds of formula (I) as piperidinyl indole derivatives, the preparation method thereof, pharmaceutical compositions comprising the compounds, and the pharmaceutical uses for the treatment a disease or disorder mediated by complement activation.

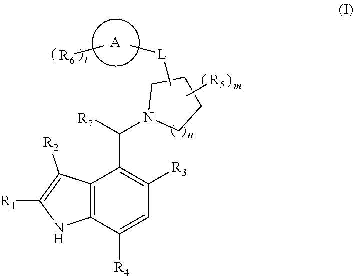

### FIELD OF THE INVENTION

The present invention belongs to the field of medicine, and relates to piperidinyl indole derivatives, preparation methods thereof, pharmaceutical compositions comprising the compounds, and medical uses thereof.

### BACKGROUND FOR INVENTION

The complement system is a part of the innate immunosurveillance, playing a critical role in eliminating pathogens and in the tissue homeostasis. The complement cascade can be activated by three different pathways including classical (CP), lectin (LP), and alternative pathway (AP). The CP and LP are initiated on target surfaces by immune complexes and binding of mannan-binding lectin or ficolin to a particular of microbial sugar moiety pattern, respectively. However, the AP does not require specific initiation. The AP cascade is initiated by spontaneous hydrolysis of C3 (tick-over) and subsequent deposition of C3b on an activating surface. The three complement activation pathways converge on two major events, C3 cleavage and C5 cleavage. C3 convertases split C3 into C3a and C3b. C3b forms additional AP C3 convertases (amplification) as well as C5 convertases. C5 convertases cleave C5 into C5a and C5b. The produced C5b initiates the formation of the C5b-9 membrane attack complex (MAC) with C6-C9, leading to lysis of bacteria and cells by insertion into a membrane. The split products C3a and C5a function as anaphylatoxins to promote pro-inflammatory responses through activation and chemotaxis of leukocytes. C3b also plays a key role in removing bacteria and cellular waste such as immune complexes and apoptotic cells through promoting phagocytosis by opsonization. (Front Immunol. 2015 Jun. 2; 6:262. doi: 10.3389/fimmu.2015.00262. eCollection 2015. Complement System Part I—Molecular Mechanisms of Activation and Regulation. Nicolas S Merle, Sarah Elizabeth Church, Veronique Fremeaux-Bacchi, Lubka T Roumenina). The AP maintains the basal complement activity through a “tick-over process. Moreover, the AP contributes more than 80% of terminal lysis pathway activation (MAC formation) through an amplification loop even if initiated via the other CP or LP. (Harboe, M., Garred, P., Karlstrom, E., Lindstad, J. K., Stahl, G. L., Mollnes, T. E., 2009. The down-stream effects of mannan-induced lectin complement pathway activation depend quantitatively on alternative pathway amplification. Mol. Immunol. 47, 373-380. https://doi.org/10.1016/j.molimm.2009.09.005). The spontaneous activated C3 forms C3 convertase by binding with factor B (FB). After cleavage of FB into Bb by factor D, C3b and Bb generate the AP C3 convertase (C3bBb). The newly formed C3bBb cleaves more C3 to generate more AP C3 convertases, leading to the amplification of complement cascade. As the AP is ready to exert full complement activity within seconds, it can lead to normal tissue injury if not controlled properly. (J Clin Invest. 2020 May 1; 130(5):2152-2163. doi: 10.1172/JCI136094. Complementopathies and precision medicine. Eleni Gavriilaki, Robert A Brodsky). Dysregulated complement activation has been shown to be associated with diseases in various organs including paroxysmal nocturnal hemoglobinuria, age-related macular degeneration, rheumatoid arthritis, hemolytic uremic syndrome, myasthenia gravis, and C3 glomerulo-nephriti. (J Clin Invest. 2020 May 1; 130(5):2152-2163. doi: 10.1172/JCI136094.). Therefore, controlling the AP through FB inhibition may be a powerful strategy for limiting the overactivation of the Complement pathway.

Currently, there are no small-molecules approved for modulating the Complement pathways. Examples of Factor B inhibitors are described in the following disclosures: Advanced Vision Therapies Inc. patent publication WO2008/106644 titled “Treatment of diseases characterized by inflammation”; Wellstate Immunotherapeutics patent publication WO2012/151468 titled “Complement Factor B analogs and their uses”; William Marsh Rice University patent publication WO2014/035876 titled “Heat-inactivated Complement Factor B compositions and methods”; Muse. Foundation for Research Development patent publication US1999/023485 titled “Blocking factor b to treat complement-mediated immune disease”; and Novartis patent publication WO2013/192345 and US2015/126592 titled “Complement pathway modulators and uses thereof”. Additional Factor B inhibitors are described in Novartis patent publications WO2015/066241, US2016/311779, WO2015/009616, US2016/152605, WO2014/143638, and US2016/024079. Another example of Factor B inhibitors is the IONIS Pharmaceuticals Inc. patent publication WO2015/038939 titled “Modulators of Complement Factor B”. Examples of granted patents covering Factor B inhibitors include U.S. Pat. Nos. 9,452,990; 9,676,728; 9,682,968; and 9,475,806.

Given the large array of diseases that are driven by an overactive complement pathway there is a high unmet need for patients of Complement diseases. This invention aims to provide compounds which modulate Factor B and treat disorders associated with the dysregulation of the Complement pathway.

### SUMMARY OF THE INVENTION

The present invention, in one aspect, provides a compound of formula (I), or tautomer, or pharmaceutically acceptable salt thereof,

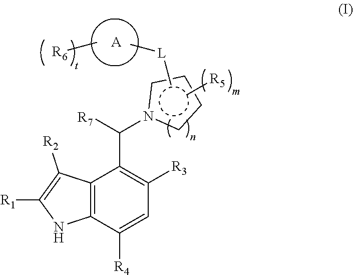

wherein:

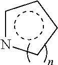

is saturated ring or unsaturated ring;
A is cycloalkyl, heterocyclyl, aryl or heteroaryl;
L is bond, (CRₐR_{b})ₚ or absent;
Rₐ and R_{b} are independently selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl and heteroaryl;
R₁ and R₂ are independently selected from the group consisting of hydrogen, deuterium, halogen, amino, cyano, hydroxy, alkyl, alkoxy, alkylthio, haloalkyl and hydroxyalkyl;
R₃ and R₄ are independently selected from the group consisting of hydrogen, deuterium, halogen, amino, cyano, hydroxy, alkyl, alkoxy, alkylthio, haloalkyl, haloalkenyl, hydroxyalkyl, deuterated alkoxy, haloalkoxy, cycloalkyl, heterocyclyl, aryl, heteroaryl, cycloalkylxoy, heterocyclylxoy, arylxoy and heteroarylxoy, optionally the hydroxy, alkyl, alkoxy, alkylthio, haloalkyl, hydroxyalkyl, deuterated alkoxy, haloalkoxy, cycloalkyl, heterocyclyl, aryl, heteroaryl, cycloalkylxoy, heterocyclylxoy, arylxoy and heteroarylxoy substituted with one or more substituents selected from deuterium, halogen, amino, cyano, hydroxy, alkyl, alkoxy, alkylthio, haloalkyl, hydroxyalkyl, cycloalkyl, heterocyclyl, aryl and heteroaryl;
R₅ is independently selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl and heteroaryl, optionally the amino, alkyl, alkoxy, alkylthio, haloalkyl, hydroxyalkyl, cycloalkyl, heterocyclyl, aryl and heteroaryl substituted with one or more substituents selected from deuterium, halogen, amino, cyano, hydroxy, alkyl, alkoxy, alkylthio, haloalkyl and hydroxyalkyl;
or, two of R₅ are together with the C atom to which they are attached form cycloalkyl or heterocyclyl, optionally the cycloalkyl or heterocyclyl substituted with one or more substituents selected from deuterium, halogen, amino, cyano, hydroxy, alkyl, alkenyl, alkynyl, alkoxy, alkylalkoxy, alkoxyalkyl, alkylthio, haloalkyl and hydroxyalkyl;
R₆ is selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl and heteroaryl, —(CH₂)ᵣOR₈, —(CH₂)ᵣC(O)R₈, —S(O)NHalkyl, —SO₂alkyl, —C(O)NHSO₂alkyl and —SO₂NHC(O)alkyl;
or, R₆ together with the C atom in

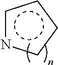

to form cycloalkyl or heterocyclyl, optionally the cycloalkyl or heterocyclyl substituted with one or more substituents selected from deuterium, halogen, amino, cyano, hydroxy, alkyl, alkoxy, alkylalkoxy, alkoxyalkyl, alkylthio, haloalkyl and hydroxyalkyl;
R₇ is selected from the group consisting of hydrogen, deuterium, halogen, amino, cyano, hydroxy, alkyl, alkoxy, alkylthio, haloalkyl and hydroxyalkyl;
R₈ is selected from the group consisting of hydrogen, deuterium, halogen, amino, cyano, hydroxy, alkyl, alkoxy, alkylthio, haloalkyl and hydroxyalkyl;
p is 1, 2 or 3;
r is 0, 1, 2 or 3;
t is 1, 2 or 3;
m is 1, 2 or 3; and
n is 0, 1, 2 or 3;
provided that if
R₁ and R₂ is hydrogen, R₃ is cyclopropyl or methoxy, R₄ is methyl, L is bond, R₆ is —COOH or —COOCH₃, R₇ is hydrogen or trifluoromethyl, A is phenyl, and n is 1, 2 or 3, R₅ is not hydrogen or

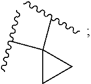 [SMILES: *C1(*)CC1]

R₁ and R₂ is hydrogen, R₄ is methyl, L is bond, R₇ is hydrogen, A is phenyl, pyridine or thiazolyl, m is 1, and n is 2, R₅ is not hydrogen, amino, hydroxy, methyl, ethyl, methoxy, ethyoxyl, propoxy, methylol, ethoxyl, cyanomethyl and methylamino; and,
R₁ and R₂ is hydrogen, R₄ is methyl, L is bond, R₇ is hydrogen, A is phenyl, m is 2 or 3, and n is 2, R₅ is not hydrogen or methyl.

In an embodiment, A is C₆₋₁₀ aryl or 5-10 membered heteroaryl.

In a preferred embodiment, A is phenyl, naphthyl or 5-8 membered heteroaryl containing 1, 2 or 3 ring heteroatoms independently selected from N, O or S.

In a preferred embodiment, A is phenyl, benzocycloalkyl, or 5-8 membered heteroaryl containing 1, 2 or 3 of N heteroatoms.

In a more preferred embodiment, A is

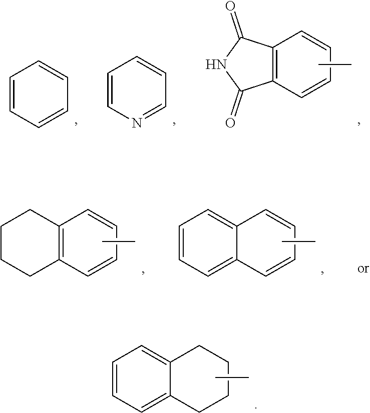

In a preferred embodiment, L is bond, CH₂ or absent.

In a preferred embodiment, L is bond.

In a preferred embodiment, R₁ and R₂ are independently selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl and C₁₋₆ hydroxyalkyl.

In a more preferred embodiment, R₁ and R₂ are hydrogen.

In a preferred embodiment, R₃ and R₄ are independently selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ haloalkenyl, C₁₋₆ hydroxyalkyl, deuterated C₁₋₆ alkoxy, C₁₋₆ haloalkoxy, C₃₋₆ cycloalkyl, 4-10 membered heterocyclyl, C₆₋₁₀ aryl, 5-10 membered heteroaryl, C₃₋₆ cycloalkyloxy, 4-10 membered heterocyclyloxy, C₆₋₁₀ aryloxy and 5-10 membered heteroaryloxy, optionally the C₁₋₆ alkyl, C₁₋₆ alkoxy, C₁₋₆ alkylthio, C₁₋₆ haloalkyl, C₁₋₆ hydroxyalkyl, deuterated C₁₋₆ alkoxy, C₁₋₆ haloalkoxy substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₆ cycloalkyl, 4-10 membered heterocyclyl, C₆₋₁₀ aryl and 5-10 membered heteroaryl.

In a more preferred embodiment, R₃ and R₄ are independently selected from the group consisting of C₁₋₃ alkyl, C₁₋₃ alkoxy, deuterium, halogen, deuterated C₁₋₃ alkoxy, C₁₋₃ haloalkoxy, C₃₋₆ cycloalkyl and C₃₋₆ cycloalkyloxy, optionally the C₁₋₃ alkyl, C₁₋₃ alkoxy, deuterated C₁₋₃ alkoxy, C₁₋₃ haloalkoxy substituted with one or more substituents selected from C₃₋₆ cycloalkyl, 4-6 membered heterocyclyl, C₆₋₁₀ aryl and 5-10 membered heteroaryl.

In a more preferred embodiment, R₃ and R₄ are independently selected from the group consisting of deuterium, halogen, C₁₋₃ alkyl, C₁₋₃ alkoxy, deuterated C₁₋₃ alkoxy and C₁₋₃ haloalkoxy.

In a preferred embodiment, R₆ is selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, —(CH₂)ᵣC₁₋₆ alkoxy, —(CH₂)ᵣC(O)OH, —S(O)NHC₁₋₆ alkyl, —SO₂C₁₋₆ alkyl, —C(O)NHSO₂C₁₋₆ alkyl and —SO₂NHC(O)C₁₋₆ alkyl.

In a preferred embodiment, R₆ is selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 5-6membered heterocyclyl containing 1-3 of heteroatom selected from N, O and S, C₅₋₁₀ aryl and 5-6 membered heteroaryl containing 1-3 of heteroatom selected from N, O and S, —(CH₂)ᵣC₁₋₆ alkoxy, —(CH₂)ᵣC(O)OH, —S(O)NHC₁₋₆ alkyl, —SO₂C₁₋₆ alkyl, —C(O)NHSO₂C₁₋₆ alkyl and —SO₂NHC(O)C₁₋₆ alkyl.

In a more preferred embodiment, R₆ is —COOH, 5-6membered heterocyclyl containing 1-3 of heteroatom selected from N, O and S, or 5-6 membered heteroaryl containing 1-3 of heteroatom selected from N, O and S.

In a more preferred embodiment, R₆ is —F, -OMe, —CH₂OH, —CH₂OCH₃, —CH₂F, —CF₂H, —CF₃, —COOH, —C(O)NHSO₂CH₃ or —S(O)NHCH₃.

In a more preferred embodiment, R₆ is —COOH or —S(O)NHCH₃.

In a more preferred embodiment, R₆ is —COOH,

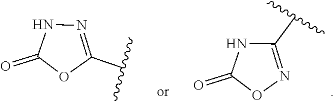 [SMILES: *c1n[nH]c(=O)o1 ; *c1noc(=O)[nH]1]

In a more preferred embodiment, R₆ is

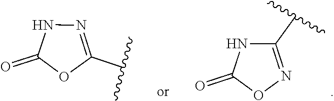 [SMILES: *c1n[nH]c(=O)o1 ; *c1noc(=O)[nH]1]

In a more preferred embodiment, R₆ is —COOH.

In a preferred embodiment, R₅ is independently selected from the group consisting of hydrogen, deuterium, halogen, amino, cyano, hydroxy, C₁₋₆ alkyl, C₂₋₆ alkenyl, C₂₋₆ alkynyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, optionally the C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl and C₁₋₆ hydroxyalkyl;
or, two of R₅ are together with the C atom to which they are attached form C₃₋₆ cycloalkyl or 4-6 membered heterocyclyl containing 1, 2 or 3 ring heteroatoms independently selected from N, O or S, optionally substituted with one or more substituents selected from deuterium, halogen, amino, cyano, hydroxy, C₁₋₆ alkyl, C₁₋₆ alkoxy, C₁₋₆ alkylC₁₋₆ alkoxy, C₁₋₆ alkoxyC₁₋₆ alkyl, C₁₋₆ alkylthio, C₁₋₆ haloalkyl and C₁₋₆ hydroxyalkyl.

In a preferred embodiment, R₇ is selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl and C₁₋₆ hydroxyalkyl.

In a preferred embodiment, R₇ is hydrogen or C₁₋₃ alkyl.

In a preferred embodiment, R₈ is selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl and C₁₋₆ hydroxyalkyl.

In a preferred embodiment, R₅ is s independently selected from the group consisting of hydrogen, deuterium, halogen, amino, cyano, hydroxy, C₁₋₃ alkyl, C₂₋₄ alkenyl, C₂₋₄ alkynyl, C₁₋₃ alkoxy, C₁₋₃ alkylthio, C₁₋₃ haloalkyl, C₁₋₃ hydroxyalkyl, C₃₋₆ cycloalkyl, 4-6 membered heterocyclyl containing 1, 2 or 3 ring heteroatoms independently selected from N, O or S, C₅₋₁₀ aryl and 5-6 membered heteroaryl containing 1, 2 or 3 ring heteroatoms independently selected from N, O or S, optionally the C₁₋₃ alkyl, C₁₋₃ alkoxy, C₁₋₃ alkylthio, C₁₋₃ haloalkyl, C₁₋₃ hydroxyalkyl, C₃₋₆ cycloalkyl, 4-6 membered heterocyclyl, C₅₋₁₀ aryl and 5-6 membered heteroaryl substituted with one or more substituents selected from deuterium, halogen, amino, cyano, hydroxy, C₁₋₃ alkyl, C₁₋₃ alkoxy, C₁₋₃ alkylthio, C₁₋₃ haloalkyl and C₁₋₃ hydroxyalkyl; or, two of R₅ are together with the C atom to which they are attached form C₃₋₆ cycloalkyl or 4-6 membered heterocyclyl containing 1, 2 or 3 ring heteroatoms independently selected from N, O or S, optionally substituted with one or more substituents selected from deuterium, halogen, amino, cyano, hydroxy, C₁₋₃ alkyl, C₁₋₃ alkoxy, C₁₋₃ alkyl C₁₋₃ alkoxy, C₁₋₃ alkoxy C₁₋₃ alkyl, C₁₋₃ alkylthio, C₁₋₃ haloalkyl and C₁₋₃ hydroxyalkyl.

In a preferred embodiment, the compound of formula (I) may be compounds of formula (II-a)-(II-e), or tautomer, or pharmaceutically acceptable salt thereof,

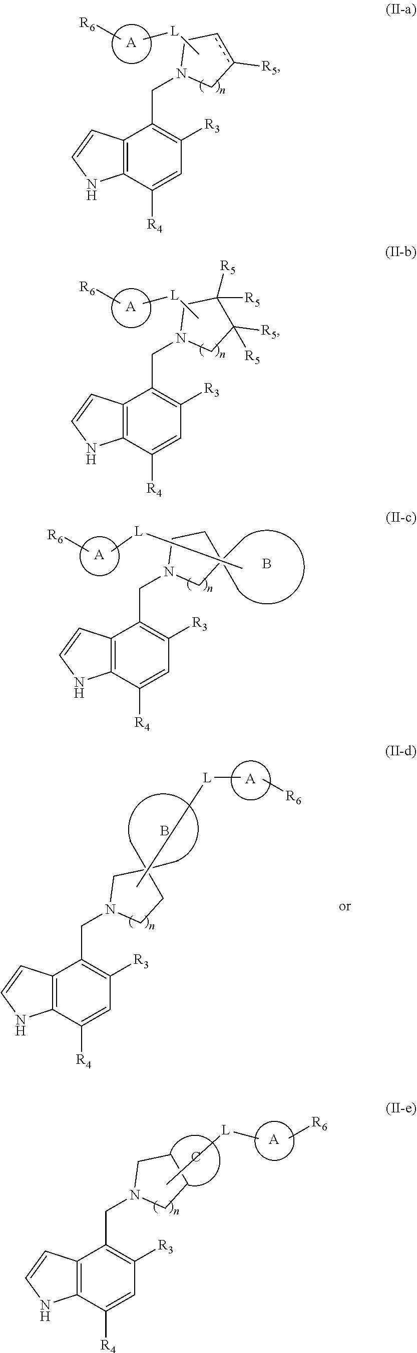

wherein, [symbol] is single or double bond;
R₅ is independently selected from the group consisting of hydrogen, deuterium, halogen, amino, cyano, hydroxy, C₁₋₃ alkyl, C₁₋₃ alkoxy, C₁₋₃ alkylthio, C₁₋₃ haloalkyl, C₁₋₃ hydroxyalkyl, C₃₋₆ cycloalkyl, 4-6 membered heterocyclyl containing 1, 2 or 3 ring heteroatoms independently selected from N, O or S, C₅₋₁₀ aryl, and 5-6 membered heteroaryl containing 1, 2 or 3 ring heteroatoms independently selected from N, O or S, optionally the C₁₋₃ alkyl, C₁₋₃ alkoxy, C₁₋₃ alkylthio, C₁₋₃ haloalkyl, C₁₋₃ hydroxyalkyl, C₃₋₆ cycloalkyl, 4-6 membered heterocyclyl, C₅₋₁₀ aryl and 5-6 membered heteroaryl substituted with one or more substituents selected from deuterium, halogen, amino, cyano, hydroxy, C₁₋₃ alkyl, C₁₋₃ alkoxy, C₁₋₃ alkylthio, C₁₋₃ haloalkyl, and C₁₋₃ hydroxyalkyl;
B is

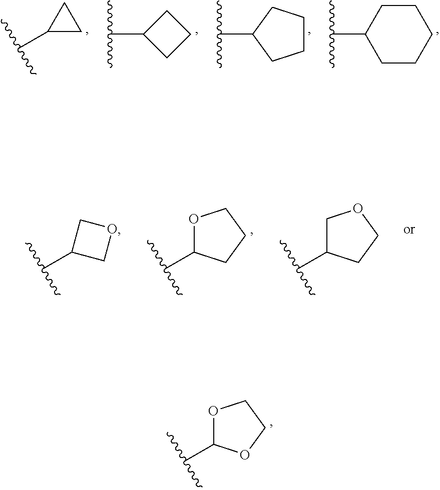

optionally the B is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl, and C₁₋₃ hydroxyalkyl;
C is

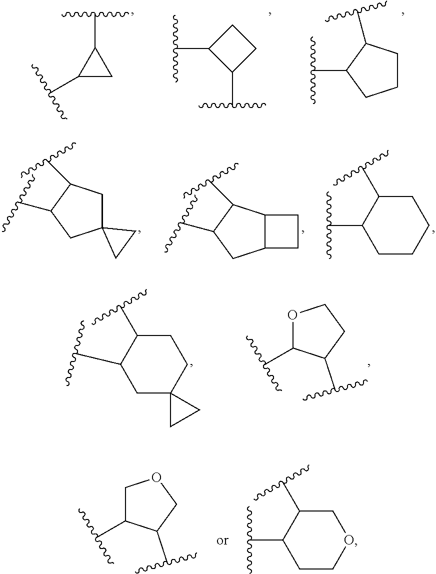

optionally the C is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl, and C₁₋₃ hydroxyalkyl.

In a preferred embodiment, the compound of formula (II-a)-(II-e), or tautomer, or pharmaceutically acceptable salt thereof,
B is

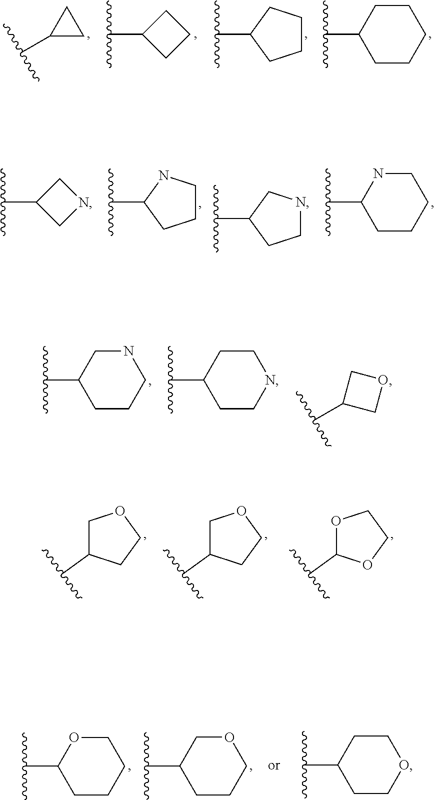

optionally the B is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl;
C is

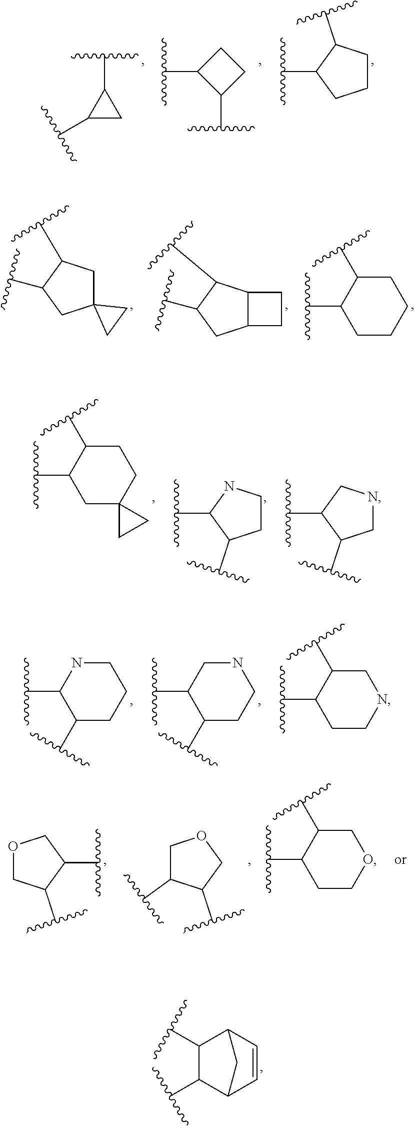

optionally the C is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl.

In a preferred embodiment, C is

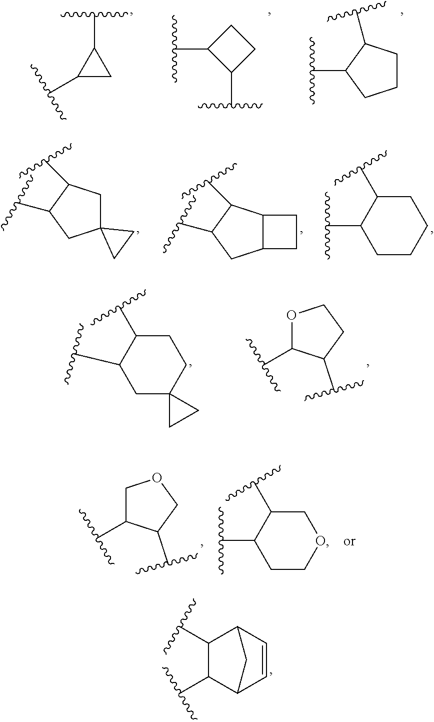

optionally the C is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl.

In a more preferred embodiment, the compound of formula (II-a)-(II-e) may be compounds of formula (III-a)-(III-e), or tautomer, or pharmaceutically acceptable salt thereof,

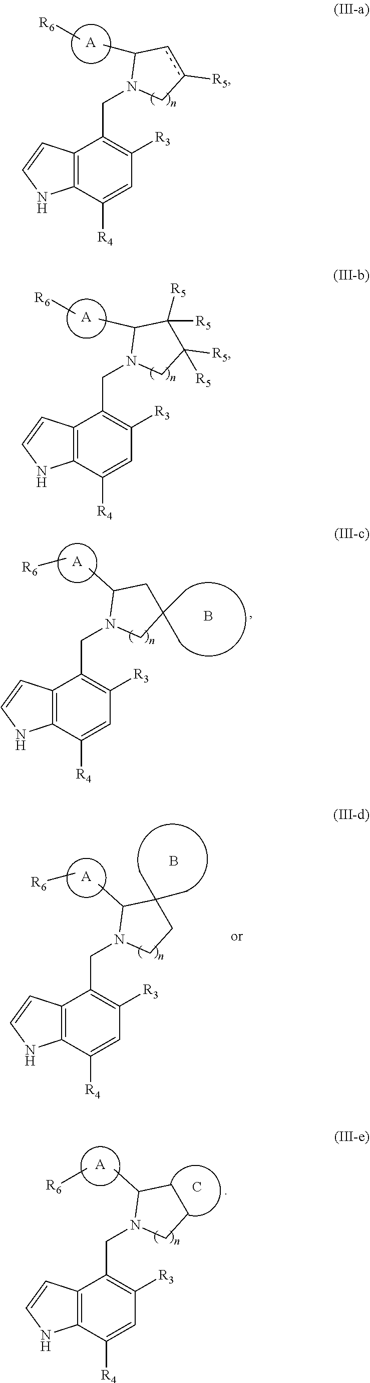

In a more preferred embodiment, n is

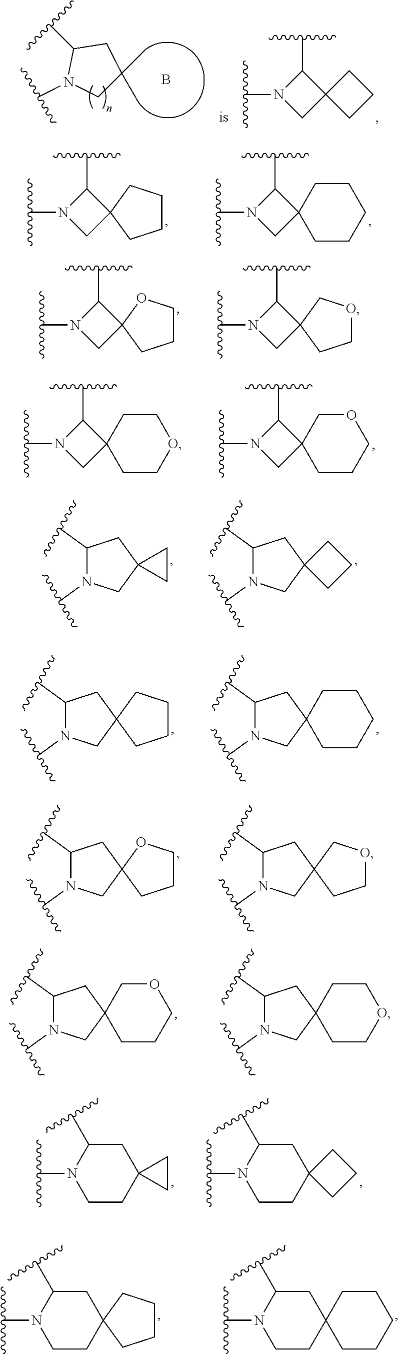

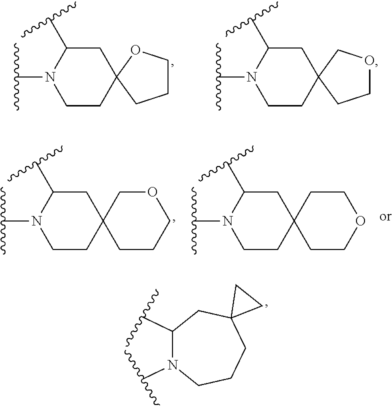

optionally the

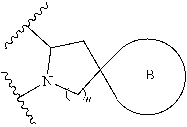

is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃ alkylthio, C₁₋₃ haloalkyl and C₁₋₃ hydroxyalkyl;

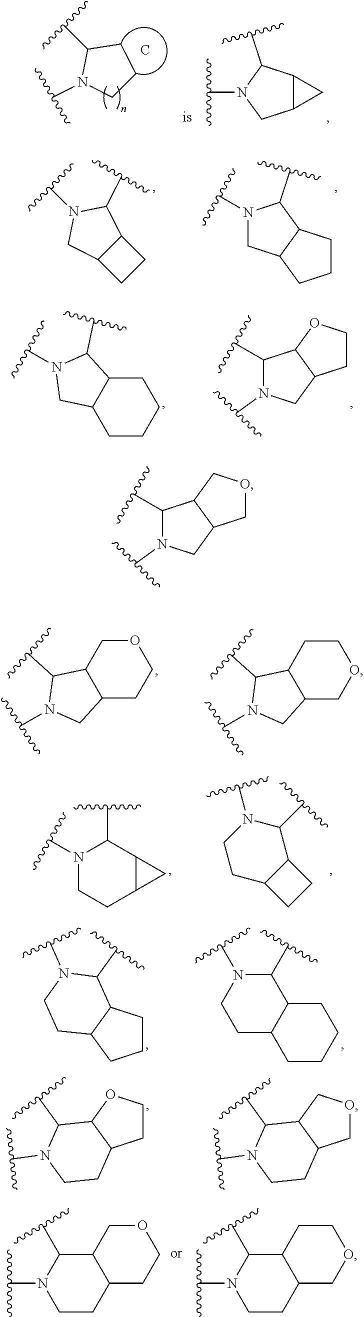

optionally the

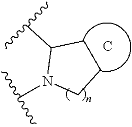

is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl.

In a more preferred embodiment,

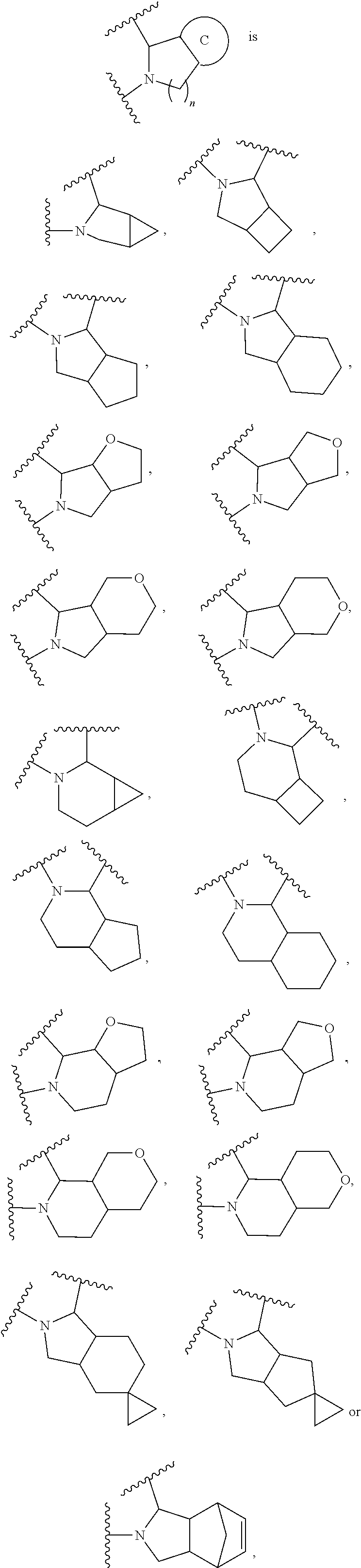

optionally the

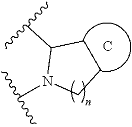

is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxy alkyl.

In a more preferred embodiment,

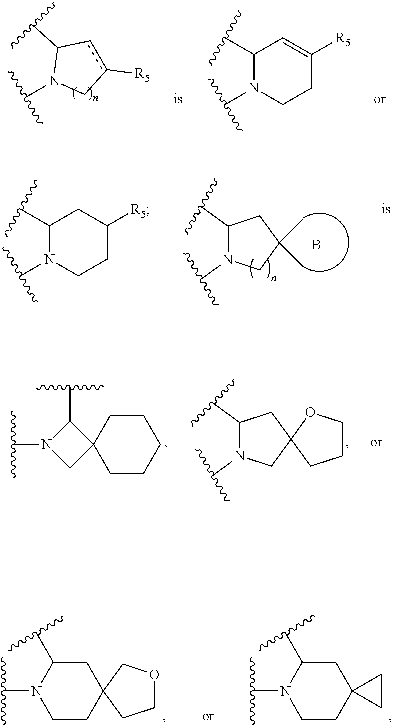

optionally

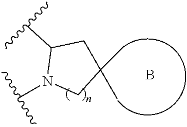

is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl;

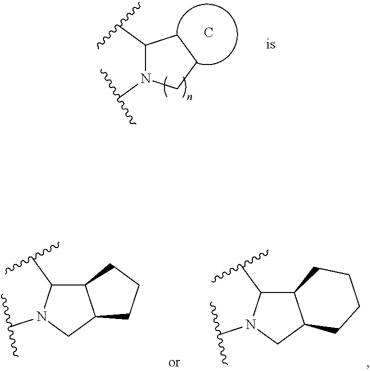

optionally the

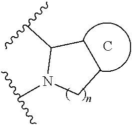

is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl.

In a more preferred embodiment,

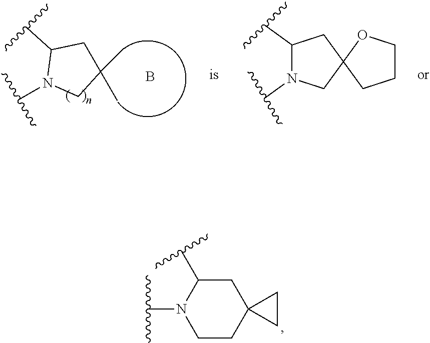

optionally the

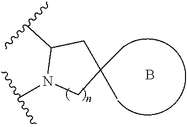

is substituted with one or or, more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl;

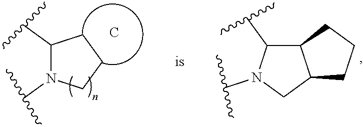

optionally the

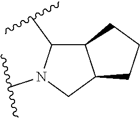 [SMILES: *C1[C@H]2CCC[C@H]2CN1*]

is substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl.

In a more preferred embodiment, the compound of formula (I) may be compounds of formula (IV), or tautomer, or pharmaceutically acceptable salt thereof,

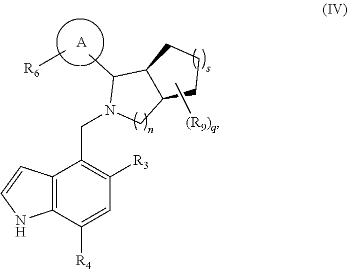

R₉ is hydrogen, halogen, amino, cyano, hydroxy, C₁₋₃ alkyl, C₂₋₄ alkenyl, C₂₋₄ alkynyl, C₁₋₃ alkoxy, C₁₋₃alkylthio, C₁₋₃ haloalkyl and C₁₋₃ hydroxyalkyl, optionally substituted with one or more substituents selected from halogen, amino, hydroxy, C₁₋₃ alkyl, C₁₋₃ alkoxy, C₁₋₃ alkylamino, C₃₋₆ cycloalkyl and 5-6 membered heterocyclyl containing 1 or 2 ring heteroatoms independently selected from N or O;
or, two of R₉ together with the C atom to which they are attached from C₃₋₆ cycloalkyl, optionally substituted with one or more substituents selected from deuterium, halogen, amino, cyano, hydroxy, C₁₋₃ alkyl, C₂₋₄ alkenyl, C₂₋₄ alkynyl, C₁₋₃ alkylamino, C₁₋₃ alkoxy, C₁₋₃ alkylCl₁₋₃ alkoxy, C₁₋₃ alkoxyC₁₋₃ alkyl, C₁₋₃alkylthio, C₁₋₃ haloalkyl and C₁₋₃ hydroxyalkyl;
n is 1 or 2;
q 1, 2 or 3, and
s is 0, 1 or 2.

In a more preferred embodiment,

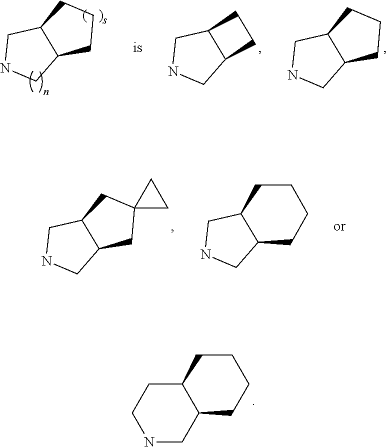

In a more preferred embodiment,
A is

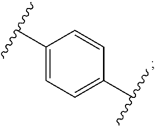 [SMILES: *c1ccc(*)cc1]

or, each of R₃ and R₄ is methyl or methoxy;
or, R₅ is hydrogen, halogen, cyano, C₁₋₃ alkyl, C₂₋₄ alkenyl, C₂₋₄ alkynyl, C₁₋₃ alkoxy, C₁₋₃ haloalkyl, C₁₋₃ alkylC₁₋₃ alkoxy, C₁₋₃ alkoxyCl₁₋₃ alkyl, C₃₋₆ cycloalkyl, 5 membered heteroaryl containing 1 or 2 ring heteroatoms independently selected from N or O; or, R₆ is —COOH or —S(O)NHCH₃.

In a more preferred embodiment, the compound of formula (I) may be compounds of formula (V-a)-(V-c), or tautomer, or pharmaceutically acceptable salt thereof,

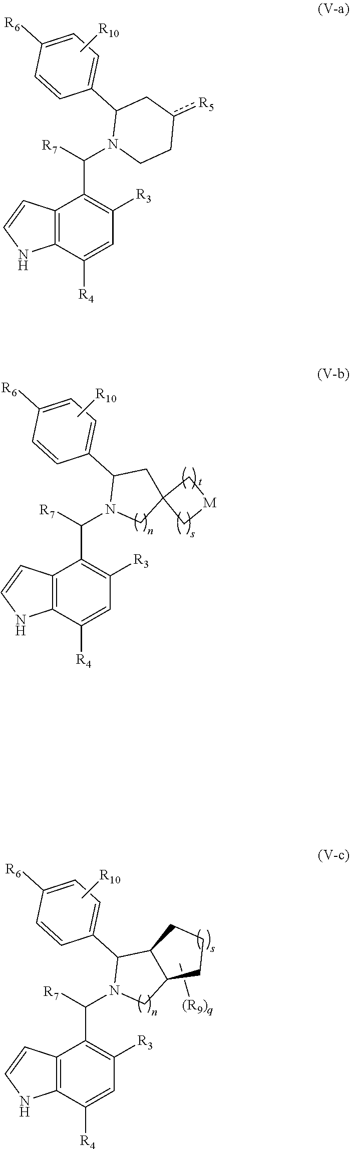

is single bond or double bond;
M is O or CR_{c}R_{d};
R_{c} and R_{d} are independently selected from hydrogen, halogen or C₁₋₃ alkyl;
R₃ and R₄ are independently selected from C₁₋₃ alkyl, C₁₋₃ alkoxy or C₃₋₆ cycloalkyl;
R₅ is hydrogen, halogen, C₁₋₃ alkyl or C₁₋₃ haloalkyl;
R₆ is —COOH, —C(O)NHSO₂CH₃ or —S(O)NHCH₃;
R₇ is hydrogen, C₁₋₃ alkyl or C₁₋₃ hydroxyalkyl;
R₉ is hydrogen, halogen, C₁₋₃ alkyl or C₁₋₃ haloalkyl;
or, two of R₉ together with the C atom to which they are attached form C₃₋₆ cycloalkyl; R₁₀ is hydrogen, C₁₋₃ alkyl or C₁₋₃ haloalkyl;
n is 1 or 2;
q is 1, 2 or 3,
s is 0, 1 or 2, and
t is 1 or 2.

In a more preferred embodiment, the compound of formula (I) may be compounds of formula (V-a)-(V-c), or tautomer, or pharmaceutically acceptable salt thereof,

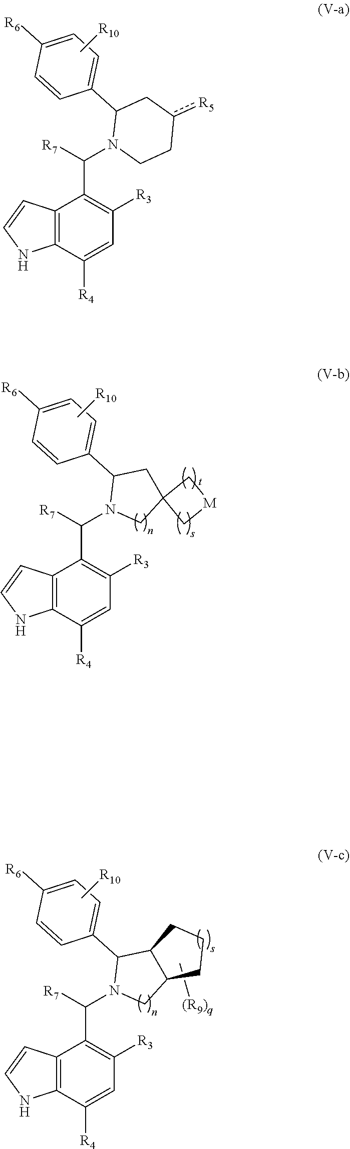

is single bond or double bond;
M is O or CR_{c}R_{d};
R_{c} and R_{d} are independently selected from hydrogen, halogen or C₁₋₃ alkyl;
R₃ and R₄ are independently selected from deuterium, halogen, C₁₋₃ alkyl, C₁₋₃ alkoxy, C₃₋₆ cycloalkyl, C₃₋₆ cycloalkyloxy, deuterated C₁₋₃ alkoxy, C₁₋₃ haloalkoxy and C₃₋₆ cycloalkylC₁₋₃ alkoxy;
R₅ is hydrogen, halogen, C₁₋₃ alkyl or C₁₋₃ haloalkyl;
R₆ is —COOH, —C(O)NHSO₂CH₃ or —S(O)NHCH₃;
R₇ is hydrogen, C₁₋₃ alkyl or C₁₋₃ hydroxyalkyl;
R₉ is hydrogen, halogen, C₁₋₃ alkyl or C₁₋₃ haloalkyl;
or, two of R₉ together with the C atom to which they are attached form C₃₋₆ cycloalkyl;
R₁₀ is hydrogen, C₁₋₃ alkyl or C₁₋₃ haloalkyl;
n is 1 or 2;
q is 1, 2 or 3,
s is 0, 1 or 2, and
t is 1 or 2.

In a more preferred embodiment, the compound of formula (I) may be compounds of formula (VI), or tautomer, or pharmaceutically acceptable salt thereof,

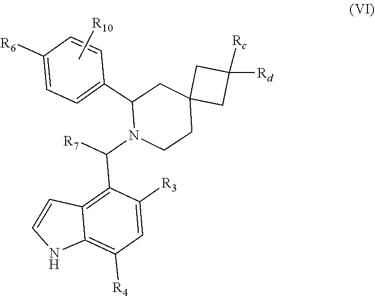

R₃ and R₄ are independently selected from deuterium, halogen, C₁₋₃ alkyl, C₁₋₃ alkoxy, C₃₋₆ cycloalkyl, C₃₋₆ cycloalkyloxy, deuterated C₁₋₃ alkoxy, C₁₋₃ haloalkoxy and C₃₋₆ cycloalkylC₁₋₃ alkoxy;
R₆ is —COOH, —C(O)NHSO₂CH₃, —S(O)NHCH₃,

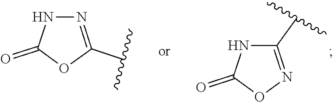 [SMILES: *c1n[nH]c(=O)o1 ; *c1noc(=O)[nH]1]

R₇ is hydrogen, C₁₋₃ alkyl or C₁₋₃ hydroxyalkyl;
R₁₀ is hydrogen, C₁₋₃ alkyl or C₁₋₃ haloalkyl;
each of R_{c} and R_{d} is independently selected from hydrogen, halogen, C₁₋₃ alkyl and C₁₋₃ haloalkyl.

In a preferred embodiment, for the formula (VI), or tautomer, or pharmaceutically acceptable salt thereof, wherein:
R₃ and R₄ are independently selected from deuterium, halogen, C₁₋₃ alkyl, C₁₋₃ alkoxy, C₃₋₆ cycloalkyl, C₃₋₆ cycloalkyloxy, deuterated C₁₋₃ alkoxy, C₁₋₃ haloalkoxy and C₃₋₆ cycloalkylCl₁₋₃ alkoxy;
R₆ is —COOH, —C(O)NHSO₂CH₃, —S(O)NHCH₃,

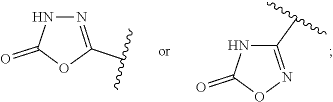 [SMILES: *c1n[nH]c(=O)o1 ; *c1noc(=O)[nH]1]

R₇ is hydrogen, C₁₋₃ alkyl or C₁₋₃ hydroxyalkyl;
R₁₀ is hydrogen, C₁₋₃ alkyl or C₁₋₃ haloalkyl;
each of R_{c} and R_{d} is F.

In a preferred embodiment, for the formula (VI), wherein:
R₃ and R₄ are independently selected from deuterium, halogen, C₁₋₃ alkyl, C₁₋₃ alkoxy, C₃₋₆ cycloalkyl, C₃₋₆ cycloalkyloxy, deuterated C₁₋₃ alkoxy, C₁₋₃ haloalkoxy and C₃₋₆ cycloalkylC₁₋₃ alkoxy;
R₆ is

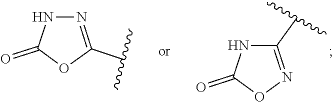 [SMILES: *c1n[nH]c(=O)o1 ; *c1noc(=O)[nH]1]

R₇ is hydrogen, C₁₋₃ alkyl or C₁₋₃ hydroxyalkyl;
R₁₀ is hydrogen, C₁₋₃ alkyl or C₁₋₃ haloalkyl;
each of R_{c} and R_{d} is F.

In a preferred embodiment, the compound of formula (VI) may be compounds of formula (VI-a), or tautomer, or pharmaceutically acceptable salt thereof,

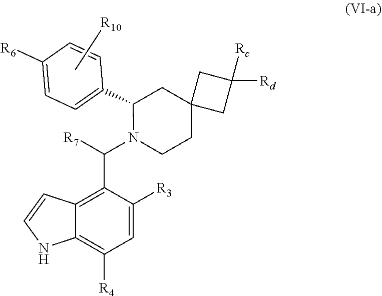

In a preferred embodiment, for the formula (VI-a), wherein:
R₃ and R₄ are dependently selected from deuterium, halogen, C₁₋₃ alkyl, C₁₋₃ alkoxy, C₃₋₆ cycloalkyl;
R₆ is —COOH, —C(O)NHSO₂CH₃, —S(O)NHCH₃;
R₇ is hydrogen, C₁₋₃ alkyl or C₁₋₃ hydroxyalkyl;
R₁₀ is hydrogen, C₁₋₃ alkyl or C₁₋₃ haloalkyl;
each of R_{c} and R_{d} is independently selected from hydrogen, halogen, C₁₋₃ alkyl and C₁₋₃ haloalkyl.

In a preferred embodiment, the compound of formula (VI-a) may be compounds of formula (VI-b), or tautomer, or pharmaceutically acceptable salt thereof,

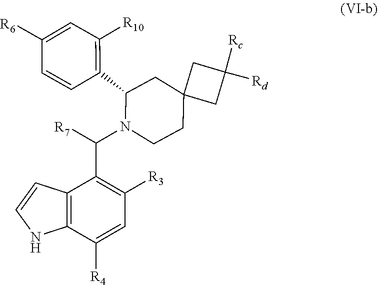

wherein,
R₃ and R₄ are independently selected from deuterium, halogen, C₁₋₃ alkyl, C₁₋₃ alkoxy, cyclopropyl, cyclobutyl;
R₆ is —COOH, —C(O)NHSO₂CH₃, —S(O)NHCH₃;
R₇ is hydrogen, C₁₋₃ alkyl or C₁₋₃ hydroxyalkyl;
R₁₀ is hydrogen, C₁₋₃ alkyl or C₁₋₃ haloalkyl, wherein the haloalkyl group contains at least two halogen atoms selected from F;
each of R_{c} and R_{d} is independently selected from hydrogen, halogen, C₁₋₃ alkyl and C₁₋₃ haloalkyl.

The present invention also provides a pharmaceutical composition, comprising a therapeutically effective amount of a compound of any formula (I)-(VI-b), or tautomer, or pharmaceutically acceptable salt thereof, together with one or more pharmaceutically acceptable carriers, diluents or excipients.

In another aspect, the present invention relates to a method of modulating complement alternative pathway activity, comprising administering to a subject in need thereof an effective amount of a compound of any formula (I)-(VI-b), or a pharmaceutical composition comprising the same.

In an embodiment, the amount of the compound, tautomer, cis- or trans-isomer, mesomer, racemate, enantiomer, diastereomer, or mixture thereof, or pharmaceutically acceptable salts thereof, is about 0.1-99%, 0.2-98.5%, 0.3-98%, 0.4-97.5%, 0.5-97%, 0.6-96.5%, 0.7-96%, 0.8-95.5%, 0.9-95%, 1-94.5%, 1.1-94%, 1.2-93.5%, 1.3-93%, 1.4-92.5%, 1.5-92%, 1.6-91.5%, 1.7-91%, 1.8-90.5%, 1.9-90%, 2-89.5%, 2.1-89%, 2.2-88.5%, 2.3-88%, 2.4-87.5%, 2.5-87%, 2.6-86.5%, 2.7-86%, 2.8-85.5%, 2.9-85%, 3-84.5%, 3.1-84%, 3.2-83.5%, 3.3-83%, 3.4-82.5%, 3.5-82%, 3.6-81.5%, 3.7-81%, 3.8-80.5%, 3.9-80%, 4-79.5%, 4.1-79%, 4.2-78.5%, 4.3-78%, 4.4-77.5%, 4.5-77%, 4.6-76.5%, 4.7-76%, 4.8-75.5%, 4.9-75%, 5-74.5%, 5.1-74%, 5.2-73.5%, 5.3-73%, 5.4-72.5%, 5.5-72%, 5.6-71.5%, 5.7-71%, 5.8-70.5%, 5.9-70%, 6-69.5%, 6.1-69%, 6.2-68.5%, 6.3-68%, 6.4-67.5%, 6.5-67%, 6.6-66.5%, 6.7-66%, 6.8-65.5%, 6.9-65%, 7-64.5%, 7.1-64%, 7.2-63.5%, 7.3-63%, 7.4-62.5%, 7.5-62%, 7.6-61.5%, 7.7-61%, 7.8-60.5%, 7.9-60%, 8-59.5%, 8.1-59%, 8.2-58.5%, 8.3-58%, 8.4-57.5%, 8.5-57%, 8.6-56.5%, 8.7-56%, 8.8-55.5%, 8.9-55%, 9-54.5%, 9.1-54%, 9.2-53.5%, 9.3-53%, 9.4-52.5%, 9.5-52%, 9.6-51.5%, 9.7-51%, 9.8-50.5%, 9.9-50%, 10-49.5%, 10.1-49%, 10.2-48.5%, 10.3-48% 10.4-47.5%, 10.5-47%, 10.6-46.5%, 10.7-46%, 10.8-45.5%, 10.9-45%, 11-44.5%, 11.1-44%, 11.2-43.5%, 11.3-43%, 11.4-42.5%, 11.5-42%, 11.6-41.5%, 11.7-41%, 11.8-40.5%, 11.9-40%, 12-39.5%, 12.1-39%, 12.2-38.5%, 12.3-38%, 12.4-37.5%, 12.5-37% 12.6-36.5%, 12.7-36%, 12.8-35.5%, 12.9-35%, 13-34.5%, 13.1-34%, 13.2-33.5%, 13.3-33%, 13.4-32.5%, 13.5-32%, 13.6-31.5%, 13.7-31%, 13.8-30.5%, 13.9-30%, 14-29.5%, 14.1-29%, 14.2-28.5%, 14.3-28%, 14.4-27.5%, 14.5-27%, 14.6-26.5%, 14.7-26%, 14.8-25.5%, 14.9-25%, 15-24.5%, 15.1-24%, 15.2-23.5%, 15.3-23%, 15.4-22.5%, 15.5-22%, 15.6-21.5%, 15.7-21%, 15.8-20.5%, 15.9-20%, 16-19.5%, 16.1-19%, 16.2-18.5%, 16.3-18%, 16.4-17.5% or 16.5-17% by weight of free base.

In an embodiment, the amount of the compound, tautomer, cis- or trans-isomer, mesomer, racemate, enantiomer, diastereomer, or mixture thereof, or pharmaceutically acceptable salts thereof, is about 15-30%, 15.1-29.9%, 15.2-29.8%, 15.3-29.7%, 15.4-29.6%, 15.5-29.5%, 15.6-29.4%, 15.7-29.3%, 15.8-29.2%, 15.9-29.1%, 16-29%, 16.1-28.9%, 16.2-28.8% 16.3-28.7%, 16.4-28.6%, 16.5-28.5%, 16.6-28.4%, 16.7-28.3%, 16.8-28.2%, 16.9-28.1%, 17-28%, 17.1-27.9%, 17.2-27.8%, 17.3-27.7%, 17.4-27.6%, 17.5-27.5%, 17.6-27.4%, 17.7-27.3%, 17.8-27.2%, 17.9-27.1%, 18-27%, 18.1-26.9%, 18.2-26.8%, 18.3-26.7%, 18.4-26.6%, 18.5-26.5%, 18.6-26.4%, 18.7-26.3%, 18.8-26.2%, 18.9-26.1%, 19-26%, 19.1-25.9%, 19.2-25.8%, 19.3-25.7%, 19.4-25.6%, 19.5-25.5%, 19.6-25.4%, 19.7-25.3%, 19.8-25.2%, 19.9-25.1%, 20-25%, 20.1-24.9%, 20.2-24.8%, 20.3-24.7%, 20.4-24.6%, 20.5-24.5%, 20.6-24.4%, 20.7-24.3%, 20.8-24.2%, 20.9-24.1%, 21-24%, 21.1-23.9%, 21.2-23.8%, 21.3-23.7%, 21.4-23.6%, 21.5-23.5%, 21.6-23.4%, 21.7-23.3%, 21.8-23.2%, 21.9-23.1%, 22-23%, 22.1-22.9%, 22.2-22.8%, 22.3-22.7% or 22.4-22.6% by weight of free base.

In an embodiment, the unit dosage of the compound, tautomer, cis- or trans-isomer, mesomer, racemate, enantiomer, diastereomer, or mixture thereof, or pharmaceutically acceptable salts thereof, is 1 mg, 2 mg, 3 mg, 4 mg, 5 mg, 6 mg, 7 mg, 8 mg, 9 mg, 10 mg, 11 mg, 12 mg 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 52.5 mg, 55 mg, 60 mg, 65 mg, 70 mg, 75 mg, 80 mg, 85 mg, 90 mg, 95 mg, 100 mg, 105 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 m by weight of free base.

In an embodiment, the compound, tautomer, cis- or trans-isomer, mesomer, racemate, enantiomer, diastereomer, or mixture thereof, or pharmaceutically acceptable salts thereof is can be administered by any suitable route of administration, e.g. oral, parenteral, buccal, sublingual, nasal, rectal, intrathecal or transdermal administration, and the pharmaceutical compositions adapted accordingly. In an embodiment, the compound, tautomer, cis- or trans-isomer, mesomer, racemate, enantiomer, diastereomer, or mixture thereof, or pharmaceutically acceptable salts is formulated as a solid or liquid form, e.g. of tablets, pills, powders, lozenges, sachets, cachets, elixirs, suspensions, emulsions, solutions, syrups, granules.

In some embodiment, the compound is selected from the following structure:

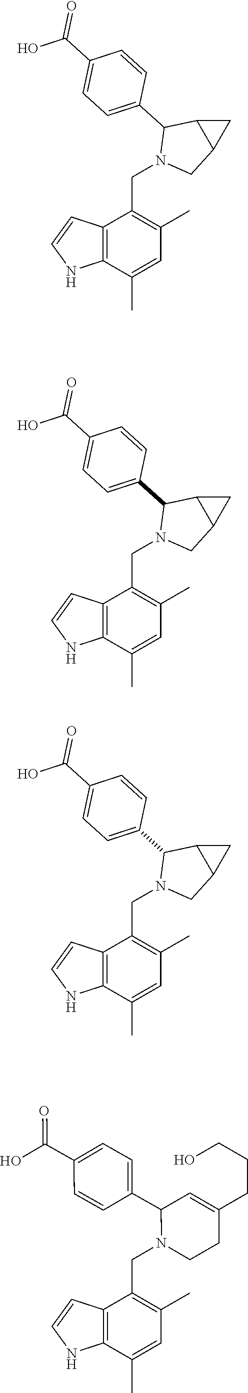 [SMILES: Cc1cc(C)c2[nH]ccc2c1CN1CC2CC2C1c1ccc(C(=O)O)cc1 ; Cc1cc(C)c2[nH]ccc2c1CN1CC2CC2[C@@H]1c1ccc(C(=O)O)cc1 ; Cc1cc(C)c2[nH]ccc2c1CN1CC2CC2[C@H]1c1ccc(C(=O)O)cc1 ; Cc1cc(C)c2[nH]ccc2c1CN1CCC(CCCO)=CC1c1ccc(C(=O)O)cc1]

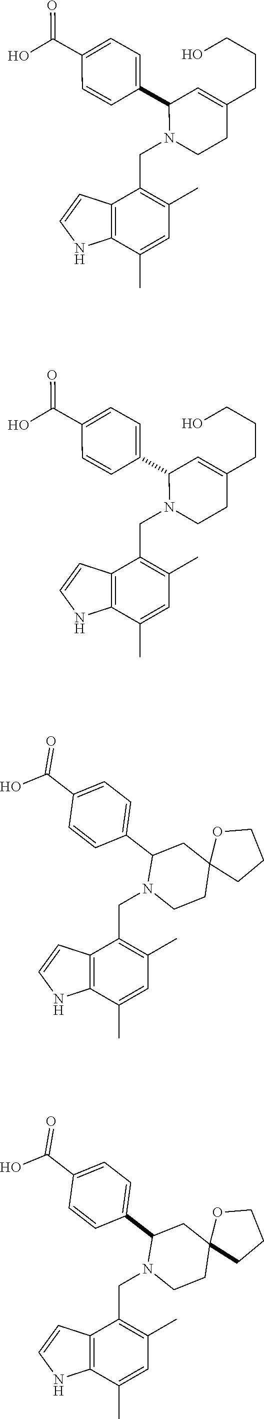

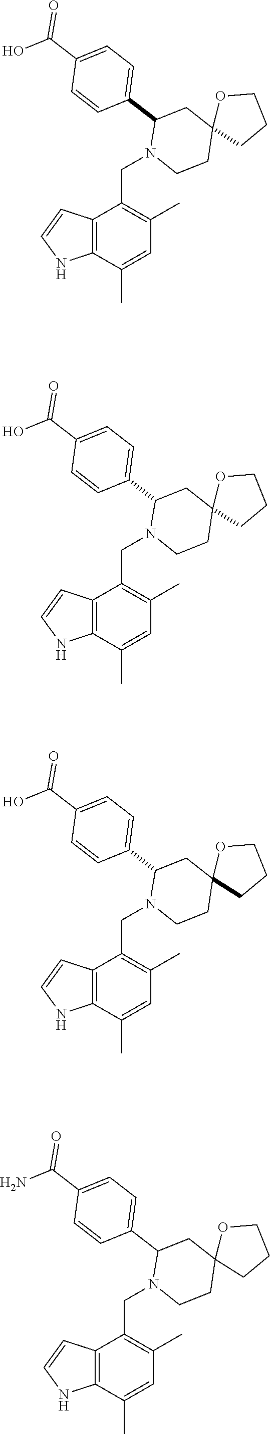

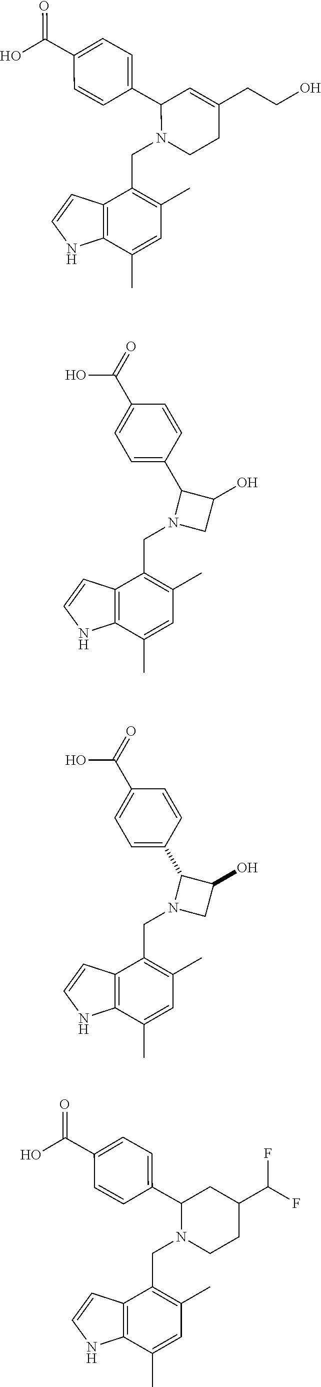

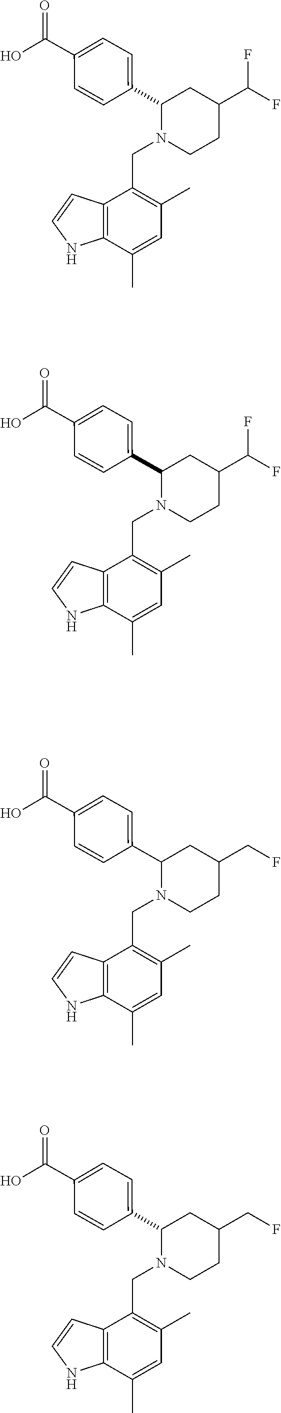

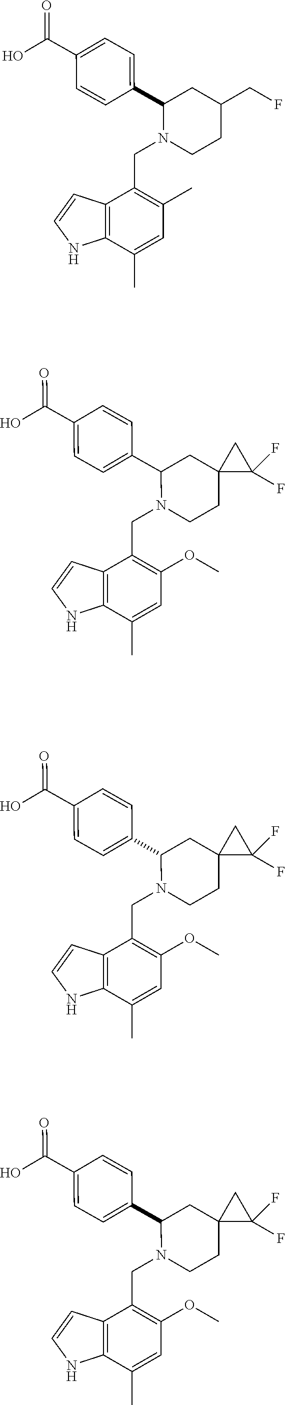

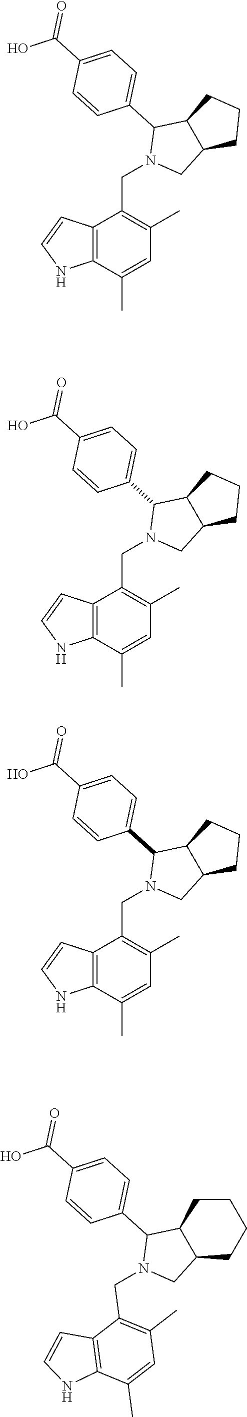 [SMILES: Cc1cc(C)c2[nH]ccc2c1CN1C[C@@H]2CCC[C@@H]2C1c1ccc(C(=O)O)cc1 ; Cc1cc(C)c2[nH]ccc2c1CN1C[C@@H]2CCC[C@@H]2[C@H]1c1ccc(C(=O)O)cc1 ; Cc1cc(C)c2[nH]ccc2c1CN1C[C@@H]2CCC[C@@H]2[C@@H]1c1ccc(C(=O)O)cc1 ; Cc1cc(C)c2[nH]ccc2c1CN1C[C@@H]2CCCC[C@@H]2C1c1ccc(C(=O)O)cc1]

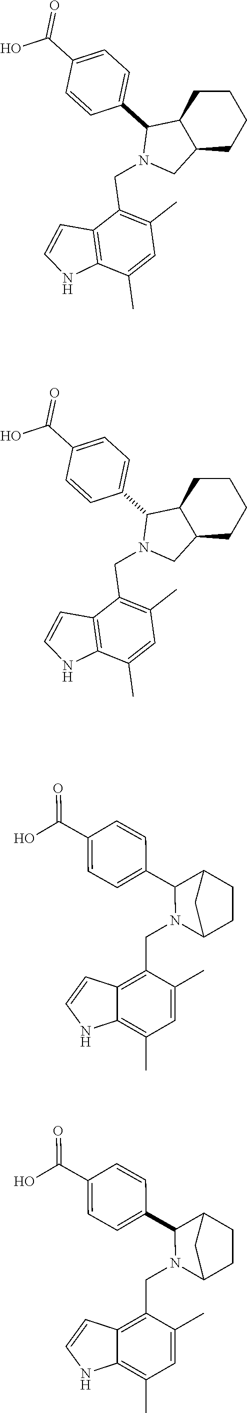

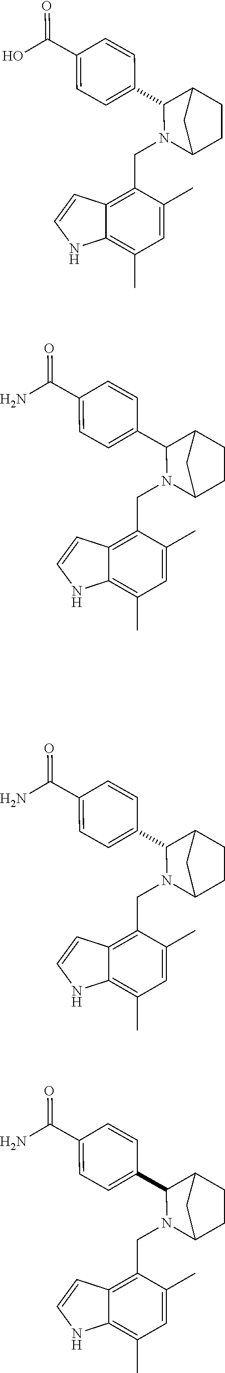

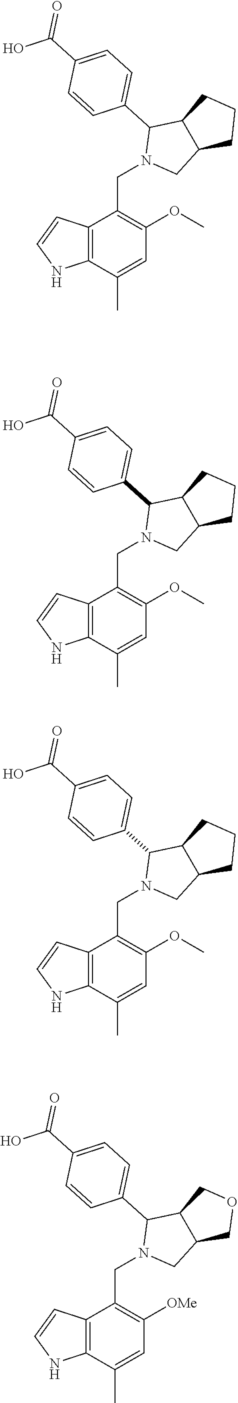 [SMILES: COc1cc(C)c2[nH]ccc2c1CN1C[C@@H]2CCC[C@@H]2C1c1ccc(C(=O)O)cc1 ; COc1cc(C)c2[nH]ccc2c1CN1C[C@@H]2CCC[C@@H]2[C@@H]1c1ccc(C(=O)O)cc1 ; COc1cc(C)c2[nH]ccc2c1CN1C[C@@H]2CCC[C@@H]2[C@H]1c1ccc(C(=O)O)cc1 ; COc1cc(C)c2[nH]ccc2c1CN1C[C@@H]2COC[C@@H]2C1c1ccc(C(=O)O)cc1]

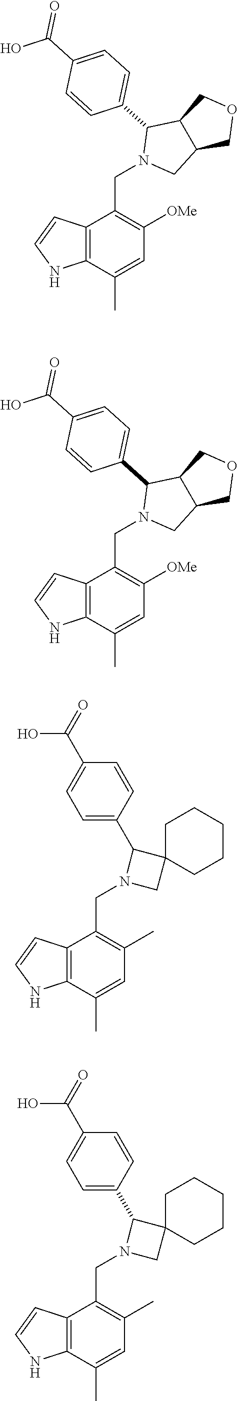

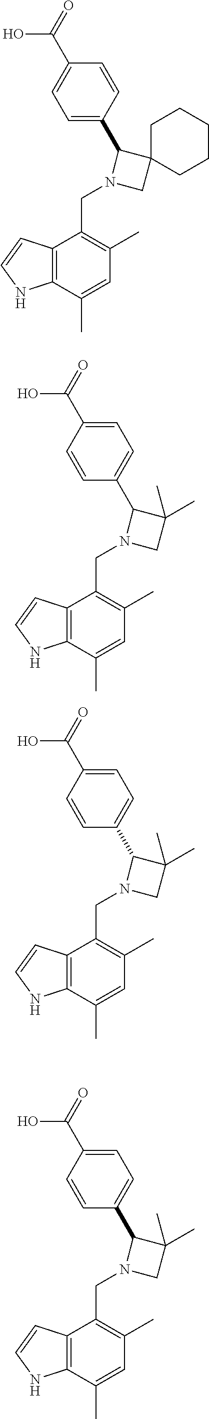

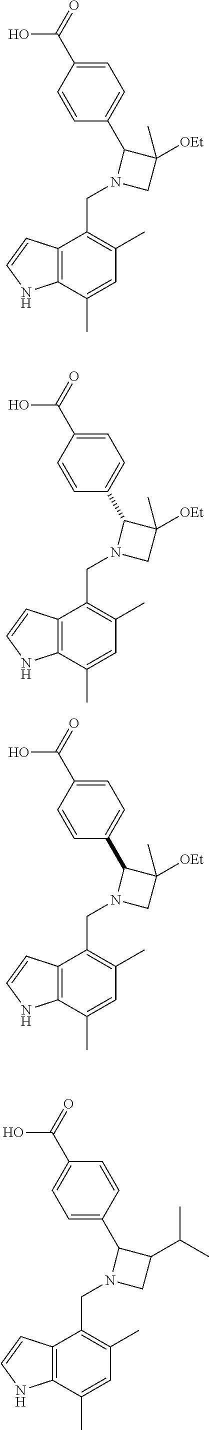

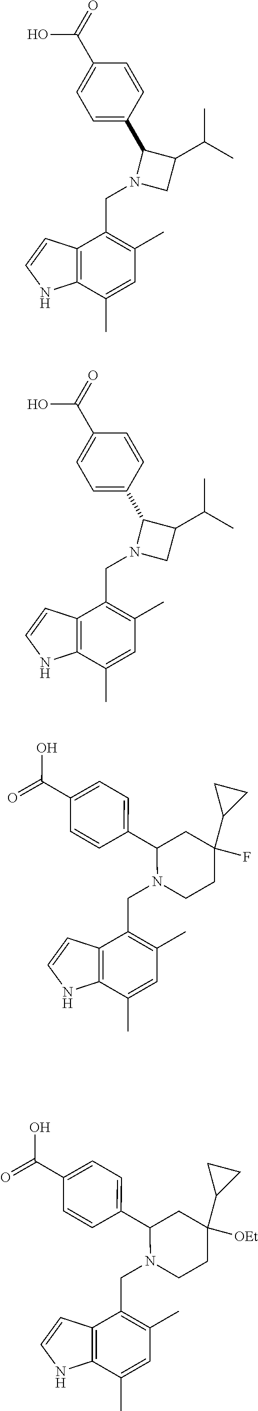

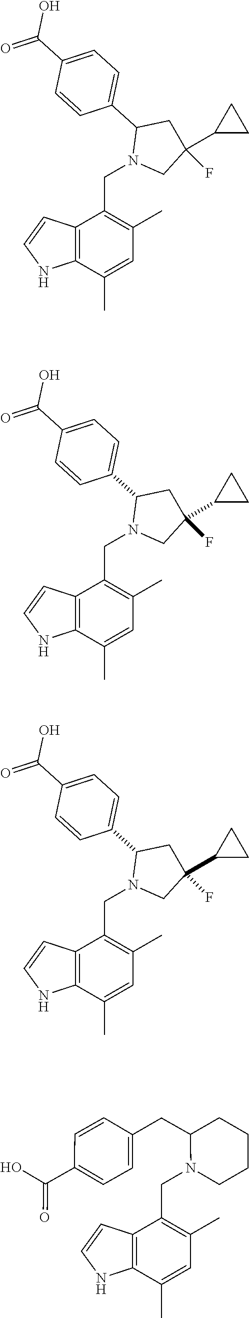 [SMILES: Cc1cc(C)c2[nH]ccc2c1CN1CC(F)(C2CC2)CC1c1ccc(C(=O)O)cc1 ; Cc1cc(C)c2[nH]ccc2c1CN1C[C@@](F)(C2CC2)C[C@H]1c1ccc(C(=O)O)cc1 ; Cc1cc(C)c2[nH]ccc2c1CN1C[C@](F)(C2CC2)C[C@H]1c1ccc(C(=O)O)cc1 ; Cc1cc(C)c2[nH]ccc2c1CN1CCCCC1Cc1ccc(C(=O)O)cc1]

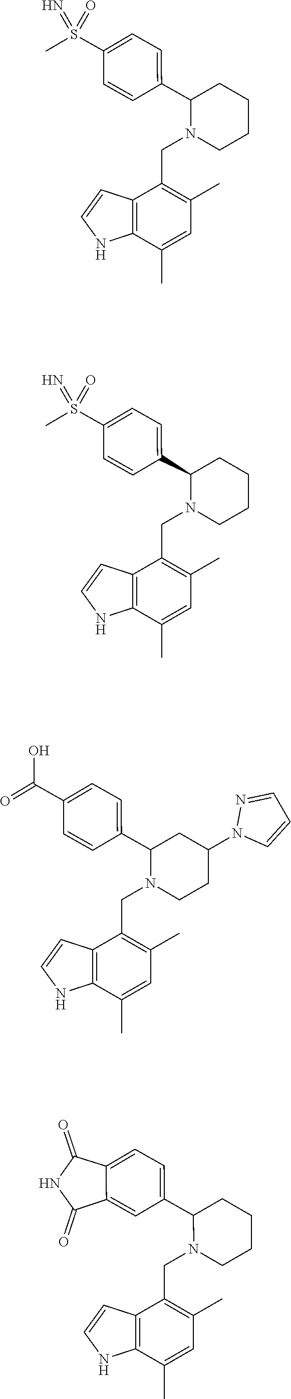 [SMILES: Cc1cc(C)c2[nH]ccc2c1CN1CCCCC1c1ccc(S(C)(=N)=O)cc1 ; Cc1cc(C)c2[nH]ccc2c1CN1CCCC[C@@H]1c1ccc(S(C)(=N)=O)cc1 ; Cc1cc(C)c2[nH]ccc2c1CN1CCC(n2cccn2)CC1c1ccc(C(=O)O)cc1 ; Cc1cc(C)c2[nH]ccc2c1CN1CCCCC1c1ccc2c(c1)C(=O)NC2=O]

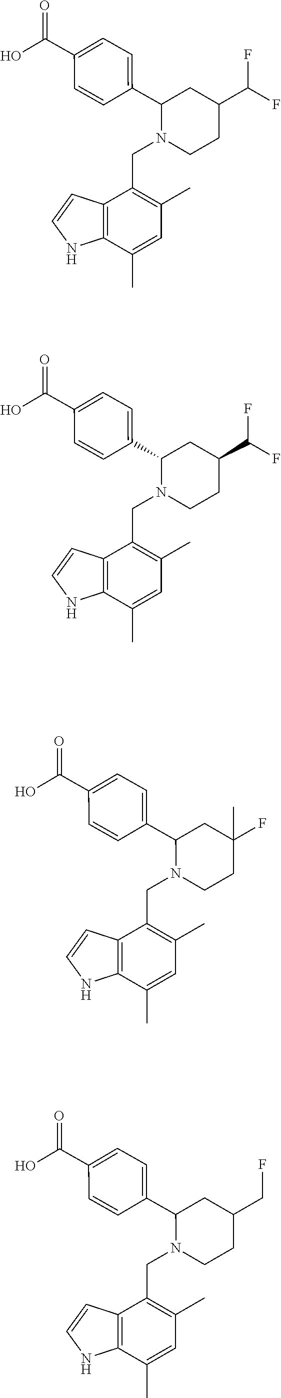 [SMILES: Cc1cc(C)c2[nH]ccc2c1CN1CCC(C(F)F)CC1c1ccc(C(=O)O)cc1 ; Cc1cc(C)c2[nH]ccc2c1CN1CC[C@H](C(F)F)C[C@H]1c1ccc(C(=O)O)cc1 ; Cc1cc(C)c2[nH]ccc2c1CN1CCC(C)(F)CC1c1ccc(C(=O)O)cc1 ; Cc1cc(C)c2[nH]ccc2c1CN1CCC(CF)CC1c1ccc(C(=O)O)cc1]

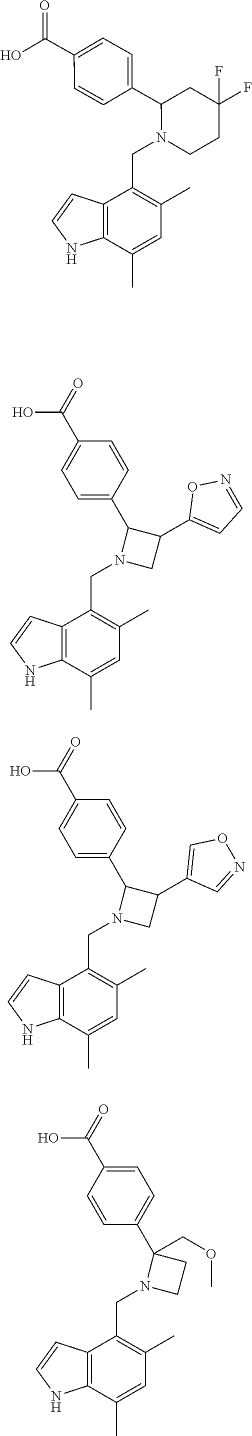

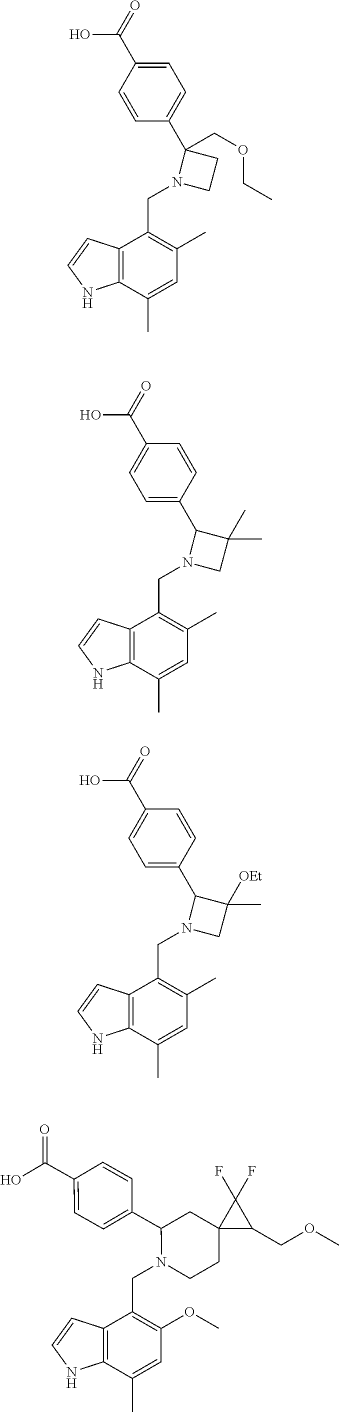

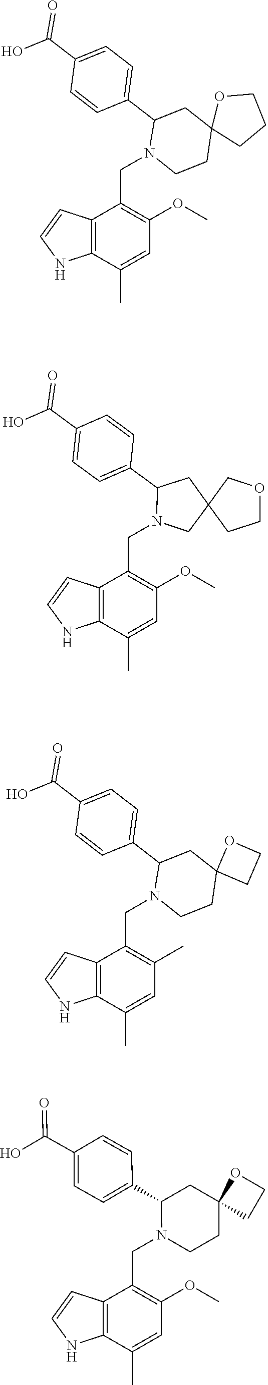

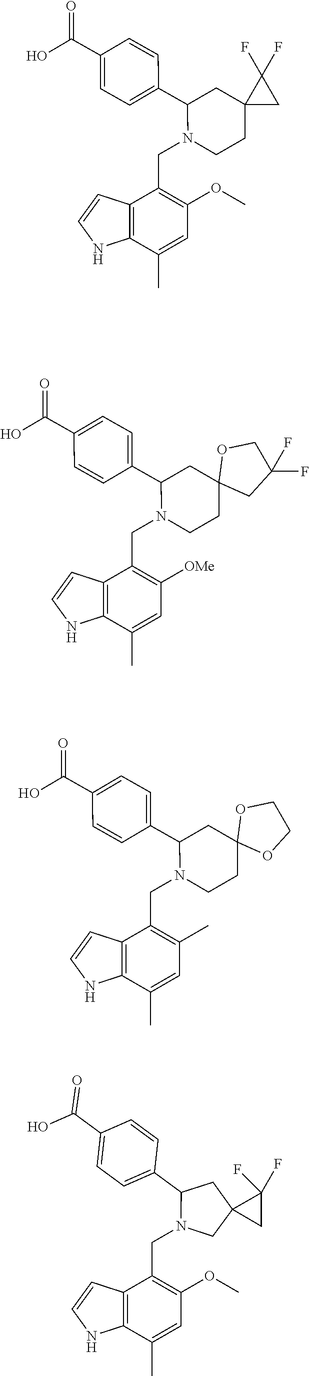 [SMILES: COc1cc(C)c2[nH]ccc2c1CN1CCC2(CC1c1ccc(C(=O)O)cc1)CC2(F)F ; COc1cc(C)c2[nH]ccc2c1CN1CCC2(CC1c1ccc(C(=O)O)cc1)CC(F)(F)CO2 ; Cc1cc(C)c2[nH]ccc2c1CN1CCC2(CC1c1ccc(C(=O)O)cc1)OCCO2 ; COc1cc(C)c2[nH]ccc2c1CN1CC2(CC1c1ccc(C(=O)O)cc1)CC2(F)F]

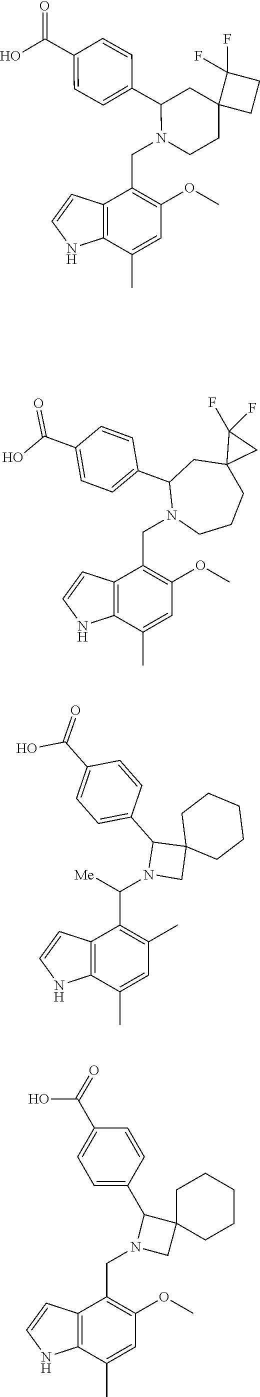 [SMILES: COc1cc(C)c2[nH]ccc2c1CN1CCC2(CCC2(F)F)CC1c1ccc(C(=O)O)cc1 ; COc1cc(C)c2[nH]ccc2c1CN1CCCC2(CC1c1ccc(C(=O)O)cc1)CC2(F)F ; Cc1cc(C)c2[nH]ccc2c1C(C)N1CC2(CCCCC2)C1c1ccc(C(=O)O)cc1 ; COc1cc(C)c2[nH]ccc2c1CN1CC2(CCCCC2)C1c1ccc(C(=O)O)cc1]

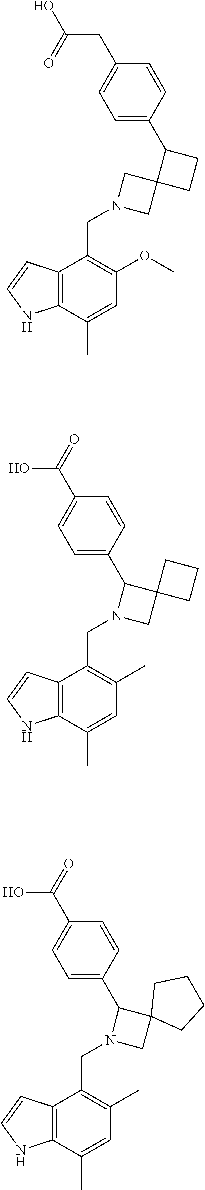 [SMILES: COc1cc(C)c2[nH]ccc2c1CN1CC2(CCC2c2ccc(CC(=O)O)cc2)C1 ; Cc1cc(C)c2[nH]ccc2c1CN1CC2(CCC2)C1c1ccc(C(=O)O)cc1 ; Cc1cc(C)c2[nH]ccc2c1CN1CC2(CCCC2)C1c1ccc(C(=O)O)cc1]

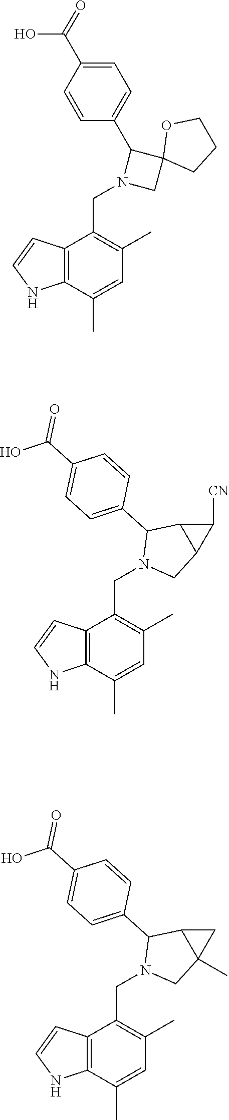 [SMILES: Cc1cc(C)c2[nH]ccc2c1CN1CC2(CCCO2)C1c1ccc(C(=O)O)cc1 ; Cc1cc(C)c2[nH]ccc2c1CN1CC2C(C#N)C2C1c1ccc(C(=O)O)cc1 ; Cc1cc(C)c2[nH]ccc2c1CN1CC2(C)CC2C1c1ccc(C(=O)O)cc1]

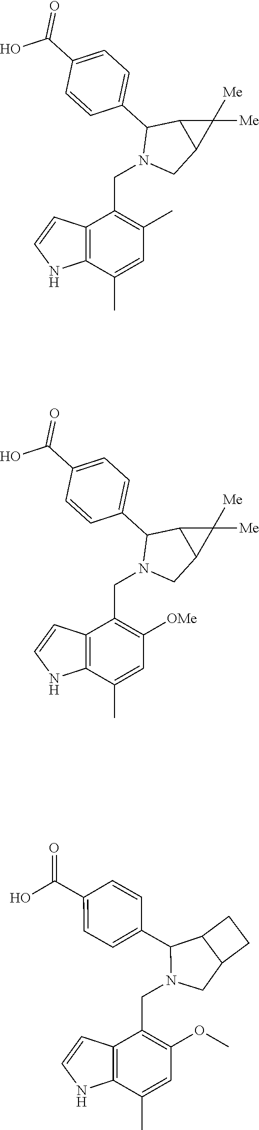 [SMILES: Cc1cc(C)c2[nH]ccc2c1CN1CC2C(C1c1ccc(C(=O)O)cc1)C2(C)C ; COc1cc(C)c2[nH]ccc2c1CN1CC2C(C1c1ccc(C(=O)O)cc1)C2(C)C ; COc1cc(C)c2[nH]ccc2c1CN1CC2CCC2C1c1ccc(C(=O)O)cc1]

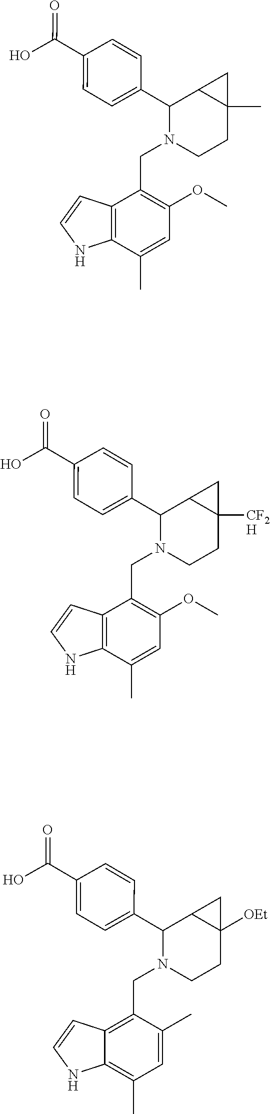 [SMILES: COc1cc(C)c2[nH]ccc2c1CN1CCC2(C)CC2C1c1ccc(C(=O)O)cc1 ; COc1cc(C)c2[nH]ccc2c1CN1CCC2(C(F)F)CC2C1c1ccc(C(=O)O)cc1 ; CCOC12CCN(Cc3c(C)cc(C)c4[nH]ccc34)C(c3ccc(C(=O)O)cc3)C1C2]

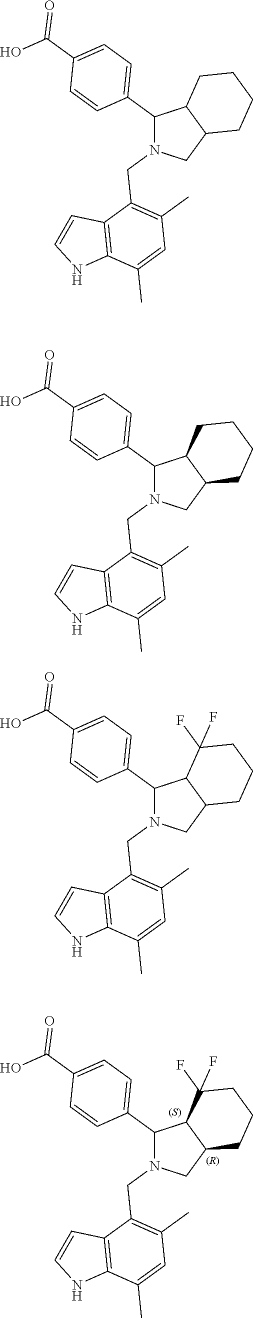

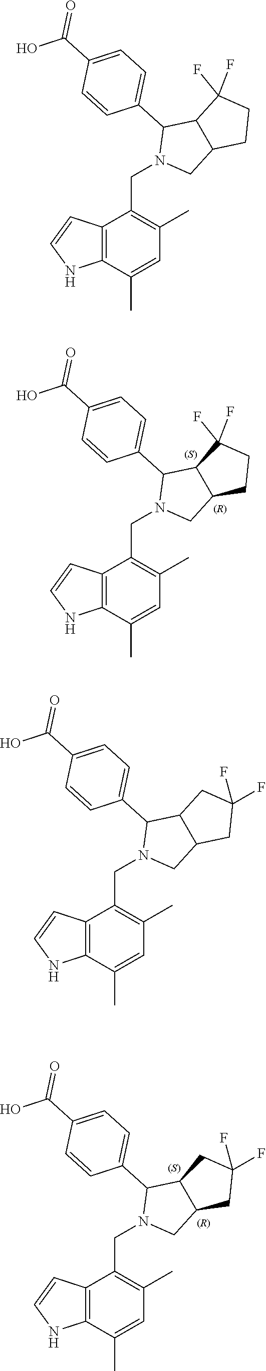

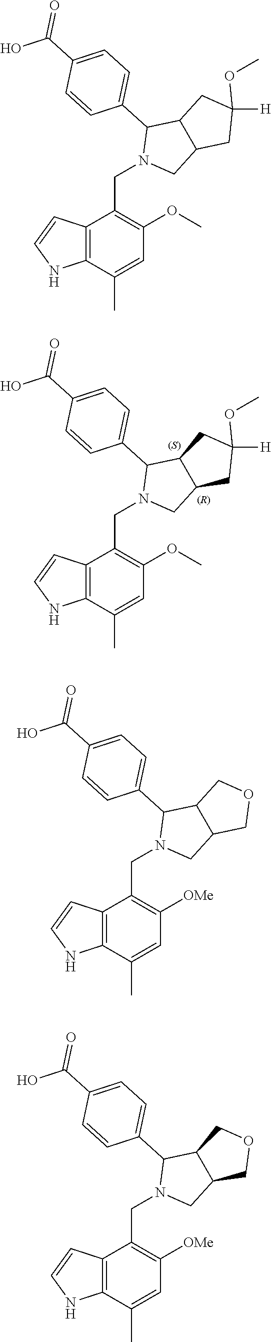

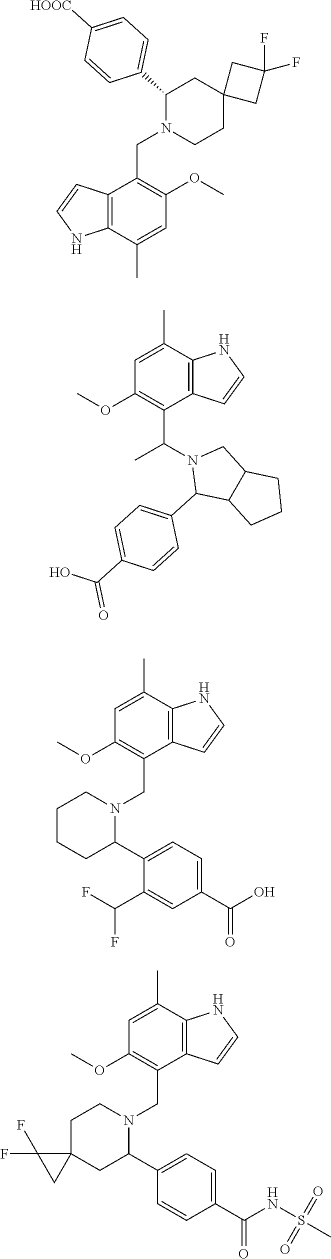

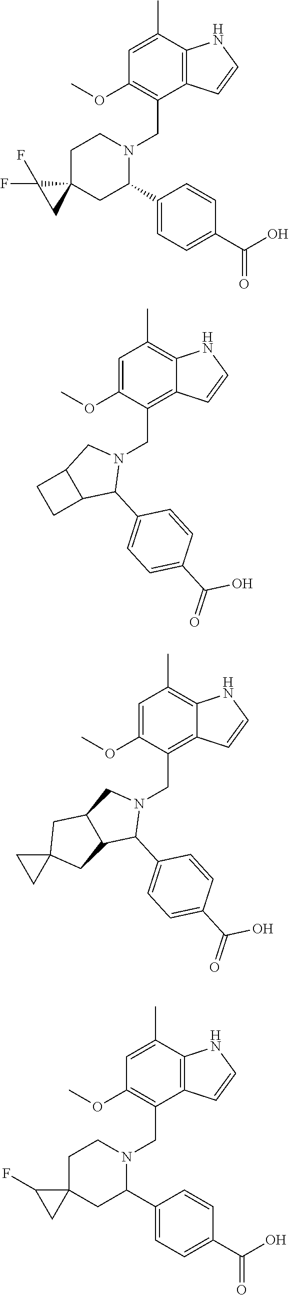

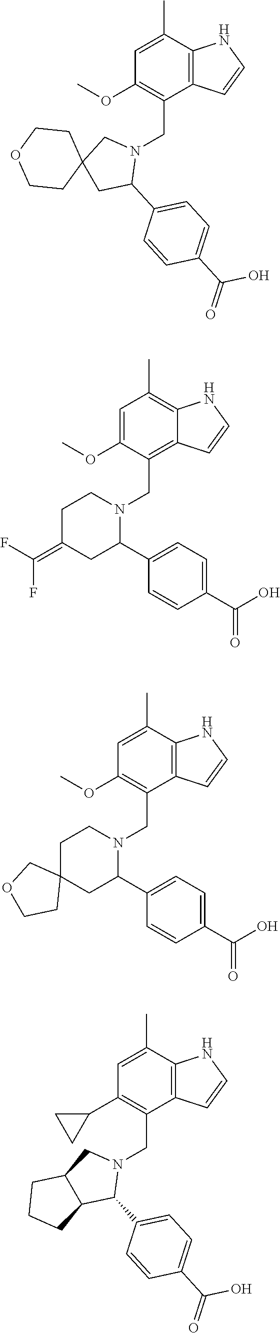 [SMILES: COc1cc(C)c2[nH]ccc2c1CN1CC2(CCOCC2)CC1c1ccc(C(=O)O)cc1 ; COc1cc(C)c2[nH]ccc2c1CN1CCC(=C(F)F)CC1c1ccc(C(=O)O)cc1 ; COc1cc(C)c2[nH]ccc2c1CN1CCC2(CCOC2)CC1c1ccc(C(=O)O)cc1 ; Cc1cc(C2CC2)c(CN2C[C@@H]3CCC[C@@H]3[C@H]2c2ccc(C(=O)O)cc2)c2cc[nH]c12]

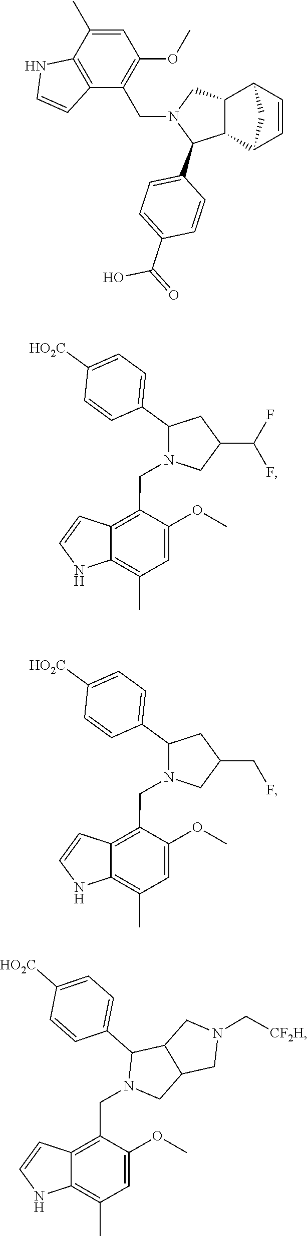 [SMILES: COc1cc(C)c2[nH]ccc2c1CN1C[C@H]2[C@H]([C@@H]3C=C[C@H]2C3)[C@H]1c1ccc(C(=O)O)cc1 ; COc1cc(C)c2[nH]ccc2c1CN1CC(C(F)F)CC1c1ccc(C(=O)O)cc1 ; COc1cc(C)c2[nH]ccc2c1CN1CC(CF)CC1c1ccc(C(=O)O)cc1 ; COc1cc(C)c2[nH]ccc2c1CN1CC2CN(CC(F)F)CC2C1c1ccc(C(=O)O)cc1]

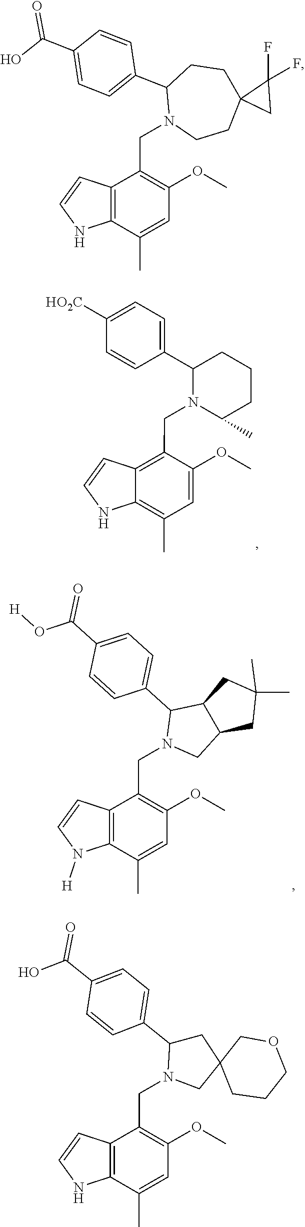

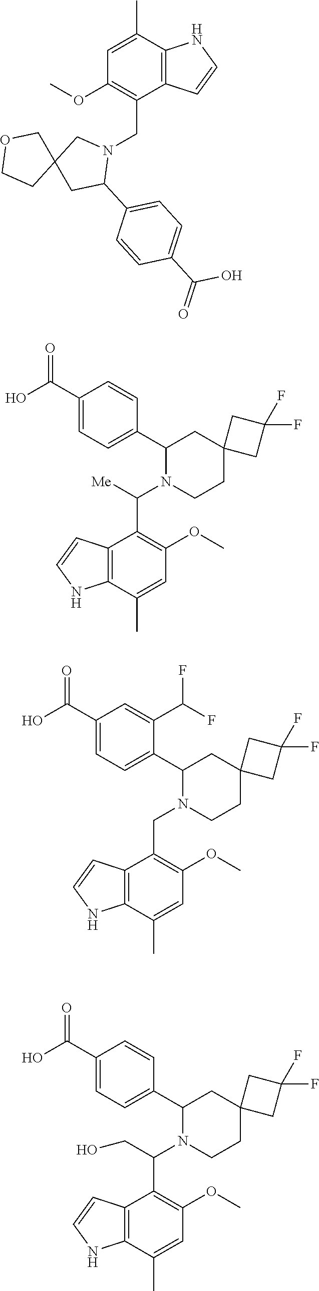 [SMILES: COc1cc(C)c2[nH]ccc2c1CN1CC2(CCOC2)CC1c1ccc(C(=O)O)cc1 ; COc1cc(C)c2[nH]ccc2c1C(C)N1CCC2(CC1c1ccc(C(=O)O)cc1)CC(F)(F)C2 ; COc1cc(C)c2[nH]ccc2c1CN1CCC2(CC1c1ccc(C(=O)O)cc1C(F)F)CC(F)(F)C2 ; COc1cc(C)c2[nH]ccc2c1C(CO)N1CCC2(CC1c1ccc(C(=O)O)cc1)CC(F)(F)C2]

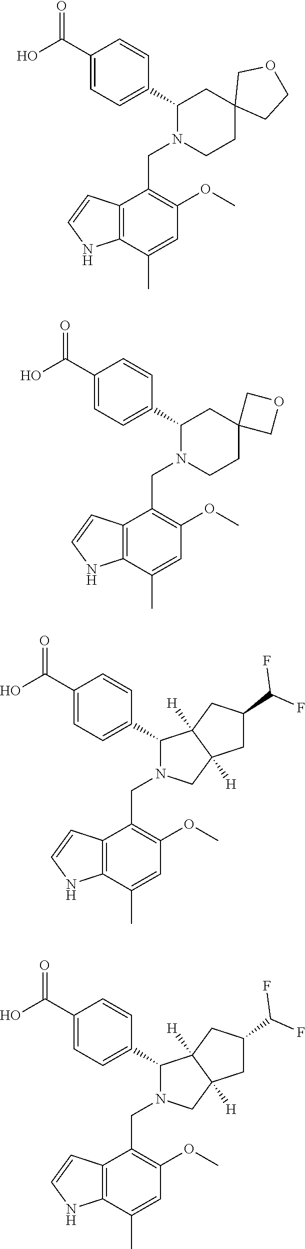 [SMILES: COc1cc(C)c2[nH]ccc2c1CN1CCC2(CCOC2)C[C@H]1c1ccc(C(=O)O)cc1 ; COc1cc(C)c2[nH]ccc2c1CN1CCC2(COC2)C[C@H]1c1ccc(C(=O)O)cc1 ; COc1cc(C)c2[nH]ccc2c1CN1C[C@@H]2C[C@H](C(F)F)C[C@@H]2[C@H]1c1ccc(C(=O)O)cc1 ; COc1cc(C)c2[nH]ccc2c1CN1C[C@@H]2C[C@@H](C(F)F)C[C@@H]2[C@H]1c1ccc(C(=O)O)cc1]

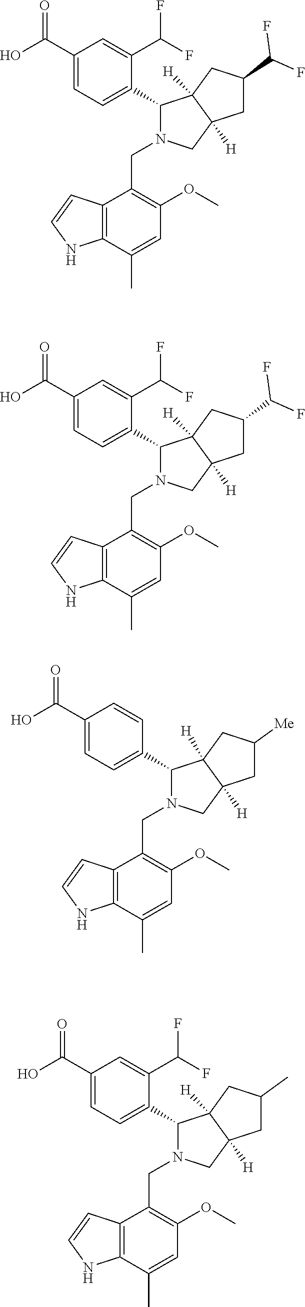 [SMILES: COc1cc(C)c2[nH]ccc2c1CN1C[C@@H]2C[C@H](C(F)F)C[C@@H]2[C@H]1c1ccc(C(=O)O)cc1C(F)F ; COc1cc(C)c2[nH]ccc2c1CN1C[C@@H]2C[C@@H](C(F)F)C[C@@H]2[C@H]1c1ccc(C(=O)O)cc1C(F)F ; COc1cc(C)c2[nH]ccc2c1CN1C[C@@H]2CC(C)C[C@@H]2[C@H]1c1ccc(C(=O)O)cc1 ; COc1cc(C)c2[nH]ccc2c1CN1C[C@@H]2CC(C)C[C@@H]2[C@H]1c1ccc(C(=O)O)cc1C(F)F]

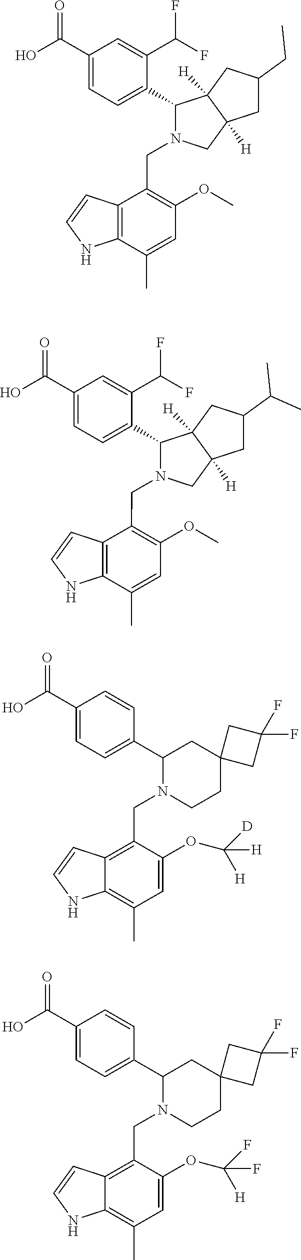 [SMILES: CCC1C[C@H]2CN(Cc3c(OC)cc(C)c4[nH]ccc34)[C@H](c3ccc(C(=O)O)cc3C(F)F)[C@H]2C1 ; COc1cc(C)c2[nH]ccc2c1CN1C[C@@H]2CC(C(C)C)C[C@@H]2[C@H]1c1ccc(C(=O)O)cc1C(F)F ; [2H]COc1cc(C)c2[nH]ccc2c1CN1CCC2(CC1c1ccc(C(=O)O)cc1)CC(F)(F)C2 ; Cc1cc(OC(F)F)c(CN2CCC3(CC2c2ccc(C(=O)O)cc2)CC(F)(F)C3)c2cc[nH]c12]

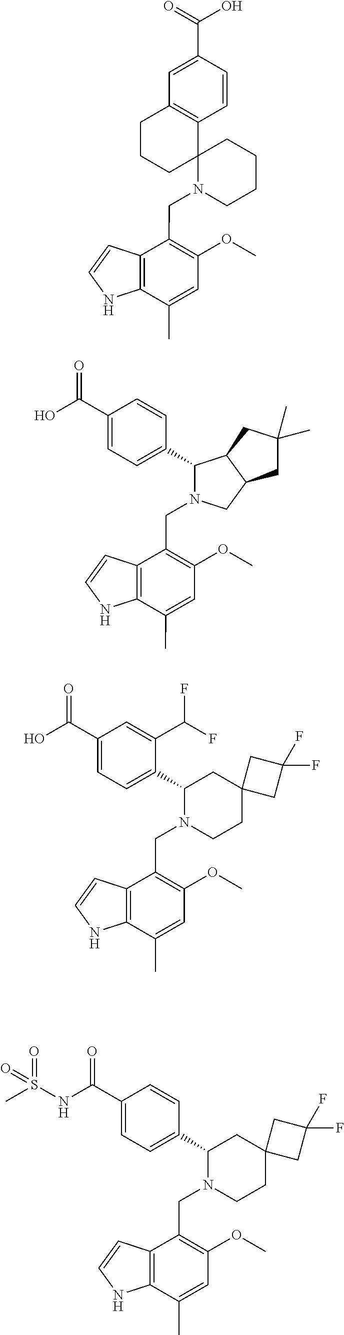

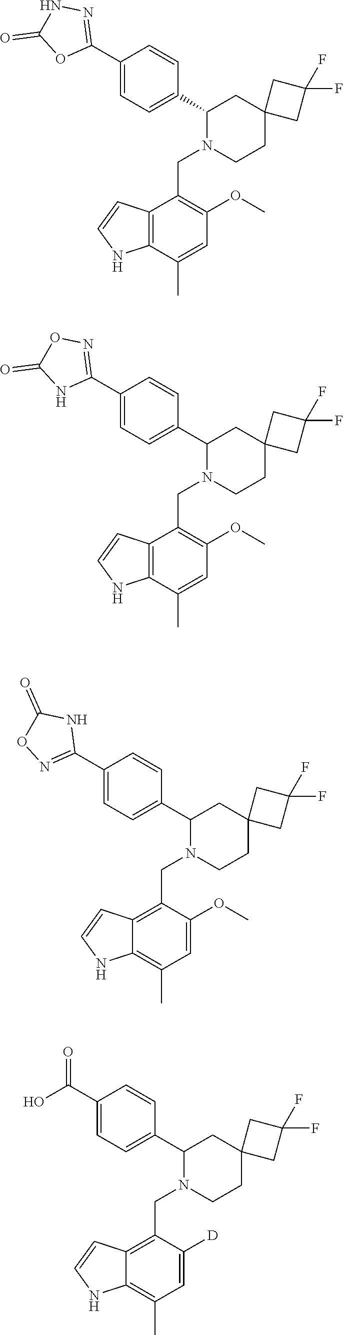 [SMILES: COc1cc(C)c2[nH]ccc2c1CN1CCC2(C[C@H]1c1ccc(-c3n[nH]c(=O)o3)cc1)CC(F)(F)C2 ; COc1cc(C)c2[nH]ccc2c1CN1CCC2(CC1c1ccc(-c3noc(=O)[nH]3)cc1)CC(F)(F)C2 ; COc1cc(C)c2[nH]ccc2c1CN1CCC2(CC1c1ccc(-c3noc(=O)[nH]3)cc1)CC(F)(F)C2 ; [2H]c1cc(C)c2[nH]ccc2c1CN1CCC2(CC1c1ccc(C(=O)O)cc1)CC(F)(F)C2]

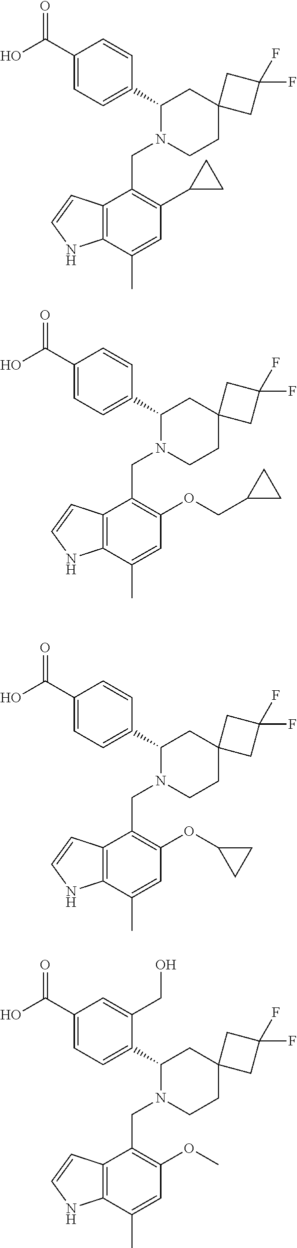

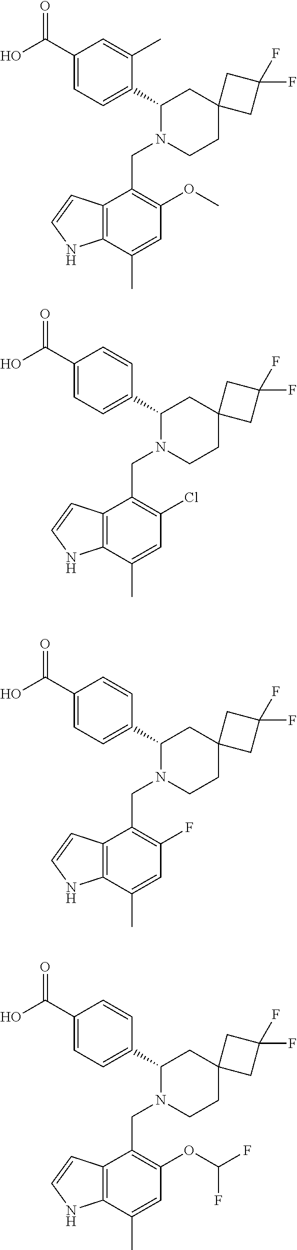 [SMILES: COc1cc(C)c2[nH]ccc2c1CN1CCC2(C[C@H]1c1ccc(C(=O)O)cc1C)CC(F)(F)C2 ; Cc1cc(Cl)c(CN2CCC3(C[C@H]2c2ccc(C(=O)O)cc2)CC(F)(F)C3)c2cc[nH]c12 ; Cc1cc(F)c(CN2CCC3(C[C@H]2c2ccc(C(=O)O)cc2)CC(F)(F)C3)c2cc[nH]c12 ; Cc1cc(OC(F)F)c(CN2CCC3(C[C@H]2c2ccc(C(=O)O)cc2)CC(F)(F)C3)c2cc[nH]c12]

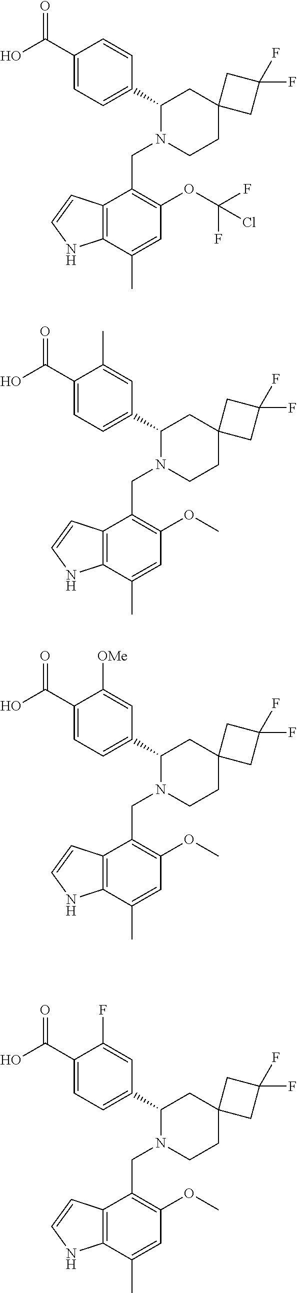 [SMILES: Cc1cc(OC(F)(F)Cl)c(CN2CCC3(C[C@H]2c2ccc(C(=O)O)cc2)CC(F)(F)C3)c2cc[nH]c12 ; COc1cc(C)c2[nH]ccc2c1CN1CCC2(C[C@H]1c1ccc(C(=O)O)c(C)c1)CC(F)(F)C2 ; COc1cc([C@@H]2CC3(CCN2Cc2c(OC)cc(C)c4[nH]ccc24)CC(F)(F)C3)ccc1C(=O)O ; COc1cc(C)c2[nH]ccc2c1CN1CCC2(C[C@H]1c1ccc(C(=O)O)c(F)c1)CC(F)(F)C2]

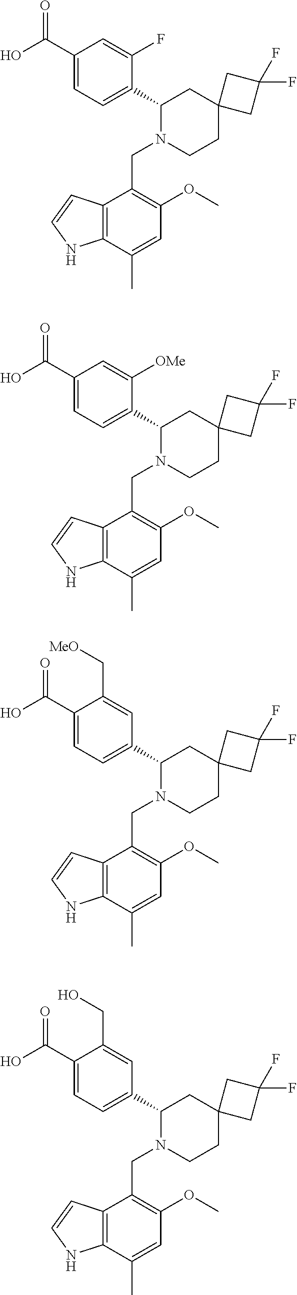 [SMILES: COc1cc(C)c2[nH]ccc2c1CN1CCC2(C[C@H]1c1ccc(C(=O)O)cc1F)CC(F)(F)C2 ; COc1cc(C(=O)O)ccc1[C@@H]1CC2(CCN1Cc1c(OC)cc(C)c3[nH]ccc13)CC(F)(F)C2 ; COCc1cc([C@@H]2CC3(CCN2Cc2c(OC)cc(C)c4[nH]ccc24)CC(F)(F)C3)ccc1C(=O)O ; COc1cc(C)c2[nH]ccc2c1CN1CCC2(C[C@H]1c1ccc(C(=O)O)c(CO)c1)CC(F)(F)C2]

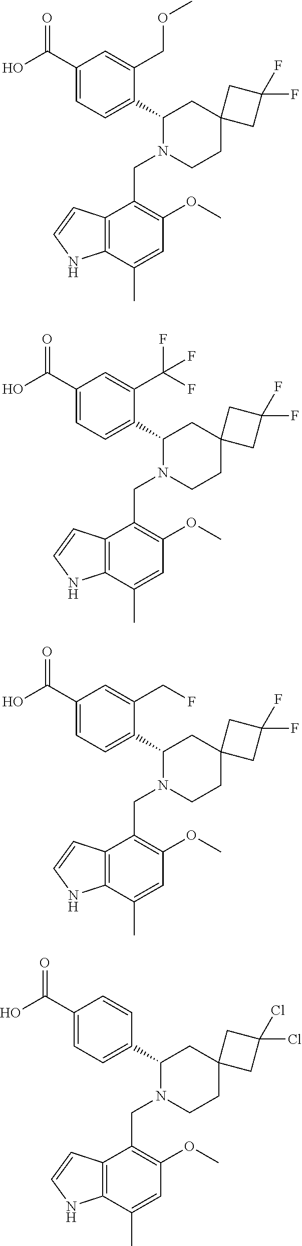 [SMILES: COCc1cc(C(=O)O)ccc1[C@@H]1CC2(CCN1Cc1c(OC)cc(C)c3[nH]ccc13)CC(F)(F)C2 ; COc1cc(C)c2[nH]ccc2c1CN1CCC2(C[C@H]1c1ccc(C(=O)O)cc1C(F)(F)F)CC(F)(F)C2 ; COc1cc(C)c2[nH]ccc2c1CN1CCC2(C[C@H]1c1ccc(C(=O)O)cc1CF)CC(F)(F)C2 ; COc1cc(C)c2[nH]ccc2c1CN1CCC2(C[C@H]1c1ccc(C(=O)O)cc1)CC(Cl)(Cl)C2]

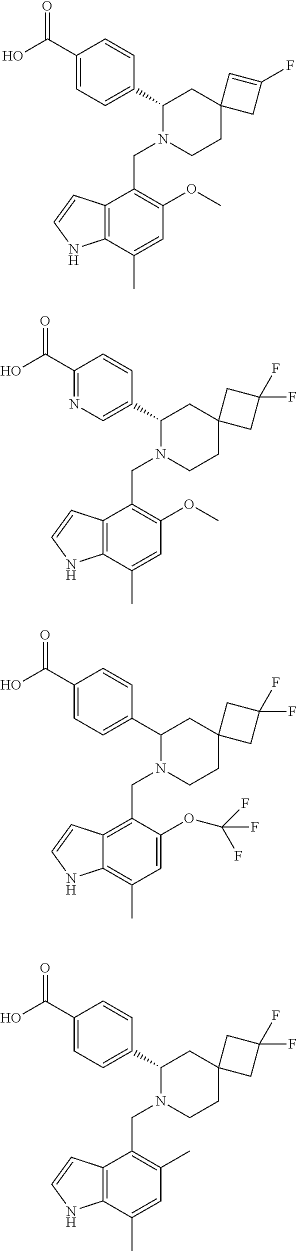 [SMILES: COc1cc(C)c2[nH]ccc2c1CN1CCC2(C=C(F)C2)C[C@H]1c1ccc(C(=O)O)cc1 ; COc1cc(C)c2[nH]ccc2c1CN1CCC2(C[C@H]1c1ccc(C(=O)O)nc1)CC(F)(F)C2 ; Cc1cc(OC(F)(F)F)c(CN2CCC3(CC2c2ccc(C(=O)O)cc2)CC(F)(F)C3)c2cc[nH]c12 ; Cc1cc(C)c2[nH]ccc2c1CN1CCC2(C[C@H]1c1ccc(C(=O)O)cc1)CC(F)(F)C2]

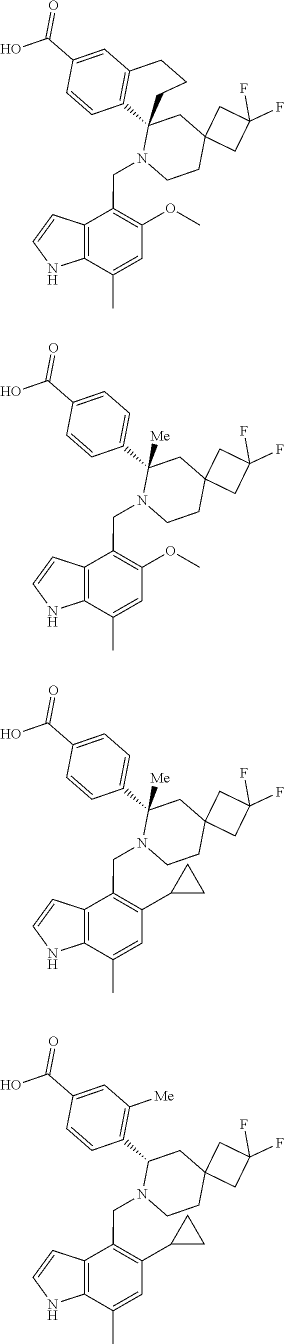

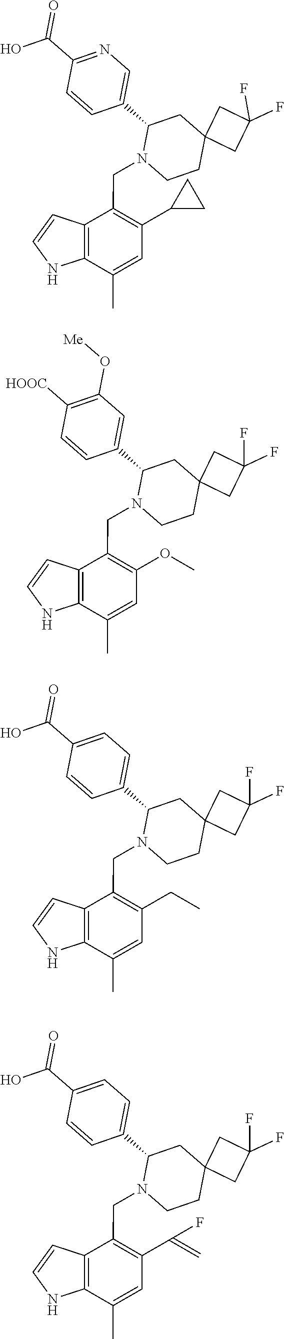

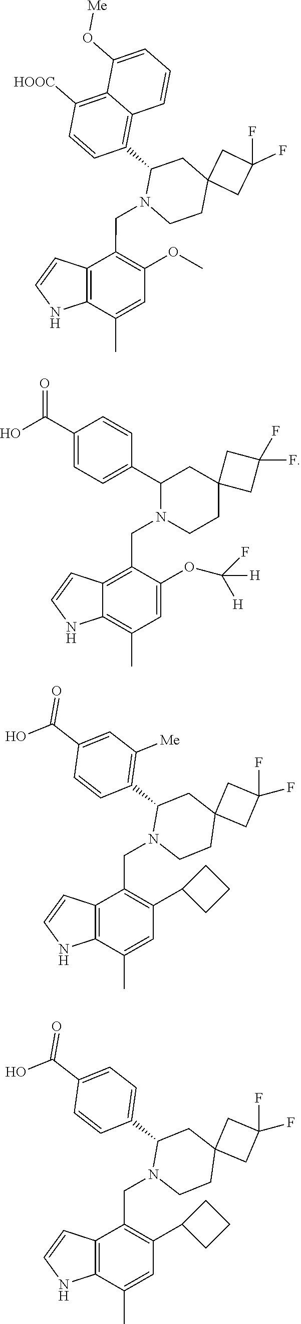 [SMILES: COc1cc(C)c2[nH]ccc2c1CN1CCC2(C[C@H]1c1ccc(C(=O)O)c3c(OC)cccc13)CC(F)(F)C2 ; Cc1cc(OCF)c(CN2CCC3(CC2c2ccc(C(=O)O)cc2)CC(F)(F)C3)c2cc[nH]c12 ; Cc1cc(C(=O)O)ccc1[C@@H]1CC2(CCN1Cc1c(C3CCC3)cc(C)c3[nH]ccc13)CC(F)(F)C2 ; Cc1cc(C2CCC2)c(CN2CCC3(C[C@H]2c2ccc(C(=O)O)cc2)CC(F)(F)C3)c2cc[nH]c12]

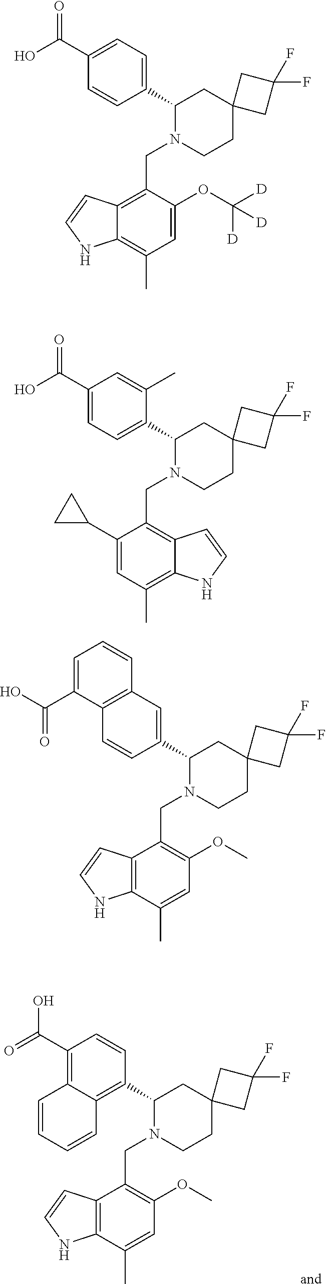

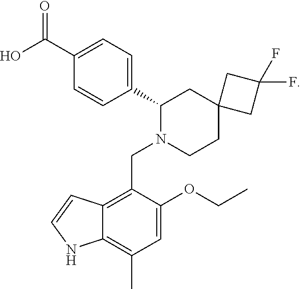 [SMILES: CCOc1cc(C)c2[nH]ccc2c1CN1CCC2(C[C@H]1c1ccc(C(=O)O)cc1)CC(F)(F)C2]

In another aspect, the present invention relates to a method of treating a disorder or a disease in a subject mediated by complement activation, in particular mediated by activation of the complement alternative pathway, comprising administering to a subject in need thereof an effective amount of a compound any formula (I)-(VI-b), or a pharmaceutical composition comprising the same.

In a preferred embodiment, the disease or disorder is selected from the group consisting of age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus (SLE), SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease (COPD), emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, membrane nephropathy, paroxysmal sleep hemoglobin urine, IgA nephropathy, glomerulonephritis and obesity.

### DESCRIPTION OF THE DRAWINGS

FIG. 1 Ex vivo assessment of Plasma PD inhibition on mouse

### DETAILED DESCRIPTION OF THE INVENTION

Various publications, articles and patents are cited or described through the specification; each of these references is herein incorporated by references in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Given below are definitions of terms used in this application. Any term not defined herein takes the normal meaning as the skilled person would understand the term.

“Alkyl” refers to a saturated aliphatic hydrocarbon group including C₁-C₂₀ straight chain and branched chain groups. Preferably an alkyl group is an alkyl having 1 to 12, sometimes preferably 1 to 6, sometimes more preferably 1 to 4, carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl,etc. The alkyl group can be substituted or unsubstituted. When substituted, the substituent group(s) can be substituted at any available connection point, preferably the substituent group(s) is one or more substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2—, or 3-butenyl, etc., preferably C₂₋₂₀ alkenyl, more preferably C₂₋₁₂ alkenyl, and most preferably C₂₋₆ alkenyl.

The alkenyl group can be substituted or unsubstituted. When substituted, the substituent group(s) is preferably one or more, sometimes preferably one to five, sometimes more preferably one to three, group(s) independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2—, or 3-butynyl etc., preferably C₂₋₂₀ alkynyl, more preferably C₂₋₁₂ alkynyl, and most preferably C₂₋₆ alkynyl.

The alkynyl group can be substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 carbon atoms, more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂-etc. The alkenylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkynylene” refers to an alkynyl defined as above that has at least two carbon atoms and at least one carbon-carbon triple bond, preferably C₂₋₂₀ alkynylene, more preferably C₂₋₁₂ alkynylene, and most preferably C₂₋₆ alkynylene. Non-limiting examples of alkenylene groups include, but are not limited to, —CH—CH—, —CH—CHCH₂—, —CH—CHCH₂CH₂—, —CH₂CH—CHCH₂-etc. The alkynylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds, but none of the rings has a completely conjugated pi-electron system. Preferably a spiro cycloalkyl is 6 to 14 membered, more preferably 7 to 10 membered, and most preferably 0.7 to 8 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following substituents;

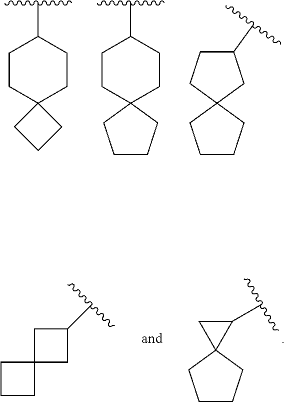

“Fused Cycloalkyl” refers to a 5 to 20 membered polycyclic hydrocarbon group, wherein each ring in the system shares an adjacent pair of carbon atoms with another ring, wherein one or more rings can contain one or more double bonds, but none of the rings has a completely conjugated pi-electron system. Preferably, a fused cycloalkyl group is 6 to 14 membered, more preferably 7 to 10 membered, and most preferably 0.7 to 8 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following substituents;

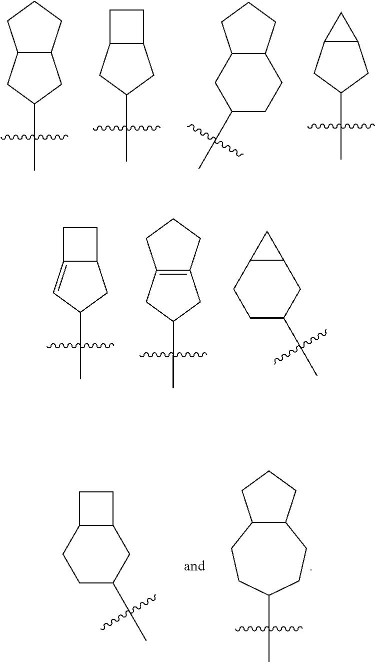

“Bridged Cycloalkyl” refers to a 5 to 20 membered polycyclic hydrocarbon group, wherein every two rings in the system share two disconnected carbon atoms. The rings can have one or more double bonds, but have no completely conjugated pi-electron system. Preferably, a bridged cycloalkyl is 6 to 14 membered, more preferably 7 to 10 membered, and most preferably 7 to 8 membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following substituents;

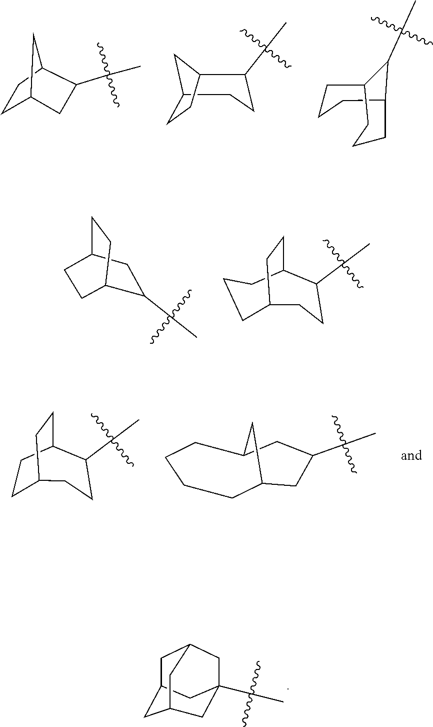

The cycloalkyl can be fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably one to five, sometimes more preferably one to three, substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group. Representative examples include, but are not limited to, the following substituents:

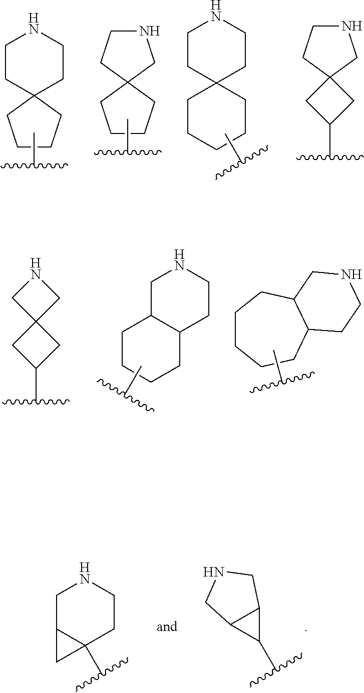

“Heterocyclyl” refers to a 3 to 20 membered saturated and/or partially unsaturated monocyclic or polycyclic hydrocarbon group having one or more, sometimes preferably one to five, sometimes more preferably one to three, heteroatoms selected from the group consisting of N, O, and S(O)ₘ (wherein m is 0, 1, or 2) as ring atoms, but excluding —O—O—, —O—S—or —S—S— in the ring, the remaining ring atoms being C. Preferably, heterocyclyl is a 3 to 12 membered having 1 to 4 heteroatoms; more preferably a 3 to 10 membered having 1 to 3 heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more, sometimes preferably one to five, sometimes more preferably one to three, heteroatoms selected from the group consisting of N, O, and S(O)ₘ (wherein m is 0, 1 or 2) as ring atoms, the remaining ring atoms being C, wherein one or more rings can contain one or more double bonds, but none of the rings has a completely conjugated pi-electron system. Preferably a spiro heterocyclyl is 6 to 14 membered, more preferably 7 to 10 membered, and most preferably 7 to 8 membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following substituents:

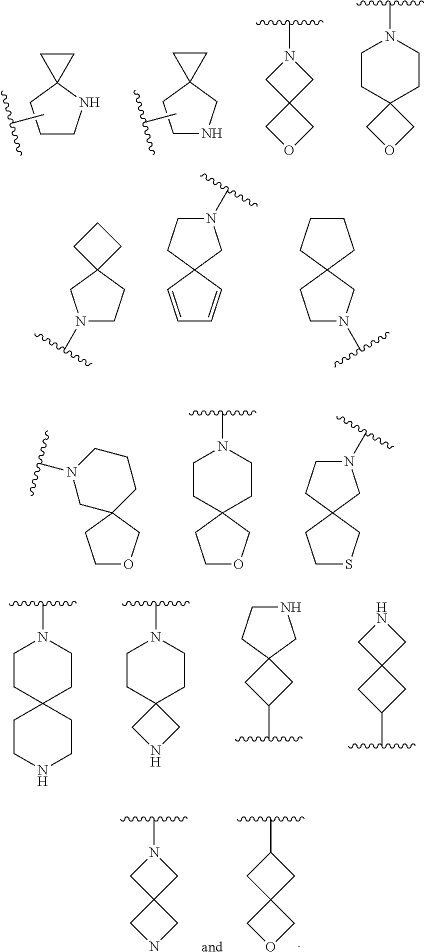

“Fused Heterocyclyl” refers to a 5 to 20 membered polycyclic heterocyclyl group, wherein each ring in the system shares an adjacent pair of carbon atoms with the other ring, wherein one or more rings can contain one or more double bonds, but none of the rings has a completely conjugated pi-electron system, and wherein said rings have one or more, sometimes preferably one to five, sometimes more preferably one to three, heteroatoms selected from the group consisting of N, O, and S(O)ₚ (wherein p is 0, 1, or 2) as ring atoms, the remaining ring atoms being C. Preferably a fused heterocyclyl is 6 to 14 membered, more preferably 7 to 10 membered, and most preferably 7 to 8 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following substituents:

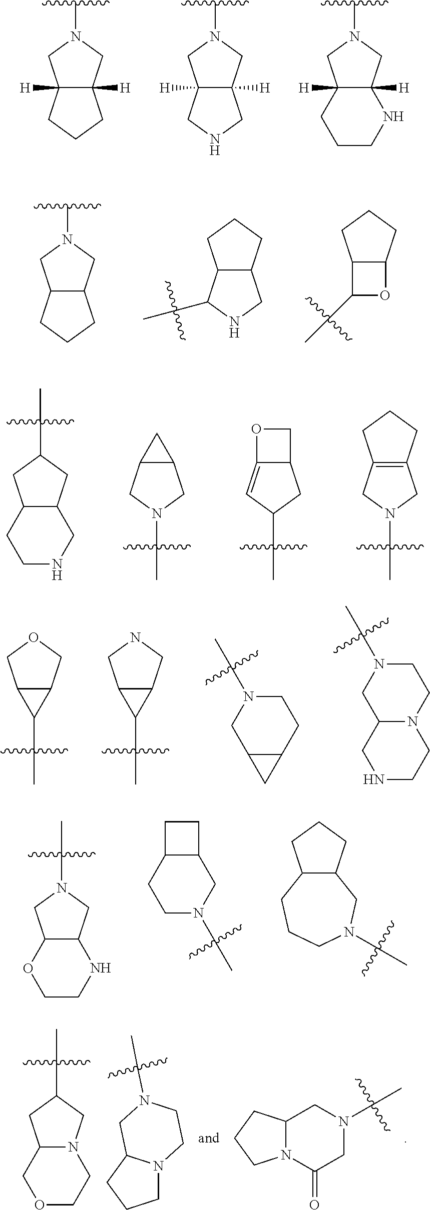

“Bridged Heterocyclyl” refers to a 5 to 14 membered polycyclic heterocyclic alkyl group, wherein every two rings in the system share two disconnected atoms, the rings can have one or more double bonds, but have no completely conjugated pi-electron system, and the rings have one or more heteroatoms selected from the group consisting of N, O, and S (O)ₘ (wherein m is 0, 1, or 2) as ring atoms, the remaining ring atoms being C. Preferably a bridged heterocyclyl is 6 to 14 membered, more preferably 7 to 10 membered, and most preferably 7 to 8 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following substituents:

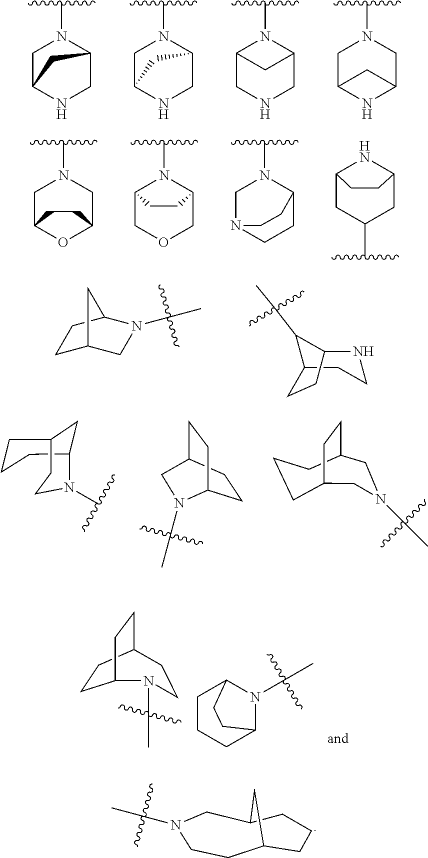

The ring of said heterocyclyl can be fused to the ring of an aryl, heteroaryl or cycloalkyl, wherein the ring bound to the parent structure is heterocyclyl. Representative examples include, but are not limited to the following substituents:

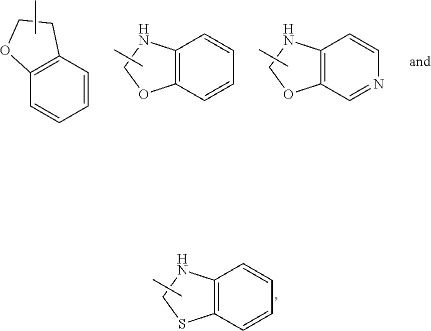

The heterocyclyl is optionally substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic and alkylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl can be fused to the ring of heteroaryl, heterocyclyl or cycloalkyl, wherein the ring bound to parent structure is aryl. Representative examples include, but are not limited to, the following substituents:

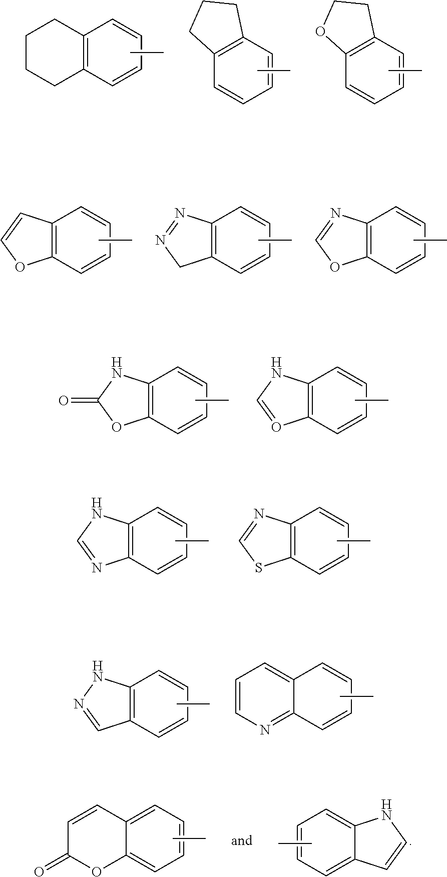

The aryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic and alkylthio.

“Heteroaryl” refers to an aryl system having 1 to 4 heteroatoms selected from the group consisting of O, S and N as ring atoms and having 5 to 14 annular atoms. Preferably a heteroaryl is 5- to 10-membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, isoxazolyl and the like. The heteroaryl can be fused with the ring of an aryl, heterocyclyl or cycloalkyl, wherein the ring bound to parent structure is heteroaryl. Representative examples include, but are not limited to, the following substituents:

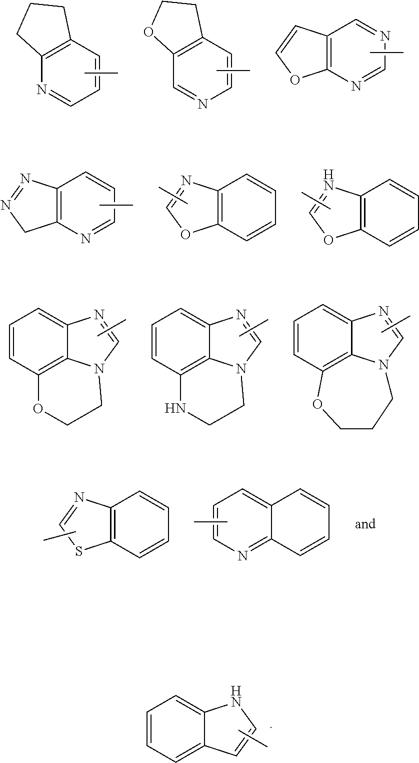

The heteroaryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Haloalkoxy” refers to an alkoxy group substituted by one or more halogen(s), wherein the alkoxy is as defined above.

The hydrogen atom of the present invention can be substituted by its isotope deuterium. Any of the hydrogen atoms in the compounds of the examples of the present invention can also be substituted by deuterium atom.

“Bond” refers to a covalent bond using a sign of “—”.

“Hydroxyalkyl” refers to an alkyl group substituted by a hydroxy group, wherein alkyl is as defined above.

“Hydroxyl” or “hydroxy” refers to an —OH group.

“Halogen” or “halo” refers to fluoro, chloro, bromo or iodo atoms.

“Amino” refers to a —NH₂ group.

“Cyano” refers to a —CN group.

“Nitro” refers to a —NO₂ group.

“Oxo group” refers to a═O group.

“Carboxyl” refers to a —C(O)OH group.

“Alkoxycarbonyl” refers to a —C(O)O(alkyl) or (cycloalkyl) group, wherein the alkyl and cycloalkyl are defined as above.

Where it is stated that groups or substituents are “independently selected from” (and variants thereof) a list of choices, it is meant that the choice for any one of such groups or substituents does not determine the choice for any other one of such groups or substituents. By way of an illustration, but not as a limitation, the term “A and B are independently selected from a and b” or “each of A and B is independently selected from a and b” is meant to encompass selections where A is a and B is a, A is b and B is b, A is a and B is b, and A is b and B is a.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen a members in a group independently substituted with a corresponding number of substituents. In some embodiments, the number of such hydrogen members is up to 5. In other embodiments it si between 1 and 3. It goes without saying that the substituents exist in their only possible chemical position. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present invention or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the invention, such salts being safe and effective when used in a mammal and have corresponding biological activity.

### EXAMPLES

The following examples serve to illustrate the invention, but the examples should not be considered as limiting the scope of the invention. If specific conditions for the experimental method are not specified in the examples of the present invention, they are generally in accordance with conventional conditions or recommended conditions of the raw materials and the product manufacturer. The reagents without a specific source indicated are commercially available, conventional reagents.

The structure of each compound is identified by nuclear magnetic resonance (NMR) and/or mass spectrometry (MS). NMR chemical shifts (6) are given in 10⁻⁶ (ppm). NMR is determined by Varian Mercury 300 MHz Bruker Avance III 400 MHz machine. The solvents used are deuterated-dimethyl sulfoxide (DMSO-d₆), deuterated-chloroform (CDCl₃) and deuterated-methanol (CD₃OD).

High performance liquid chromatography (HPLC) is determined on an Agilent 1200DAD high pressure liquid chromatography spectrometer (Sunfire C18 150×4.6 mm chromatographic column) and a Waters 2695-2996 high pressure liquid chromatography spectrometer (Gimini C18 150×4.6 mm chromatographic column). Liquid Chromatography Mass Spectrometry (LCMS) is determined on an Agilent 1200 high pressure liquid chromatography spectrometer & mass spectrometry (Sunfire C18 4.6*50 mm 3.5 um chromatographic column) and an Agilent 19091S-433 HP-5 high pressure liquid chromatography spectrometer & mass spectrometry (XBridge C18 4.6*50 mm 3.5 um chromatographic column).

Chiral High performance liquid chromatography (HPLC) is determined on SFC Thar 80 & 150 & 200 (waters.)

The average rates of ATPase inhibition, and the IC₅₀ values are determined by Victor Nivo multimode plate reader (PerkinElmer, USA).

The thin-layer silica gel plates used in thin-layer chromatography are Yantai Xinnuo silica gel plate. The dimension of the plates used in TLC is 0.15 mm to 0.2 mm, and the dimension of the plates used in thin-layer chromatography for product purification was 0.4 mm to 0.5 mm.

Column chromatography generally uses Qingdao Haiyang 200 to 300 mesh silica gel as carrier.

The known starting material of the invention can be prepared by the conventional synthesis method in the prior art, or can be purchased from ABCR GmbH & Co. KG, Acros Organics, Aldrich Chemical Company, Accela ChemBio Inc or Dari chemical Company, etc.

Unless otherwise stated in the examples, the following reactions are placed under argon atmosphere or nitrogen atmosphere.

The term “argon atmosphere” or “nitrogen atmosphere” means that a reaction flask is equipped with a balloon having 1 L of argon or nitrogen.

The term “hydrogen atmosphere” means that a reaction flask was equipped with a balloon having 1 L of hydrogen.

MS is mass spectroscopy with (+) referring to the positive mode which generally gives a M+1 (or M+H) absorption where M=the molecular mass.

### Synthetic Procedure

### Synthesis of Intermediate A8

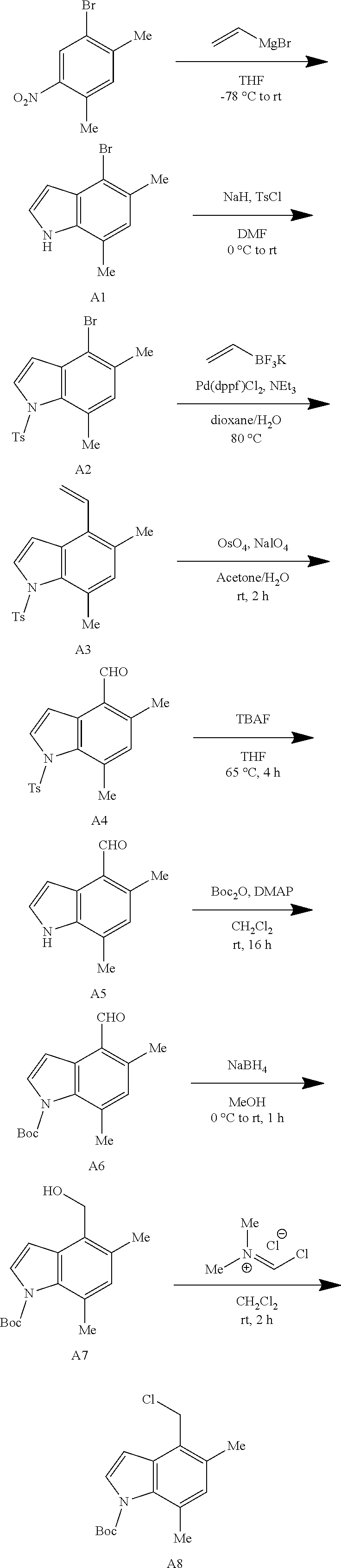

### Step 1: Synthesis of 4-bromo-5,7-dimethyl-1H-indole (A1)

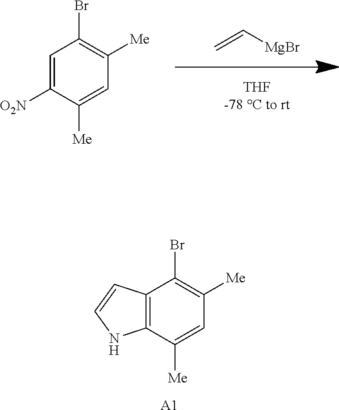

A solution of 1-bromo-2,4-dimethyl-5-nitrobenzene (60.0 g, 0.260 mol) in THF (800 mL) was cooled to −78° C. and vinylmagnesium bromide (880 mL, 1.0 M solution in THF, 0.880 mol) was added dropwise. The reaction mixture was warmed slowly to −40° C., then stirred for 4 h at that temperature. Water was added and the reaction mixture warmed to room temperature. The aqueous layer was extracted with ethyl acetate (3×600 mL) and the combined organic layers washed with brine (2×600 mL) and dried over anhydrous Na₂SO₄, filtered, and concentrated. The crude product was purified by flash column chromatography (SiO₂, 20:1 petroleum ether/ethyl acetate) to give the title compound, 4-bromo-5,7-dimethyl-1H-indole (18.0 g) as a brown solid. LCMS (m/z): [M+H]⁺ calc'd for C₁₀H₁₁BrN, 224/226; found, 224/226.

| Structure and Name | Intermediate# | LCMS |
|---|---|---|
| 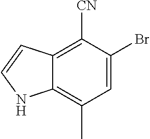 | A11 | LCMS (m/z): [M + H]⁺ calc'd for C₁₀H₈BrN₂, 235.0; found, 235.1 |
| 5-bromo-7-methyl-1H-indole-4-carbonitrile | | |

### Step 2: Synthesis of 4-bromo-5,7-dimethyl-1-tosyl-1H-indole (A2)

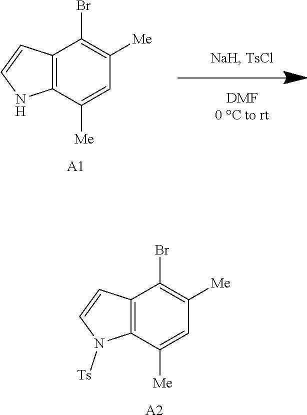

A solution of 4-bromo-5,7-dimethyl-1H-indole (18.0 g, 80.0 mmol) in DMF (200 mL) was cooled to 0° C. and NaH (4.8 g, 0.12 mol, 60%) was added portionwise. The mixture was warmed to room temperature and stirred for 30 min at that temperature, before being re-cooled to 0° C. TsCl (22.8 g, 0.120 mol) was added portionwise, prior to warming to room temperature and stirring at that temperature overnight. The reaction mixture was quenched with water (200 mL) and the aqueous phase extracted with ethyl acetate (3×200 mL). The combine organic layers were washed with brine (200 mL), dried over anhydrous Na₂SO₄, filtered, and concentrated. The crude product was purified by flash column chromatography (SiO₂, 30:1 petroleum ether/ethyl acetate) to give the title compound, 4-bromo-5,7-dimethyl-1-tosyl-1H-indole (12.4 g, 13% over two steps) as a brown solid. LCMS (m/z): [M+H]⁺ calc'd for C₁₇H₁₇N₂OS, 378/380; found, 378/380.

| Structure and Name | Intermediate# | LCMS |
|---|---|---|
| 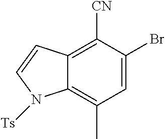 [SMILES: Cc1ccc(S(=O)(=O)n2ccc3c(C#N)c(Br)cc(C)c32)cc1] | A12 (from A11) | LCMS (m/z): [M + H]⁺ calc'd for C₁₇H₁₄BrN₂O₂S, 389.0; found, 389.0 |
| 5-bromo-7-methyl-1-tosyl-1H-indole-4-carbonitrile | | |

### Step 3: Synthesis of 5,7-dimethyl-1-tosyl-4-vinyl-1H-indole (A3)

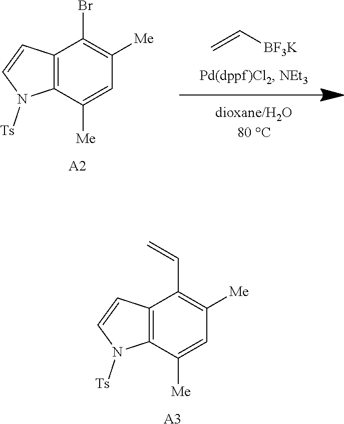

To a solution of 4-bromo-5,7-dimethyl-1-tosyl-1H-indole (12.4 g, 32.8 mmol) in dioxane (120 mL) and H₂O (30 mL) was added potassium vinyltrifluoroborate (8.8 g, 66 mmol), Et₃N (20.8 g, 206 mmol) and Pd(dppf)Cl₂ (1.2 g, 1.6 mmol) and the reaction mixture stirred at 80° C. overnight. The reaction was quenched by the addition of H₂O (100 mL) and extracted with ethyl acetate (3×100 mL). The combined organic layers were washed with brine (300 mL), dried over anhydrous Na₂SO₄, filtered, and concentrated. The crude product was purified by flash column chromatography (SiO₂, 20:1 petroleum ether/ethyl acetate) to give the title compound, 5,7-dimethyl-1-tosyl-4-vinyl-1H-indole (7.0 g, 65%) as a colorless oil. LCMS (m/z): [M+H]⁺ calc'd for C₁₉H₂₀NO₂S, 326; found, 326.

| Structure and Name | Intermediate# | LCMS |
|---|---|---|
| 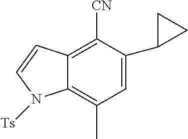 [SMILES: Cc1ccc(S(=O)(=O)n2ccc3c(C#N)c(C4CC4)cc(C)c32)cc1] | A13 (from A12) | LCMS (m/z): [M + H]⁺ calc'd for C₂₀H₁₉N₂O₂S, 351.1; found, 351.0 |
| 5-cyclopropyl-7-methyl-1-tosyl-1H-indole-4- | | |
| carbonitrile | | |

### Step 4: Synthesis of 5,7-dimethyl-1-tosyl-1H-indole-4-carbaldehyde (A4)

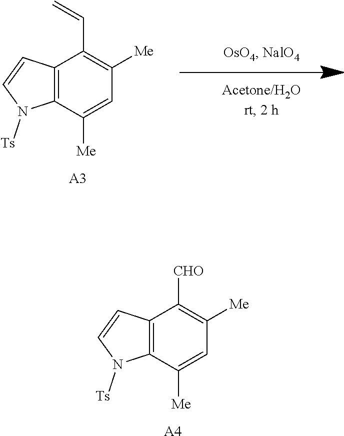

To a solution of 5,7-dimethyl-1-tosyl-4-vinyl-1H-indole (7.0 g, 22 mmol) in acetone (150 mL) and H₂O (30 mL) was added OsO₄ (173 mg, 0.680 mmol) and NaIO₄ (23.0 g, 108 mmol) and the reaction mixture stirred at room temperature for 2 h. The mixture was concentrated to remove volatiles and the remaining aqueous layer extracted with CH₂Cl₂ (3×60 mL). The combined organic layers were washed with brine (100 mL), dried over anhydrous Na₂SO₄, filtered, and concentrated. The crude product was purified by flash column chromatography (SiO₂, 5:1 petroleum ether/ethyl acetate) to give 5,7-dimethyl-1-tosyl-1H-indole-4-carbaldehyde (3.8 g, 54%) as a white solid. LCMS (m/z): [M+H]⁺ calc'd for C₁₈H₁₈NO₃S, 328; found, 328.

### Step 5: Synthesis of 5,7-dimethyl-1H-indole-4-carbaldehyde (A5)

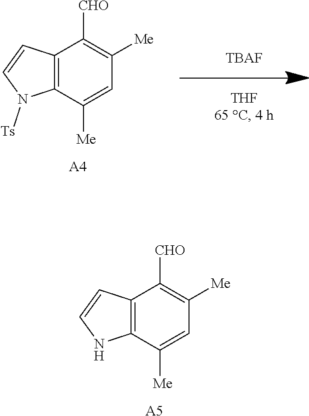

To a solution of 5,7-dimethyl-1-tosyl-1H-indole-4-carbaldehyde (2.3 g, 7.0 mmol) in THF (25 mL) was added TBAF (10.5 mL, 10.5 mmol, 1.0 M solution in THF) and the mixture stirred at 65° C. for 4 h. The reaction was quenched by the addition of H₂O (50 mL) and extracted with ethyl acetate (3×50 mL). The combined organic layers were washed with brine (50 mL), dried over anhydrous Na₂SO₄, filtered, and concentrated to give crude 5,7-dimethyl-1H-indole-4-carbaldehyde (1.4 g) as a brown oil. LCMS (m/z): [M+H]⁺ calc'd for C₁₁H₁₂NO, 174; found, 174.

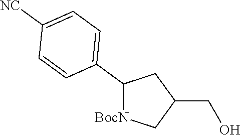 [SMILES: CC(C)(C)OC(=O)N1CC(CO)CC1c1ccc(C#N)cc1]

### Step 6: Synthesis of tert-butyl 4-formyl-5,7-dimethyl-1H-indole-1-carboxylate (A6)

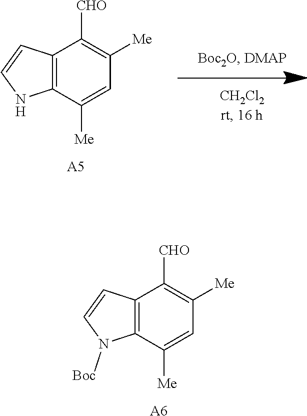

To a solution of 5,7-dimethyl-1H-indole-4-carbaldehyde (1.4 g, 8.1 mmol) and DMAP (1.1 g, 8.9 mmol) in CH₂Cl₂ (15 mL) was added Boc₂O (3.8 g, 12 mmol) and the mixture stirred at room temperature for 16 h. The reaction was quenched by the addition of H₂O (30 mL) and extracted with CH₂Cl₂ (3×30 mL). The combined organic layers were washed with brine (30 mL), dried over anhydrous Na₂SO₄, filtered, and concentrated. The crude product was purified by flash column chromatography (SiO₂, 10:1 petroleum ether/ethyl acetate) to give tert-butyl 4-formyl-5,7-dimethyl-1H-indole-1-carboxylate (1.5 g, 68%) as a colorless oil. LCMS (m/z): [M+H]⁺ calc'd for C₁₆H₂₀NO₃, 274; found 274.

| Structure and Name | Intermediate# | LCMS |
|---|---|---|
| 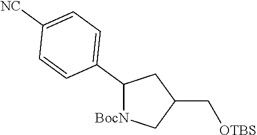 [SMILES: CC(C)(C)OC(=O)N1CC(CO[Si](C)(C)C(C)(C)C)CC1c1ccc(C#N)cc1] | A15 (from C25) | LCMS (m/z): [M − ^{t}Bu]⁺ calc'd for C₁₉H₂₉N₂O₃Si, 361.2; found, 361.1 |
| tert-butyl 4-(((tert-butyldimethylsilyl)oxy)methyl)- | | |
| 2-(4-cyanophenyl)pyrrolidine-1-carboxylate | | |

### Step 7: Synthesis of tert-butyl 4-(hydroxymethyl)-5,7-dimethyl-1H-indole-1-carboxylate (A7)

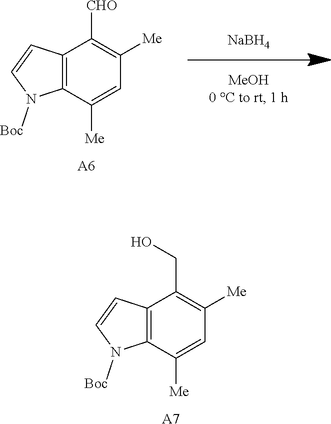

To a solution of tert-butyl 4-formyl-5,7-dimethyl-1H-indole-1-carboxylate (1.00 g, 3.66 mmol) in MeOH (10 mL) was added NaBH₄(318 mg, 8.41 mmol) portionwise at 0° C. and the mixture was stirred at room temperature for 1 h. The reaction was quenched with half saturated aqueous KHSO₄, diluted with water (10 mL), and extracted with ethyl acetate (3×20 mL). The organic layers were combined, washed with brine (20 mL), dried over anhydrous Na₂SO₄, filtered, and concentrated to obtain crude tert-butyl [4-(hydroxymethyl)-5,7-dimethylindol-1-yl]formate (950 mg) as a yellow oil. LCMS (m/z): [M-OH]⁺ calc'd for C₁₆H₂₀NO₂, 258; found, 258.

The following intermediate was synthesized using similar conditions as those described in step 7, above, along with appropriate starting materials.

| Structure and Name | Intermediate# | LCMS |
|---|---|---|
| 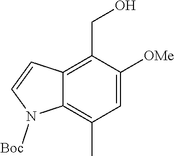 [SMILES: COc1cc(C)c2c(ccn2C(=O)OC(C)(C)C)c1CO] | A9 From CAS: 1481631-51-9 | LCMS (m/z): [M − OH]⁺ calc'd for C₁₆H₂₀NO₃, 274.1; found, 274 |
| tert-butyl 4-(hydroxymethyl)-5-methoxy-7-methyl- | | |
| 1H-indole-1-carboxylate | | |

### Step 8: Synthesis of tert-butyl 4-(chloromethyl)-5,7-dimethyl-1H-indole-1-carboxylate (A8)

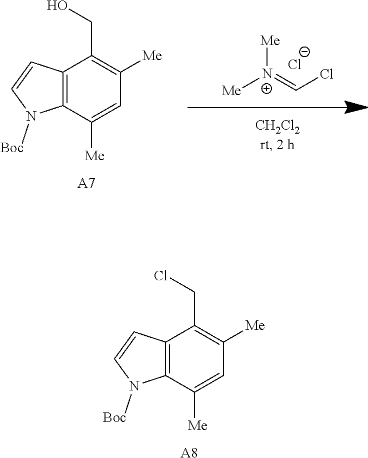

To a solution of tert-butyl 4-(hydroxymethyl)-5,7-dimethyl-1H-indole-1-carboxylate (950 mg, 3.45 mmol) in CH₂Cl₂ (10 mL) was added (chloromethylene)dimethyliminium chloride (711 mg, 5.56 mmol) in one portion at room temperature under nitrogen and the mixture stirred at that temperature for 2 h. The reaction mixture was cooled to 0° C., then quenched with 5% aq. NaHCO₃. The mixture was extracted with CH₂Cl₂ (3×20 mL). The combined organic layers were washed with brine (20 mL), dried over anhydrous Na₂SO₄, filtered, and concentrated. The crude product, tert-butyl 4-(chloromethyl)-5,7-dimethyl-1H-indole-1-carboxylate (900 mg), was obtained as a yellow oil. LCMS (m/z): [M−Cl]⁺ calc'd for C₁₆H₂₀NO₂, 258; found, 258.

The following intermediate was synthesized using similar conditions as those described in step 8, above, along with appropriate starting materials.

| Structure and Name | Intermediate# | LCMS |
|---|---|---|
| 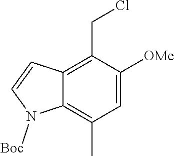 | A10 | LCMS (m/z): [M − Cl]⁺ calc'd for C₁₆H₂₀NO₃, 274.1; found, 274 |
| tert-butyl 4-(chloromethyl)-5-methoxy-7-methyl- | | |
| 1H-indole-1-carboxylate | | |
| 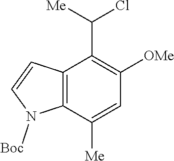 [SMILES: COc1cc(C)c2c(ccn2C(=O)OC(C)(C)C)c1C(C)Cl] | A12 | LCMS (m/z): [M − Cl]⁺ calc'd for C₁₇H₂₂NO₃, 288; found, 288 |
| tert-butyl 4-(1-hydroxyethyl)-5-methoxy-7-methyl- | | |
| 1H-indole-1-carboxylate | | |

### Synthesis of tert-butyl 4-(1-chloroethyl)-5-methoxy-7-methyl-1H-indole-1-carboxylate (A12)

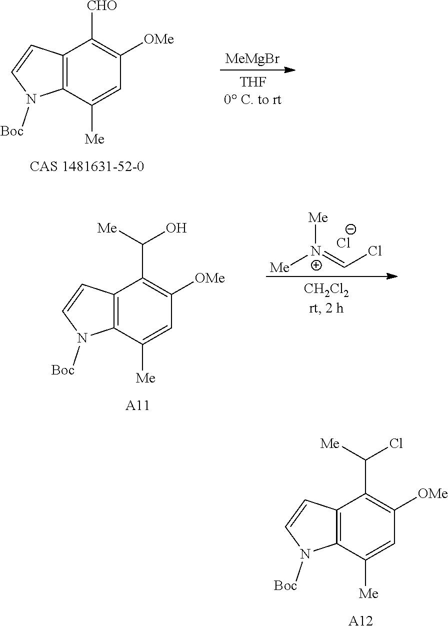

### Step 1: Synthesis of tert-butyl 4-(1-hydroxyethyl)-5-methoxy-7-methyl-1H-indole-1-carboxylate (A11)

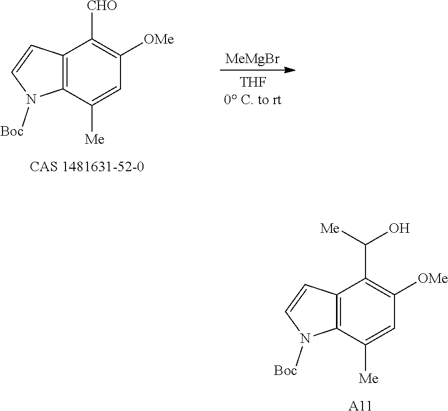

To a solution of tert-butyl 4-formyl-5-methoxy-7-methyl-1H-indole-1-carboxylate (20 g, 69.20 mmol) in THF (200 mL) at 0° C. was added CH3MgBr (240 mL, 138.4 mmol). The mixture was warmed to rt while stirring for 3 h. The organic layer was separated, and the aqueous layer was extracted twice with CH₂Cl₂ (3×100 mL). The combined organic layers were dried over anhydrous Na2SO4, filtered, and concentrated. The filtrate was concentrated under vacuum and the residue was purified by flash column chromatography (3:1 petroleum ether/ethyl acetate) to afford product A11 (18 g, 59.02 mmol, 85% yield) as a white solid. LCMS (m/z): [M-Boc-OH]⁺ calc'd for C₁₇H₂₄N2O₄, 306; found, 186.

### Synthesis of 3-azabicyclo[3.1.0]hexane (B1)

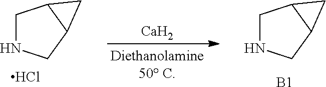

To a 50 mL round-bottom flask was added diethanolamine (7.92 g, 75.3 mmol) and 3-azabicyclo[3.1.0]hexane hydrochloride (3.00 g, 25.1 mmol). Small portions of CaH₂ were added until gas evolution was no longer observed. The flask was equipped with a stir bar and a shortpath distillation head with a 25 mL round-bottom flask as a receiver. The mixture was heated to 50° C. with an oil bath under reduced pressure. The receiver was cooled with a dry ice/EtOH bath. Vaporized amine was driven over into the receiver by gentle warming with a heat gun. The title product was obtained as a colorless liquid (1.0 g, 45%). ¹H NMR (300 MHz, CDCl₃): δ 2.87 (q, J=11.5 Hz, 4H), 1.44-1.25 (m, 2H), 0.44 (dd, J=12.9, 7.7 Hz, 1H), 0.11 (dd, J=8.7, 4.2 Hz, 1H).

### Synthesis of 1-oxa-8-azaspiro[4.5]decane (B2)

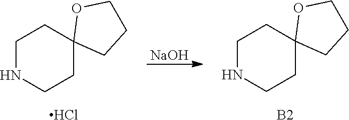

1-oxa-8-azaspiro[4.5]decane hydrochloride (5.00 g, 28.0 mmol) was added to a solution of NaOH (aq., 1 M). The mixture was extracted with CH₂Cl₂ (4×30 mL). The organic layers were combined, dried over anhydrous Na₂SO₄, and concentrated in vacuo to get the product (3.30 g, 83%) as a yellow oil. LCMS (m/z): [M+H]⁺ calc'd for C₈H₁₆NO, 142.1; found, 142.1.

The following intermediate was synthesized using similar conditions as those described above along with appropriate starting materials.

The following intermediate was synthesized using similar conditions as those described above along with appropriate starting materials.

| Structure and Name | Intermediate# | LCMS |
|---|---|---|
| 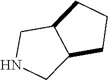 | B3 From CAS 926276-10-0 | LCMS (m/z): [M + H]⁺ calc'd for C₇H₁₄N, 112.1; found, 112.1 |
| octahydrocyclo- | | |
| penta[c]pyrrole | | |

### Synthesis of (((tert-butyldimethylsilyl)oxy)methyl)pyrrolidine (B4)

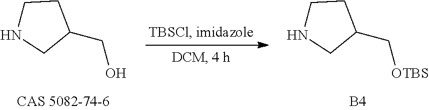

A 50 mL round bottom flask equipped with pyrrolidin-3-ylmethanol (3 g, 30 mmol) and imidazole (6.06 g, 90 mmol) in DCM (20 mL) was stirred at 0° C. for 20 minutes. Then a solution of TBSCI (6.7 g, 45 mmol) in DCM (10 mL) was added dropwise. The resultant solution was stirred at room temperature for 4 h. The mixture was concentrated and the residue diluted with 1N NaOH (20 mL), extracted with ethyl acetate (40 mL×3). The combined organic extracts were washed with water (3×10 mL) and concentrated. The crude product was purified by flash column chromatography (dichloromethane:methanol=10:1) to give 3-(((tert-butyldimethylsilyl)oxy)methyl)pyrrolidine (3.44 g, 51%) as a light yellow liquid. LCMS (m/z): [M+H]⁺ calc'd for C₁₁H₂₆NOSi, 216.1; found 216. ¹H NMR (400 MHz, CDCl₃) δ 3.50 (ddd, J=16.9, 9.9, 6.5 Hz, 2H), 3.04-2.87 (m, 2H), 2.83 (dt, J=10.9, 7.3 Hz, 1H), 2.68 (dd, J=11.2, 5.7 Hz, 1H), 2.41 (br, 1H), 2.25 (dt, J=14.4, 7.3 Hz, 1H), 1.91-1.71 (m, 1H), 1.48-1.32 (m, 1H), 0.87 (s, 9H), 0.03 (s, 6H).

### Synthesis of tert-butyl 2-(4-cyanophenyl)-4-formylpyrrolidine-1-carboxylate (B5)

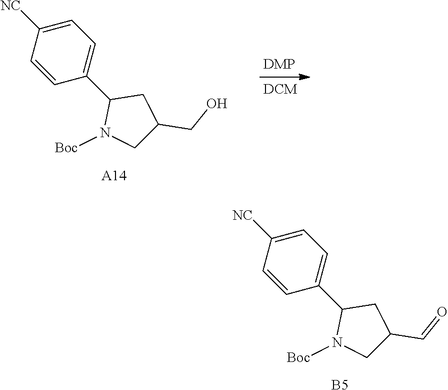

To a solution tert-butyl 2-(4-cyanophenyl)-4-(hydroxymethyl)pyrrolidine-1-carboxylate (120 mg, 0.4 mmol) in dry DCM (5 mL) was added DMP (252 mg, 0.6 mmol) under an atmosphere of N₂ and the reaction stirred at room temperature for 2 h. The mixture was concentrated, and the crude product was purified directly by silica-gel column chromatography (0-60% ethyl acetate in petroleum ether) to obtain tert-butyl 2-(4-cyanophenyl)-4-formylpyrrolidine-1-carboxylate (68 mg, 53%). LCMS (m/z): [M-tBu]⁺ calc'd for C₁₃H₁₃N₂O₃, 245.1; found 245.

### Synthesis of tert-butyl 2-(4-(methoxycarbonyl)phenyl)-3-azabicyclo[3.2.0]heptane-3-carboxylate (B6)

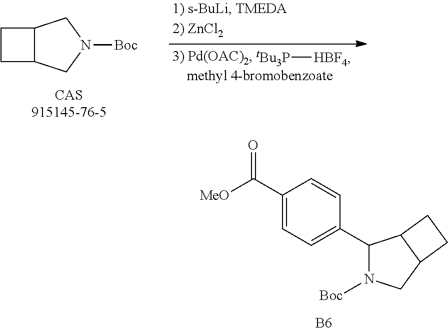

To a solution of tert-butyl 3-azabicyclo[3.2.0]heptane-3-carboxylate (200 mg, 1 mmol) and TMEDA (603 mg, 5.20 mmol) in Et₂O (5 mL) at −78° C. was added a solution of sec-BuLi (1.3 N in hexane, 4 mL) dropwise, at a rate to maintain the temperature below −78° C. The resulting solution was aged for 3 h at −78° C. A solution of ZnCl₂ (2.8 mL, 2.8 mmol, 1M in Et₂O) was added to the reaction dropwise with rapid stirring, maintaining the temperature below −78° C. The resulting light suspension was aged at −78° C. for 30 minutes and then warmed to 20° C. The resulting homogeneous solution was aged for 30 minutes at 20° C., prior to addition of methyl 4-bromobenzoate (428 mg, 2.00 mmol) followed by Pd(OAc)₂ (135 mg 0.6 mmol), and ^{t}Bu₃P-HBF₄ (348 mg 1.2 mmol) in one portion. The mixture, which precipitated zinc salts during the course of the reaction, was aged overnight in a water bath at 20° C. The reaction mixture was quenched by water (5 mL), concentrated and extracted by ethyl acetate (3×40 mL). The combined organic extracts were concentrated, and the crude product was purified by silica-gel column chromatography (20% ethyl acetate in petroleum ether) to obtain tert-butyl 2-(4-(methoxycarbonyl)phenyl)-3-azabicyclo[3.2.0]heptane-3-carboxylate (28 mg, 8%). LCMS (m/z): [M-tBu]⁺ calc'd for C₁₅H₁₈NO₄, 276.1; found 276.

### Synthesis of (3aR,6aS)-2-benzyl-5-(2,2-difluoroethyl)octahydropyrrolo[3,4-c]pyrrole

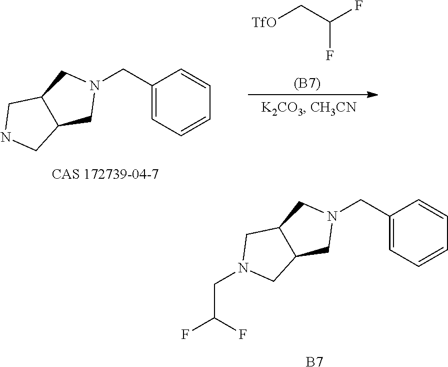

To a solution of the (3aS,6aR)-2-benzyl-octahydropyrrolo[3,4-c]pyrrole (1 g, 5 mmol) in anhydrous acetonitrile (40 mL) was added K₂CO₃ (1 g, 10 mmol) and 2,2-difluoroethyl trifluoromethanesulfonate (1.3 g, 6.0 mmol). The mixture was stirred for 4 h at room temperature. The reaction was then concentrated, and the residue was dissolved in ethyl acetate (50 mL) and washed with water (2×20 mL), dried over Na₂SO₄, and concentrated under vacuum to provide (3aR,6aS)-2-benzyl-5-(2,2-difluoroethyl)octahydropyrrolo[3,4-c]pyrrole (1.62 g, 93%) as a colorless oil. LCMS (m/z): [M+H]⁺ calc'd for C₁₅H₂₁F₂N₂, 267.2; found 267.2.

### Synthesis of (3aR,6aS)-2-(2,2-difluoroethyl)-octahydropyrrolo[3,4-c]pyrrole (B8)

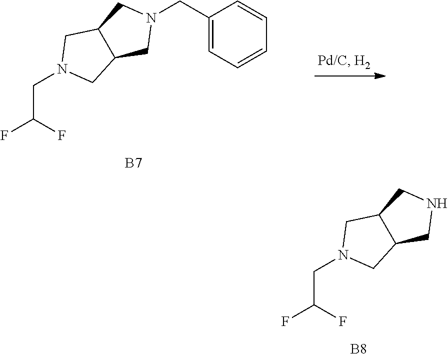

To a solution of (3aR,6aS)-2-benzyl-5-(2,2-difluoroethyl)-octahydropyrrolo[3,4-c]pyrrole (1.2 g, 4.5 mmol) in MeOH (50 mL) was added Pd/C (120 mg, 1.12 mmol, 10% loading on carbon). The reaction mixture was stirred for 3 h at room temperature under H₂ atmosphere. After filtering the solid, the filtrate was concentrated under vacuum to provide (3aR,6aS)-2-(2,2-difluoroethyl)-octahydropyrrolo[3,4-c]pyrrole (660 mg, 83%) as a yellow oil. LCMS (m/z): [M+H]⁺ calc'd for C₈H₁₅F₂N₂, 177.1; found 177.2.

The following intermediate was synthesized using similar conditions as those described above along with appropriate starting materials.

| Structure and Name | Intermediate# | LCMS |
|---|---|---|
| 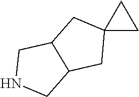 | B9 | LCMS (m/z): [M + H]⁺ calc'd for C₉H₁₆N, 138.1; found, 138.2 |
| hexahydro-1H-spiro[cyclopenta[c]pyrrole-5,1′- | | |
| cyclopropane] | | |
| 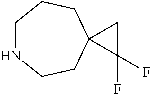 [SMILES: FC1(F)CC12CCCNCC2] | B10 | LCMS (m/z): [M + H]⁺ calc'd for C₈H₁₄F₂N, 162.1; found, 162.1 |
| 1,1-difluoro-6-azaspiro[2.6]nonane | | |
| 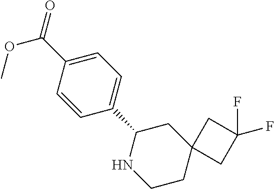 [SMILES: COC(=O)c1ccc([C@@H]2CC3(CCN2)CC(F)(F)C3)cc1] | B11 | LCMS (m/z): [M + H]⁺ calc'd for C₁₆H₂₀F₂NO₂, 296.1; found, 296 |
| methyl (S)-4-(2,2-difluoro-7-azaspiro[3.5]nonan-6- | | |
| yl)benzoate | | |
| 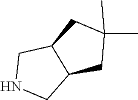 | B12 | LCMS (m/z): [M + H]⁺ calc'd for C₉H₁₈N, 140.1; found, 140 |
| (3aR,6aS)-5,5- | | |
| dimethyloctahydrocyclopenta[c]pyrrole | | |
| 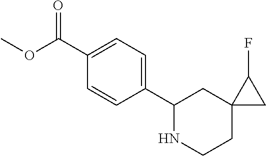 [SMILES: COC(=O)c1ccc(C2CC3(CCN2)CC3F)cc1] | B13 | LCMS (m/z): [M + H]⁺ calc'd for C₁₅H₁₉FNO₂, 264.1; found, 264 |
| methyl 4-(1-fluoro-6-azaspiro[2.5]octan-5- | | |
| yl)benzoate | | |
| 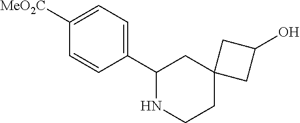 [SMILES: COC(=O)c1ccc(C2CC3(CCN2)CC(O)C3)cc1] | B19 (From B18) | LCMS (m/z): [M + H]⁺ calc'd for C₁₆H22NO₃, 276.2; found, 276 |
| 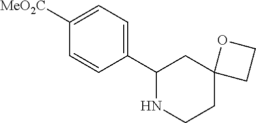 [SMILES: COC(=O)c1ccc(C2CC3(CCN2)CCO3)cc1] | D7 (From D6) | LCMS (m/z): [M + H]⁺ calc'd for C₁₅H₁₉NO₃, 261.1; found, 261.1 |

### Synthesis of benzyl 6-(4-(methoxycarbonyl)phenyl)-2-oxo-7-azaspiro[3.5]nonane-7-carboxylate (B14)

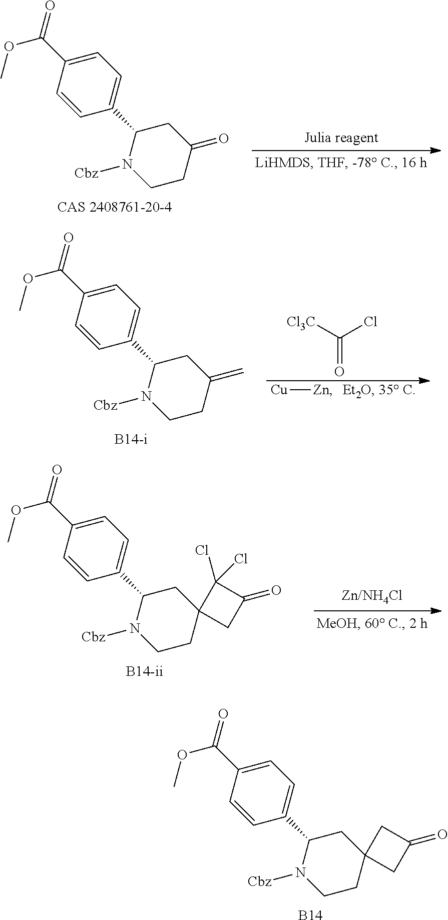

### Step 1: Synthesis of benzyl 2-(4-(methoxycarbonyl)phenyl)-4-methylenepiperidine-1-carboxylate (B14-i)

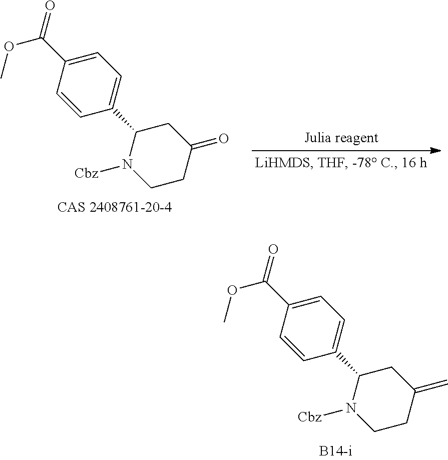

To a solution of methyl-Julia reagent (1.716 g, 8.16 mmol) in THF (50 mL), was added LiHMDS (4.74 ml, 7.62 mol) at −78° C. under N₂ atmosphere (balloon), The mixture was stirred at −78° C. for 30 minutes, benzyl (S)-2-(4-(methoxycarbonyl)phenyl)-4-oxopiperidine-1-carboxylate (1 g, 2.72 mmol) [obtained by chiral separation of the commercial racemate (CAS: 2238811-87-3), retention time=4.85 min on IG-H column −0.46 cm I. D.×15 cm L at 2.5 mL/min] in THF (10 ml) was added dropwise to the mixture. The mixture was stirred at room temperature for 16 h. The mixture was then quenched with water and concentrated under reduced pressure. The residue was dissolved in ethyl acetate (30 mL), washed with brine (3×20 mL), dried over Na₂SO₄ and concentrated. The crude product was purified by flash column chromatography (Petroleum ether/ethyl acetate=16/1) on silica gel to obtain benzyl 2-(4-(methoxycarbonyl)phenyl)-4-methylenepiperidine-1-carboxylate (600 mg, 60%) as a colorless oil. LCMS (m/z): [M+H]⁺ calc'd for C₂₂H₂₄NO₄, 366.2; found 366.

The following intermediates were synthesized using similar conditions as those described above along with appropriate starting materials.

| Structure and Name | Intermediate# | LCMS |
|---|---|---|
| 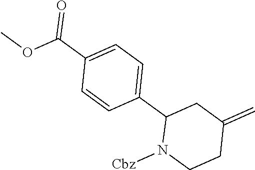 | B14-I rac | LCMS (m/z): [M + H]⁺ calc'd for C₂₂H₂₄NO₄, 366.2; found 366. |
| 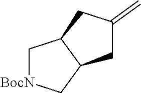 [SMILES: C=C1C[C@@H]2CN(C(=O)OC(C)(C)C)C[C@@H]2C1] | B15 | LCMS (m/z): [M + H]⁺ calc'd for C₁₃H₂₂NO₂, 224.2; found, 224.3 |
| tert-butyl (3aR,6aS)-5- | | |
| methylenehexahydrocyclopenta[c]pyrrole-2(1H)- | | |
| carboxylate | | |

### Step 2: Synthesis of benzyl 1,1-dichloro-6-(4-(methoxycarbonyl)phenyl)-2-oxo-7-azaspiro[3.5]nonane-7-carboxylate (B14-ii)

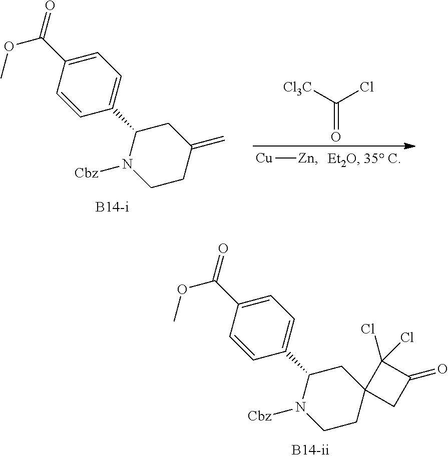

To a suspension of Cu—Zn (1.08 g, 16.2 mmol) and benzyl 2-(4-(methoxycarbonyl)phenyl)-4-methylenepiperidine-1-carboxylate (300 mg, 0.81 mmol) in Et₂O (35 mL) was added a solution of trichloroacetyl chloride (1.5 g, 8.1 mmol) in Et₂O (5 mL) dropwise at room temperature under a nitrogen atmosphere. After stirring of the mixture at 40° C. for 2 h, the reaction mixture was poured into an aqueous solution of NaHCO₃ at 0° C. and filtered. The filtrate was extracted with ethyl acetate (3×20 mL). The combined organic extracts were washed with brine (3×10 mL) and concentrated to provide crude benzyl 1,1-dichloro-6-(4-(methoxycarbonyl)phenyl)-2-oxo-7-azaspiro[3.5]nonane-7-carboxylate (400 mg) as an oil, which was used in the next step without further purification. LCMS (m/z): [M+H]⁺ calc'd for C₂₄H₂₄Cl₂NO₅, 476.1; found, 476.

### Step 3:Synthesis of benzyl 6-(4-(methoxycarbonyl)phenyl)-2-oxo-7-azaspiro[3.5]nonane-7-carboxylate (B14)

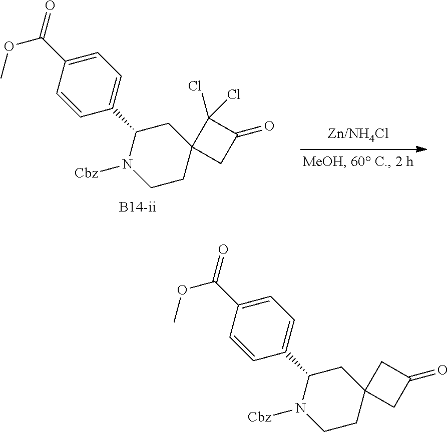 [SMILES: COC(=O)c1ccc([C@@H]2CC3(CCN2C(=O)OCc2ccccc2)CC(=O)C3(Cl)Cl)cc1 ; COC(=O)c1ccc([C@@H]2CC3(CCN2C(=O)OCc2ccccc2)CC(=O)C3)cc1]

To a solution of crude benzyl 1,1-dichloro-6-(4-(methoxycarbonyl)phenyl)-2-oxo-7-azaspiro[3.5]nonane-7-carboxylate (800 mg, 1.7 mmol) in MeOH (24 mL) was added NH₄Cl (800 mg, 16.8 mmol) and Zinc (760 mg, 12 mmol) in portions at room temperature. The reaction mixture was stirred at 60° C. for 2 h, and then filtered. The filtrate was concentrated in vacuo and the resulting residue was purified by flash column chromatography (EA/PE=4/1) on silica gel to afford benzyl 6-(4-(methoxycarbonyl)phenyl)-2-oxo-7-azaspiro[3.5]nonane-7-carboxylate (133 mg, 40% over 2 steps). LCMS (m/z): [M+H]⁺ calc'd for C₂₄H₂₆NO₅, 408.2; found 408.

The following intermediate was synthesized using similar conditions as those described above along with appropriate starting materials.

| Structure and Name | Intermediate# | LCMS |
|---|---|---|
| 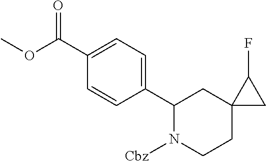 [SMILES: COC(=O)c1ccc(C2CC3(CCN2C(=O)OCc2ccccc2)CC3F)cc1] | B16 From reduction of B17 | LCMS (m/z): [M + H]⁺ calc'd for C₂₃H₂₅FNO₄, 398.2; found, 398 |
| benzyl 1-fluoro-5-(4-(methoxycarbonyl)phenyl)-6- | | |
| azaspiro[2.5]octane-6-carboxylate | | |

### Synthesis of benzyl 1-bromo-1-fluoro-5-(4-(methoxycarbonyl)phenyl)-6-azaspiro[2.5]octane-6-carboxylate (B17)

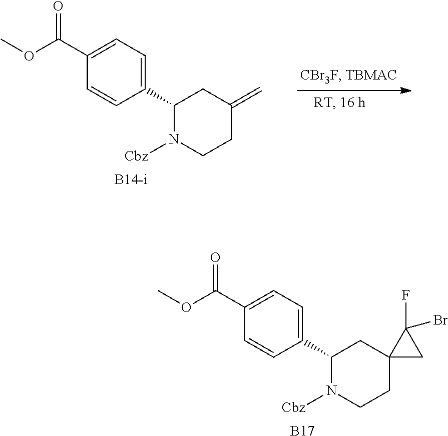

A solution of benzyl 2-(4-(methoxycarbonyl)phenyl)-4-methylenepiperidine-1-carboxylate (600 mg, 1.64 mmol), CBr₃F (1.33 g, 4.92 mmol) and NaOH (196 mg, 4.92 mmol) in a mixture of H₂O (3 mL) and dichloromethane (3 mL) stirred at room temperature for 16 h. The mixture was diluted with additional H₂O (5 mL) and extracted with ethyl acetate (3×10 mL). The combined organic extracts were concentrated and the crude residue purified by column chromatography (petroleum ether:ethyl acetate=3:1) to provide benzyl 1-bromo-1-fluoro-5-(4-(methoxycarbonyl)phenyl)-6-azaspiro[2.5]octane-6-carboxylate (545 mg, 70%). LCMS (m/z): [M+H]⁺ calc'd for C₂₃H₂₄BrFNO₄, 476.1; found, 476.

### Synthesis of methyl 4-(2-hydroxy-7-azaspiro[3.5]nonan-6-yl)benzoate (B19)

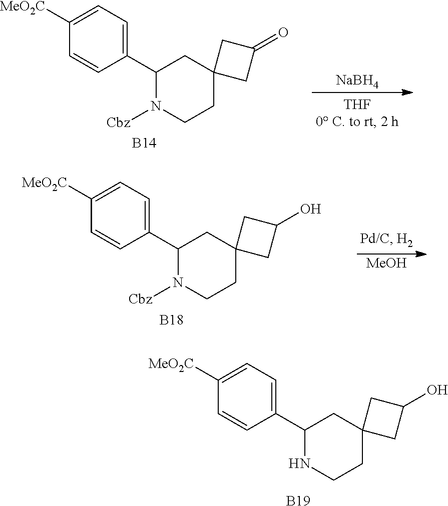

### Step 1: Synthesis of benzyl 2-hydroxy-6-(4-(methoxycarbonyl)phenyl)-7-azaspiro[3.5]nonane-7-carboxylate (B18)

To a solution of B14 (300 mg, 0.74 mmol) in THF (5 mL) was added NaBH₄ (56 mg) at 0° C. The mixture was stirred for 2 h at rt. The reaction mixture was quenched by ice water (3 mL), extracted twice with CH₂Cl₂ (2×5 mL) and washed with brine (10 mL). The combined organic layers were dried over Na₂SO₄, filtered, and concentrated to afford product B18 (170 mg, 0.42 mmol, yield 57%) as yellow oil. LCMS (m/z): [M+H]⁺ calc'd for C₂₄H₂₈NO₅, 410.2; found, 410.

### Synthesis of ethyl 4-(3-azabicyclo[3.1.0]hexan-2-yl)benzoate (C2

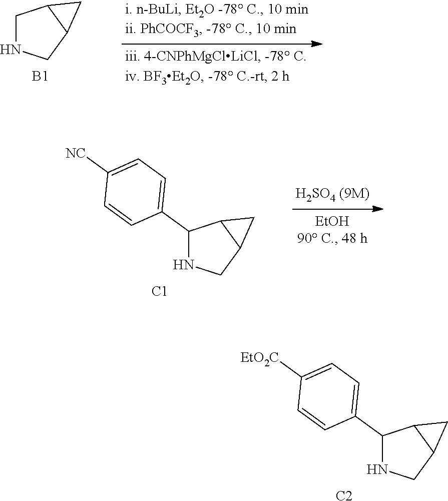

### Step 1: Synthesis of 4-(3-azabicyclo[3.1.0]hexan-2-yl)benzonitrile (C1)

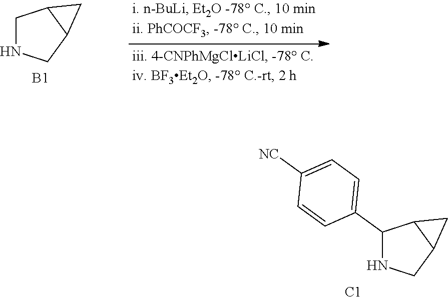

i-PrMgCl•LiCl (6.9 mL, 9.0 mmol, 1.3 M in hexane) was cooled to 0° C. and a solution of 4-bromobenzonitrile (2.19 g, 12.0 mmol) in THF (6.9 mL) was added dropwise under nitrogen. The resulting solution was stirred at 0° C. for 2 h.

Separately, a solution of 3-azabicyclo[3.1.0]hexane (500 mg, 6.02 mmol) in anhydrous ether (12 mL) was cooled to −78° C. and n-BuLi (2.5 mL, 6.0 mmol, 2.4 M in hexanes) was added dropwise under a nitrogen atmosphere. The resulting solution was stirred at that temperature for 10 min prior to the addition of a solution of PhCOCF₃ (1.26 g, 7.22 mmol) in anhydrous ether (6 mL). The resulting mixture was stirred at −78° C. for 10 min prior to the addition of the previously prepared organometallic nucleophile (13.8 mL, 9.02 mmol) in one portion, followed immediately by the addition of boron trifluoride etherate (1.02 g, 7.22 mmol). Subsequently, the reaction vessel was taken out of the low temperature bath and stirred at room temperature for 2 h. The reaction mixture was then cooled to 0° C. and quenched by the addition of methanol (10 mL). The mixture was diluted with 2M sodium hydroxide (50 mL) and extracted with ethyl acetate (3×100 mL). The combined organic layers were washed with brine, dried over anhydrous Na₂SO₄, filtered, and concentrated in vacuo to provide a crude residue. The residue was purified by flash column chromatography on silica gel to afford product (180 mg, 9%) as a colorless oil.

¹H NMR (300 MHz, CDCl₃): δ 7.62 (d, J=8.3 Hz, 2H), 7.46 (d, J=8.4 Hz, 2H), 4.25 (br s, 1H), 3.07 (dt, J=17.9, 7.2 Hz, 2H), 1.62-1.51 (m, 2H), 0.71 (td, J=7.9, 5.6 Hz, 1H), 0.37 (dd, J=8.9, 4.2 Hz, 1H).

The following intermediates were synthesized using similar conditions as those described in step 1, above, along with appropriate starting materials.

| Structure and Name | Intermediate# | LCMS |
|---|---|---|
| 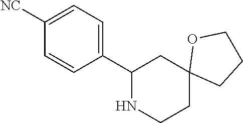 [SMILES: N#Cc1ccc(C2CC3(CCCO3)CCN2)cc1] | C3 | LCMS (m/z): [M + H]⁺ calc'd for C₁₅H₁₉N₂O, 243.1; found, 243.0 |
| 4-(1-oxa-8-azaspiro[4.5]decan-7-yl)benzonitrile | | |
| 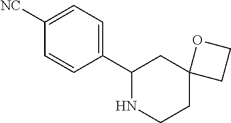 [SMILES: N#Cc1ccc(C2CC3(CCN2)CCO3)cc1] | C4 | LCMS (m/z): [M + H]⁺ calc'd for C₁₄H₁₇N₂O, 229.1; found, 229.0 |
| 4-(1-oxa-7-azaspiro[3.5]nonan-6-yl)benzonitrile | | |
| 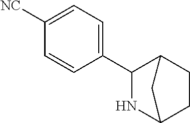 [SMILES: N#Cc1ccc(C2NC3CCC2C3)cc1] | C5 | LCMS (m/z): [M + H]⁺ calc'd for C₁₃H₁₅N₂, 199.1; found, 198.9 |
| 4-(2-azabicyclo[2.2.1]heptan-3-yl)benzonitrile | | |
| 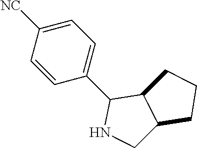 | C6 | LCMS (m/z): [M + H]⁺ calc'd for C₁₄H₁₇N₂, 213.1; found, 213.0 |
| 4-(octahydrocyclopenta[c]pyrrol-1-yl)benzonitrile | | |
| 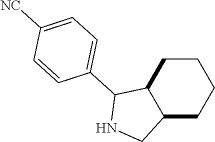 | C7 | LCMS (m/z): [M + H]⁺ calc'd for C₁₅H₁₉N₂, 227.2; found, 227.0 |
| 4-(octahydro-1H-isoindol-1-yl)benzonitrile | | |
| 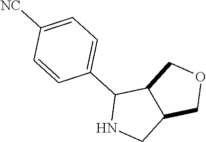 | C8 | LCMS (m/z): [M + H]⁺ calc'd for C₁₃H₁₅N₂O, 215.1; found, 215.2 |
| 4-(hexahydro-1H-furo[3,4-c]pyrrol-4-yl)benzonitrile | | |
| 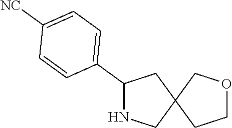 [SMILES: N#Cc1ccc(C2CC3(CCOC3)CN2)cc1] | C24 (from CAS 175-97-3) | LCMS (m/z): [M + H]⁺ calc'd for C₁₄H₁₇N₂O, 229.1; found, 229.2 |
| 4-(2-oxa-7-azaspiro[4.4]nonan-8-yl)benzonitrile | | |
| 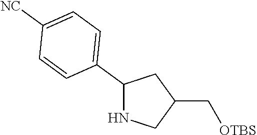 [SMILES: CC(C)(C)[Si](C)(C)OCC1CNC(c2ccc(C#N)cc2)C1] | C25 (from B4) | LCMS (m/z): [M + H]⁺ calc'd for C₁₈H₂₉N₂OSi, 317.2; found, 317.1 |
| 4-(4-(((tert- | | |
| butyldimethylsilyl)oxy)methyl)pyrrolidin-2- | | |
| yl)benzonitrile | | |
| 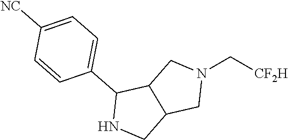 [SMILES: N#Cc1ccc(C2NCC3CN(CC(F)F)CC32)cc1] | C26 | LCMS (m/z): [M + H]⁺ calc'd for C₁₅H₁₈F₂N₃, 278.1; found, 278.1 |
| 4-(5-(2,2-difluoroethyl)octahydropyrrolo[3,4- | | |
| c]pyrrol-1-yl)benzonitrile | | |
| 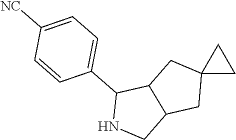 [SMILES: N#Cc1ccc(C2NCC3CC4(CC4)CC32)cc1] | C27 (from B9) | LCMS (m/z): [M + H]⁺ calc'd for C₁₆H₁₉N₂, 239.2; found, 239.1 |
| 4-(hexahydro-1H-spiro[cyclopenta[c]pyrrole-5,1′- | | |
| cyclopropan]-1-yl)benzonitrile | | |
| 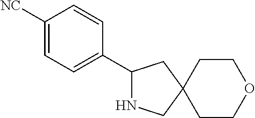 [SMILES: N#Cc1ccc(C2CC3(CCOCC3)CN2)cc1] | C28 | LCMS (m/z): [M + H]⁺ calc'd for C₁₅H₁₉N₂O, 243.1; found, 243.1 |
| 4-(8-oxa-2-azaspiro[4.5]decan-3-yl)benzonitrile | | |
| 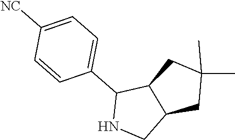 [SMILES: CC1(C)C[C@H]2CNC(c3ccc(C#N)cc3)[C@H]2C1] | C29 | LCMS (m/z): [M + H]⁺ calc'd for C₁₆H₂₁N₂, 241.2; found, 241 |
| 4-((3aR,6aS)-5,5- | | |
| dimethyloctahydrocyclopenta[c]pyrrol-1- | | |
| yl)benzonitrile | | |
| 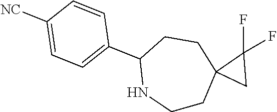 [SMILES: N#Cc1ccc(C2CCC3(CCN2)CC3(F)F)cc1] | C30 (from B10) | LCMS (m/z): [M + H]⁺ calc'd for C₁₅H₁₇F₂N₂, 263.1; found, 263.1 |
| 4-(1,1-difluoro-6-azaspiro[2.6]nonan-7- | | |
| yl)benzonitrile | | |
| 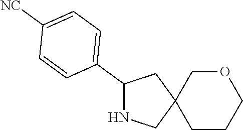 [SMILES: N#Cc1ccc(C2CC3(CCCOC3)CN2)cc1] | C31 | LCMS (m/z): [M + H]⁺ calc'd for C₁₅H₁₉N₂O, 243.1; found, 243.1 |
| 4-(7-oxa-2-azaspiro[4.5]decan-3-yl)benzonitrile | | |
| 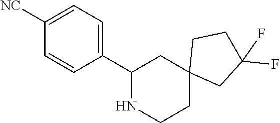 [SMILES: N#Cc1ccc(C2CC3(CCN2)CCC(F)(F)C3)cc1] | F22 | LCMS (m/z): [M + H]+ calc'd for C₁₆H₁₉F₂N₂, 243.1; found, 243.1 |
| 4-(2,2-difluoro-8-azaspiro[4.5]decan-7- | | |
| yl)benzonitrile | | |
| 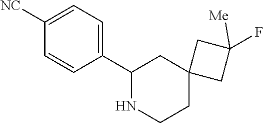 [SMILES: CC1(F)CC2(CCNC(c3ccc(C#N)cc3)C2)C1] | F28 (from F27) | LCMS (m/z): [M + H]+ calc'd for C₁₆H₂₀FN₂, 259; found, 259 |
| 4-(2-fluoro-2-methyl-7-azaspiro[3.5]nonan-6- | | |
| yl)benzonitrile | | |

### Step 2: Synthesis of ethyl 4-(3-azabicyclo[3.1.0]hexan-2-yl)benzoate (C2)

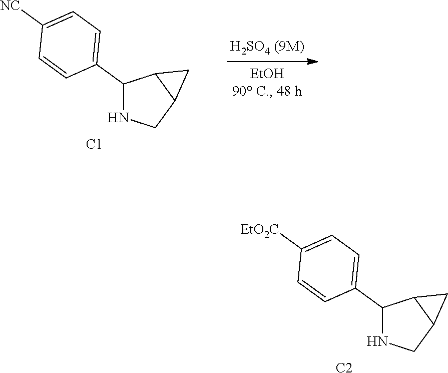

To a solution of 4-(3-azabicyclo[3.1.0]hexan-2-yl)benzonitrile (160 mg, 0.870 mmol) in ethanol (5 mL) was added sulfuric acid (2 mL, 9 M) and the reaction mixture heated to 90° C. and stirred at that temperature for 48 h. The reaction was quenched by the addition of saturated sodium carbonate solution (50 mL) and extracted with CH₂Cl₂(3×30 mL). The combined organic layers were washed with brine, dried over anhydrous Na₂SO₄, filtered, and concentrated in vacuo to obtain a crude residue. The residue was purified by flash column chromatography (SiO₂, 10:1 CH₂Cl₂/MeOH) to afford product (90 mg, 400) as a colorless oil. LCMS (m/z): [M+H]⁺ calc'd for C₁₄H₁₈N20, 232.1; found, 232.1.

The following intermediates were synthesized using similar conditions as those described in step 2, above, along with appropriate starting materials.

| | | Isomer Separation Method and | |
|---|---|---|---|
| Structure and Name | Intermediate# | Retention Time (if any) | LCMS |
| 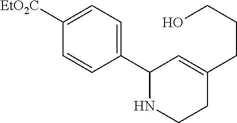 [SMILES: CCOC(=O)c1ccc(C2C=C(CCCO)CCN2)cc1] | C9 | | LCMS (m/z): [M + H]⁺ calc'd for C₁₇H₂₄NO₃, 290.2; found, 290.1 |
| ethyl 4-(4-(3-hydroxypropyl)- | | | |
| 1,2,5,6-tetrahydropyridin-2- | | | |
| yl)benzoate | | | |
| 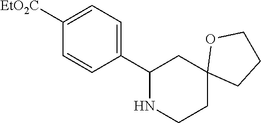 [SMILES: CCOC(=O)c1ccc(C2CC3(CCCO3)CCN2)cc1] | C10 | | LCMS (m/z): [M + H]⁺ calc'd for C₁₇H₂₄NO₃, 290.2; found, 290.1 |
| ethyl 4-(1-oxa-8- | | | |
| azaspiro[4.5]decan-7- | | | |
| yl)benzoate | | | |
| 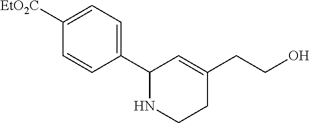 [SMILES: CCOC(=O)c1ccc(C2C=C(CCO)CCN2)cc1] | C11 | | LCMS (m/z): [M + H]⁺ calc'd for C₁₆H₂₂NO₃ , 276.2; found, 276.1 |
| ethyl 4-(4-(2-hydroxyethyl)- | | | |
| 1,2,5,6-tetrahydropyridin-2- | | | |
| yl)benzoate | | | |
| 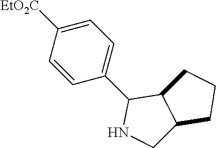 | C12 (Isomer 1) | Column: CHIRAL OJ 4.6 × 250 mm mm I.D., 5 μm Mobile phase: n- Hexane(0.1% DEA):EtOH(0.1% DEA) = 90:10 Flow rate: 1.0 mL/min, Run time: 30 min, Column temperature: 40° C. Retention time = 15.681 min | LCMS (m/z): [M + H]⁺ calc'd for C₁₆H₂₂NO₂, 260.2; found, 260.0 |
| ethyl 4- | | | |
| (octahydrocyclopenta[c]pyrrol- | | | |
| 1-yl)benzoate | | | |
| 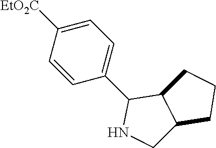 | C12 (Isomer 2) | Column: CHIRAL OJ 4.6 × 500 mm mm I.D., 3 μm Mobile phase: n- Hexane(0.1% DEA):EtOH(0.1% DEA) = 90:10 Flow rate: 1.0 mL/min, Run time: 30 min, Column temperature: 40° C. Retention time = 14.452 min | LCMS (m/z): [M + H]⁺ calc'd for C₁₆H₂₂NO₂, 260.2; found, 260.0 |
| ethyl 4- | | | |
| (octahydrocyclopenta[c]pyrrol- | | | |
| 1-yl)benzoate | | | |
| 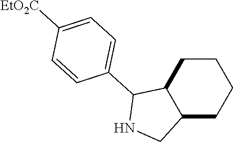 | C13 | | LCMS (m/z): [M + H]⁺ calc'd for C₁₇H₂₄NO₂, 274.2; found, 274.0 |
| ethyl 4-(octahydro-1H-isoindol- | | | |
| 1-yl)benzoate | | | |
| 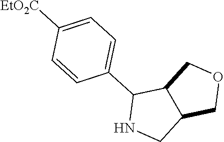 | C14 | | LCMS (m/z): [M + H]⁺ calc'd for C₁₅H₂₀NO₃, 262.1; found, 262 |
| ethyl 4-(hexahydro-1H- | | | |
| furo[3,4-c]pyrrol-4-yl)benzoate | | | |
| 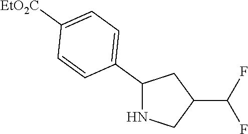 [SMILES: CCOC(=O)c1ccc(C2CC(C(F)F)CN2)cc1] | C32 (From F5) | | LCMS (m/z): [M + H]⁺ calc'd for C₁₄H₁₈F₂NO₂, 270.1; found, 270.1 |
| ethyl 4-(4- | | | |
| (difluoromethyl)pyrrolidin-2- | | | |
| yl)benzoate | | | |
| 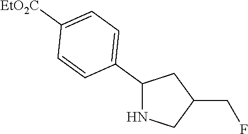 [SMILES: CCOC(=O)c1ccc(C2CC(CF)CN2)cc1] | C33 (From F6) | | LCMS (m/z): [M − H]⁺ calc'd for C₁₄H₁₉FNO₂, 252.1; found, 252.1 |
| ethyl 4-(4- | | | |
| (fluoromethyl)pyrrolidin-2- | | | |
| yl)benzoate | | | |
| 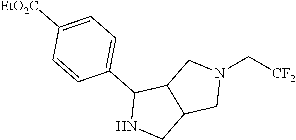 | C34 (From C26) | | LCMS (m/z): [M + H]⁺ calc'd for C₁₇H₂₃F₂N₂O₂, 325.2; found, 325.1 |
| ethyl 4-(5-(2,2- | | | |
| difluoroethyl)octahydropyrrolo[3,4- | | | |
| c]pyrrol-1-yl)benzoate | | | |
| 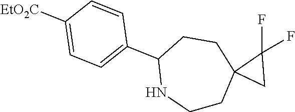 [SMILES: CCOC(=O)c1ccc(C2CCC3(CCN2)CC3(F)F)cc1] | C35 (From C30) | | LCMS (m/z): [M + H]⁺ calc'd for C₁₇H₂₂F₂NO₂, 310.1; found, 310.1 |
| ethyl 4-(1,1-difluoro-6- | | | |
| azaspiro[2.6]nonan-7- | | | |
| yl)benzoate | | | |
| 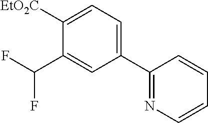 [SMILES: CCOC(=O)c1ccc(-c2ccccn2)cc1C(F)F] | C36 (from F10) | | LCMS (m/z): [M + H]⁺ calc'd for C₁₅H₁₄F₂NO₂, 278.1; found, 278.1 |
| ethyl 2-(difluoromethyl)-4- | | | |
| (pyridin-2-yl)benzoate | | | |
| 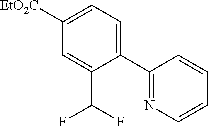 [SMILES: CCOC(=O)c1ccc(-c2ccccn2)c(C(F)F)c1] | C37 (from F11) | | LCMS (m/z): [M + H]⁺ calc'd for C₁₅H₁₄F₂NO₂, 278.1; found, 278.1 |
| ethyl 3-(difluoromethyl)-4- | | | |
| (pyridin-2-yl)benzoate | | | |

### Synthesis of 4-(2-oxa-8-azaspiro[4.5]decan-7-yl)benzonitrile (C38)

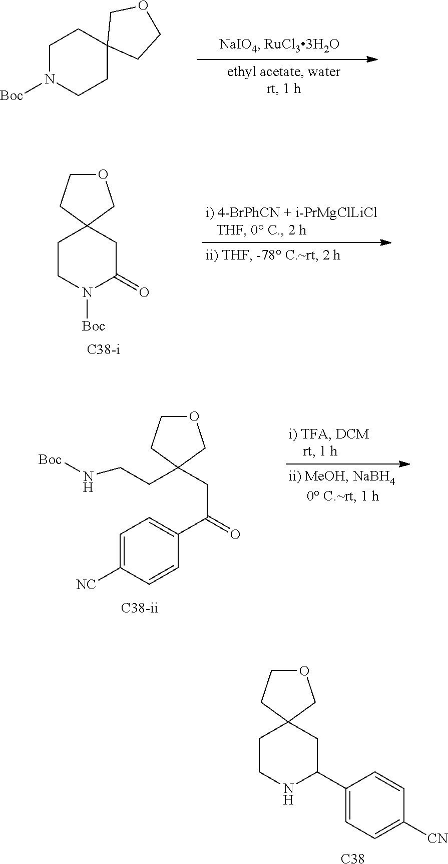 [SMILES: CC(C)(C)OC(=O)N1CCC2(CCOC2)CC1 ; CC(C)(C)OC(=O)N1CCC2(CCOC2)CC1=O ; CC(C)(C)OC(=O)NCCC1(CC(=O)c2ccc(C#N)cc2)CCOC1 ; N#Cc1ccc(C2CC3(CCN2)CCOC3)cc1 ; "C38"]

### Step 1: Synthesis of tert-butyl 7-oxo-2-oxa-8-azaspiro[4.5]decane-8-carboxylate (C38-i)

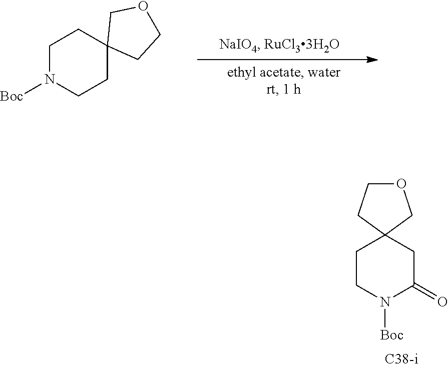 [SMILES: CC(C)(C)OC(=O)N1CCC2(CCOC2)CC1 ; CC(C)(C)OC(=O)N1CCC2(CCOC2)CC1=O]

To a solution of NaIO₄ (2.93 g, 13.7 mmol) in water (75 mL) was added RuCl₃·3H₂O (715 mg, 2.74 mmol). To the resultant yellow solution was added a solution of tert-butyl 2-oxa-8-azaspiro[4.5]decane-8-carboxylate (3.30 g, 13.7 mmol) in ethyl acetate (120 mL) and this mixture stirred at room temperature for 1 h. The organic layer was separated, and the aqueous layer was extracted twice with ethyl acetate. The combined organic layers were dried over Na2SO₄, filtered and concentrated. The residue was purified by column chromatography with (ethyl acetate/petroleum ether=1/1) to afford tert-butyl 7-oxo-2-oxa-8-azaspiro[4.5]decane-8-carboxylate (1.0 g, 29%) as a yellow oil. LCMS (m/z): [M-tBu]⁺ calc'd for C₉H₁₄NO₄, 200.1; found, 200.

### Step 2: Synthesis of tert-butyl (2-(3-(2-(4-cyanophenyl)-2-oxoethyl)tetrahydrofuran-3-yl)ethyl)carbamate (C38-ii)

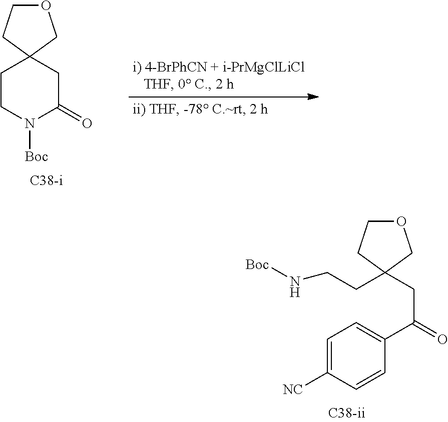 [SMILES: CC(C)(C)OC(=O)N1CCC2(CCOC2)CC1=O ; CC(C)(C)OC(=O)NCCC1(CC(=O)c2ccc(C#N)cc2)CCOC1]

To a solution of i-PrMgCl•LiC1 (19.3 mL, 25.0 mmol, 1.3 M in THF) at 0° C. was added a solution of 4-BrPhCN (5.26 g, 28.9 mmol) in THF (25 mL). This mixture was then stirred at 0° C. for 2 h before being added carefully (over 15 min) to a solution of tert-butyl 7-oxo-2-oxa-8-azaspiro[4.5]decane-8-carboxylate (983 mg, 3.85 mmol) in THF (15 mL) at −78° C. The resultant mixture was stirred at −78° C. for 15 min, warmed to 0° C. and stirred for 1 h. The reaction was quenched with ice water (30 mL), extracted with ethyl acetate (3×50 mL), dried with Na₂SO₄ and concentrated under reduced pressure. The residue was purified by column chromatography with (EA/PE=1/2) to afford tert-butyl (2-(3-(2-(4-cyanophenyl)-2-oxoethyl)tetrahydrofuran-3-yl)ethyl)carbamate (255 mg, 18%) as yellow oil. LCMS (m/z): [M-Boc]⁺ calc'd for C₁₅H₁₉N₂O₂, 259.1; found, 259.

### Step 3: Synthesis of 4-(2-oxa-8-azaspiro[4.5]decan-7-yl)benzonitrile (C38)

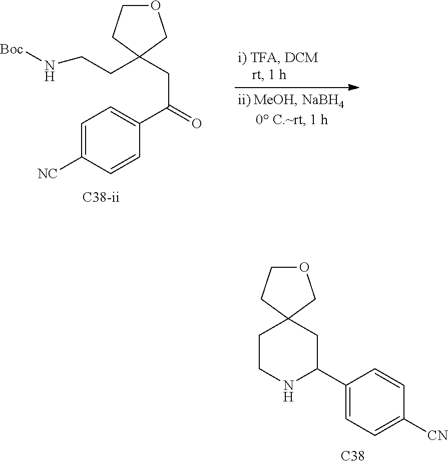 [SMILES: CC(C)(C)OC(=O)NCCC1(CC(=O)c2ccc(C#N)cc2)CCOC1 ; N#Cc1ccc(C2CC3(CCN2)CCOC3)cc1 ; "C38"]

To a solution of tert-butyl (2-(3-(2-(4-cyanophenyl)-2-oxoethyl)tetrahydrofuran-3-yl)ethyl)carbamate (100 mg, 0.28 mmol) in DCM (10 mL) was added TFA (50 μL). The mixture was stirred at room temperature for 1 h. The mixture was concentrated under N₂. The residue was dissolved in MeOH (10 mL) and NaBH₄ (106 mg, 2.8 mmol) was added at 0° C. The resulting solution was stirred for 1 h and quenched by water (10 mL), extracted with DCM (3×15 mL), dried with Na₂SO₄ and concentrated to provide 4-(2-oxa-8-azaspiro[4.5]decan-7-yl)benzonitrile (64 mg, crude) as yellow solid. LCMS (m/z): [M+H]⁺ calc'd for C₁₅H₁₉N₂O, 243.1; found, 243.

### Synthesis of benzyl (5S)-1,1-difluoro-5-(4-(methoxycarbonyl)phenyl)-6-azaspiro[2.5]octane-6-carboxylate (D2)

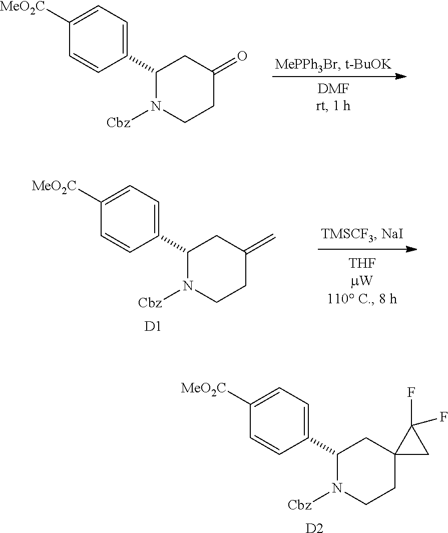

### Step 1: Synthesis of benzyl (S)-2-(4-(methoxycarbonyl)phenyl)-4-methylenepiperidine-1-carboxylate (D1)

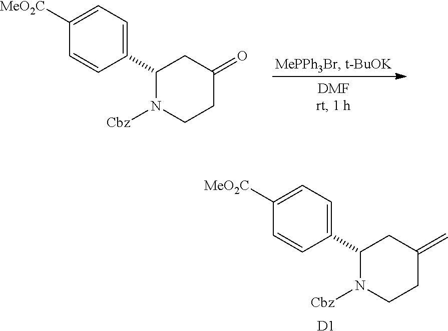

Benzyl (S)-2-(4-(methoxycarbonyl)phenyl)-4-oxopiperidine-1-carboxylate (racemate CAS: 2238811-87-3, 200 mg, 0.550 mmol), which was separated on a chiral column (retention time=4.855 min on IG-H column −0.46 cm I. D.×15 cm L at 2.5 mL/min), methyltriphenylphosphonium bromide (207 mg, 0.980 mmol) and t-BuOK (122 mg, 1.10 mol) were dissolved in DMF (10 mL) and stirred at room temperature for 1 h. The mixture was diluted with H₂O and extracted with ethyl acetate. The combined organic phases were dried over anhydrous Na₂SO₄, filtered, and concentrated in vacuo. The resulting residue was purified by flash column chromatography (SiO₂, 5:1 petroleum ether/ethyl acetate) to afford the pure desired product (90 mg, 45%) as colorless oil. LCMS (m/z): [M+H]⁺ calc'd for C₂₂H₂₄NO₂, 366.2; found, 366.

| Structure and Name | Intermediate | LCMS |
|---|---|---|
| 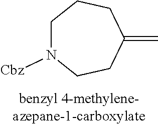 [SMILES: C=C1CCCN(C(=O)OCc2ccccc2)CC1] | D3 (from CAS 83621-33-4) | LCMS (m/z): [M + H]⁺ calc'd for C₁₅H₂₀NO₂, 246.1; found, 246.1 |

### Step 2: Synthesis of benzyl (5S)-1,1-difluoro-5-(4-(methoxycarbonyl)phenyl)-6-azaspiro[2.5]octane-6-carboxylate (D2)

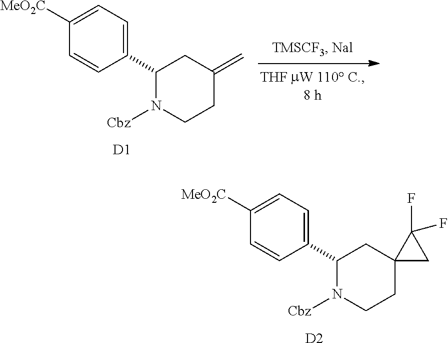

Benzyl (S)-2-(4-(methoxycarbonyl)phenyl)-4-methylenepiperidine-1-carboxylate (90 mg, 0.24 mmol), TMSCF₃ (140 mg, 0.980 mmol) and NaI (36 mg, 0.24 mol) was dissolved in THF (2 mL) in microwave tube and stirred at 110° C. for 8 h. The mixture was diluted with H₂O and extracted with ethyl acetate. The combined organic phases were dried over anhydrous Na₂SO₄, filtered, and concentrated in vacuo. The resulting crude residue was purified by flash column chromatography (SiO₂, 8:1 petroleum ether/ethyl acetate) to afford the desired product (80 mg, 80%) as yellow oil. LCMS (m/z): [M+H]⁺ calc'd for C₂₃H₂₄F₂NO₄, 416.2; found, 416.

The following intermediates were synthesized using similar conditions as those described above, along with appropriate starting materials.

| Structure and Name | Intermediate | LCMS |
|---|---|---|
| 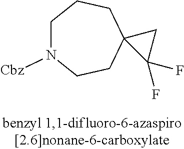 [SMILES: O=C(OCc1ccccc1)N1CCCC2(CC1)CC2(F)F] | D4 (from D3) | LCMS (m/z): [M + H]⁺ calc'd for C₁₆H₂₀F₂NO₂, 296.1; found, 296.1 |

### Synthesis of methyl 4-(1-oxa-7-azaspiro[3.5]nonan-6-yl)benzoate (D7)

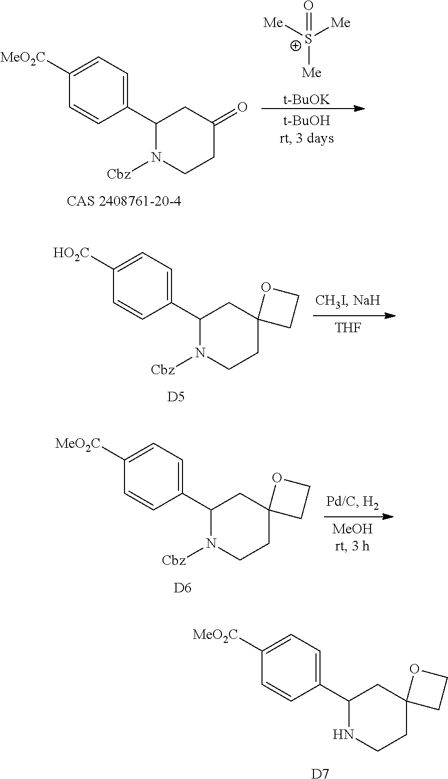

### Step 1: Synthesis of 4-(7-((benzyloxy)carbonyl)-1-oxa-7-azaspiro[3.5]nonan-6-yl)benzoic acid (D5)

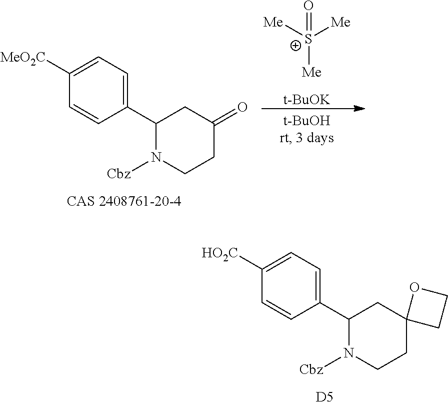

Trimethylsulfoxonium iodide (12 g, 54.5 mmol) and t-BuOK (6.1 g, 54.5 mmol) were dissolved in THF (50 mL) and the misture stirred at 50° C. for 1 h. Then benzyl 2-(4-(methoxycarbonyl)phenyl)-4-oxopiperidine-1-carboxylate (5 g, 13.6 mmol) was added and stirred overnight. The mixture was evaporated and afford crude product D5 (7 g).

### Step 2: Synthesis of benzyl 6-(4-(methoxycarbonyl)phenyl)-1-oxa-7-azaspiro[3.5]nonane-7-carboxylate (D6)

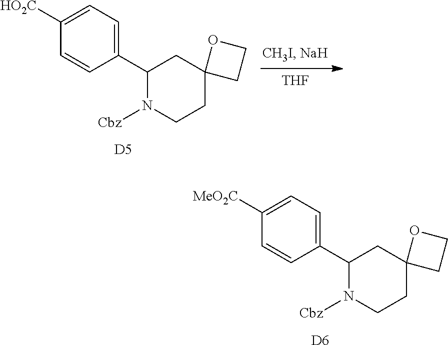

Benzyl 2-(4-(methoxycarbonyl)phenyl)-4-oxopiperidine-1-carboxylate (7 g, 18.3 mmol) and NaH (3.67 g, 91.8 mmol) were dissolved in THF (50 mL) at 0° C. and the mixture stirred for 30 min. Methyl iodide (26 g, 1 mol) was added and the mixture warmed to rt and stirred at that temperature for 8 h. The mixture was diluted with H₂O and extracted with ethyl acetate (50 mL×3). The combined organic phases were dried over anhydrous Na₂SO₄, filtered, and evaporated in vacuo and the resulting residue purified by flash column chromatography (silica gel, 8:1 petroleum ether/ethyl acetate) to afford pure D6 (2 g, 37% yield over two steps).

### Synthesis of methyl 4-((2R,3S)-3-hydroxyazetidin-2-yl)benzoate (E2)

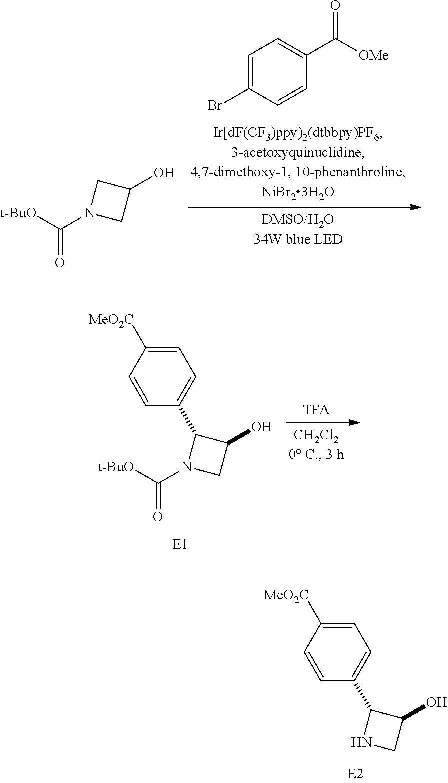 [SMILES: COC(=O)c1ccc(Br)cc1 ; CC(C)(C)OC(=O)N1CC(O)C1 ; COC(=O)c1ccc([C@@H]2[C@@H](O)CN2C(=O)OC(C)(C)C)cc1 ; COC(=O)c1ccc([C@H]2NC[C@@H]2O)cc1]

### Step 1: Synthesis of tert-butyl (2R,3S)-3-hydroxy-2-(4-(methoxycarbonyl)phenyl)azetidine-1-carboxylate (E1)

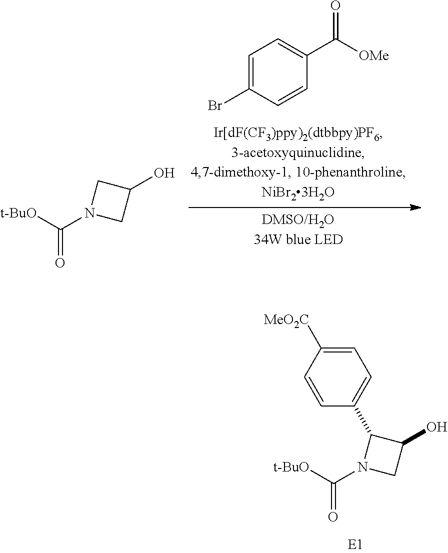 [SMILES: COC(=O)c1ccc(Br)cc1 ; CC(C)(C)OC(=O)N1CC(O)C1 ; COC(=O)c1ccc([C@@H]2[C@@H](O)CN2C(=O)OC(C)(C)C)cc1]

To a solution of methyl 4-bromobenzoate (2.16 g, 10.0 mmol) and tert-butyl 3-hydroxyazetidine-1-carboxylate (2.60 g, 15.0 mmol) in DMSO (22 mL) and H₂O (9.03 g, 502 mmol) was added Ir[dF(CF₃)ppy]₂(dtbbpy)PF₆ (112 mg, 0.100 mmol), 3-acetoxyquinuclidine (1.86 g, 11.0 mmol), 4,7-dimethoxy-1,10-phenanthroline (24 mg, 0.10 mmol) and NiBr₂•3H₂O (27 mg, 0.10 mmol). The mixture was stirred at room temperature for 16 h under 34 W blue LED, then diluted with H₂O (50 mL), extracted with ethyl acetate (3×50 mL). The combined organic layers were washed with brine (3×20 mL), dried over anhydrous Na₂SO₄ and concentrated to obtain crude desired product. The crude residue was purified by prep-HPLC (MeCN/H₂O+0.1% FA) to give pure desired compound (380 mg, 12%) as a white solid. LCMS (m/z): [M+H]⁺ calc'd for C₁₆H₂₂N₂O₅, 308.1; found, 308.

### Step 2: Synthesis of methyl 4-(3-hydroxyazetidin-2-yl)benzoate (E2)

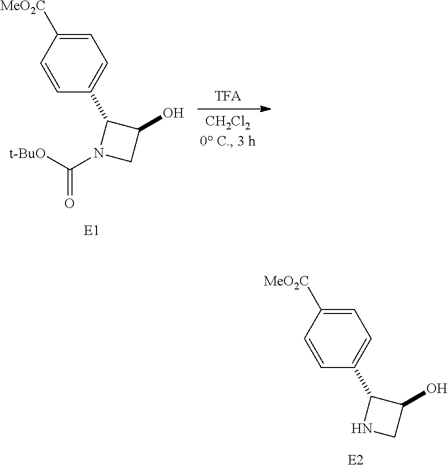 [SMILES: COC(=O)c1ccc([C@@H]2[C@@H](O)CN2C(=O)OC(C)(C)C)cc1 ; COC(=O)c1ccc([C@H]2NC[C@@H]2O)cc1]

To a solution of tert-butyl-3-hydroxy-2-(4-(methoxycarbonyl)phenyl)azetidine-1-carboxylate (300 mg, 0.240 mmol) in CH₂Cl₂ (5 mL) at 0° C. was added TFA (1 mL) under nitrogen. The mixture was stirred for 3 h, then the mixture was diluted with CH₂Cl₂ (20 mL) and washed with saturated aqueous NaHCO₃ (10 mL), then brine (10 mL), dried over anhydrous Na₂SO₄, and concentrated in vacuo to obtain a crude residue. Crude material was purified by flash column chromatography (SiO₂, 0-10% CH₂Cl₂ in ethyl acetate) to give compound pure desired compound (45 mg, 75% o) as a yellow oil. LCMS (m/z): [M+H]⁺ calc'd for C₁₁H₁₄NO₃, 208.1; found, 208.0.

The following intermediate was synthesized using similar conditions as those described in Step 2, above, along with appropriate starting materials.

| Structure and Name | Intermediate | LCMS |
|---|---|---|
| 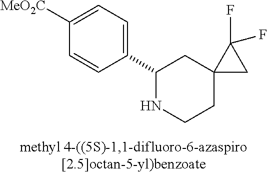 | E3 (From D2) | LCMS (m/z): [M + H]⁺ calc'd for C₁₅H₁₈F₂NO₂, 282.1; found, 282.0 |
| 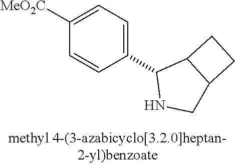 | E4 | LCMS (m/z): [M + H]⁺ calc'd for C₁₄H₁₈NO₂, 232.1; found, 232.1 |
| 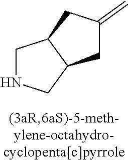 | E5 (from CAS 139228-12-9) | LCMS (m/z): [M + H]⁺ calc'd for C₈H₁₄N, 124.1; found, 124.3 |
| 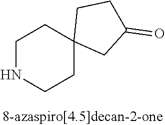 [SMILES: O=C1CCC2(CCNCC2)C1] | F18 (from F17) | LCMS (m/z): [M + H]⁺ calc'd for C₉H₁₆NO, 154; found, 154 |
| 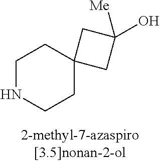 [SMILES: CC1(O)CC2(CCNCC2)C1] | F24 (from CAS 1460229-47-3) | LCMS (m/z): [M + H]⁺ calc'd for C₉H₁₈NO, 156; found, 156 |

### Synthesis of benzyl (3aR,6aS)-tetrahydro-1H-spiro[cyclopenta[c]pyrrole-5,1′-cyclopropane]-2(3H)-carboxylate (E6)

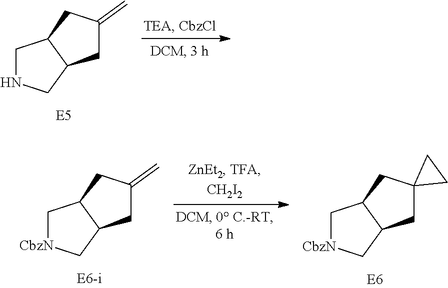

### Step 1: Synthesis of benzyl (3aR,6aS)-5-methylenehexahydrocyclopenta[c]pyrrole-2(1H)-carboxylate (E6-i)

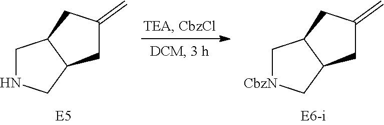

To a solution of (3aR,6aS)-5-methyleneoctahydrocyclopenta[c]pyrrole (750 mg, 6.10 mmol) and triethylamine (1.85 g, 18.3 mmol) in DCM (20 mL) was added CbzCl (1.04 g, 6.10 mmol). The resulting solution stirred at room temperature for 3 h. Then the mixture was purified by column chromatography to afford benzyl (3aR,6aS)-5-methylenehexahydrocyclopenta[c]pyrrole-2(1H)-carboxylate (1.3 g, 95%) as yellow liquid. LCMS (m/z): [M+H]⁺ calc'd for C₁₆H₂₀NO₂, 258.1; found, 258.3.

The following intermediate was synthesized using similar conditions as those described in Step 1, above, along with appropriate starting materials.

| Structure and Name | Intermediate | LCMS |
|---|---|---|
| 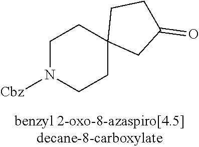 [SMILES: O=C1CCC2(CCN(C(=O)OCc3ccccc3)CC2)C1] | F19 (From F18) | LCMS (m/z): [M + H]⁺ calc'd for C₁₇H₂₂NO₃, 288; found, 288 |
| 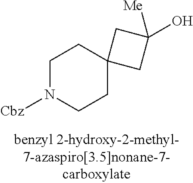 [SMILES: CC1(O)CC2(CCN(C(=O)OCc3ccccc3)CC2)C1] | F25 (from F24) | LCMS (m/z): [M + H]⁺ calc'd for C₁₇H₂₄NO₃, 290.2; found, 290.1 |

### Synthesis of benzyl (3aR,6aS)-tetrahydro-1H-spiro[cyclopenta[c]pyrrole-5,1′-cyclopropane]-2(3H)-carboxylate (E6)

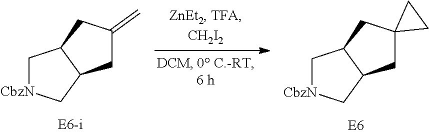 [SMILES: C=C1C[C@@H]2CN(C(=O)OCc3ccccc3)C[C@@H]2C1 ; O=C(OCc1ccccc1)N1C[C@@H]2CC3(CC3)C[C@@H]2C1]

To a solution of diethyl zinc (3.6 g, 29 mmol) in dichloromethane (20 mL) cooled to −60° C. was slowly added diiodomethane (10.2 g, 58.0 mmol). The resulting solution was stirred at −60° C. for 1 h, then a solution of benzyl (3aR,6aS)-5-methylenehexahydrocyclopenta[c]pyrrole-2(1H)-carboxylate (1.3 g, 5.80 mmol) in dichloromethane (20 mL) was added. The resultant mixture was stirred at room temperature for 5 h. Then water (50 mL) was added and the aqueous solution was extracted by ethyl acetate (3×20 mL). The combined organic extracts were evaporated and purified by column chromatography to provide benzyl (3aR,6aS)-tetrahydro-1H-spiro[cyclopenta[c]pyrrole-5,1′-cyclopropane]-2(3H)-carboxylate (1.15 g, 88%) as a white solid. LCMS (m/z): [M+H]⁺ calc'd for C₁₇H₂₂NO₂, 272.2; found, 272.1.

### Synthesis of benzyl 5-(difluoromethyl)hexahydrocyclopenta[c]pyrrole-2(1H)-carboxylate (E10)

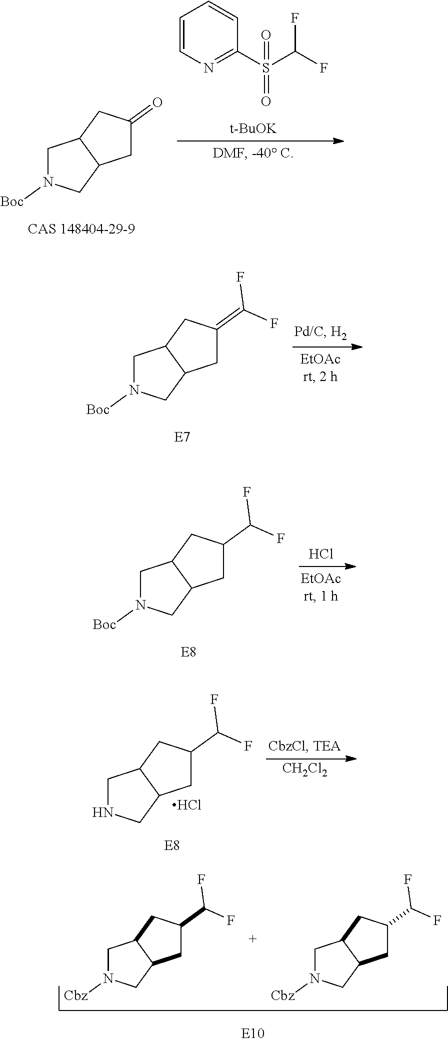

### Step 1: Synthesis of tert-butyl 5-(difluoromethylene)hexahydrocyclopenta[c]pyrrole-2(1H)-carboxylate (E7)

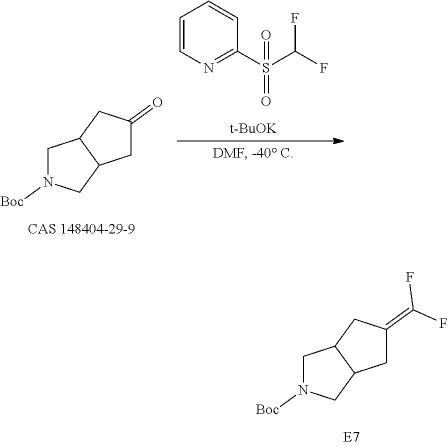 [SMILES: "t-BuOK" ; O=S(=O)(c1ccccn1)C(F)F ; CC(C)(C)OC(=O)N1CC2CC(=O)CC2C1 ; CC(C)(C)OC(=O)N1CC2CC(=C(F)F)CC2C1]

A mixture of tert-butyl 5-oxohexahydrocyclopenta[c]pyrrole-2(1H)-carboxylate (225 mg, 1 mmol), 2-((difluoromethyl)sulfonyl)pyridine (193 mg, 1 mmol), KOtBu (112 mg, 1 mmol) in DMF (5 mL) was stirred at −40° C. for 2 hours. The reaction mixture was poured into water and the residue was extracted with ethyl acetate (50 mL×2). The combined organic layers were dried over anhydrous Na₂SO₄, filtered, and concentrated. The residue was purified by flash chromatography (SiO2, 1:9 ethyl acetate/petroleum ether) to provide pure E7 (130 mg, 50% yield) as a colorless oil. LCMS (m/z): [M−55]⁺ calc'd for C₉H₁₂F₂NO₂, 204.1; found, 204.0.

### Step 2: Synthesis of tert-butyl 5-(difluoromethyl)hexahydrocyclopenta[c]pyrrole-2(1H)-carboxylate (E8)

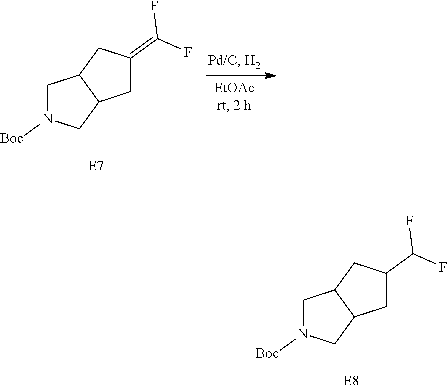 [SMILES: CC(C)(C)OC(=O)N1CC2CC(=C(F)F)CC2C1 ; CC(C)(C)OC(=O)N1CC2CC(C(F)F)CC2C1]

A mixture of E7 (259 mg, 1 mmol) and Pd/C (10 mg) in ethyl acetate (5 mL) was stirred at rt for 2 hours under H₂. The reaction mixture was filtered and concentrated to provide the product E8 (248 mg, 95% yield) as a yellow oil. LCMS (m/z): [M−55]⁺ calc'd for C₉H₁₄F₂NO₂, 206.1; found, 206.0.

### Synthesis of 5-(difluoromethyl)octahydrocyclopenta[c]pyrrol-2-ium chloride (E9)

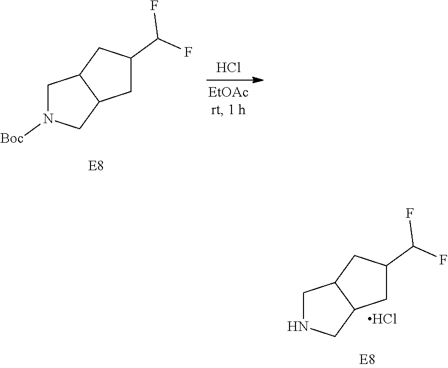 [SMILES: CC(C)(C)OC(=O)N1CC2CC(C(F)F)CC2C1 ; Cl ; FC(F)C1CC2CNCC2C1 ; Cl]

A mixture of E8 (261 mg, 1 mmol) in 4 M HCl in ethyl acetate (5 mL) was stirred at rt for 1 hour. The reaction mixture was concentrated to provide the product E9 (153 mg, 95% yield) as a yellow oil. LCMS (m/z): [M+H]⁺ calc'd for C₈H₁₄F₂N, 162.1; found, 162.0.

### Synthesis of benzyl 5-(difluoromethyl)hexahydrocyclopenta[c]pyrrole-2(1H)-carboxylate (E10)

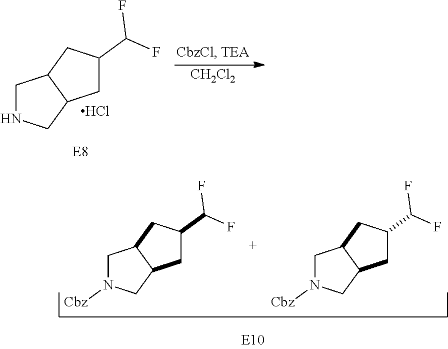

A mixture of E9 (161 mg, 1 mmol), CbzCl (170 mg, 1 mmol) and TEA (202 mg, 2 mmol) in CH₂Cl₂ (5 mL) was stirred at 0° C. to room temperature for 2 hours. The reaction mixture was poured into water and the residue was extracted with ethyl acetate (50 mL×2). The combined organic layers were dried over anhydrous Na₂SO₄, filtered, and concentrated. The residue was purified by flash chromatography (SiO2, 1:9 ethyl acetate/petroleum ether) to provide the product mixture E10 (264 mg, 90% yield) as a colorless oil. LCMS (m/z): [M+H]⁺ calc'd for C₁₆H₂₀F₂NO₂, 296.1; found, 296.0.

The purified racemate of E10 (26 g, 88 mmol) was separated by SFC (Instrument: SFC-150 (Waters); Column: AD-H 4.6×100 mm, 5 μm (Daicel); Column temperature: 40° C.; Mobile phase: CO₂/MeOH(0.2% Ammonia); Flow rate: 4 mL/min; Back pressure: 120 bar; Detection wavelength: 214 nm; Cycle time: 4.0 min; Injection volume: 5 μl to give the E10 isomer 1 (11 g, 42% yield) at retention time of 1.38 min as a colorless oil. E10 isomer 2 (8 g, 31% yield) at 1.68 min as a colorless oil. LCMS (LCMS) (m/z): [M+H]⁺ calc'd for C₁₆H₂₀F₂NO₂, 296.1; found, 296.0 (E10 isomer 1) and m/z=296.0 (E10 isomer 2).

### Synthesis of methyl 4-(4-(difluoromethyl)piperidin-2-yl)benzoate (F₃) and methyl 4-(4-(fluoromethyl)piperidin-2-yl)benzoate (F₄)

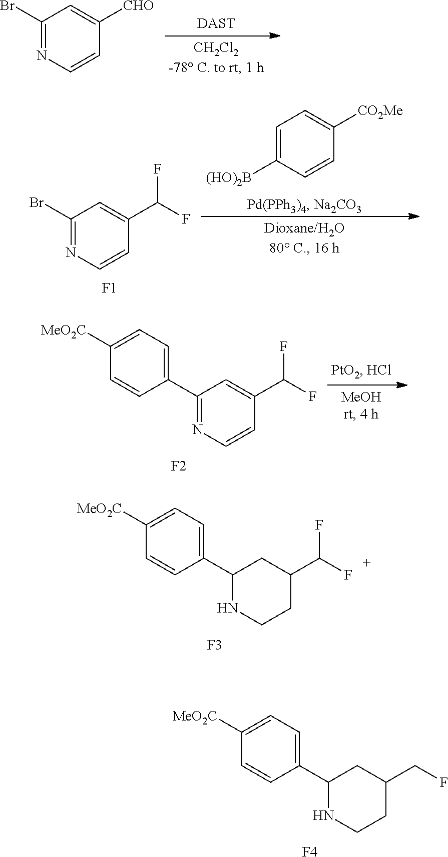

### Step 1: Synthesis of 2-bromo-4-(difluoromethyl)pyridine (F₁)

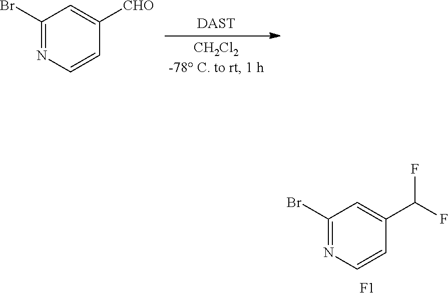

To a solution of 2-bromopyridine-4-carbaldehyde (5.00 g, 26.9 mmol) in CH₂Cl₂ (50 mL) at −78° C. was added DAST (13.0 g, 80.6 mmol). The reaction mixture was warmed to room temperature and stirred at that temp for 1 h prior to being quenched with saturated aqueous NH₄Cl (30 mL). The mixture was extracted with CH₂Cl₂ (3×30 mL). The combined organic layers were washed with brine (2×30 mL), dried over anhydrous Na₂SO₄, filtered and concentrated to obtain crude desired product. The crude product was purified by flash column chromatography (SiO₂, 10:1 petroleum ether/ethyl acetate) to give pure desired compound (3.8 g, 67%) as a yellow oil. LCMS (m/z): [M+H]⁺ calc'd for C₆H₅BrF₂N, 208.0; found, 208.7.

The following intermediate was synthesized using similar conditions as those described in Step 1, above, along with appropriate starting materials.

| Structure and Name | Intermediate | LCMS |
|---|---|---|
| 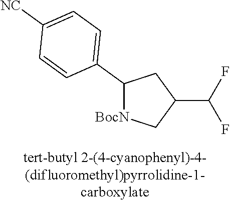 [SMILES: CC(C)(C)OC(=O)N1CC(C(F)F)CC1c1ccc(C#N)cc1] | F5 | LCMS (m/z): [M − ^{t}Bu]⁺ calc'd for C₁₃H₁₃F₂N₂O₂, 267.1; found, 267.1 |
| 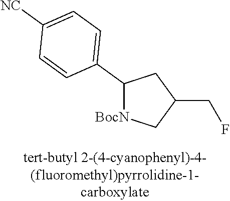 [SMILES: CC(C)(C)OC(=O)N1CC(CF)CC1c1ccc(C#N)cc1] | F6 | LCMS (m/z): [M − ^{t}Bu]⁺ calc'd for C₁₃H₁₄FN₂O₂, 249.1; found, 248.1 |
| 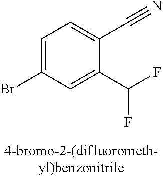 [SMILES: N#Cc1ccc(Br)cc1C(F)F] | F7 (from CAS 713141-12-9) | LCMS (m/z): [M + H]⁺ calc'd for C₈H₅BrF₂N, 232.0; found, 232.0 |
| 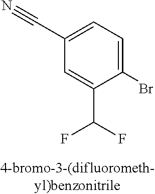 [SMILES: N#Cc1ccc(Br)c(C(F)F)c1] | F8 (from CAS 89003-95-2) | LCMS (m/z): [M + H]⁺ calc'd for C₈H₅BrF₂N, 232.0; found, 232.0 |
| 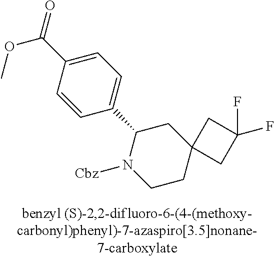 [SMILES: COC(=O)c1ccc([C@@H]2CC3(CCN2C(=O)OCc2ccccc2)CC(F)(F)C3)cc1] | F9 | LCMS (m/z): [M + H]⁺ calc'd for C₂₄H₂₆F₂NO₄, 430.2; found, 430 |
| 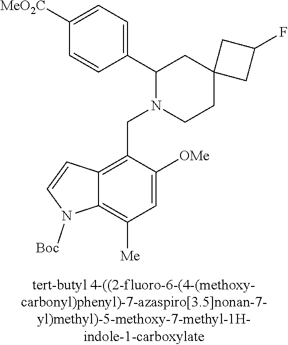 [SMILES: COC(=O)c1ccc(C2CC3(CCN2Cc2c(OC)cc(C)c4c2ccn4C(=O)OC(C)(C)C)CC(F)C3)cc1] | F15 | LCMS (m/z): [M + H]⁺ calc'd for C₃₂H₄₀FN₂O₅, 551; found, 551 |
| 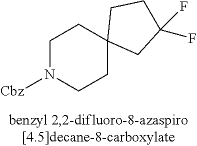 [SMILES: O=C(OCc1ccccc1)N1CCC2(CC1)CCC(F)(F)C2] | F20 | LCMS (m/z): [M + H]⁺ calc'd for C₁₇H₂₂F₂NO₂, 310; found, 310 |
| 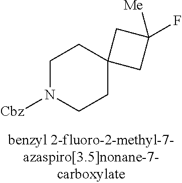 [SMILES: CC1(F)CC2(CCN(C(=O)OCc3ccccc3)CC2)C1] | F26 (from F25) | LCMS (m/z): [M + H]⁺ calc'd for C₁₇H₂₃FNO₃, 292.2; found, 292.1 |

### Step 2: Synthesis of methyl 4-(4-(difluoromethyl)pyridin-2-yl)benzoate (F₂)

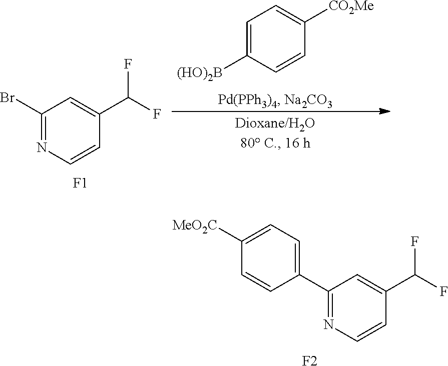

To a solution of 2-bromo-4-(difluoromethyl)pyridine (3.80 g, 18.4 mmol) and (4-(methoxycarbonyl)phenyl)boronic acid (6.59 g, 36.7 mmol) in dioxane (40 mL) and H₂O (10 mL) was added Na₂CO₃ (3.88 g, 36.7 mmol) and Pd(PPh₃)₄ (2.11 g, 1.84 mmol) and the resulting mixture stirred at 80° C. for 16 h. The reaction mixture was cooled to rt, diluted with H₂O (50 mL), and extracted with CH₂Cl₂ (3×50 mL). The combined organic layers were washed with brine (2×50 mL), dried over anhydrous Na₂SO₄, filtered, and concentrated. The crude product was purified by flash column chromatography (SiO₂, 10:1 petroleum ether/ethyl acetate) to give the desired pure compound (3.1 g, 47%) as a yellow oil. LCMS (m/z): [M+H]⁺ calc'd for C₁₄H₁₂F₂NO₂, 264.1; found, 264.0.

The following intermediate was synthesized using similar conditions as those described in Step 2, above, along with appropriate starting materials.

| Structure and Name | Intermediate | LCMS |
|---|---|---|
| 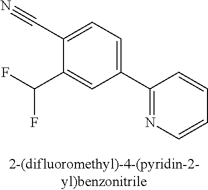 [SMILES: N#Cc1ccc(-c2ccccn2)cc1C(F)F] | F10 (from F7 and 2-pyridyl trimethyl- stannane) | LCMS (m/z): [M − ^{t}Bu]⁺ calc'd for C₁₃H₉F₂N₂, 231.1; found, 231.1 |
| 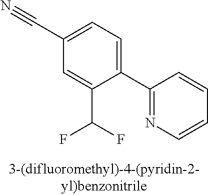 [SMILES: N#Cc1ccc(-c2ccccn2)c(C(F)F)c1] | F11 (from F8 and 2-pyridyl trimethyl- stannane) | LCMS (m/z): [M − ^{t}Bu]⁺ calc'd for C₁₃H₉F₂N₂, 231.1; found, 231.1 |

### Step 3: Synthesis of methyl 4-(4-(difluoromethyl)piperidin-2-yl)benzoate (F₃) and methyl 4-(4-(fluoromethyl)piperidin-2-yl)benzoate (F₄)

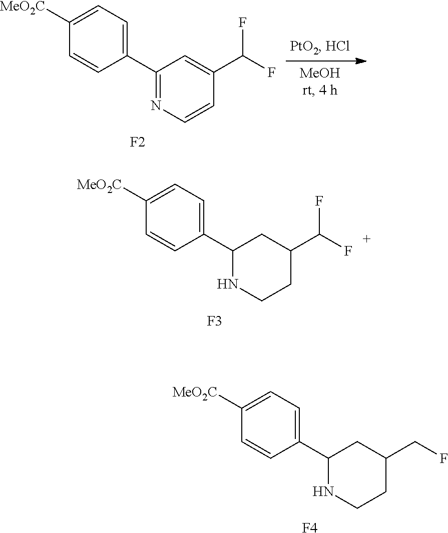

To a solution of methyl 4-(4-(difluoromethyl)pyridin-2-yl)benzoate (1.2 g, 4.6 mmol) in MeOH (10 mL) was added a solution of HCl/MeOH (0.5 mL), then PtO₂ (310 mg, 1.37 mmol). The reaction mixture was stirred at room temperature for 4 h under hydrogen. The suspension was filtered and evaporated to furnish the crude product, which was purified by flash column chromatography (SiO₂, 80:1 CH₂Cl₂/MeOH) to give pure methyl 4-(4-(difluoromethyl)piperidin-2-yl)benzoate (F₃, 240 mg, 20%) as a yellow oil. LCMS (m/z): [M+H]⁺ calc'd for C₁₄H₁₈F₂NO₂, 270.1; found, 269.9 and methyl 4-(4-(fluoromethyl)piperidin-2-yl)benzoate F4, 30 mg, 3%) as a yellow oil. LCMS (m/z): [M+H]⁺ calc'd for C₁₄H₁₉FNO₂, 252.1; found, 251.9.

The following intermediates were synthesized using similar conditions as those described above along with appropriate starting materials.

| Structure and Name | Intermediate | LCMS |
|---|---|---|
| 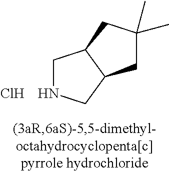 | F12 (from E6) | LCMS (m/z): [M + H]⁺ calc'd for C₉H₁₈N, 140.1; found, 140.2 |

### Synthesis of ethyl 2-(difluoromethyl)-4-(piperidin-2-yl)benzoate (F₁₃)

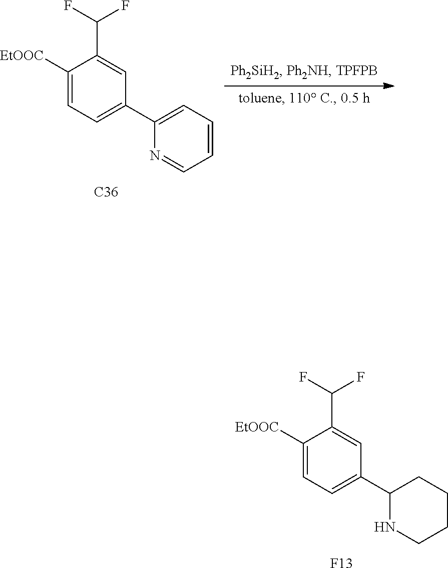 [SMILES: CCOC(=O)c1ccc(-c2ccccn2)cc1C(F)F ; "C36" ; CCOC(=O)c1ccc(C2CCCCN2)cc1C(F)F ; "F13"]

To a solution of ethyl 2-(difluoromethyl)-4-(pyridin-2-yl)benzoate (139 mg, 0.50 mmol) in toluene (5 mL) was added Ph₂NH (338 mg, 2.00 mmol), Ph₂SiH₂ (460 mg, 2.50 mmol) and TPFPB (51 mg, 0.10 mmol). The mixture was stirred at 110° C. for 0.5 h under N₂. LCMS showed product formed mostly and the mixture was concentrated. The residue was purified by flash column (EA/PE=1/1) to give ethyl 2-(difluoromethyl)-4-(piperidin-2-yl)benzoate (81 mg, 58%) as yellow oil. LCMS (m/z): [M+H]⁺ calc'd for C₁₅H₂₀F₂NO₂, 284.1; found, 284.

The following intermediates were synthesized using similar conditions as those described above along with appropriate starting materials.

| Structure and Name | Intermediate | LCMS |
|---|---|---|
| 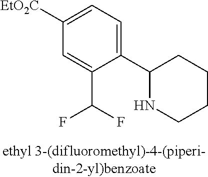 [SMILES: CCOC(=O)c1ccc(C2CCCCN2)c(C(F)F)c1] | F14 (from C37) | LCMS (m/z): [M + H]⁺ calc'd for C₁₅H₂₀F₂NO₂, 284.1; found, 284.1 |

### Synthesis of benzyl (S)-2,2-difluoro-6-(4-(5-oxo-4,5-dihydro-1H-1,2,4-triazol-3-yl)phenyl)-7-azaspiro[3.5]nonane-7-carboxylate (F9′)

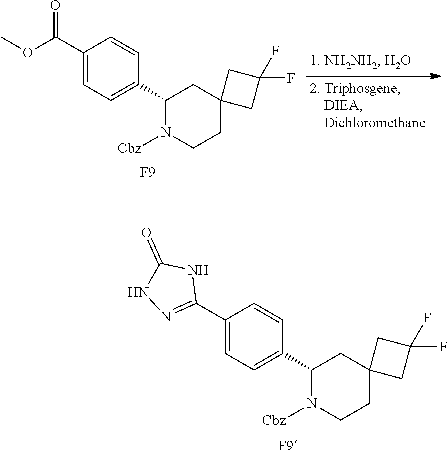

### Step 1: benzyl (S)-2,2-difluoro-6-(4-(hydrazinecarbonyl)phenyl)-7-azaspiro[3.5]nonane-7-carboxylate

To a solution benzyl (S)-2,2-difluoro-6-(4-(methoxycarbonyl)phenyl)-7-azaspiro[3.5]nonane-7-carboxylate F₉ (240 mg, 0.559 mmol) in MeOH (2 mL) was added NH₂NH₂•H₂O (4 mL) at room temperature, then the mixture was stirred at 100° C. for 1 h. The LCMS of aliquot showed that product was formed. The solution was concentrated to obtain crude product (260 mg) which was used for next step without further purification. MS: m/z=430 (M+1, ESI+).

### Step 2: benzyl (S)-2,2-difluoro-6-(4-(5-oxo-4,5-dihydro-1H-1,2,4-triazol-3-yl)phenyl)-7-azaspiro[3.5]nonane-7-carboxylate

Crude benzyl (S)-2,2-difluoro-6-(4-(hydrazinecarbonyl)phenyl)-7-azaspiro[3.5]nonane-7-carboxylate (260 mg), Triphosgene (309 mg, 1.05 mmol) and DIEPA (406 mg, 3.15 mmol) was dissolved in DCM (10 mL) and the stirred at room temperature for 2 h. The mixture was diluted with H₂O and extracted with EA (20 mL×3). The combined organic phases were dried on Na2SO₄, filtered and evaporated in vacuo and the residue was purified by silica gel to afford the benzyl (S)-2,2-difluoro-6-(4-(5-oxo-4,5-dihydro-1H-1,2,4-triazol-3-yl)phenyl)-7-azaspiro[3.5]nonane-7-carboxylate, F9′ (240 mg). MS: m/z=456 (M+1, ESI+).

### Synthesis of tert-butyl 4-((7-(4-cyanophenyl)-2,2-difluoro-8-azaspiro[4.5]decan-8-yl)methyl)-5-methoxy-7-methyl-1H-indole-1-carboxylate (compound 41)

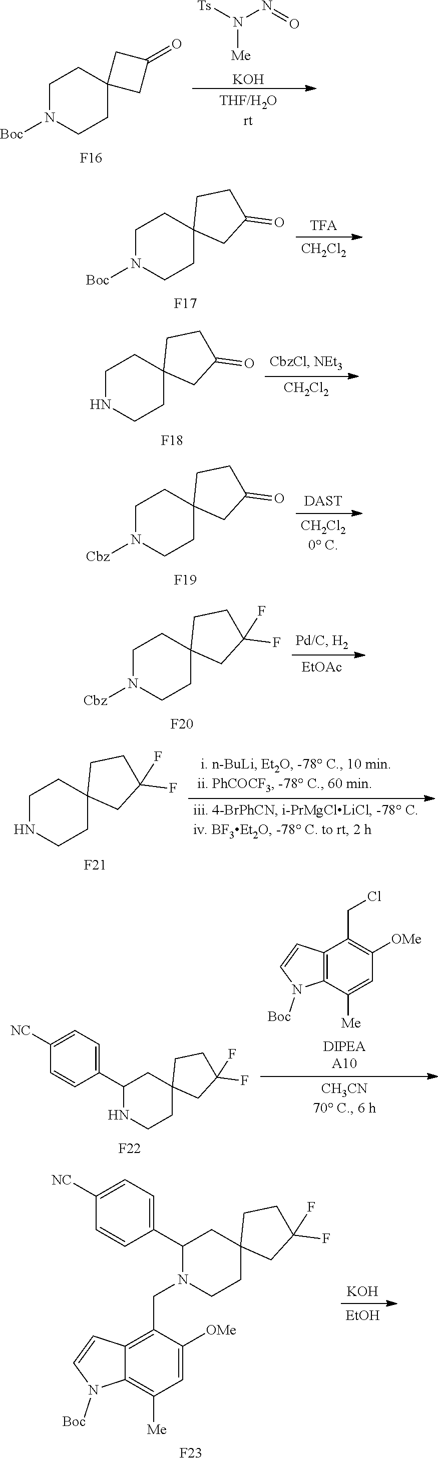

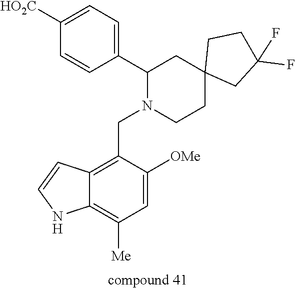

### Step 1: Synthesis of tert-butyl 2-oxo-8-azaspiro[4.5]decane-8-carboxylate (F₁₇)

N,4-dimethyl-N-nitrosobenzenesulfonamide (13 g, 63 mmol), F₁₆ (10 g, 42 mmol), and t-BuOK (7 g, 126 mmol) was dissolved in a solution of 10:1 THF/H₂O (200 mL) and stirred at rt overnight. The mixture was diluted with H₂O and extracted with ethyl acetate (3×500 ml). The combined organic layers were dried over Na₂SO₄, filtered, and evaporated in vacuo and the residue was purified by flash column chromatography (silica gel) to afford the product (8 g, 74% yield). LCMS (m/z): [M+H]⁺ calc'd for C₁₄H₂₄F₂NO₃, 254; found, 254.

### Synthesis of 4-(3-((5,7-dimethyl-1H-indol-4-yl)methyl)-3-azabicyclo[3.1.0]hexan-2-yl)benzoic acid (Example 1)

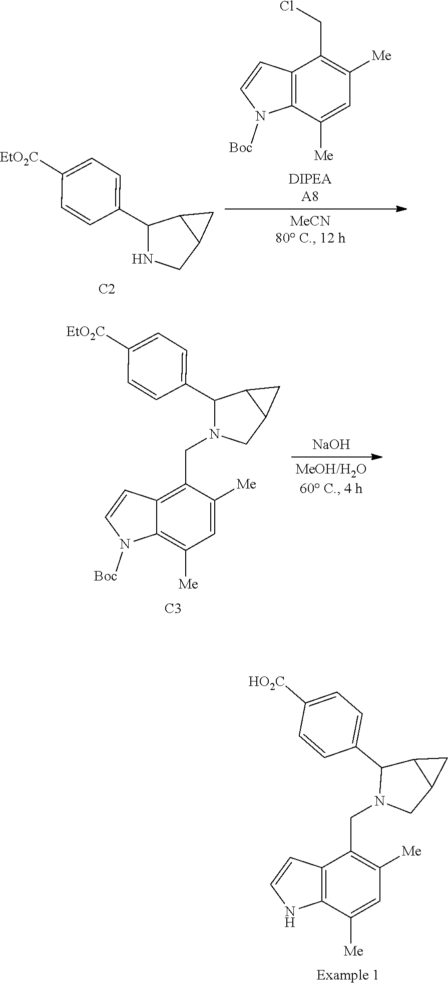

### Step 1: Synthesis of tert-butyl 4-((2-(4-(ethoxycarbonyl)phenyl)-3-azabicyclo[3.1.0]hexan-3-yl)methyl)-5,7-dimethyl-1H-indole-1-carboxylate (C3)

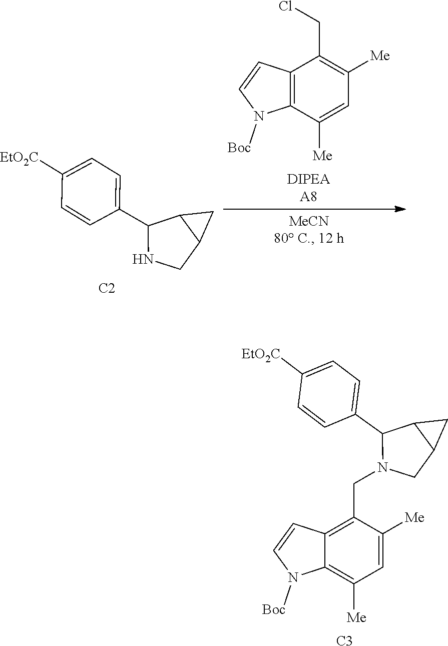

To a solution of ethyl 4-(3-azabicyclo[3.1.0]hexan-2-yl)benzoate (70 mg, 0.30 mmol) in MeCN was added a tert-butyl 4-(chloromethyl)-5,7-dimethyl-1H-indole-1-carboxylate (93 mg, 0.33 mmol) and then DIPEA (296 mg, 0.910 mmol). The reaction mixture was stirred under reflux for 16 h. The reaction was concentrated and purified by flash column chromatography (SiO₂, 10:1 ethyl acetate/petroleum ether) to afford the desired product (70 mg, 42%) as a white solid. LCMS (m/z): [M+H]⁺ calc'd for C₃₀H₃₇N2O₄, 489.3; found, 489.0.

The following intermediates were synthesized using similar conditions as those described in Step 1, above, along with appropriate starting materials.

| | | Isomer | |
|---|---|---|---|
| | | Separation | |
| | | Method | |
| | | and | |
| | | Retention | |
| Structure and Name | Intermediate | Time (if any) | LCMS |
| 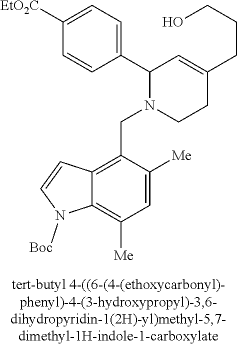 [SMILES: CCOC(=O)c1ccc(C2C=C(CCCO)CCN2Cc2c(C)cc(C)c3c2ccn3C(=O)OC(C)(C)C)cc1] | C15 | | LCMS (m/z): [M + H]⁺ calc'd for C₃₃H₄₃N₂O₅, 547.3; found, 547.1 |
| 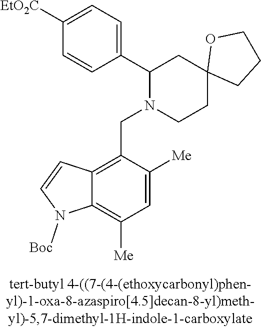 [SMILES: CCOC(=O)c1ccc(C2CC3(CCCO3)CCN2Cc2c(C)cc(C)c3c2ccn3C(=O)OC(C)(C)C)cc1] | C16 | | LCMS (m/z): [M + H]⁺ calc'd for C₃₃H₄₃N₂O₅, 547.3; found, 547.1 |
| 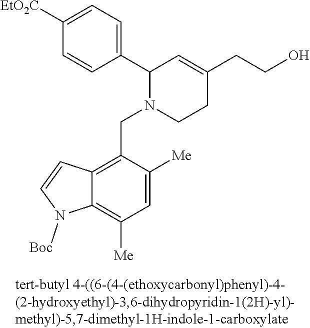 [SMILES: CCOC(=O)c1ccc(C2C=C(CCO)CCN2Cc2c(C)cc(C)c3c2ccn3C(=O)OC(C)(C)C)cc1] | C17 | | LCMS (m/z): [M + H]⁺ calc'd for C₃₂H₄₁N₂O₅, 533.3; found, 533.1 |
| 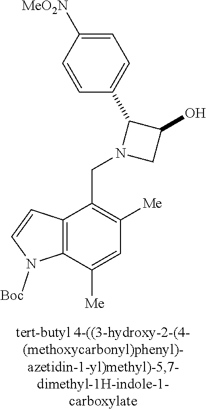 | C18 (From D2) | | LCMS (m/z): [M + H]⁺ calc'd for C₂₇H₃₃N₂O₅, 465.2; found, 464.7 |
| 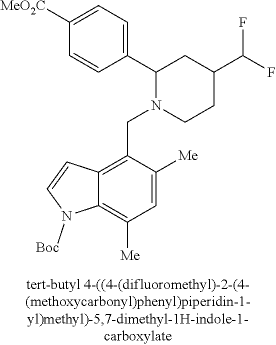 [SMILES: COC(=O)c1ccc(C2CC(C(F)F)CCN2Cc2c(C)cc(C)c3c2ccn3C(=O)OC(C)(C)C)cc1] | C19 (From E3) | | LCMS (m/z): [M + H]⁺ calc'd for C₃₀H₃₇F₂N₂O₄, 527.3; found, 526.7 |
| 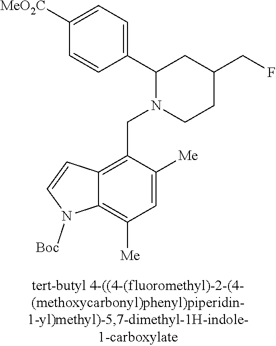 [SMILES: COC(=O)c1ccc(C2CC(CF)CCN2Cc2c(C)cc(C)c3c2ccn3C(=O)OC(C)(C)C)cc1] | C20 (From E4) | | LCMS (m/z): [M + H]⁺ calc'd for C₃₀H₃₇FN₂O₄, 509.3; found, 509.1 |
| 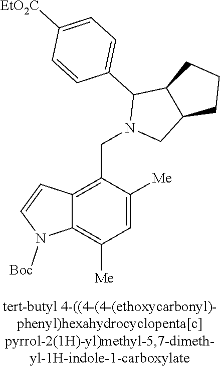 [SMILES: CCOC(=O)c1ccc(C2[C@H]3CCC[C@H]3CN2Cc2c(C)cc(C)c3c2ccn3C(=O)OC(C)(C)C)cc1] | C21 | | LCMS (m/z): [M + H]⁺ calc'd for C₃₂H₄₁N₂O₄, 517.3; found, 517.3 |
| 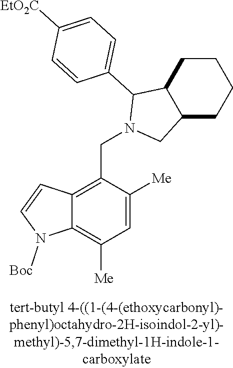 | C22 | | LCMS (m/z): [M + H]⁺ calc'd for C₃₃H₄₃N₂O₄, 531.3; found, 531.1 |
| 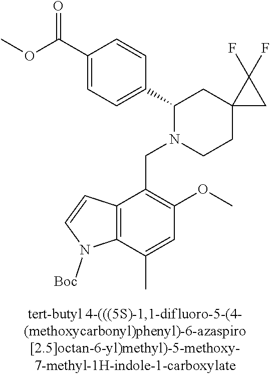 [SMILES: COC(=O)c1ccc([C@@H]2CC3(CCN2Cc2c(OC)cc(C)c4c2ccn4C(=O)OC(C)(C)C)CC3(F)F)cc1] | C23 | | LCMS (m/z): [M + H]⁺ calc'd for C₃₁H₃₇F₂N₂O₅, 555.3; found, 555 |
| 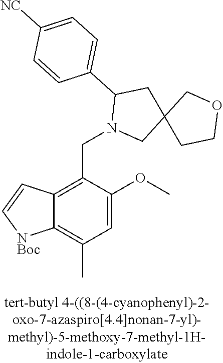 [SMILES: COc1cc(C)c2c(ccn2C(=O)OC(C)(C)C)c1CN1CC2(CCOC2)CC1c1ccc(C#N)cc1] | C39 (From C24) | | LCMS (m/z): [M + H]⁺ calc'd for C₃₀H₃₆N₃O₄, 502.3; found, 502.1 |
| 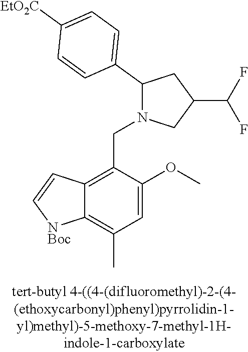 [SMILES: CCOC(=O)c1ccc(C2CC(C(F)F)CN2Cc2c(OC)cc(C)c3c2ccn3C(=O)OC(C)(C)C)cc1] | C40 (From C32) | | LCMS (m/z): [M + H]⁺ calc'd for C₃₀H₃₇F₂N₂O₅, 543.3; found, 543.1 |
| 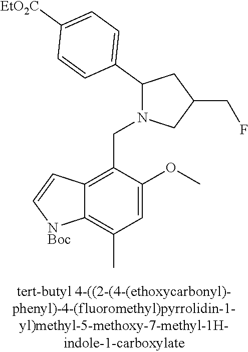 [SMILES: CCOC(=O)c1ccc(C2CC(CF)CN2Cc2c(OC)cc(C)c3c2ccn3C(=O)OC(C)(C)C)cc1] | C41 (From C33) | | LCMS (m/z): [M + H]⁺ calc'd for C₃₀H₃₈FN₂O₅, 525.3; found, 525.1 |
| 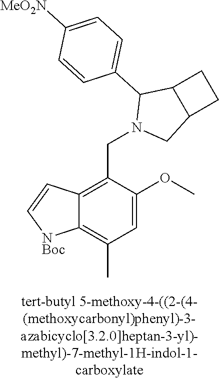 | C42 (From E4) | | LCMS (m/z): [M + H]⁺ calc'd for C₃₀H₃₇N₂O₅, 505.3; found, 505.1 |
| 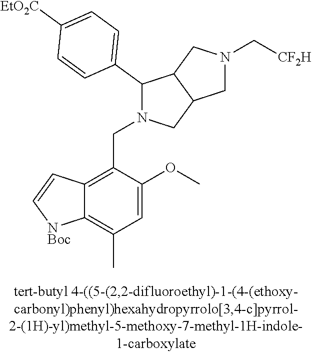 [SMILES: CCOC(=O)c1ccc(C2C3CN(CC(F)F)CC3CN2Cc2c(OC)cc(C)c3c2ccn3C(=O)OC(C)(C)C)cc1] | C43 (From C34) | | LCMS (m/z): [M + H]⁺ calc'd for C₃₃H₄₂F₂N₃O₅, 598.3; found, 597.1 |
| 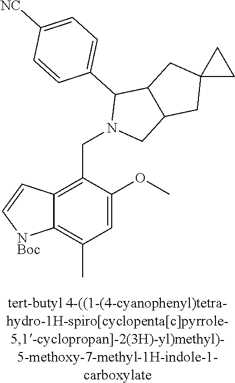 [SMILES: COc1cc(C)c2c(ccn2C(=O)OC(C)(C)C)c1CN1CC2CC3(CC3)CC2C1c1ccc(C#N)cc1] | C44 (From C27) | | LCMS (m/z): [M + H]⁺ calc'd for C₃₂H₃₈N₃O₃, 512.3; found, 512.1 |
| 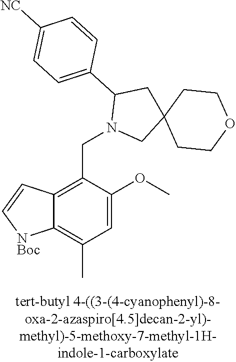 [SMILES: COc1cc(C)c2c(ccn2C(=O)OC(C)(C)C)c1CN1CC2(CCOCC2)CC1c1ccc(C#N)cc1] | C45 (From C28) | | LCMS (m/z): [M + H]⁺ calc'd for C₃₁H₃₈N₃O₄, 516.3; found, 516.1 |
| 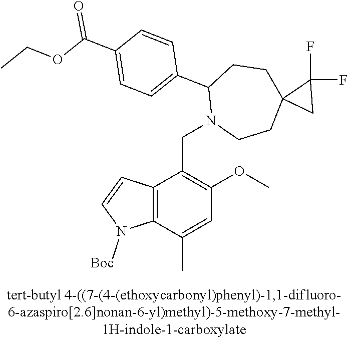 [SMILES: CCOC(=O)c1ccc(C2CCC3(CCN2Cc2c(OC)cc(C)c4c2ccn4C(=O)OC(C)(C)C)CC3(F)F)cc1] | C46 (From C35) | | LCMS (m/z): [M + H]⁺ calc'd for C₃₃H₄₁F₂N₂O₅, 583.3; found, 583.1 |
| 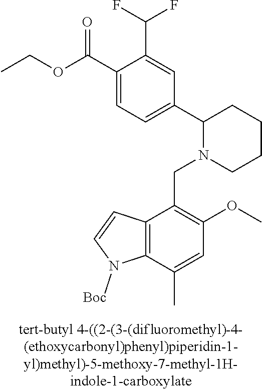 [SMILES: CCOC(=O)c1ccc(C2CCCCN2Cc2c(OC)cc(C)c3c2ccn3C(=O)OC(C)(C)C)cc1C(F)F] | C47 (From F13) | | LCMS (m/z): [M + H]⁺ calc'd for C₃₁H₃₉F₂N₂O₅, 557.3; found, 557.1 |
| 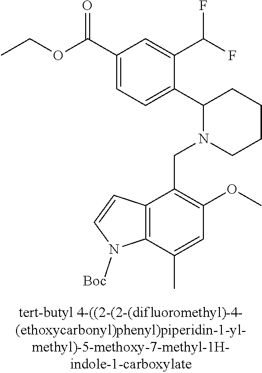 [SMILES: CCOC(=O)c1ccc(C2CCCCN2Cc2c(OC)cc(C)c3c2ccn3C(=O)OC(C)(C)C)c(C(F)F)c1] | C48 (From F14) | | LCMS (m/z): [M + H]⁺ calc'd for C₃₁H₃₉F₂N₂O₅, 557.3; found, 557.1 |
| 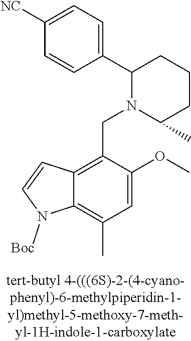 [SMILES: COc1cc(C)c2c(ccn2C(=O)OC(C)(C)C)c1CN1C(c2ccc(C#N)cc2)CCC[C@@H]1C] | C49 | | LCMS (m/z): [M + H]⁺ calc'd for C₂₉H₃₆N₃O₃, 474.3; found, 474.1 |
| 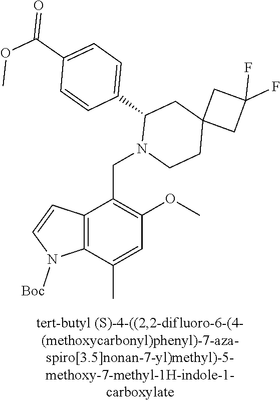 [SMILES: COC(=O)c1ccc([C@@H]2CC3(CCN2Cc2c(OC)cc(C)c4c2ccn4C(=O)OC(C)(C)C)CC(F)(F)C3)cc1] | C50 | | LCMS (m/z): [M + H]⁺ calc'd for C₃₂H₃₉F₂N₂O₅, 569.3; found, 569 |
| 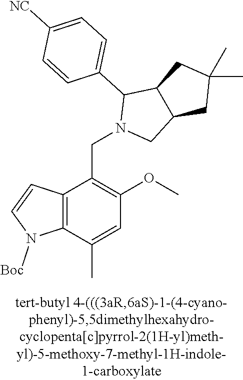 [SMILES: COc1cc(C)c2c(ccn2C(=O)OC(C)(C)C)c1CN1C[C@@H]2CC(C)(C)C[C@@H]2C1c1ccc(C#N)cc1] | C51 | | LCMS (m/z): [M + H]⁺ calc'd for C₃₂H₄₀N₃O₃, 514.3; found, 514 |
| 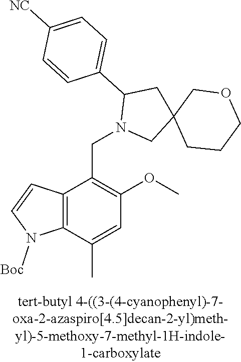 [SMILES: COc1cc(C)c2c(ccn2C(=O)OC(C)(C)C)c1CN1CC2(CCCOC2)CC1c1ccc(C#N)cc1] | C52 (From C31) | | LCMS (m/z): [M + H]⁺ calc'd for C₃₁H₃₈N₃O₄, 516.3; found, 516 |
| 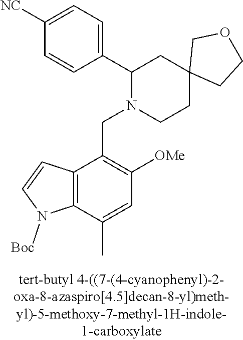 [SMILES: COc1cc(C)c2c(ccn2C(=O)OC(C)(C)C)c1CN1CCC2(CCOC2)CC1c1ccc(C#N)cc1] | C53 | | LCMS (m/z): [M + H]⁺ calc'd for C₃₁H₃₈N₃O₄, 516.3; found, 516 |
| 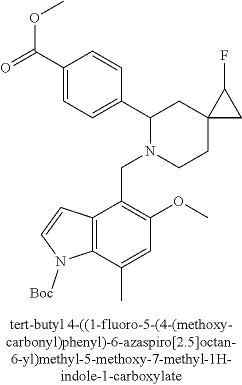 [SMILES: COC(=O)c1ccc(C2CC3(CCN2Cc2c(OC)cc(C)c4c2ccn4C(=O)OC(C)(C)C)CC3F)cc1] | C54 | | LCMS (m/z): [M + H]⁺ calc'd for C₃₁H₃₈FN₂O₅, 537.3; found, 537 |
| 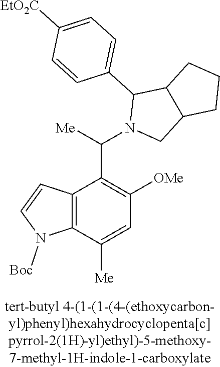 [SMILES: CCOC(=O)c1ccc(C2C3CCCC3CN2C(C)c2c(OC)cc(C)c3c2ccn3C(=O)OC(C)(C)C)cc1] | C55 (From A12 and C12 Isomer 1 OR Isomer 2) | | LCMS (m/z): [M + H]⁺ calc'd for C₃₃H₄₃N₂O₅, 547; found, 547 |
| 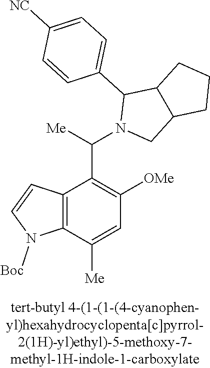 [SMILES: COc1cc(C)c2c(ccn2C(=O)OC(C)(C)C)c1C(C)N1CC2CCCC2C1c1ccc(C#N)cc1] | C56 (From A12 and C6) | | LCMS (m/z): [M + H]⁺ calc'd for C₃₁H₃₈N₃O₃, 500; found, 500 |
| 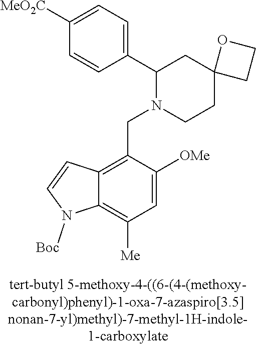 [SMILES: COC(=O)c1ccc(C2CC3(CCO3)CCN2Cc2c(OC)cc(C)c3c2ccn3C(=O)OC(C)(C)C)cc1] | C57 (Isomer Mixture 1) | Column: Waters SunFire 10 μm C18 250 × 19 mm, 10 μm Mobile phase: acetonitrile Flow rate: 30 mL/min Linear gradient | LCMS (m/z): [M + H]⁺ calc'd for C₃₁H₃₉N₂O₆, 535.3; found, 535.2 |
| 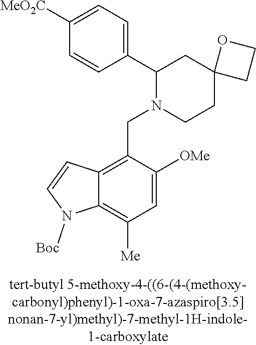 [SMILES: COC(=O)c1ccc(C2CC3(CCO3)CCN2Cc2c(OC)cc(C)c3c2ccn3C(=O)OC(C)(C)C)cc1] | C57 (Isomer Mixture 2) | Column: Waters SunFire 10 μm C18 250 × 19 mm, 10 μm Mobile phase: acetonitrile Flow rate: 30 mL/min Linear gradient | LCMS (m/z): [M + H]⁺ calc'd for C₃₁H₃₉N₂O₆, 535.3; found, 535.2 |
| 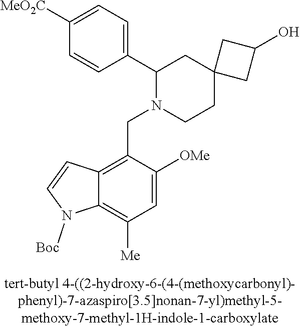 [SMILES: COC(=O)c1ccc(C2CC3(CCN2Cc2c(OC)cc(C)c4c2ccn4C(=O)OC(C)(C)C)CC(O)C3)cc1] | C58 | | LCMS (m/z): [M + H]⁺ calc'd for C₃₂H₄₁N₂O₆, 549; found, 549 |
| 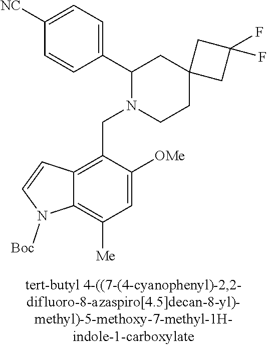 [SMILES: COc1cc(C)c2c(ccn2C(=O)OC(C)(C)C)c1CN1CCC2(CC1c1ccc(C#N)cc1)CC(F)(F)C2] | F23 | | LCMS (m/z): [M + H]⁺ calc'd for C₃₂H₃₈F₂N₃O₃, 550.3; found, 550.1 |
| 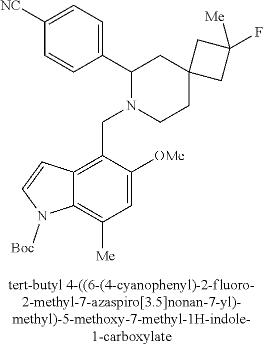 [SMILES: COc1cc(C)c2c(ccn2C(=O)OC(C)(C)C)c1CN1CCC2(CC1c1ccc(C#N)cc1)CC(C)(F)C2] | F29 (from F28) | | LCMS (m/z): [M + H]⁺ calc'd for C₃₂H₃₉FN₃O₃, 532; found, 532 |

### Step 2: Synthesis of 4-(3-((5,7-dimethyl-1H-indol-4-yl)methyl)-3-azabicyclo[3.1.0]hexan-2-yl)benzoic acid (Example 1)

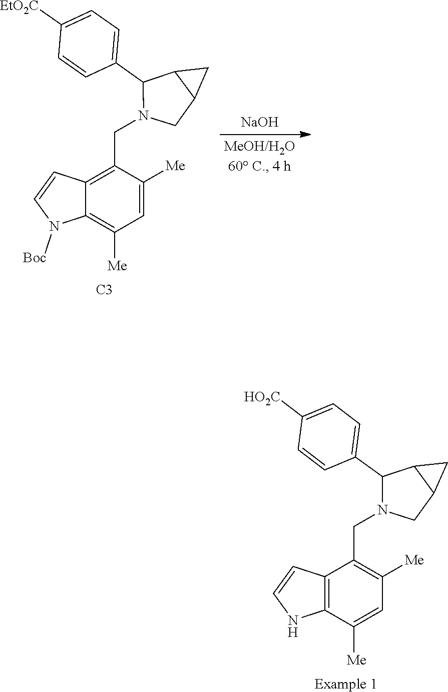

To a solution of tert-butyl 4-((2-(4-(ethoxycarbonyl)phenyl)-3-azabicyclo[3.1.0]hexan-3-yl)methyl)-5,7-dimethyl-1H-indole-1-carboxylate (70 mg, 0.14 mmol) in MeOH (3 mL), was added NaOH (28 mg, 0.70 mmol) in water (0.3 mL) and the mixture stirred at 60° C. for 4 h, then cooled to room temperature and concentrated in vacuo. A solution of citric acid (1 M in H₂O) was added to adjust the pH to 6.4-6.7 and the mixture was extracted with CH₂Cl₂ (3×20 mL). The combined organic layers were washed with brine (10 mL) and then concentrated in vacuo. The residue was purified by prep-HPLC (column: Waters™ XBridge 2.1×50 mm 3.5 μm; mobile phase A [water (0.05% trifluoroacetic acid v/v)] and B [acetonitrile (0.05% trifluoroacetic acid)]; gradient B: 10-100% over 7 min).

Example 1: 4-(3-((5,7-dimethyl-1H-indol-4-yl)methyl)-3-azabicyclo[3.1.0]hexan-2-yl)benzoic acid (11.9 mg, 23%) was obtained as a white solid. LCMS (LCMS) (m/z): [M+H]⁺ calc'd for C₂₃H₂₅N₂O₂, 361.2; found, 361.9. ¹H NMR (400 MHz, CD₃OD) δ 8.29 (br s, 0.8H), 8.09 (d, J=8.1 Hz, 2H), 7.55 (d, J=7.9 Hz, 2H), 7.19 (d, J=3.0 Hz, 1H), 6.71 (s, 1H), 6.17 (d, J=0.6 Hz, 1H), 4.27 (m, 1H), 4.18 (d, J=12.5 Hz, 1H), 3.94 (d, J=13.0 Hz, 1H), 3.48 (m, 1H), 3.03 (d, J=11.0 Hz, 1H), 2.42 (s, 3H), 2.05 (s, 3H), 1.89 (d, J=18.3 Hz, 2H), 1.09 (m, 2H).

The following examples were synthesized using the ester hydrolysis procedure described above using appropriate starting materials:

| | | Isomer Separation | |
|---|---|---|---|
| | | Method and | |
| | | Retention Time | |
| Name and Structure | Ex. # | (if any) | LCMS + ¹H NMR |
| 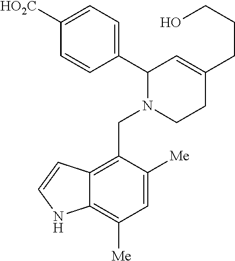 [SMILES: Cc1cc(C)c2[nH]ccc2c1CN1CCC(CCCO)=CC1c1ccc(C(=O)O)cc1] | 2 (Isomer 1) | Column: CHIRALPAK IC 250 × 20 mm I.D., 5 μm Mobile phase: 40% IPA (0.2% NH₄OH) in CO₂ Flow rate: 12.5 mL/min Column temperature: 40.4° C. Retention time = 3.35 min | LCMS (m/z): [M + H]⁺ calc'd for C₂₆H₃₁N₂O₃, 419.2; found, 419.0 ¹H NMR (400 MHz, CD₃OD): δ 8.03 (d, J = 7.2 Hz, 2H), 7.47 (d, J = 7.4 Hz, 2H), 7.21 (d, J = 2.3 Hz, 1H), 6.74 (s, 1H), 6.27 (br s, 1H), 5.39 (br s, 1H), 4.70 (br s, 1H), 4.33 (d, J = 12.7 Hz, 1H), 4.14-4.04 (m, 1H), 3.56 (t, J = 6.4 Hz, 2H), 3.40-3.34 (m, 1H), 3.15-3.08 (m, 1H), 2.41 (s, 3H), 2.39-2.34 (m, 1H), 2.21-2.15 (m, 3H), 2.03 (s, 3H), 1.72-1.61 (m, 2H). |
| 4-(1-((5,7-dimethyl-1H-indol-4- | | | |
| yl)methyl)-4-(3-hydroxypropyl)- | | | |
| 1,2,5,6-tetrahydropyridin-2- | | | |
| yl)benzoic acid | | | |
| 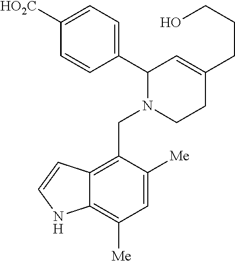 [SMILES: Cc1cc(C)c2[nH]ccc2c1CN1CCC(CCCO)=CC1c1ccc(C(=O)O)cc1] | 2 (Isomer 2) | Column: CHIRALPAK IC 250 × 20 mm I.D., 5 μm Mobile phase: 40% IPA (0.2% NH₄OH) in CO₂ Flow rate: 12.5 mL/min Column temperature: 39.5° C. Retention time = 6.2 min | LCMS (m/z): [M + H]⁺ calc'd for C₂₆H₃₁N₂O₃, 419.2; found, 419.0 ¹H NMR (400 MHz, CD₃OD): δ 8.04 (d, J = 8.2 Hz, 2H), 7.48 (dd, J = 8.1, 2.4 Hz, 2H), 7.22 (d, J = 3.1 Hz, 1H), 6.74 (s, 1H), 6.27 (br s, 1H), 5.40 (br s, 1H), 4.72 (br s, 1H), 4.41-4.28 (m, 1H), 4.19- 4.05 (m, 1H), 3.56 (t, J = 6.4 Hz, 2H), 3.44-3.33 (m, 1H), 3.14- 3.09 (m, 1H), 2.41 (s, 3H), 2.39- 2.37 (m, 1H), 2.23-2.15 (m, 3H), 2.03 (s, 3H), 1.71-1.65 (m, 2H). |
| 4-(1-((5,7-dimethyl-1H-indol-4- | | | |
| yl)methyl)-4-(3-hydroxypropyl)- | | | |
| 1,2,5,6-tetrahydropyridin-2- | | | |
| yl)benzoic acid | | | |
| 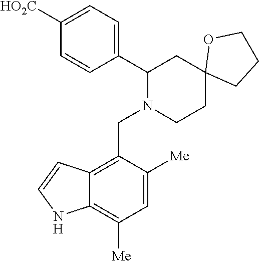 [SMILES: Cc1cc(C)c2[nH]ccc2c1CN1CCC2(CCCO2)CC1c1ccc(C(=O)O)cc1] | 2 (Isomer 3) | Column: CHIRALPAK IC 250 × 20 mm I.D., 5 μm Mobile phase: 35% EtOH (0.2% NH₄OH) in CO₂ Flow rate: 12.5 mL/min Column temperature: 40.2° C. Retention time = 2.28 min | LCMS (m/z): [M + H]⁺ calc'd for C₂₆H₃₁N₂O₃, 419.2; found, 419.0 ¹H NMR (400 MHz, CD₃OD): δ 7.98 (d, J = 7.6 Hz, 2H), 7.54 (d, J = 7.7 Hz, 2H), 7.13 (d, J = 3.0 Hz, 1H), 6.65 (s, 1H), 6.34 (s, 1H), 4.06- 3.76 (m, 2H), 3.71 (t, J = 6.3 Hz, 2H), 3.67-3.48 (m 1H), 3.31- 3.22 (m, 1H), 3.17-2.96 (m, 1H), 2.32 (s, 3H), 2.10 (s, 3H), 2.07- 1.87 (m, 5H), 1.87-1.68 (m, 2H), 1.68-1.51 (m, 1H). |
| 4-(8-((5,7-dimethyl-1H-indol-4- | | | |
| yl)methyl)-1-oxa-8- | | | |
| azaspiro[4.5]decan-7-yl)benzoic | | | |
| acid | | | |
| 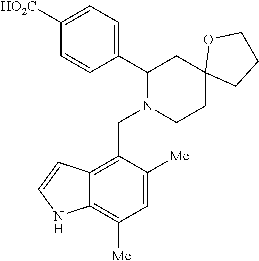 [SMILES: Cc1cc(C)c2[nH]ccc2c1CN1CCC2(CCCO2)CC1c1ccc(C(=O)O)cc1] | 2 (Isomer 4) | Column: CHIRALPAK IC 250 × 20 mm I.D., 5 μm Mobile phase: 35% EtOH (0.2% NH₄OH) in CO₂ Flow rate: 12.5 mL/min Column temperature: 39.6° C. Retention time = 3.61 min | LCMS (m/z): [M + H]⁺ calc'd for C₂₆H₃₁N₂O₃, 419.2; found, 419.0 ¹H NMR (400 MHz, CD₃OD): δ 8.00 (d, J = 7.9 Hz, 2H), 7.55 (d, J = 7.8 Hz, 2H), 7.14 (d, J = 3.1 Hz, 1H), 6.66 (s, 1H), 6.33 (s, 1H), 4.08- 3.80 (m, 2H), 3.76-3.64 (m, 3H), 3.31-3.22 (m, 1H), 3.16- 3.03 (m, 1H), 2.33 (s, 3H), 2.08 (s, 3H), 2.05-1.87 (m, 5H), 1.87- 1.70 (m, 2H), 1.69-1.52 (m, 1H). |
| 4-(8-((5,7-dimethyl-1H-indol-4- | | | |
| yl)methyl)-1-oxa-8- | | | |
| azaspiro[4.5]decan-7-yl)benzoic | | | |
| acid | | | |
| 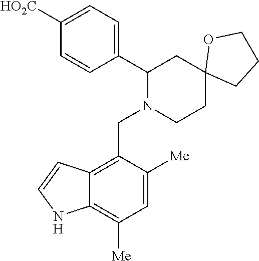 [SMILES: Cc1cc(C)c2[nH]ccc2c1CN1CCC2(CCCO2)CC1c1ccc(C(=O)O)cc1] | 2 (Isomer 5) | Column: CHIRALPAK IC 250 × 20 mm I.D., 5 μm Mobile phase: 40% MeOH (0.2% NH₄OH) in CO₂ Flow rate: 12.5 mL/min Column temperature: 40.5° C. Retention time = 3.25 min | LCMS (m/z): [M + H]⁺ calc'd for C₂₆H₃₁N₂O₃, 419.2; found, 419.0 ¹H NMR (400 MHz, CD₃OD): δ 8.07 (d, J = 8.1 Hz, 2H), 7.60 (d, J = 7.7 Hz, 2H), 7.22 (d, J = 3.1 Hz, 1H), 6.75 (s, 1H), 6.36 (s, 1H), 4.47- 4.25 (m, 1H), 4.20-4.03 (m, 1H), 4.03-3.79 (m, 3H), 3.30-3.34 (m, 1H), 3.25-3.08 (m, 1H), 2.40 (s, 3H), 2.14 (s, 3H), 2.08-2.11 (m, 1H), 1.99-1.88 (m, 3H), 1.82- 1.65 (m, 4H). |
| 4-(8-((5,7-dimethyl-1H-indol-4- | | | |
| yl)methyl)-1-oxa-8- | | | |
| azaspiro[4.5]decan-7-yl)benzoic | | | |
| acid | | | |
| 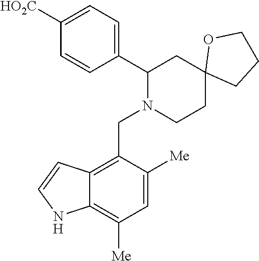 [SMILES: Cc1cc(C)c2[nH]ccc2c1CN1CCC2(CCCO2)CC1c1ccc(C(=O)O)cc1] | 2 (Isomer 6) | Column: CHIRALPAK IC 250 × 20 mm I.D., 5 μm Mobile phase: 40% MeOH (0.2% NH₄OH) in CO₂ Flow rate: 12.5 mL/min Column temperature: 39.5° C. Retention time = 5.03 min | LCMS (m/z): [M + H]⁺ calc'd for C₂₆H₃₁N₂O₃, 419.2; found 419.0 ¹H NMR (400 MHz, CD₃OD): δ 8.48 (br s, 1H), 8.09 (d, J = 8.0 Hz, 2H), 7.62 (d, J = 7.8 Hz, 2H), 7.24 (d, J = 3.2 Hz, 1H), 6.77 (s, 1H), 4.41-4.62 (m, 1H), 4.23-4.01 (m, 2H), 3.99-3.83 (m, 2H), 3.40- 3.31 (m, 1H), 3.25-3.08 (m, 1H), 2.41 (s, 3H), 2.26-2.09 (m, 4H), 2.00-1.90 (m, 3H), 1.86- 1.68 (m, 4H). |
| 4-(8-((5,7-dimethyl-1H-indol-4- | | | |
| yl)methyl)-1-oxa-8- | | | |
| azaspiro[4.5]decan-7-yl)benzoic | | | |
| acid | | | |
| 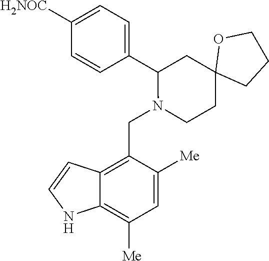 [SMILES: Cc1cc(C)c2[nH]ccc2c1CN1CCC2(CCCO2)CC1c1ccc(C(N)=O)cc1] | 3 | N/A | LCMS (m/z): [M + H]⁺ calc'd for C26H32N3O2, 418.2; found, 418.0 ¹H NMR (400 MHz, CD₃OD): δ 8.49 (br s, 1H), 8.00-7.80 (m, 2H), 7.60-7.45 (m, 2H), 7.21- 7.15 (m, 1H), 6.71 (s, 1H), 6.40- 6.30 (m, 1H), 5.45-5.30 (m, 1H), 4.40-4.20 (m, 1H), 4.10-3.90 (m, 1H), 3.80-3.60 (m, 1H), 3.85- 3.35 (m, 3H), 3.15-2.90 (m, 2H), 2.41 (s, 3H), 2.30-2.15 (m, 2H), 2.12 (s, 3H), 2.07-1.95 (m, 2H), 1.75-1.55 (m, 2H). |
| 4-(8-((5,7-dimethyl-1H-indol-4- | | | |
| yl)methyl)-1-oxa-8- | | | |
| azaspiro[4.5]decan-7- | | | |
| yl))benzamide | | | |
| 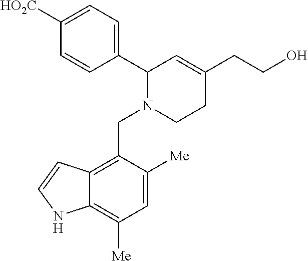 [SMILES: Cc1cc(C)c2[nH]ccc2c1CN1CCC(CCO)=CC1c1ccc(C(=O)O)cc1] | 4 | N/A | LCMS (m/z): [M + H]⁺ calc'd for C₂₅H₂₉N₂O₃, 405.2; found, 405.0 ¹H NMR (400 MHz, CD₃OD): δ 8.42 (br s, 0.4H), 8.06 (d, J = 8.2 Hz, 2H), 7.52 (d, J = 8.2 Hz, 2h), 7.24 (d, J = 3.1 Hz, 1H), 6.78 (s, 1H), 6.31 (d, J = 2.4 Hz, 1H), 5.48 (br s, 1H), 4.92-4.87 (m, 1H), 4.84-4.73 (m, 1H), 4.44-4.34 (m, 1H), 4.23-4.13 (m, 1H), 3.74- 3.66 (m, 2H), 3.46-3.37 (m, 1H), 3.25-3.14 (m, 1H), 2.62- 2.45 (m, 1H), 2.44 (s, 3H), 2.37- 2.32 (m, 2H), 2.31-2.21 (m, 1H), 2.08 (s, 3H). |
| 4-(1-((5,7-dimethyl-1H-indol-4- | | | |
| yl)methyl)-4-(2-hydroxyethyl)- | | | |
| 1,2,5,6-tetrahydropyridin-2- | | | |
| yl)benzoic acid | | | |
| 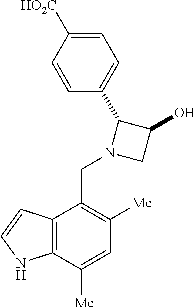 | 5 | N/A | LCMS (m/z): [M + H]⁺ calc'd for C₂₁H₂₃N₂O₃, 351.2; found, 351.0 ¹H NMR (400 MHz, CD₃OD): δ 7.90 (d, J = 8.2 Hz, 2H), 7.30 (d, J = 8.1 Hz, 2H), 7.23 (d, J = 3.1 Hz, 1H), 6.71 (s, 1H), 6.49 (d, J = 3.1 Hz, 1H), 4.68-4.59 (m, 1H), 4.41 (s, 2H), 4.41-4.35 (m, 1H), 3.86- 3.84 (m, 1H), 3.56-3.54 (m, 1H), 2.40 (s, 3H), 2.30 (s, 3H). |
| 4-(1-((5,7-dimethyl-1H-indol-4- | | | |
| yl)methyl)-3-hydroxyazetidin-2- | | | |
| yl)benzoic acid | | | |
| 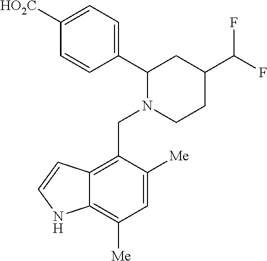 [SMILES: Cc1cc(C)c2[nH]ccc2c1CN1CCC(C(F)F)CC1c1ccc(C(=O)O)cc1] | 6 (Isomer 1) | Column: CHIRALPAK AD- H 250 × 4.6 mm I.D., 5 μm Mobile phase: 40% MeOH (0.2% NH₄OH) in CO₂ Flow rate: 12.5 mL/min Column temperature: 41.2° C. Retention time = 3.02 min | LCMS (m/z): [M + H]⁺ calc'd for C₂₄H₂₇F₂N₂O₂, 413.2; found, 413.1 ¹H NMR (400 MHz, CD₃OD): δ 8.35 (s, 0.35H), 8.11 (d, J = 8.3 Hz, 2H), 7.67 (d, J = 7.8 Hz, 2H), 7.23 (d, J = 3.1 Hz, 1H), 6.75 (s, 1H), 6.42 (d, J = 2.6 Hz, 1H), 5.74 (td, J = 56.6, 4.0 Hz, 1H), 4.02 (d, J = 12.8 Hz, 1H), 3.97-3.94 (m, 1H), 3.76 (d, J = 12.4 Hz, 1H), 3.26- 3.23 (m, 1H), 2.83-2.69 (m, 1H), 2.42 (s, 3H), 2.28-2.21 (m, 1H), 2.20 (s, 3H), 2.06-1.98 (m, 1H), 1.93-1.82 (m, 2H), 1.65-1.55 (m, 1H). |
| 4-(4-(difluoromethyl)-1-((5,7- | | | |
| dimethyl-1H-indol-4- | | | |
| yl)methyl)piperidin-2-yl)benzoic | | | |
| acid | | | |
| 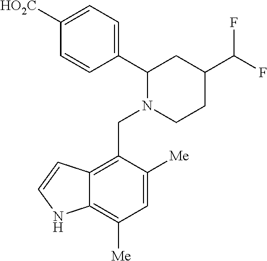 [SMILES: Cc1cc(C)c2[nH]ccc2c1CN1CCC(C(F)F)CC1c1ccc(C(=O)O)cc1] | 6 (Isomer 2) | Column: CHIRALPAK AD- H 250 × 4.6 mm I.D., 5 μm Mobile phase: 40% MeOH (0.2% NH₄OH) in CO₂ Flow rate: 12.5 mL/min Column temperature: 41.2° C. Retention time = 2.22 min | LCMS (m/z): [M + H]⁺ calc'd for C₂₄H₂₇F₂N₂O₂, 413.2; found, 413.1 ¹H NMR (400 MHz, CD₃OD): δ 8.34 (s, 0.35 H), 8.12 (d, J = 8.2 Hz, 2H), 7.67 (d, J = 7.8 Hz, 2H), 7.24 (d, J = 3.1 Hz, 1H), 6.76 (s, 1H), 6.42 (d, J = 2.8 Hz, 1H), 5.74 (td, J = 56.6, 4.0 Hz, 1H), 4.03 (d, J = 12.8 Hz, 1H), 3.99-3.97 (m, 1H), 3.78 (d, J = 12.3 Hz, 1H), 3.27- 3.24 (m, 1H), 2.81-2.75 (m, 1H), 2.42 (s, 3H), 2.26-2.22 (m, 1H), 2.20 (s, 3H), 2.08-1.99 (m, 1H), 1.96-1.79 (m, 2H), 1.65- 1.57 (m, 1H). |
| 4-(4-(difluoromethyl)-1-((5,7- | | | |
| dimethyl-1H-indol-4- | | | |
| yl)methyl)piperidin-2-yl)benzoic | | | |
| acid | | | |
| 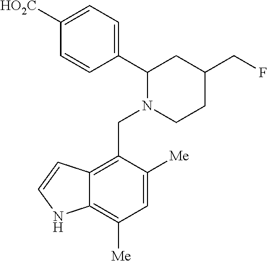 [SMILES: Cc1cc(C)c2[nH]ccc2c1CN1CCC(CF)CC1c1ccc(C(=O)O)cc1] | 7 (Isomer 1) | Column: XBRIDGE IC 2.1 × 50 mm I.D., 3.5 μm Mobile phase: 10- 100% MeCN (0.05% TFA) in H₂O (0.05% TFA) over 7 min Flow rate: 0.8 mL/min Column temperature: 45° C. Retention time = 2.88 min | LCMS (m/z): [M + H]⁺ calc'd for C₂₄H₂₈FN₂O₂, 395.2; found, 394.8 ¹H NMR (400 MHz, CD₃OD): δ 8.41 (s, 0.46H), 8.13 (d, J = 7.9 Hz, 2H), 7.66 (d, J = 7.7 Hz, 2H), 7.27 (d, J = 3.1 Hz, 1H), 6.79 (s, 1H), 6.38 (d, J = 1.0 Hz, 1H), 4.33 (dd, J = 47.4, 5.3 Hz, 2H), 4.19-3.94 (m, 3H), 3.40-3.37 (m, 1H), 3.13- 3.07 (m, 1H), 2.43 (s, 3H), 2.27- 2.18 (m, 1H), 2.16 (s, 3H), 2.09- 2.06 (m, 1H), 1.89-1.86 (m, 2H), 1.59-1.56 (m, 1H). |
| 4-(1-((5,7-dimethyl-1H-indol-4- | | | |
| yl)methyl)-4- | | | |
| (fluoromethyl)piperidin-2- | | | |
| yl)benzoic acid | | | |
| 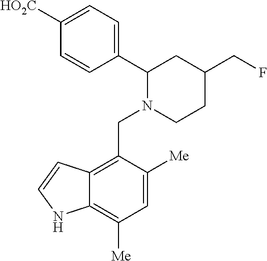 [SMILES: Cc1cc(C)c2[nH]ccc2c1CN1CCC(CF)CC1c1ccc(C(=O)O)cc1] | 7 (Isomer 2) | Column: XBRIDGE IC 2.1 × 50 mm I.D., 3.5 μm Mobile phase: 10- 100% MeCN (0.05% TFA) in H₂O (0.05% TFA) over 7 min Flow rate: 0.8 mL/min Column temperature: 45° C. Retention time = 2.95 min | LCMS (m/z): [M + H]⁺ calc'd for C₂₄H₂₈FN₂O₂, 395.2; found, 394.8 ¹H NMR (400 MHz, CD₃OD): δ 8.47 (s, 1H), 8.10 (d, J = 7.5 Hz, 2H), 7.63 (d, J = 7.5 Hz, 2H), 7.23 (d, J = 3.1 Hz, 1H), 6.76 (s, 1H), 6.38 (d, J = 2.4 Hz, 1H), 4.71 (dd, J = 47.1, 6.7 Hz, 2H), 4.59- 3.93 (m, 3H), 3.21-2.98 (m, 2H), 2.41 (s, 3H), 2.34-2.25 (m, 2H), 2.16 (s, 3H), 2.04-1.91 (m, 2H),. 1.85-1.81 (m, 1H). |
| 4-(1-((5,7-dimethyl-1H-indol-4- | | | |
| yl)methyl)-4- | | | |
| (fluoromethyl)piperidin-2- | | | |
| yl)benzoic acid | | | |
| 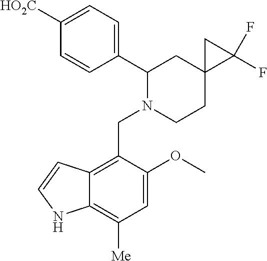 [SMILES: COc1cc(C)c2[nH]ccc2c1CN1CCC2(CC1c1ccc(C(=O)O)cc1)CC2(F)F] | 8 (Isomer 1) | Column: AD-3 IC 4.6 × 100 mm I.D., 3 μm Mobile phase: 30% MeOH [0.2% NH₃ (7M in MeOH)] Flow rate: 3.0 mL/min Column temperature: 40° C. Retention time = 0.89 min | LCMS (m/z): [M + H]⁺ calc'd for C₂₅H₂₇F₂N₂O₃, 441.2; found, 441.0 ¹H NMR (400 MHz, CD₃OD): δ 8.13 (d, J = 8.3 Hz, 2H), 7.64 (d, J = 8.1 Hz, 2H), 7.29 (d, J = 3.1 Hz, 1H), 6.74 (s, 1H), 6.34 (s, 1H), 4.20 (s, 2H), 3.94 (s, 1H), 3.76 (s, 3H), 3.45 (d, J = 10.4 Hz, 1H), 2.49 (s, 3H), 2.45-2.35 (m, 1H), 2.20 (s, 2H), 1.82 (d, J = 15.2 Hz, 1H), 1.66 (d, J = 13.6 Hz, 1H), 1.32 (s, 2H) |
| 4-(1,1-difluoro-6-((5-methoxy-7- | | | |
| methyl-1H-indol-4-yl)methyl)-6- | | | |
| azaspiro[2.5]octan-5-yl)benzoic | | | |
| acid | | | |
| Chemical Formula: C₂₅H₂₆F₂N₂O₃ | | | |
| Exact Mass: 440.2 | | | |
| 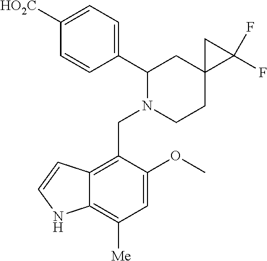 | 8 (Isomer 2) | Column: AD-3 IC 4.6 × 100 mm I.D., 3 μm Mobile phase: 30% MeOH [0.2% NH₃ (7M in MeOH)] Flow rate: 3.0 mL/min Column temperature: 40° C. Retention time = 1.40 min | LCMS (m/z): [M + H]⁺ calc'd for C₂₄H₂₈FN₂O₂, 395.2; found, 394.8 ¹H NMR (400 MHz, CD₃OD): δ 8.44 (s, 1H), 8.15 (d, J = 8.1 Hz, 2H), 7.66 (d, J = 7.9 Hz, 2H), 7.30 (d, J = 3.1 Hz, 1H), 6.76 (s, 1H), 6.34 (s, 1H), 4.35 (d, J = 50.4 Hz, 2H), 4.05 (s, 1H), 3.76 (s, 3H), 3.48 (s, 1H), 2.50 (s, 3H), 2.48-2.38 (m, 1H), 2.22 (s, 1H), 1.73 (d, J = 15.9 Hz, 1H), 1.49 (dd, J = 33.1, 12.3 Hz, 2H). |
| 4-(1,1-difluoro-6-((5-methoxy-7- | | | |
| methyl-1H-indol-4-yl)methyl)-6- | | | |
| azaspiro[2.5]octan-5-yl)benzoic | | | |
| acid | | | |
| Chemical Formula: C₂₅H₂₆F₂N₂O₃ | | | |
| Exact Mass: 440.2 | | | |
| 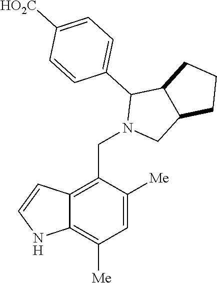 | 9 (Isomer 1) | Column: CHIRALPAK OJ-H 20 × 250 mm, 5 μm Mobile phase: 35% MeOH (0.2% NH₄OH) in CO₂ Flow rate: 12.5 mL/min Column temperature: 39.3° C. Retention time = 2.94 min | LCMS (m/z): [M + H]⁺ calc'd for C₂₅H₂₉N₂O₂, 389.2; found, 389.1 ¹H NMR (400 MHz, CD₃OD): δ 8.49 (s, 0.09H), 8.11 (d, J = 8.2 Hz, 2H), 7.62 (d, J = 8.3 Hz, 2H), 7.26 (d, J = 3.1 Hz, 1H), 6.77 (s, 1H), 6.26 (br s, 1H), 4.23 (dd, J = 24.1, 12.9 Hz, 2H), 4.00 (d, J = 9.7 Hz, 1H), 3.59 (t, J = 8.7 Hz, 1H), 2.99- 2.81 (m, 3H), 2.43 (s, 3H), 2.10 (s, 3H), 1.98-1.88 (m, 1H), 1.85- 1.78 (m, 1H), 1.73-1.55 (m, 4H). |
| 4-(2-((5,7-dimethyl-1H-indol-4- | | | |
| yl)methyl)octahydrocyclopenta[c] | | | |
| pyrrol-1-yl)benzoic acid | | | |
| 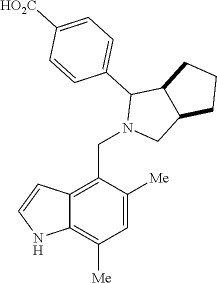 | 9 (Isomer 2) | Column: CHIRALPAK OJ-H 20 × 250 mm, 5 μm Mobile phase: 35% MeOH (0.2% NH₄OH) in CO₂ Flow rate: 12.5 mL/min Column temperature: 40.9° C. Retention time = 3.70 min | LCMS (m/z): [M + H]⁺ calc'd for C₂₅H₂₉N₂O₂, 389.2; found, 389.0 ¹H NMR (400 MHz, CD₃OD): δ 8.42 (s, 0.5H), 8.13 (d, J = 8.2 Hz, 2H), 7.64 (d, J = 8.2 Hz, 2H), 7.27 (d, J = 3.1 Hz, 1H), 6.78 (s, 1H), 6.26 (br s, 1H), 4.33 (d, J = 13.0 Hz, 1H), 4.24 (d, J = 12.8 Hz, 1H), 4.08 (d, J = 9.7 Hz, 1H), 3.70- 3.58 (m, 1H), 3.02-2.93 (m, 2H), 2.92-2.84 (m, 1H), 2.43 (s, 3H), 2.10 (s, 3H), 1.97-1.89 (m, 1H), 1.86-1.79 (m, 1H), 1.74-1.61 (m, 4H). |
| 4-(2-((5,7-dimethyl-1H-indol-4- | | | |
| yl)methyl)octahydrocyclopenta[c] | | | |
| pyrrol-1-yl)benzoic acid | | | |
| 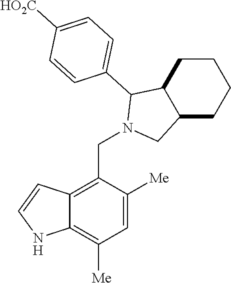 | 10 | | LCMS (m/z): [M + H]⁺ calc'd for C₂₆H₃₁N₂O₂, 403.2; found, 403.1 ¹H NMR (400 MHz, CD₃OD): δ 8.43 (br s, 0.5H), 8.10 (d, J = 8.3 Hz, 2H), 7.54 (d, J = 8.2 Hz, 2H), 7.31 (d, J = 3.2 Hz, 1H), 6.77 (s, 1H), 6.32 (s, 1H), 4.70 (d, J = 11.1 Hz, 1H), 4.59 (dd, J = 29.4, 13.3 Hz, 2H), 3.65 (dd, J = 11.8, 7.2 Hz, 1H), 3.36 (d, J = 4.4 Hz, 1H), 2.69- 2.60 (m, 1H), 2.58-2.49 (m, 1H), 2.45 (s, 3H), 2.11 (s, 3H), 1.97- 1.90 (m, 1H), 1.86-1.79 (m, 1H), 1.78-1.41 (m, 6H). |
| 4-(2-((5,7-dimethyl-1H-indol-4- | | | |
| yl)methyl)octahydro-1H-isoindol- | | | |
| 1-yl)benzoic acid | | | |
| 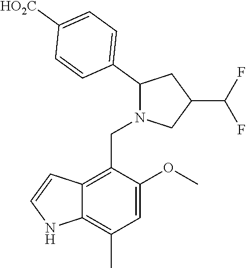 [SMILES: COc1cc(C)c2[nH]ccc2c1CN1CC(C(F)F)CC1c1ccc(C(=O)O)cc1] | 20 (Isomer 1) | | LCMS (m/z): [M + H]⁺ calc'd for C₂₃H₂₅F₂N₂O₃, 415.2; found, 415.1 ¹H NMR (400 MHz, CD₃OD): δ 8.05 (d, J = 8.2 Hz, 2H), 7.57 (d, J = 8.3 Hz, 2H), 7.22 (d, J = 3.1 Hz, 1H), 6.70 (s, 1H), 6.23 (d, J = 3.1 Hz, 1H), 5.94 (td, J = 56.6, 4.3 Hz, 1H), 4.59 (s, 1H), 4.05 (d, J = 12.2 Hz, 1H), 3.99 (s, 1H), 3.87 (d, J = 12.4 Hz, 1H), 3.76 (s, 3H), 2.84 (dd, J = 28.7, 18.8 Hz, 2H), 2.47 (s, 3H), 2.39-2.28 (m, 1H), 2.08 (dd, J = 24.1, 10.6 Hz, 1H). |
| 4-(4-(difluoromethyl)-1-((5- | | | |
| methoxy-7-methyl-1H-indol-4- | | | |
| yl)methyl)pyrrolidin-2-yl)benzoic | | | |
| acid | | | |
| 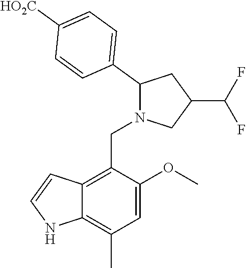 [SMILES: COc1cc(C)c2[nH]ccc2c1CN1CC(C(F)F)CC1c1ccc(C(=O)O)cc1] | 20 (Isomer 2) | | LCMS (m/z): [M + H]⁺ calc'd for C₂₃H₂₅F₂N₂O₃, 415.2; found, 415.1 ¹H NMR (400 MHz, CD₃OD): δ 8.05 (d, J = 8.3 Hz, 2H), 7.56 (t, J = 7.2 Hz, 2H), 7.23 (d, J = 3.1 Hz, 1H), 6.69 (d, J = 8.4 Hz, 1H), 6.23 (dd, J = 6.2, 3.2 Hz, 1H), 5.94 (td, J = 56.0, 3.9 Hz, 1H), 4.63 (s, 1H), 4.10 (d, J = 12.0 Hz, 1H), 3.97 (d, J = 12.4 Hz, 1H), 3.72 (s, 3H), 3.26 (s, 1H), 3.00 (dd, J = 19.0, 9.5 Hz, 1H), 2.88-2.72 (m, 1H), 2.47 (s, 3H), 2.18 (dt, J = 18.6, 10.3 Hz, 1H), 2.05 (dd, J = 12.5, 6.7 Hz, 1H). |
| 4-(4-(difluoromethyl)-1-((5- | | | |
| methoxy-7-methyl-1H-indol-4- | | | |
| yl)methyl)pyrrolidin-2-yl)benzoic | | | |
| acid | | | |
| 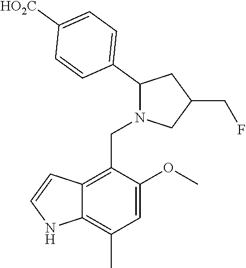 [SMILES: COc1cc(C)c2[nH]ccc2c1CN1CC(CF)CC1c1ccc(C(=O)O)cc1] | 21 | | LCMS (m/z): [M + H]⁺ calc'd for C₂₃H₂₆FN₂O₃, 397.2; found, 397.1 ¹H NMR (400 MHz, CD₃OD): δ 8.51 (s, 1H), 8.10 (d, J = 7.5 Hz, 2H), 7.56 (dd, J = 12.0, 7.7 Hz, 2H), 7.29 (d, J = 1.4 Hz, 1H), 6.70 (d, J = 5.3 Hz, 1H), 6.26 (dd, J = 11.1, 3.0 Hz, 1H), 4.64-4.21 (m, 5H), 3.73 (d, J = 6.5 Hz, 3H), 3.61 (dd, J = 11.5, 8.1 Hz, 1H), 3.47 (dd, J = 25.7, 16.5 Hz, 1H), 3.25-3.19 (m, 1H), 2.84 (s, 1H), 2.48 (s, 3H), 2.38 (d, J = 8.3 Hz, 1H), 2.18 (d, J = 12.6 Hz, 1H). |
| 4-(4-(fluoromethyl)-1-((5- | | | |
| methoxy-7-methyl-1H-indol-4- | | | |
| yl)methyl)pyrrolidin-2-yl)benzoic | | | |
| acid | | | |
| 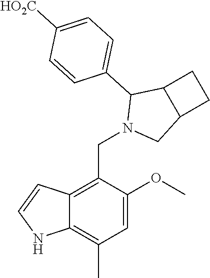 [SMILES: COc1cc(C)c2[nH]ccc2c1CN1CC2CCC2C1c1ccc(C(=O)O)cc1] | 22 | | LCMS (m/z): [M + H]⁺ calc'd for C₂₄H₂₇FN₂O₃, 391.2; found, 391.1 ¹H NMR (500 MHz, CD₃OD): δ 8.05 (d, J = 8.2 Hz, 2H), 7.55 (d, J = 8.1 Hz, 2H), 7.27 (d, J = 3.1 Hz, 1H), 6.75 (s, 1H), 6.43 (s, 1H), 4.57 (s, 1H), 4.46 (d, J = 14.8 Hz, 1H), 4.17 (dd, J = 67.1, 8.9 Hz, 2H), 3.80 (s, 3H), 3.24-3.05 (m, 2H), 2.98 (s, 1H), 2.50 (s, 3H), 2.21- 2.13 (m, 1H), 1.81 (t, J = 10.5 Hz, 2H), 1.71 (t, J = 11.4 Hz, 1H). |
| 4-(3-((5-methoxy-7-methyl-1H- | | | |
| indol-4-yl)methyl)-3- | | | |
| azabicyclo[3.2.0]heptan-2- | | | |
| yl)benzoic acid | | | |
| 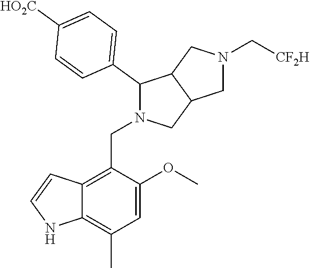 [SMILES: COc1cc(C)c2[nH]ccc2c1CN1CC2CN(CC(F)F)CC2C1c1ccc(C(=O)O)cc1] | 23 | | LCMS (m/z): [M + H]⁺ calc'd for C₂₆H₃₀F₂N₃O₃, 470.2; found, 470.1 ¹H NMR (400 MHz, CD₃OD): δ 8.16 (d, J = 6.4 Hz, 2H), 7.66 (d, J = 6.4 Hz, 2H), 7.31-7.32 (m, 1H), 6.70 (s, 1H), 6.30 (d, J = 2.4 Hz, 1H), 5.98-6.09 (m, 1H), 4.45 (d, J = 6.8 Hz, 2H), 4.34-4.37 (m, 1H), 3.80-3.82 (m, 2H), 3.74 (s, 3H), 2.96-3.07 (m, 6H), 2.47 (s, 4H), 2.42-2.43 (m, 1H). |
| 4-(5-(2,2-difluoroethyl)-2-((5- | | | |
| methoxy-7-methyl-1H-indol-4- | | | |
| yl)methyl)octahydropyrrolo[3,4- | | | |
| c]pyrrol-1-yl)benzoic acid | | | |
| 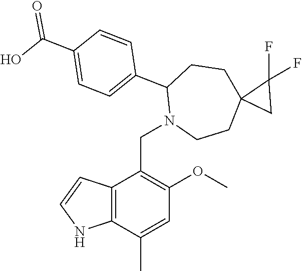 [SMILES: COc1cc(C)c2[nH]ccc2c1CN1CCC2(CCC1c1ccc(C(=O)O)cc1)CC2(F)F] | 24 (Isomer 1) | | LCMS (m/z): [M + H]⁺ calc'd for C₂₆H₂₉F₂N₂O₃, 455.2; found, 455.1 ¹H NMR (400 MHz, MeOD) 8.22 (d, J = 7.6 Hz, 2H), 7.73 (d, J = 7.6 Hz, 2H), 7.32 (s, 1H), 6.72 (s, 1H), 6.17-6.10 (m, 1H), 4.59-4.56 (m, 1H), 4.43 (d, J = 13.6 Hz, 1H), 3.61-3.53 (m, 5H), 3.47-3.46 (m, 1H), 2.78-2.74 (m, 1H), 2.49 (s, 4H), 2.20-2.11 (m, 1H), 1.94-1.90 (m, 1H), 1.76-1.70 (m, 1H), 1.43- (m, 1H), 1.76-1.70 (m, 1H), 1.43- 1.41 (m, 1H), 1.32-1.28 (m, 2H) |
| 4-(1,1-difluoro-6-((5-methoxy-7- | | | |
| methyl-1H-indol-4-yl)methyl)-6- | | | |
| azaspiro[2.6]nonan-7-yl)benzoic | | | |
| acid | | | |
| 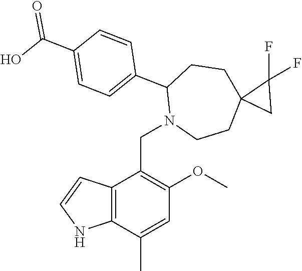 [SMILES: COc1cc(C)c2[nH]ccc2c1CN1CCC2(CCC1c1ccc(C(=O)O)cc1)CC2(F)F] | 24 (Isomer 2) | | LCMS (m/z): [M + H]⁺ calc'd for C₂₆H₂₉F₂N₂O₃, 455.2; found, 455.1 ¹H NMR (400 MHz, CD₃OD): δ 8.22 (d, J = 8.0 Hz, 2H), 7.74 (d, J = 8.0 Hz, 2H), 7.32 (s, 1H), 6.71 (s, 1H), 6.11 (s, 1H), 4.71 (d, J = 10.4 Hz, 1H), 4.56 (d, J = 12.8 Hz, 1H), 4.46 (d, J = 12.8 Hz, 1H) 3.59 (s, 3H), 3.54-3.52 (m, 2H), 2.73- 2.72 (m, 1H), 2.61-2.57 (m, 1H), 2.48 (s, 3H), 2.29-2.21 (m, 1H), 2.13-2.07 (m, 1H), 1.83- 1.71 (m, 4H), 1.28-1.25 (m, 2H) |
| 4-(1,1-difluoro-6-((5-methoxy-7- | | | |
| methyl-1H-indol-4-yl)methyl)-6- | | | |
| azaspiro[2.6]nonan-7-yl)benzoic | | | |
| acid | | | |
| 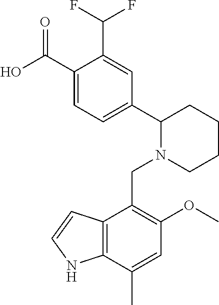 [SMILES: COc1cc(C)c2[nH]ccc2c1CN1CCCCC1c1ccc(C(=O)O)c(C(F)F)c1] | 25 | | LCMS (m/z): [M + H]⁺ calc'd for C₂₄H₂₇F₂N₂O₃, 429.2; found, 429.1 ¹H NMR (500 MHz, CD₃OD): δ 7.98-7.86 (m, 2H), 7.76-7.49 (m, 2H), 7.28 (d, J = 2.0 Hz, 1H), 6.73 (s, 1H), 6.36 (s, 1H), 4.25 (s, 2H), 3.75 (s, 3H), 3.56-3.31 (m, 3H), 2.49 (s, 3H), 2.11-1.61 (m, 6H). |
| 2-(difluoromethyl)-4-(1-((5- | | | |
| methoxy-7-methyl-1H-indol-4- | | | |
| yl)methyl)piperidin-2-yl)benzoic | | | |
| acid | | | |
| 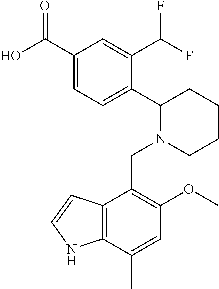 [SMILES: COc1cc(C)c2[nH]ccc2c1CN1CCCCC1c1ccc(C(=O)O)cc1C(F)F] | 26 (Isomer 1) | Column: CHIRAL OX-H 250 × 20 mm I.D., 5 μm Mobile phase: 45% MeOH [0.2% NH₃ (7M in MeOH)] Flow rate: 4 mL/ min Column temperature: 40° C. Retention time = 1.10 min | LCMS (m/z): [M + H]⁺ calc'd for C₂₄H₂₇F₂N₂O₃, 429.2; found, 429.1 ¹H NMR (400 MHz, CD₃OD): δ 8.40-8.20 (m, 2H), 7.88 (d, J = 8.1 Hz, 1H), 7.31 (d, J = 3.1 Hz, 1H), 7.10 (d, J = 54.9 Hz, 1H), 6.75 (s, 1H), 6.38 (d, J = 3.2 Hz, 1H), 4.71 (s, 1H), 4.24 (d, J = 12.5 Hz, 1H), 4.02 (d, J = 11.8 Hz, 1H), 3.76 (s, 3H), 3.51 (d, J = 12.3 Hz, 1H), 2.50 (s, 3H), 2.22-1.61 (m, 7H). |
| 3-(difluoromethyl)-4-(1-((5- | | | |
| methoxy-7-methyl-1H-indol-4- | | | |
| yl)methyl)piperidin-2-yl)benzoic | | | |
| acid | | | |
| 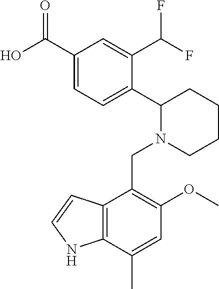 [SMILES: COc1cc(C)c2[nH]ccc2c1CN1CCCCC1c1ccc(C(=O)O)cc1C(F)F] | 26 (Isomer 2) | Column: CHIRAL OX-H 250 × 20 mm I.D., 5 μm Mobile phase: 45% MeOH [0.2% NH₃ (7M in MeOH)] Flow rate: 4 mL/min Column temperature: 40° C. Retention time = 2.10 min | LCMS (m/z): [M + H]⁺ calc'd for C₂₄H₂₇F₂N₂O₃, 429.2; found, 429.1 ¹H NMR (400 MHz, CD₃OD): δ 8.40-8.20 (m, 2H), 7.88 (d, J = 8.1 Hz, 1H), 7.31 (d, J = 3.1 Hz, 1H), 7.10 (d, J = 54.9 Hz, 1H), 6.75 (s, 1H), 6.38 (d, J = 3.2 Hz, 1H), 4.71 (s, 1H), 4.24 (d, J = 12.5 Hz, 1H), 4.02 (d, J = 11.8 Hz, 1H), 3.76 (s, 3H), 3.51 (d, J = 12.3 Hz, 1H), 2.50 (s, 3H), 2.22-1.61 (m, 7H). |
| 3-(difluoromethyl)-4-(1-((5- | | | |
| methoxy-7-methyl-1H-indol-4- | | | |
| yl)methyl)piperidin-2-yl)benzoic | | | |
| acid | | | |
| 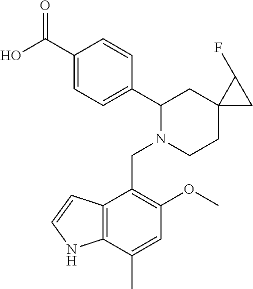 [SMILES: COc1cc(C)c2[nH]ccc2c1CN1CCC2(CC1c1ccc(C(=O)O)cc1)CC2F] | 27 (Isomer 1) | | LCMS (m/z): [M + H]⁺ calc'd for C₂₅H₂₈FN₂O₃, 423.2; found, 423 1H NMR (500 MHz, MeOD) δ 8.15 (d, J = 7.8 Hz, 2H), 7.66 (d, J = 7.8 Hz, 2H), 7.31 (d, J = 2.9 Hz, 1H), 6.77 (s, 1H), 6.33 (s, 1H), 4.74- 4.53 (m, 2H), 4.42-4.28 (m, 1H), 4.15 (d, J = 12.9 Hz, 1H), 3.76 (s, 3H), 3.60-3.50 (m, 1H), 3.28- 3.19 (m, 1H), 2.50 (s, 3H), 2.47- 2.38 (m, 1H), 2.40-2.21 (m, 1H), 1.69 (d, J = 13.9 Hz, 1H), 1.33 (d, J = 14.3 Hz, 1H), 0.96-0.76 (m, 2H). |
| 4-(1-fluoro-6-((5-methoxy-7- | | | |
| methyl-1H-indol-4-yl)methyl)-6- | | | |
| azaspiro[2.5]octan-5-yl)benzoic | | | |
| acid | | | |
| 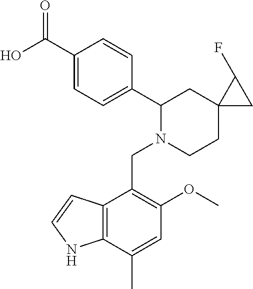 [SMILES: COc1cc(C)c2[nH]ccc2c1CN1CCC2(CC1c1ccc(C(=O)O)cc1)CC2F] | 27 (Isomer 2) | | LCMS (m/z): [M + H]⁺ calc'd for C₂₅H₂₈FN₂O₃, 423.2; found, 423 1H NMR (500 MHz, MeOD) δ 8.15 (d, J = 7.8 Hz, 2H), 7.64 (d, J = 7.7 Hz, 2H), 7.30 (d, J = 3.1 Hz, 1H), 6.82-6.71 (m, 1H), 6.36 (s, 1H), 4.69 (d, J = 64.4 Hz, 1H), 4.49- 4.26 (m, 2H), 4.18-4.06 (m, 1H), 3.77 (s, 3H), 3.53-3.35 (m, 2H), 2.61-2.52 (m, 1H), 2.50 (s, 3H), 2.25-2.08 (m, 1H), 1.83 (d, J = 13.3 Hz, 1H), 1.10 (s, 1H), 0.94- 0.76 (m, 2H). |
| 4-(1-fluoro-6-((5-methoxy-7- | | | |
| methyl-1H-indol-4-yl)methyl)-6- | | | |
| azaspiro[2.5]octan-5-yl)benzoic | | | |
| acid | | | |
| 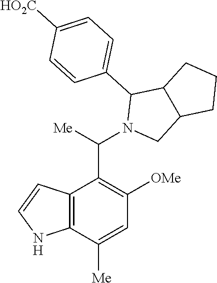 [SMILES: COc1cc(C)c2[nH]ccc2c1C(C)N1CC2CCCC2C1c1ccc(C(=O)O)cc1] | 37 (Isomer 1) | | LCMS (m/z): [M + H]+ calc'd for C₂₆H₃₁N₂O₃, 419.2; found, 419.1 ¹H NMR (400 MHz, CD₃OD) δ 10.59 (s, 1H), 7.58 (dd, J = 30.4, 8.0 Hz, 2H), 7.34 (s, 1H), 6.82 (d, J = 7.7 Hz, 2H), 6.55 (d, J = 3.0 Hz, 1H), 6.11 (s, 1H), 4.35-4.19 (m, 1H), 3.84 (t, J = 13.8 Hz, 1H), 3.82- 3.63 (m, 3H), 3.29-3.19 (m, 1H), 3.18-3.02 (m, 1H), 2.82 (dd, J = 17.1, 7.9 Hz, 1H), 2.25 (d, J = 15.9 Hz, 2H), 2.03-1.84 (m, 2H), 1.82 (t, J = 5.6 Hz, 3H), 1.78 (dd, J = 24.2, 19.9 Hz, 3H), 1.64-1.42 (m, 2H). |
| 4-(2-(1-(5-methoxy-7-methyl-1H- | | | |
| indol-4- | | | |
| yl)ethyl)octahydrocyclopenta[c] | | | |
| pyrrol-1-yl)benzoic acid | | | |
| 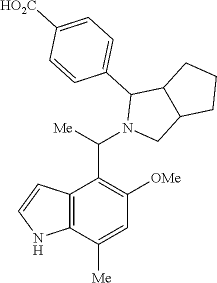 [SMILES: COc1cc(C)c2[nH]ccc2c1C(C)N1CC2CCCC2C1c1ccc(C(=O)O)cc1] | 37 (Isomer 2) | | LCMS (m/z): [M + H]+ calc'd for C₂₆H₃₁N₂O₃, 419.2; found, 419.1 ¹H NMR (400 MHz, CD₃OD) δ 10.59 (s, 1H), 7.58 (dd, J = 30.4, 8.0 Hz, 2H), 7.34 (s, 1H), 6.82 (d, J = 7.7 Hz, 2H), 6.55 (d, J = 3.0 Hz, 1H), 6.11 (s, 1H), 4.35-4.19 (m, 1H), 3.84 (t, J = 13.8 Hz, 1H), 3.82- 3.63 (m, 3H), 3.29-3.19 (m, 1H), 3.18-3.02 (m, 1H), 2.82 (dd, J = 17.1, 7.9 Hz, 1H), 2.25 (d, J = 15.9 Hz, 2H), 2.03-1.84 (m, 2H), 1.82 (t, J = 5.6 Hz, 3H), 1.78 (dd, J = 24.2, 19.9 Hz, 3H), 1.64-1.42 (m, 2H). |
| 4-(2-(1-(5-methoxy-7-methyl-1H- | | | |
| indol-4- | | | |
| yl)ethyl)octahydrocyclopenta[c] | | | |
| pyrrol-1-yl)benzoic acid | | | |
| 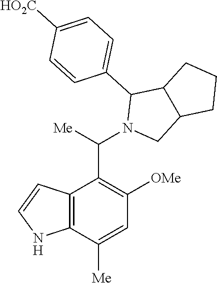 [SMILES: COc1cc(C)c2[nH]ccc2c1C(C)N1CC2CCCC2C1c1ccc(C(=O)O)cc1] | 37 (Isomer 3) | Column: CHIRAL IG 4.6 × 100 mm mm I.D., 5 μm Mobile phase: MeOH/ACN = 3/2 [0.2% NH₃ (7M in MeOH)] Flow rate: 3 mL/min Column temperature: 40° C. Retention time = 1.15 min | LCMS (m/z): [M + H]+ calc'd for C₂₆H₃₀N₂O₃, 418.2; found, 419.1 ¹H NMR (400 MHz, CD₃OD) δ 8.08 (d, J = 6.5 Hz, 2H), 7.57 (t, J = 24.6 Hz, 2H), 7.31 (s, 1H), 6.82 (s, 1H), 6.50 (s, 1H), 3.95 (s, 1H), 4.00- 3.75 (m, 3H), 3.71 (s, 1H), 2.87 (s, 2H), 2.69 (s, 1H), 2.49 (d, J = 24.9 Hz, 3H), 1.54 (dd, J = 39.5, 32.9 Hz, 6H), 1.44 (s, 1H), 1.40- 1.03 (m, 3H). |
| 4-(2-(1-(5-methoxy-7-methyl-1H- | | | |
| indol-4- | | | |
| yl)ethyl)octahydrocyclopenta[c] | | | |
| pyrrol-1-yl)benzoic acid | | | |
| 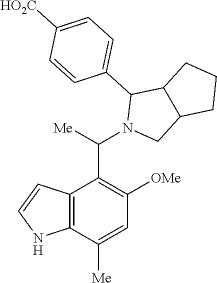 [SMILES: COc1cc(C)c2[nH]ccc2c1C(C)N1CC2CCCC2C1c1ccc(C(=O)O)cc1] | 37 (Isomer 4) | Column: CHIRAL IG 4.6 × 100 mm mm I.D., 5 μm Mobile phase: MeOH/ACN = 3/2 [0.2% NH₃ (7M in MeOH)] Flow rate: 3 mL/min Column temperature: 40° C. Retention time = 1.86 min | LCMS (m/z): [M + H]+ calc'd for C₂₆H₃₀N₂O₃, 418.2; found, 419.1 ¹H NMR (400 MHz, CD₃OD) δ 8.08 (d, J = 6.5 Hz, 2H), 7.57 (t, J = 24.6 Hz, 2H), 7.31 (s, 1H), 6.82 (s, 1H), 6.50 (s, 1H), 3.95 (s, 1H), 4.00- 3.75 (m, 3H), 3.71 (s, 1H), 2.87 (s, 2H), 2.69 (s, 1H), 2.49 (d, J = 24.9 Hz, 3H), 1.54 (dd, J = 39.5, 32.9 Hz, 6H), 1.44 (s, 1H), 1.40- 1.03 (m, 3H). |
| 4-(2-(1-(5-methoxy-7-methyl-1H- | | | |
| indol-4- | | | |
| yl)ethyl)octahydrocyclopenta[c] | | | |
| pyrrol-1-yl)benzoic acid | | | |
| 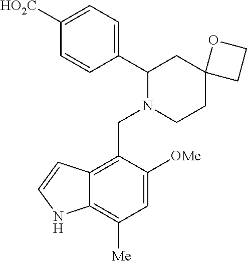 [SMILES: COc1cc(C)c2[nH]ccc2c1CN1CCC2(CCO2)CC1c1ccc(C(=O)O)cc1] | 38 (Isomer 1) | Column: CHIRALPAK IC-3 4.6 * 100 mm 3 um IC 45% B1 Mobile phase: MeOH [0.2% NH₃ (7M in MeOH)] Flow rate: 3.0 mL/min Column temperature: 40° C. Retention time = 1.44 min | LCMS (m/z): [M + H]⁺ calc'd for C₂₅H₂₉N₂O₄, 421.2; found, 421.0 ¹H NMR (500 MHz, CD₃OD) δ 8.11 (d, J = 8.0 Hz, 2H), 7.62 (d, J = 7.7 Hz, 2H), 7.27 (d, J = 3.1 Hz, 1H), 6.73 (s, 1H), 6.31 (s, 1H), 4.58 (t, J = 7.6 Hz, 2H), 4.12 (s, 2H), 3.75 (s, 3H), 2.85-2.55 (m, 3H), 2.48 (s, 3H), 2.44 (s, 1H), 2.27- 2.07 (m, 2H), 1.93 (s, 1H), 1.43- 1.26 (m, 2H) |
| 4-(7-((5-methoxy-7-methyl-1H- | | | |
| indol-4-yl)methyl)-1-oxa-7- | | | |
| azaspiro[3.5]nonan-6-yl)benzoic | | | |
| acid | | | |
| 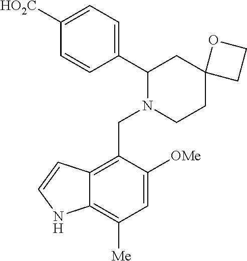 [SMILES: COc1cc(C)c2[nH]ccc2c1CN1CCC2(CCO2)CC1c1ccc(C(=O)O)cc1] | 38 (Isomer 2) | Column: CHIRALPAK IC-3 4.6 * 100 mm 3 um IC 45% B1 Mobile phase: MeOH [0.2% NH3 (7M in MeOH)] Flow rate: 3.0 mL/min Column temperature: Retention time = 5.0 min | LCMS (m/z): [M + H]⁺ calc'd for C₂₅H₂₉N₂O₄, 421.2; found, 421.0 1H NMR (400 MHz, CD3OD) δ 8.11 (d, J = 8.0 Hz, 2H), 7.62 (d, J = 7.7 Hz, 2H), 7.26 (d, J = 3.1 Hz, 1H), 6.72 (s, 1H), 6.31 (d, J = 2.9 Hz, 1H), 4.57 (t, J = 7.6 Hz, 2H), 4.12 (d, J = 12.2 Hz, 2H), 3.74 (s, 3H), 2.95-2.57 (m, 4H), 2.48 (s, 3H), 2.42 (s, 1H), 2.18 (dd, J = 25.7, 13.2 Hz, 2H), 1.93 (s, 1H), 1.44-1.24 (m, 2H). |
| 4-(7-((5-methoxy-7-methyl-1H- | | | |
| indol-4-yl)methyl)-1-oxa-7- | | | |
| azaspiro[3.5]nonan-6-yl)benzoic | | | |
| acid | | | |
| 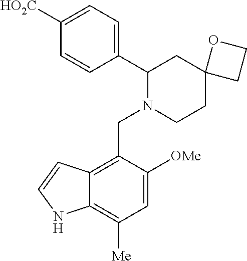 [SMILES: COc1cc(C)c2[nH]ccc2c1CN1CCC2(CCO2)CC1c1ccc(C(=O)O)cc1] | 38 (Isomer 3) | Column: AD-H (250 * 4.6 mm 5 um) Mobile phase: n- Hexane (0.1% DEA): EtOH (0.1% DEA) = 80: 20 Flow rate: 1.0 mL/min Column temperature: 40° C. Retention time = 5.39 min | LCMS (m/z): [M + H]⁺ calc'd for C₂₅H₂₉N₂O₄, 421.2; found, 421.0 1H NMR (400 MHz, CD3OD) δ 8.14 (d, J = 8.0 Hz, 2H), 7.63 (d, J = 7.7 Hz, 2H), 7.30 (d, J = 2.8 Hz, 1H), 6.75 (s, 1H), 6.32 (s, 1H), 4.70- 4.39 (m, 3H), 4.15 (d, J = 81.4 Hz, 2H), 3.75 (s, 3H), 2.49 (s, 5H), 2.28 (d, J = 14.9 Hz, 2H), 1.93 (s, 2H), 1.57-1.21 (m, 2H). |
| 4-(7-((5-methoxy-7-methyl-1H- | | | |
| indol-4-yl)methyl)-1-oxa-7- | | | |
| azaspiro[3.5]nonan-6-yl)benzoic | | | |
| acid | | | |
| 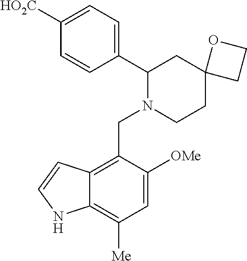 [SMILES: COc1cc(C)c2[nH]ccc2c1CN1CCC2(CCO2)CC1c1ccc(C(=O)O)cc1] | 38 (Isomer 4) | Column: AD-H (250 * 4.6 mm 5 um) Mobile phase: MeOH [0.2% NH3 (7M in MeOH)] Flow rate: 3.0 mL/min Column temperature: 40° C. Retention time = 6.9 min | LCMS (m/z): [M + H]⁺ calc'd for C₂₅H₂₉N₂O₄, 421.2; found, 421.0 1H NMR (400 MHz, CD3OD) δ 8.12 (d, J = 7.9 Hz, 2H), 7.62 (d, J = 7.7 Hz, 2H), 7.29 (d, J = 2.9 Hz, 1H), 6.74 (s, 1H), 6.32 (s, 1H), 4.54 (dd, J = 36.6, 29.3 Hz, 3H), 4.11 (d, J = 83.6 Hz, 2H), 3.75 (s, 3H), 2.49 (s, 5H), 2.32-2.12 (m, 2H), 1.93 (s, 2H), 1.50-1.25 (m, 2H). (m, 2H). |
| 4-(7-((5-methoxy-7-methyl-1H- | | | |
| indol-4-yl)methyl)-1-oxa-7- | | | |
| azaspiro[3.5]nonan-6-yl)benzoic | | | |
| acid | | | |
| 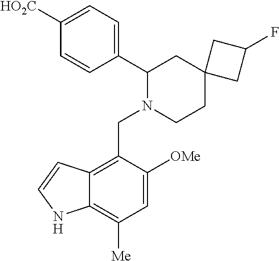 [SMILES: COc1cc(C)c2[nH]ccc2c1CN1CCC2(CC(F)C2)CC1c1ccc(C(=O)O)cc1] | 40 (isomer mixture 1) | | LCMS (m/z): [M + H]+ calc'd for C26H30FN2O3, 437; found, 437 1H NMR (400 MHz, CD3OD) δ 8.16 (d, J = 7.8 Hz, 2 H), 7.63 (d, J = 7.4 Hz, 2H), 7.31 (s, 1H), 6.75 (s, 1H), 6.33 (s, 1H), 433 (d, J = 39.8 Hz, 2H), 4.08 (d, J = 11.8 Hz, 1H), 3.74 (s, 3H), 3.54-3.35 (m, 2H), 3.13 (s, 1H), 2.67 (s, 1H), 2.50 (s, 3H), 2.27 (s, 3H), 2.06 (d, J = 14.4 Hz, 2H), 1.91 (s, 1H), 1.79 (d, J = 14.1 Hz, 1 H) |
| 4-(2-fluoro-7-((5-methoxy-7- | | | |
| methyl-1H-indol-4-yl)methyl)-7- | | | |
| azaspiro[3.5]nonan-6-yl)benzoic | | | |
| acid | | | |
| 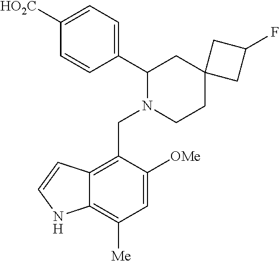 [SMILES: COc1cc(C)c2[nH]ccc2c1CN1CCC2(CC(F)C2)CC1c1ccc(C(=O)O)cc1] | 40 (isomer mixture 2) | | LCMS (m/z): [M + H]+ calc'd for C26H30FN2O3, 437; found, 437 1H NMR (400 MHz, CD3OD) δ 8.12 (d, J = 7.9 Hz, 2 H), 7.61 (d, J = 7.7 Hz, 2H), 7.30 (d, J = 3.0 Hz, 1H), 6.75 (s, 1H), 6.31 (s, 1H), 4.28 (s, 2H), 4.05 (d, J = 11.9 Hz, 1H), 3.74 (s, 3H), 3.43 (d, J = 40.4 Hz, 2H), 3.19 (s, 1H), 2.74 (s, 2H), 2.49 (s, 3H), 2.27 (s, 1H), 2.18-1.97 (m, 5H), 1.85 (d, J = 13.4 Hz, 1H). |
| 4-(2-fluoro-7-((5-methoxy-7- | | | |
| methyl-1H-indol-4-yl)methyl)-7- | | | |
| azaspiro[3.5]nonan-6-yl)benzoic | | | |
| acid | | | |

### Synthesis of 4-(2-((5,7-dimethyl-1H-indol-4-yl)methyl)-2-azabicyclo[2.2.1]heptan-3-yl)benzoic acid (Example 11) and 4-(2-((5,7-dimethyl-1H-indol-4-yl)methyl)-2-azabicyclo[2.2.1]heptan-3-yl)benzamide (Example 12)

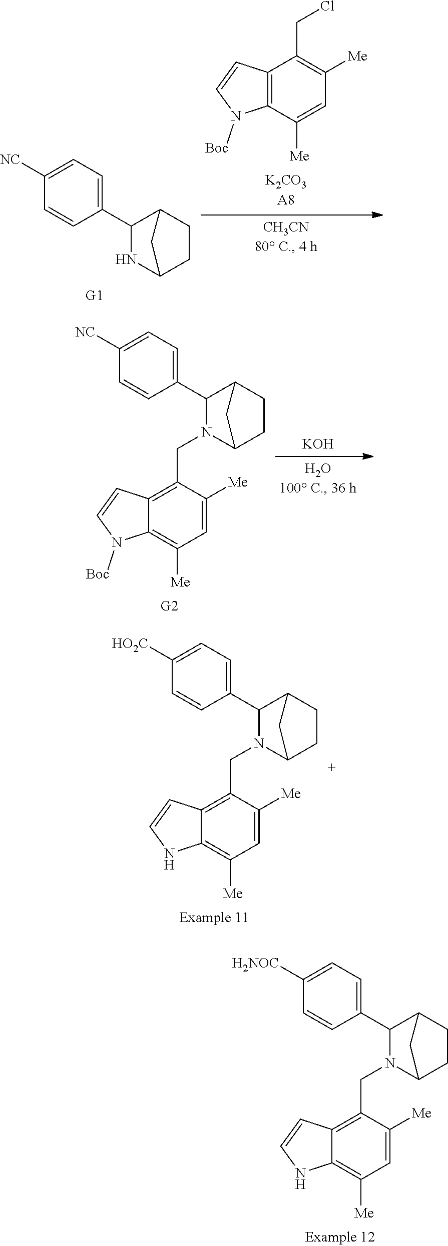

### Step 1: Synthesis of tert-butyl 4-((3-(4-cyanophenyl)-2-azabicyclo[2.2.1]heptan-2-yl)methyl)-5,7-dimethyl-1H-indole-1-carboxylate (G2)

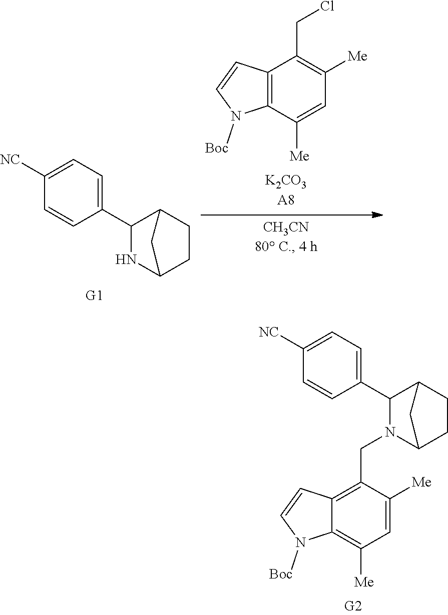 [SMILES: Cc1cc(C)c2c(ccn2C(=O)OC(C)(C)C)c1CCl ; N#Cc1ccc(C2NC3CCC2C3)cc1 ; Cc1cc(C)c2c(ccn2C(=O)OC(C)(C)C)c1CN1C2CCC(C2)C1c1ccc(C#N)cc1]

To a solution of 4-(2-azabicyclo[2.2.1]heptan-3-yl)benzonitrile (85 mg, 0.43 mmol) in CH₃CN (1.50 mL) was added K₂CO₃ (178 mg, 1.29 mmol) and the resulting mixture stirred for 10 min. tert-Butyl 4-(chloromethyl)-5,7-dimethyl-1H-indole-1-carboxylate (152 mg, 0.51 mmol) was added and the reaction mixture heated to 80° C. and kept at that temp for 4 h. The resulting mixture was cooled to rt, diluted with water (20 mL), and extracted with ethyl acetate (3×20 mL). The combined organic layers were washed with brine (30 mL), dried over anhydrous Na2SO₄, filtered, and concentrated to give a crude residue that was purified by flash column chromatography (SiO2, 10:1 petroleum ether/ethyl acetate) to give the pure desired compound (110 mg, 56%) as a colorless gum. LCMS (m/z): [M+H]⁺ calc'd for C₂₉H₃₄N₃O₂, 456.2; found, 455.8.

### Step 2: Synthesis of 4-(2-((5,7-dimethyl-1H-indol-4-yl)methyl)-2-azabicyclo[2.2.1]heptan-3-yl)benzoic acid (Example 11) and 4-(2-((5,7-dimethyl-1H-indol-4-yl)methyl)-2-azabicyclo[2.2.1]heptan-3-yl)benzamide (Example 12)

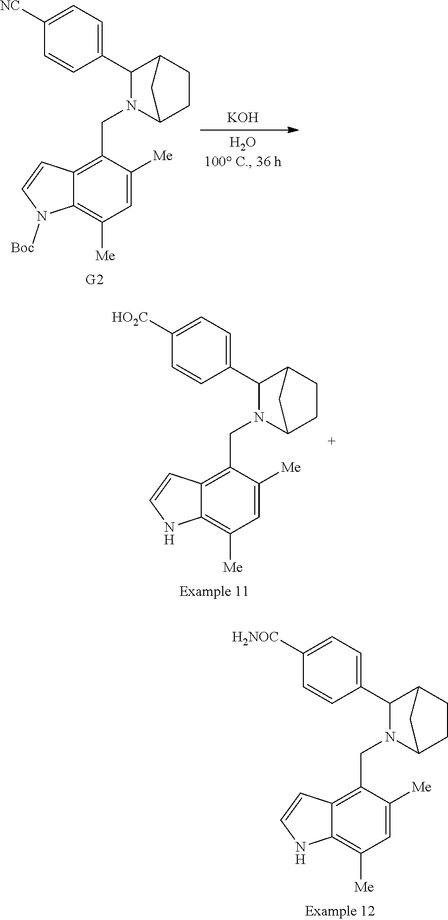

To a solution of tert-butyl 4-((3-(4-cyanophenyl)-2-azabicyclo[2.2.1]heptan-2-yl)methyl)-5,7-dimethyl-1H-indole-1-carboxylate (50 mg, 0.11 mmol) in water (0.5 mL) was added KOH (123 mg, 2.19 mmol). The reaction mixture was heated to 100° C. and stirred at that temp for 36 h. After cooling to rt, the pH was adjusted to 6.4-6.7 with citric acid (1 M) and extracted with CH₂Cl₂ (3×20 mL). The combined organic layers were washed with brine (10 mL) and concentrated in vacuo. The residue was purified by prep-HPLC (column: Waters™ XBridge 2.1×50 mm 3.5 μm; mobile phase A [water (0.05% trifluoroacetic acid v/v)] and B [acetonitrile (0.05% trifluoroacetic acid)]; gradient B: 0-60% over 7 min).

Example 11: 4-(2-((5,7-dimethyl-1H-indol-4-yl)methyl)-2-azabicyclo[2.2.1]heptan-3-yl)benzoic acid (5 mg) was obtained as a white solid. LCMS (m/z): [M+H]⁺ calc'd for C₂₄H₂₇N₂O₂, 375.2; found, 374.9. ¹H NMR (400 MHz, CD₃OD): δ 8.43 (br s, 1H), 7.92 (d, J=6.0 Hz, 2H), 7.36 (d, J=7.5 Hz, 2H), 7.26 (d, J=1.9 Hz, 1H), 6.72 (s, 1H), 6.43 (d, J=1.1 Hz, 1H), 4.56 (brs, 2H), 4.08-3.71 (m, 2H), 2.75-2.53 (m, 2H), 2.43 (s, 3H), 2.36-2.33 (m, 1H), 2.20 (s, 3H), 2.00-1.66 (m, 4H).

Isomers of Example 11 were separated by SFC. Isomer 1 and Isomer 2 were purified on a CHIRALPAK OJ-H column. The mobile phase A [CO₂] and B [Ethanol (0.2% NH₄OH)] at a flow rate of 12.5 mL/min and a column temperature of 39° C. (35% B in A). The retention time of Isomer 1 is 3.64 min. The retention time of Isomer 2 is 4.51 min.

Isomer 1: 4-(2-((5,7-dimethyl-1H-indol-4-yl)methyl)-2-azabicyclo[2.2.1]heptan-3-yl)benzoic acid (14 mg). LCMS (m/z): [M+H]⁺ calc'd for C₂₄H₂₇N₂O₂, 375.2; found, 375.2. ¹H NMR (400 MHz, CD₃OD): δ 7.77 (d, J=7.3 Hz, 2H), 7.22 (d, J=8.0 Hz, 2H), 7.12 (d, J=2.5 Hz, 1H), 6.59 (s, 1H), 6.32 (d, J=1.5 Hz, 1H), 4.37 (br s, 2H), 3.83-3.63 (m, 2H), 2.56-2.37 (m, 2H), 2.30 (s, 3H), 2.10 (s, 3H), 1.87-1.40 (m, 5H).

Isomer 2: 4-(2-((5,7-dimethyl-1H-indol-4-yl)methyl)-2-azabicyclo[2.2.1]heptan-3-yl)benzoic acid. LCMS (m/z): [M+H]⁺ calc'd for C₂₄H₂₇N₂O₂, 375.2; found, 375.2. ¹H NMR (400 MHz, CD₃OD): δ 7.89 (d, J=7.2 Hz, 2H), 7.34 (d, J=7.1 Hz, 2H), 7.24 (d, J=2.7 Hz, 1H), 6.71 (s, 1H), 6.44 (d, J=1.8 Hz, 1H), 4.45 (br s, 2H), 3.99-3.71 (m, 2H), 2.68-2.51 (m, 2H), 2.42 (s, 3H), 2.22 (s, 3H), 1.94-1.59 (m, 5H).

Example 12: 4-(2-((5,7-dimethyl-1H-indol-4-yl)methyl)-2-azabicyclo[2.2.1]heptan-3-yl)benzamiwas obtained as a white solid. LCMS (m/z): [M+H]⁺ calc'd for C₂₄H₂₈N30, 374.5; found, 373.8. ¹H NMR (400 MHz, CD₃OD): δ 8.41 (s, 0.47H), 7.70 (d, J=7.1 Hz, 2H), 7.33 (d, J=8.0 Hz, 2H), 7.17 (d, J=2.6 Hz, 1H), 6.63 (s, 1H), 6.45 (d, J=2.8 Hz, 1H), 4.25 (br s, 2H), 3.75-3.45 (m, 2H), 2.51-2.40 (m, 2H), 2.36 (s, 3H), 2.25 (s, 3H), 2.10-1.98 (m, 1H), 1.86-1.36 (m, 4H).

The following examples were synthesized using the ester hydrolysis procedure described above using appropriate starting materials:

| | | Isomer | |
|---|---|---|---|
| | | Separation | |
| | | Method and | |
| | | Retention Time | |
| Name and Stucture | Ex. # | (if any) | LCMS + ¹H NMR |
| 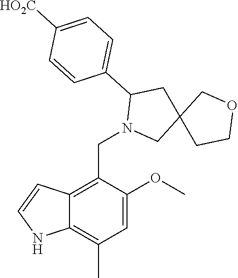 [SMILES: COc1cc(C)c2[nH]ccc2c1CN1CC2(CCOC2)CC1c1ccc(C(=O)O)cc1] | 28 (Isomer 1) | Column: AD-3 4.6 × 100 mm, 3 μm Mobile phase: 25% EtOH [1% NH₃ (7 M in MeOH)] in CO₂ Flow rate: 4 mL/min Column temperature: 40° C. Retention time = 1.85 min | LCMS (m/z): [M + H]⁺ calc'd for C₂₅H₂₉N₂O₄, 421.2; found, 421.1 ¹H NMR (400 MHz, CD₃OD): δ 8.03 (d, J = 8.3 Hz, 2H), 7.42 (d, J = 7.8 Hz, 2H), 7.27 (d, J = 3.1 Hz, 1H), 6.69 (s, 1H), 6.37 (d, J = 3.1 Hz, 1H), 4.28-4.24 (m, 1H), 4.12 (br s, 2H), 3.78 (s, 3H), 3.72-3.60 (m, 2H), 3.52- 3.48 (m, 1H), 3.40 (br s, 1H), 3.13 (br s, 1H), 2.48 (s, 3H), 2.14-2.10 (m, 1H), 2.04-1.93 (m, 1H), 1.62-1.57 (m, 1H), 1.44-1.40 (m, 1H), 1.29 (br s, 1H). |
| 4-(7-((5-methoxy-7-methyl-1H-indol-4- | | | |
| yl)methyl)-2-oxa-7-azaspiro[4.4]nonan- | | | |
| 8-yl)benzoic acid | | | |
| 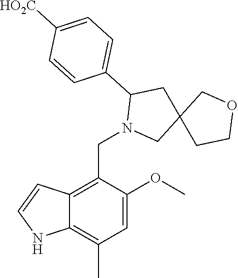 [SMILES: COc1cc(C)c2[nH]ccc2c1CN1CC2(CCOC2)CC1c1ccc(C(=O)O)cc1] | 28 (Isomer 2) | Column: AD-3 4.6 × 100 mm, 3 μm Mobile phase: 25% EtOH [1% NH₃ (7 M in MeOH)] in CO₂ Flow rate: 4 mL/min Column temperature: 40° C. Retention time = 2.30 min | LCMS (m/z): [M + H]⁺ calc'd for C₂₅H₂₉N₂O₄, 421.2; found, 421.1 ¹H NMR (400 MHz, CD₃OD): δ 8.06 (d, J = 8.2 Hz, 2H), 7.58 (d, J = 8.2 Hz, 2H), 7.25 (d, J = 3.1 Hz, 1H), 6.70 (s, 1H), 6.27 (d, J = 3.1 Hz, 1H), 4.34 (br s, 1H), 4.24-4.20 (m, 1H), 4.13- 4.09 (m, 1H), 3.85-3.76 (m, 3H), 3.75 (s, 3H), 3.68 (d, J = 8.3 Hz, 1H), 3.20-3.16 (m, 1H), 2.47 (s, 3H), 2.45-2.41 (m, 1H), 2.15-1.97 (m, 3H), 1.28 (br s, 1H). |
| 4-(7-((5-methoxy-7-methyl-1H-indol-4- | | | |
| yl)methyl)-2-oxa-7-azaspiro[4.4]nonan- | | | |
| 8-yl)benzoic acid | | | |
| 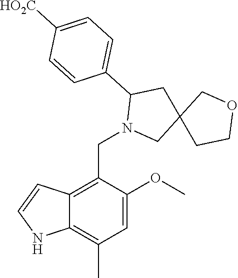 [SMILES: COc1cc(C)c2[nH]ccc2c1CN1CC2(CCOC2)CC1c1ccc(C(=O)O)cc1] | 28 (Isomer 3) | Column: AD-3 4.6 × 100 mm, 3 μm Mobile phase: 25% EtOH [1% NH₃ (7 M in MeOH)] in CO₂ Flow rate: 4 mL/min Column temperature: 40° C. Retention time = 3.55 min | LCMS (m/z): [M + H]⁺ calc'd for C₂₅H₂₉N₂O₄, 421.2; found, 421.1 ¹H NMR (400 MHz, CD₃OD): δ 8.07 (d, J = 8.2 Hz, 2H), 7.50 (d, J = 7.8 Hz, 2H), 7.26 (d, J = 3.1 Hz, 1H), 6.69 (d, J = 6.6 Hz, 1H), 6.32 (d, J = 3.1 Hz, 1H), 4.33 (br s, 1H), 4.23-4.19 (m, 1H), 4.11-4.07 (m, 1H), 3.83-3.80 (m, 2H), 3.76 (d, J = 7.0 Hz, 3H), 3.68 (d, J = 8.0 Hz, 1H), 3.62 (d, J = 8.4 Hz, 1H), 3.52-3.48 (m, 1H), 3.18-3.15 (m, 1H), 2.47 (s, 3H), 2.45-2.39 (m, 1H), 2.19-1.94 (m, 3H), 1.63-1.59 (m, 1H), 1.43-1.39 (m, 1H), 1.28 (br s, 1H). |
| 4-(7-((5-methoxy-7-methyl-1H-indol-4- | | | |
| yl)methyl)-2-oxa-7-azaspiro[4.4]nonan- | | | |
| 8-yl)benzoic acid | | | |
| 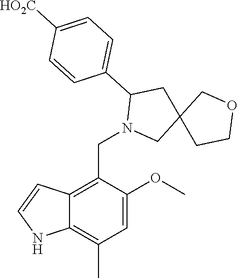 [SMILES: COc1cc(C)c2[nH]ccc2c1CN1CC2(CCOC2)CC1c1ccc(C(=O)O)cc1] | 28 (Isomer 4) | Column: AD-H 4.6 × 100 mm, 5 μm Mobile phase: 25% EtOH [1% NH₃ (7 M in MeOH)] in CO₂ Flow rate: 4 mL/min Column temperature: 40° C. Retention time = 2.25 min | LCMS (m/z): [M + H]⁺ calc'd for C₂₅H₂₉N₂O₄, 421.2; found, 421.1 ¹H NMR (400 MHz, CD₃OD): δ 8.02 (d, J = 8.2 Hz, 2H), 7.40 (d, J = 8.1 Hz, 2H), 7.28 (d, J = 3.1 Hz, 1H), 6.68 (s, 1H), 6.39 (d, J = 3.1 Hz, 1H), 4.29-4.24 (m, 2H), 4.16-4.12 (m, 1H), 3.76 (s, 3H), 3.70-3.68 (m, 2H), 3.40 (d, J = 8.6 Hz, 2H), 3.24-3.20 (m, 1H), 3.11 (d, J = 8.7 Hz, 1H), 2.47 (s, 3H), 2.18- 2.02 (m, 4H). |
| 4-(7-((5-methoxy-7-methyl-1H-indol-4- | | | |
| yl)methyl)-2-oxa-7-azaspiro[4.4]nonan- | | | |
| 8-yl)benzoic acid | | | |
| 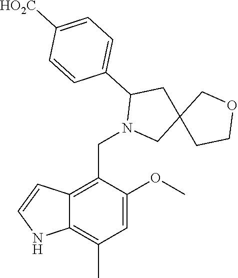 [SMILES: COc1cc(C)c2[nH]ccc2c1CN1CC2(CCOC2)CC1c1ccc(C(=O)O)cc1] | 28 (Isomer 5) | Column: AD-H 4.6 × 100 mm, 5 μm Mobile phase: 25% EtOH [1% NH₃ (7 M in MeOH)] in CO₂ Flow rate: 4 mL/min Column temperature: 40° C. Retention time = 3.95 min | LCMS (m/z): [M + H]⁺ calc'd for C₂₅H₂₉N₂O₄, 421.2; found, 421.1 ¹H NMR (400 MHz, CD₃OD): δ 8.02 (d, J = 8.2 Hz, 2H), 7.40 (d, J = 8.0 Hz, 2H), 7.28 (d, J = 3.1 Hz, 1H), 6.68 (s, 1H), 6.39 (d, J = 3.1 Hz, 1H), 4.27-4.23 (m, 2H), 4.13-4.09 (m, 1H), 3.76 (s, 3H), 3.75-3.65 (m, 2H), 3.40 (d, J = 8.6 Hz, 2H), 3.23-3.19 (s, 1H), 3.10 (d, J = 8.6 Hz, 1H), 2.47 (s, 3H), 2.15- 2.04 (m, 4H). |
| 4-(7-((5-methoxy-7-methyl-1H-indol-4- | | | |
| yl)methyl)-2-oxa-7-azaspiro[4.4]nonan- | | | |
| 8-yl)benzoic acid | | | |
| 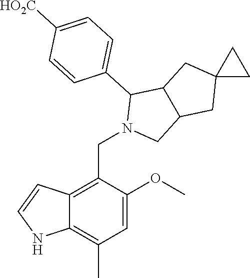 [SMILES: COc1cc(C)c2[nH]ccc2c1CN1CC2CC3(CC3)CC2C1c1ccc(C(=O)O)cc1] | 29 (Isomer 1) | | LCMS (m/z): [M + H]⁺ calc'd for C₂₇H₃₁N₂O₃, 431.2; found, 431.1 ¹H NMR (400 MHz, CD₃OD): δ 8.16 (d, J = 6.4 Hz, 2H), 7.65 (d, J = 6.4 Hz, 2H), 7.32 (d, J = 2.4 Hz, 1H), 6.69 (s, 1H), 6.33 (d, J = 2.4 Hz, 1H), 4.52 (d, J = 10.4 Hz, 1H), 4.44 (d, J = 9.2 Hz, 1H), 4.39 (d, J = 10.4 Hz, 1H), 3.69- 3.82 (m, 2H), 3.36-3.39 (m, 1H), 2.89-2.93 (m, 1H), 2.58 (d, J = 4.8 Hz, 1H), 2.47 (s, 3H), 2.29-2.33 (m, 1H), 2.17-2.20 (m, 1H), 2.03 (d, J = 4.4 Hz, 1H), 1.89-1.94 (m, 1H), 1.23-1.33 (m, 2H), 0.88-0.96 (m, 2H), 0.44- 0.47 (m, 1H), 0.31-0.33 (m, 1H). |
| 4-(2-((5-methoxy-7-methyl-1H-indol-4- | | | |
| yl)methyl)hexahydro-1H- | | | |
| spiro[cyclopenta[c]pyrrole-5,1′- | | | |
| cyclopropan]-1-yl)benzoic acid | | | |
| 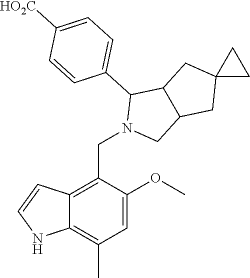 [SMILES: COc1cc(C)c2[nH]ccc2c1CN1CC2CC3(CC3)CC2C1c1ccc(C(=O)O)cc1] | 29 (Isomer 2) | | LCMS (m/z): [M + H]⁺ calc'd for C₂₇H₃₁N₂O₃, 431.2; found, 431.1 ¹H NMR (400 MHz, CD₃OD): δ 8.06-8.16 (m, 2H), 7.46-7.66 (m, 2H), 7.33 (d, J = 2.4 Hz, 1H), 6.33-6.79 (m, 2H), 4.52- 4.59 (m, 2H), 4.47-4.50 (m, 1H), 3.60-3.75 (m, 4H), 3.31- 3.34 (m, 1H), 2.97-3.01 (m, 1H), 2.67 (d, J = 7.6 Hz, 1H), 2.48 (d, J = 2.8 Hz, 3H), 2.31 (s, 1H), 2.17-2.22 (m, 1H), 1.89 (d, J = 5.2 Hz, 1H), 1.19-1.21 (m, 1H), 0.93-0.96 (m, 1H), 0.44-0.56 (m, 1H), 0.27-0.33 (m, 1H). |
| 4-(2-((5-methoxy-7-methyl-1H-indol-4- | | | |
| yl)methyl)hexahydro-1H- | | | |
| spiro[cyclopenta[c]pyrrole-5,1′- | | | |
| cyclopropan]-1-yl)benzoic acid | | | |
| 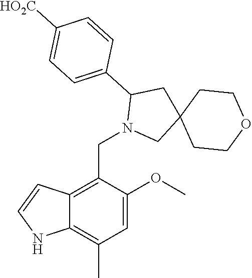 [SMILES: COc1cc(C)c2[nH]ccc2c1CN1CC2(CCOCC2)CC1c1ccc(C(=O)O)cc1] | 30 | | LCMS (m/z): [M + H]⁺ calc'd for C₂₆H₃₁N₂O₄, 435.2; found, 435.1 ¹H NMR (500 MHz, CD₃OD): δ 8.07 (d, J = 8.1 Hz, 2H), 7.57 (d, J = 8.1 Hz, 2H), 7.27 (d, J = 3.1 Hz, 1H), 6.72 (s, 1H), 6.24 (d, J = 3.0 Hz, 1H), 4.55-4.54 (m, 1H), 4.29-4.28 (m, 2H), 3.75 (s, 3H), 3.73-3.72 (m, 1H), 3.68-3.61 (m, 1H), 3.58- 3.56 (m, 2H), 3.34-3.33 (m, 1H), 3.20 (br s, 1H), 2.48 (s, 3H), 2.47-2.45 (m, 1H), 2.02 (s, 1H), 1.78-1.74 (m, 4H) |
| 4-(2-((5-methoxy-7-methyl-1H-indol-4- | | | |
| yl)methyl)-8-oxa-2-azaspiro[4.5]decan- | | | |
| 3-yl)benzoic acid | | | |
| 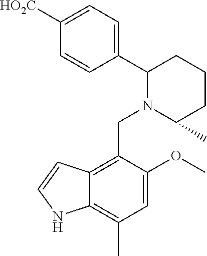 [SMILES: COc1cc(C)c2[nH]ccc2c1CN1C(c2ccc(C(=O)O)cc2)CCC[C@@H]1C] | 31 (Isomer 1) | | LCMS (m/z): [M + H]⁺ calc'd for C₂₄H₂₉N₂O₃, 393.2; found, 435.1 ¹H NMR (400 MHz, CD₃OD): δ 7.80 (d, J = 8.1 Hz, 2H), 7.45 (d, J = 8.1 Hz, 2H), 7.21 (d, J = 3.1 Hz, 1H), 6.77 (s, 1H), 6.54 (s, 1H), 4.25-4.16 (m, 1H), 4.08 (d, J = 15.2 Hz, 1H), 3.93 (dd, J = 16.1, 10.0 Hz, 1H), 3.67 (s, 3H), 3.09-2.98 (m, 1H), 2.41 (s, 3H), 2.06-1.99 (m, 1H), 1.91-1.82 (m, 2H), 1.80- 1.71 (m, 2H), 1.62 (dd, J = 8.1, 4.9 Hz, 1H), 1.03 (d, J = 6.4 Hz, 3H) |
| 4-((6S)-1-((5-methoxy-7-methyl-1H- | | | |
| indol-4-yl)methyl)-6-methylpiperidin-2- | | | |
| yl)benzoic acid | | | |
| 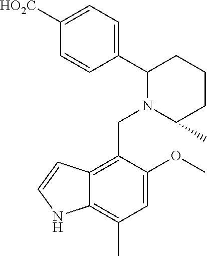 [SMILES: COc1cc(C)c2[nH]ccc2c1CN1C(c2ccc(C(=O)O)cc2)CCC[C@@H]1C] | 31 (Isomer 2) | | LCMS (m/z): [M + H]⁺ calc'd for C₂₄H₂₉N₂O₃, 393.2; found, 435.1 ¹H NMR (400 MHz, CD₃OD): δ 8.07 (d, J = 8.2 Hz, 2H), 7.62 (d, J = 8.2 Hz, 2H), 7.25 (d, J = 3.2 Hz, 1H), 6.75 (s, 1H), 6.24 (s, 1H), 4.53 (s, 1H), 4.20 (t, J = 14.1 Hz, 2H), 3.81 (s, 3H), 3.58 (s, 1H), 2.47 (d, J = 9.9 Hz, 3H), 2.21 (s, 2H), 2.00 (t, J = 13.6 Hz, 2H), 1.76 (d, J = 16.4 Hz, 2H), 1.54 (d, J = 6.8 Hz, 3H). |
| 4-((6S)-1-((5-methoxy-7-methyl-1H- | | | |
| indol-4-yl)methyl)-6-methylpiperidin-2- | | | |
| yl)benzoic acid | | | |
| 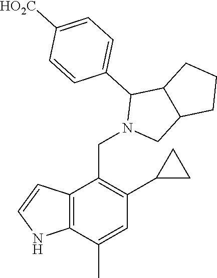 [SMILES: Cc1cc(C2CC2)c(CN2CC3CCCC3C2c2ccc(C(=O)O)cc2)c2cc[nH]c12] | 32 (Isomer 1) | Column: AD-H 4.6 × 100 mm, 5 μm Mobile phase: 25% EtOH [1% NH₃ (7 M in MeOH)] in CO₂ Flow rate: 4 mL/min Column temperature: 40° C. Retention time = 2.34 min | LCMS (m/z): [M + H]⁺ calc'd for C₂₇H₃₁N₂O₂, 415.2; found, 415.1 1H NMR (400 MHz, MeOD) δ 8.15 (d, J = 8.1 Hz, 2H), 7.69 (d, J = 8.1 Hz, 2H), 7.31 (d, J = 2.9 Hz, 1H), 6.67 (s, 1H), 6.33 (s, 1H), 4.61 (d, J = 12.8 Hz, 1H), 4.52 (d, J = 12.8 Hz, 1H), 4.22 (d, J = 9.6 Hz, 1H), 3.76-3.75 (m, 1H), 3..31- 3.29 (m, 1H), 3..12 (br s, 1H), 3.03 (br s, 2H), 2.43 (s, 3H), 1.87-1.86 (m, 1H), 1.75-1.72 (m, 1H), 1.68-1.58 (m, 4H), 1.40 (br s, 1H), 0.77 (br s, 2H), 0.22 (br s, 1H), 0.09 (br s, 1H). |
| 4-(2-((5-cyclopropyl-7-methyl-1H- | | | |
| indol-4- | | | |
| yl)methyl)octahydrocyclopenta[c]pyrrol- | | | |
| 1-yl)benzoic acid | | | |
| 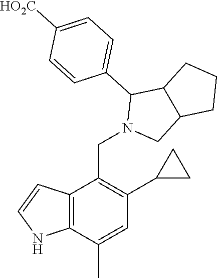 [SMILES: Cc1cc(C2CC2)c(CN2CC3CCCC3C2c2ccc(C(=O)O)cc2)c2cc[nH]c12] | 32 (Isomer 2) | Column: AD-H 4.6 × 100 mm, 5 μm Mobile phase: 25% EtOH [1% NH₃ (7 M in MeOH)] in CO₂ Flow rate: 4 mL/min Column temperature: 40° C. Retention time = 4.37 min | LCMS (m/z): [M + H]⁺ calc'd for C₂₇H₃₁N₂O₂, 415.2; found, 415.1 1H NMR (400 MHz, MeOD) δ 8.15 (d, J = 8.1 Hz, 2H), 7.69 (d, J = 8.1 Hz, 2H), 7.31 (d, J = 2.9 Hz, 1H), 6.67 (s, 1H), 6.33 (s, 1H), 4.61 (d, J = 12.8 Hz, 1H), 4.52 (d, J = 12.8 Hz, 1H), 4.22 (d, J = 9.6 Hz, 1H), 3.76-3.75 (m, 1H), 3..31- 3.29 (m, 1H), 3..12 (br s, 1H), 3.03 (br s, 2H), 2.43 (s, 3H), 1.87-1.86 (m, 1H), 1.75-1.72 (m, 1H), 1.68-1.58 (m, 4H), 1.40 (br s, 1H), 0.77 (br s, 2H), 0.22 (br s, 1H), 0.09 (br s, 1H). |
| 4-(2-((5-cyclopropyl-7-methyl-1H- | | | |
| indol-4- | | | |
| yl)methyl)octahydrocyclopenta[c]pyrrol- | | | |
| 1-yl)benzoic acid | | | |
| 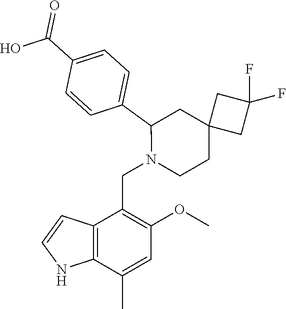 [SMILES: COc1cc(C)c2[nH]ccc2c1CN1CCC2(CC1c1ccc(C(=O)O)cc1)CC(F)(F)C2] | 33-rac CAS: 2760789- 06-6 | | LCMS (m/z): [M + H]⁺ calc'd for C₂₆H₂₉F₂N₂O₃, 455.2; found, 455. NMR (500 MHz, CD₃OD) δ 8.13 (d, J = 7.9 Hz, 2H), 7.62 (d, J = 7.5 Hz, 2H), 7.29 (s, 1H), 6.74 (s, 1H), 6.31 (s, 1H), 4.25 (s, 2H), 3.97 (s, 1H), 3.75 (s, 3H), 3.40 (s, 1H), 3.16 (s, 1H), 2.80-2.54 (m, 2H), 2.49 (s, 3H), 2.42 (s, 2H), 2.23 (s, 1H), 2.03 (s, 2H), 1.87 (s, 1H). |
| 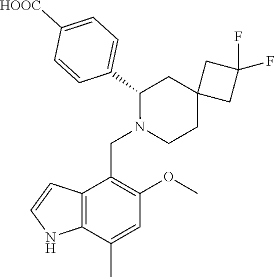 [SMILES: COc1cc(C)c2[nH]ccc2c1CN1CCC2(C[C@H]1c1ccc(C(=O)O)cc1)CC(F)(F)C2] | 33 | | LCMS (m/z): [M + H]⁺ calc'd for C₂₆H₂₉F₂N₂O₃, 455.2; found, 455. ¹H NMR (400 MHz, CD₃OD): δ 8.24 (d, J = 8.0 Hz, 2H), 7.76 (d, J = 8.0 Hz, 2H), 7.34 (d, J = 2.8 Hz, 1H), 6.77 (s, 1H), 6.35 (d, J = 3.2 Hz, 1H), 4.53-4.57 (m, 1H), 4.35 (d, J = 12.4 Hz, 1H), 4.19 (d, J = 12.4 Hz, 1H), 3.75 (s, 3H), 3.52-3.55 (m, 1H), 3.33-3.36 (m, 1H), 2.68- 2.79 (m, 2H), 2.48-2.50 (m, 3H), 2.42-2.45 (m, 2H), 2.27- 2.34 (m, 1H), 2.01-2.14 (m, 2H), 1.91-1.95 (m, 1H). |
| (S)-4-(2,2-difluoro-7-((5-methoxy-7- | | | |
| methyl-1H-indol-4-yl)methyl)-7- | | | |
| azaspiro[3.5]nonan-6-yl)benzoic acid | | | |
| 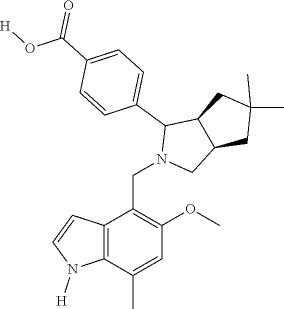 [SMILES: COc1cc(C)c2[nH]ccc2c1CN1C[C@@H]2CC(C)(C)C[C@@H]2C1c1ccc(C(=O)O)cc1] | 34 (Isomer 1) | Column: OX-H 4.6 × 100 mm, 5 μm Mobile phase: 25% EtOH [1% NH₃ (7 M in MeOH)] in CO₂ Flow rate: 4 mL/min Column temperature: 40° C. Retention time = 2.26 min | LCMS (m/z): [M + H]⁺ calc'd for C₂₇H₃₃N₂O₃, 433.2; found, 433.1 ¹H NMR (400 MHz, CD₃OD): δ 8.10 (d, J = 8.3 Hz, 2H), 7.55 (d, J = 8.2 Hz, 2H), 7.29 (d, J = 3.1 Hz, 1H), 6.73 (s, 1H), 6.22 (d, J = 3.1 Hz, 1H), 4.42-4.24 (m, 3H), 3.74 (s, 3H), 3.70- 3.66 (m, 1H), 3.26-3.17 (m, 1H), 3.05-3.01 (m, 2H), 2.49 (s, 3H), 1.83-1.79 (m, 1H), 1.72-1.61 (m, 1H), 1.57-1.45 (m, 2H), 1.17 (s, 3H), 0.96 (s, 3H). |
| 4-((3aR,6aS)-2-((5-methoxy-7-methyl- | | | |
| 1H-indol-4-yl)methyl)-5,5- | | | |
| dimethyloctahydrocyclopenta[c]pyrrol- | | | |
| 1-yl)benzoic acid | | | |
| 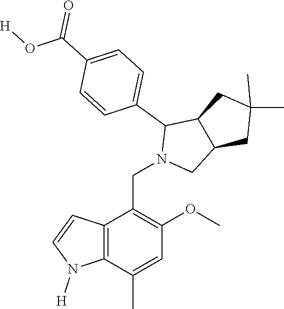 [SMILES: COc1cc(C)c2[nH]ccc2c1CN1C[C@@H]2CC(C)(C)C[C@@H]2C1c1ccc(C(=O)O)cc1] | 34 (Isomer 2) | Column: OX-H 4.6 × 100 mm, 5 μm Mobile phase: 25% EtOH [1% NH₃ (7 M in MeOH)] in CO₂ Flow rate: 4 mL/min Column temperature: 40º C. Retention time = 3.07 min | LCMS (m/z): [M + H]⁺ calc'd for C₂₇H₃₃N₂O₃, 433.2; found, 433.1 ¹H NMR (400 MHz, CD₃OD): δ 8.10 (d, J = 8.3 Hz, 2H), 7.55 (d, J = 8.2 Hz, 2H), 7.29 (d, J = 3.1 Hz, 1H), 6.73 (s, 1H), 6.23 (d, J = 3.1 Hz, 1H), 4.40-4.24 (m, 3H), 3.74 (s, 3H), 3.71- 3.67 (m, 1H), 3.25-3.16 (m, 1H), 3.05-3.01 (m, 2H), 2.49 (s, 3H), 1.82-1.80 (m, 1H), 1.72-1.61 (m, 1H), 1.58-1.45 (m, 2H), 1.17 (s, 3H), 0.96 (s, 3H). |
| 4-((3aR,6aS)-2-((5-methoxy-7-methyl- | | | |
| 1H-indol-4-yl)methyl)-5,5- | | | |
| dimethyloctahydrocyclopenta[c]pyrrol- | | | |
| 1-yl)benzoic acid | | | |
| 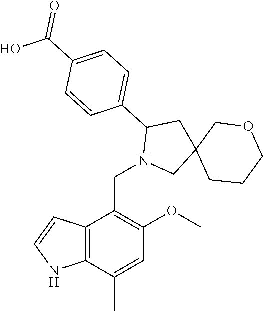 [SMILES: COc1cc(C)c2[nH]ccc2c1CN1CC2(CCCOC2)CC1c1ccc(C(=O)O)cc1] | 35 | | LCMS (m/z): [M + H]⁺ calc'd for C₂₆H₃₁N₂O₄, 435.2; found, 435 ¹H NMR (400 MHz, CD₃OD): δ 7.97 (d, J = 8.2 Hz, 2H), 7.51 (d, J = 8.2 Hz, 2H), 7.16 (d, J = 3.1 Hz, 1H), 6.67 (s, 1H), 6.36 (d, J = 3.1 Hz, 1H), 3.83-3.75 (m, 4H), 3.66-3.38 (m, 6H), 3.32 (s, 1H), 3.05 (d, J = 9.9 Hz, 1H), 2.45 (s, 3H), 2.21 (d, J = 9.9 Hz, 1H), 1.92 (dd, J = 13.1, 7.7 Hz, 1H), 1.65 (s, 1H), 1.62- 1.56 (m, 1H), 1.51 (d, J = 3.5 Hz, 2H). |
| 4-(2-((5-methoxy-7-methyl-1H-indol-4- | | | |
| yl)methyl)-7-oxa-2-azaspiro[4.5]decan- | | | |
| 3-yl)benzoic acid | | | |
| 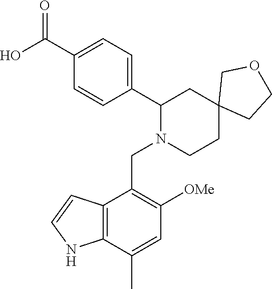 [SMILES: COc1cc(C)c2[nH]ccc2c1CN1CCC2(CCOC2)CC1c1ccc(C(=O)O)cc1] | 36 (Isomer 1) | Column: CHIRAL IG 4.6 × 100 mm I.D., 5 μm Mobile phase: MeOH/ACN = 3/2 [0.2% NH₃ (7 M in MeOH)] Flow rate: 3 mL/min Column temperature: 40º C. Retention time = 1.069 min | LCMS (m/z): [M + H]⁺ calc'd for C₂₆H₃₁N₂O₄, 435.2; found, 435.3 ¹H NMR (400 MHz, CD₃OD): δ 8.16-8.06 (m, 2H), 7.60 (d, J = 7.7 Hz, 2H), 7.28 (d, J = 3.1 Hz, 1H), 6.74 (s, 1H), 6.33 (s, 1H), 4.21 (s, 2H), 3.82 (d, J = 7.2 Hz, 5H), 3.75 (s, 3H), 3.36 (s, 1H), 3.02 (s, 1H), 2.49 (s, 3H), 2.23- 2.12 (m, 1H), 2.01-1.87 (m, 2H), 1.79 (s, 3H). |
| 4-(8-((5-methoxy-7-methyl-1H-indol-4- | | | |
| yl)methyl)-2-oxa-8-azaspiro[4.5]decan- | | | |
| 7-yl)benzoic acid | | | |
| 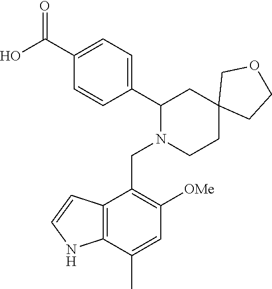 [SMILES: COc1cc(C)c2[nH]ccc2c1CN1CCC2(CCOC2)CC1c1ccc(C(=O)O)cc1] | 36 (Isomer 2) | Column: CHIRAL IG 4.6 × 100 mm I.D., 5 μm Mobile phase: MeOH/ACN = 3/2 [0.2% NH₃ (7 M in MeOH)] Flow rate: 3 mL/min Column temperature: 40° C. Retention time = 2.607 min | LCMS (m/z): [M + H]⁺ calc'd for C₂₆H₃₁N₂O₄, 435.2; found, 435.3 ¹H NMR (400 MHz, CD₃OD): δ 8.16-8.06 (m, 2H), 7.60 (d, J = 7.7 Hz, 2H), 7.28 (d, J = 3.1 Hz, 1H), 6.74 (s, 1H), 6.33 (s, 1H), 4.21 (s, 2H), 3.82 (d, J = 7.2 Hz, 5H), 3.75 (s, 3H), 3.36 (s, 1H), 3.02 (s, 1H), 2.49 (s, 3H), 2.23- 2.12 (m, 1H), 2.01-1.87 (m, 2H), 1.79 (s, 3H). |
| 4-(8-((5-methoxy-7-methyl-1H-indol-4- | | | |
| yl)methyl)-2-oxa-8-azaspiro[4.5]decan- | | | |
| 7-yl)benzoic acid | | | |
| 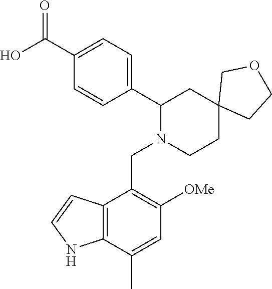 [SMILES: COc1cc(C)c2[nH]ccc2c1CN1CCC2(CCOC2)CC1c1ccc(C(=O)O)cc1] | 36 (Isomer 3) | Column: CHIRAL IG 4.6 × 100 mm I.D., 5 μm Mobile phase: EtOH [1% NH₃ (7 M in MeOH)] Flow rate: 3 mL/min Column temperature: 40° C. Retention time = 0.871 min | LCMS (m/z): [M + H]⁺ calc'd for C₂₆H₃₁N₂O₄, 435.2; found, 435.3 ¹H NMR (400 MHz, CD₃OD): δ 8.16-8.06 (m, 2H), 7.60 (d, J = 7.7 Hz, 2H), 7.28 (d, J = 3.1 Hz, 1H), 6.74 (s, 1H), 6.33 (s, 1H), 4.21 (s, 2H), 3.82 (d, J = 7.2 Hz, 5H), 3.75 (s, 3H), 3.36 (s, 1H), 3.02 (s, 1H), 2.49 (s, 3H), 2.23- 2.12 (m, 1H), 2.01-1.87 (m, 2H), 1.79 (s, 3H). |
| 4-(8-((5-methoxy-7-methyl-1H-indol-4- | | | |
| yl)methyl)-2-oxa-8-azaspiro[4.5]decan- | | | |
| 7-yl)benzoic acid | | | |
| 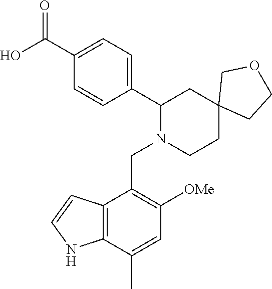 [SMILES: COc1cc(C)c2[nH]ccc2c1CN1CCC2(CCOC2)CC1c1ccc(C(=O)O)cc1] | 36 (Isomer 4) | Column: CHIRAL IG 4.6 × 100 mm I.D., 5 μm Mobile phase: EtOH [1% NH₃ (7 M in MeOH)] Flow rate: 3 mL/min Column temperature: 40° C. Retention time = 1.877 min | LCMS (m/z): [M + H]⁺ calc'd for C₂₆H₃₁N₂O₄, 435.2; found, 435.3 ¹H NMR (400 MHz, CD₃OD): δ 8.16-8.06 (m, 2H), 7.60 (d, J = 7.7 Hz, 2H), 7.28 (d, J = 3.1 Hz, 1H), 6.74 (s, 1H), 6.33 (s, 1H), 4.21 (s, 2H), 3.82 (d, J = 7.2 Hz, 5H), 3.75 (s, 3H), 3.36 (s, 1H), 3.02 (s, 1H), 2.49 (s, 3H), 2.23- 2.12 (m, 1H), 2.01-1.87 (m, 2H), 1.79 (s, 3H). |
| 4-(8-((5-methoxy-7-methyl-1H-indol-4- | | | |
| yl)methyl)-2-oxa-8-azaspiro[4.5]decan- | | | |
| 7-yl)benzoic acid | | | |

### Synthesis of 4-(2-((5-methoxy-7-methyl-1H-indol-4-yl)methyl)octahydrocyclopenta[c]pyrrol-1-yl)benzoic acid (Example 13)

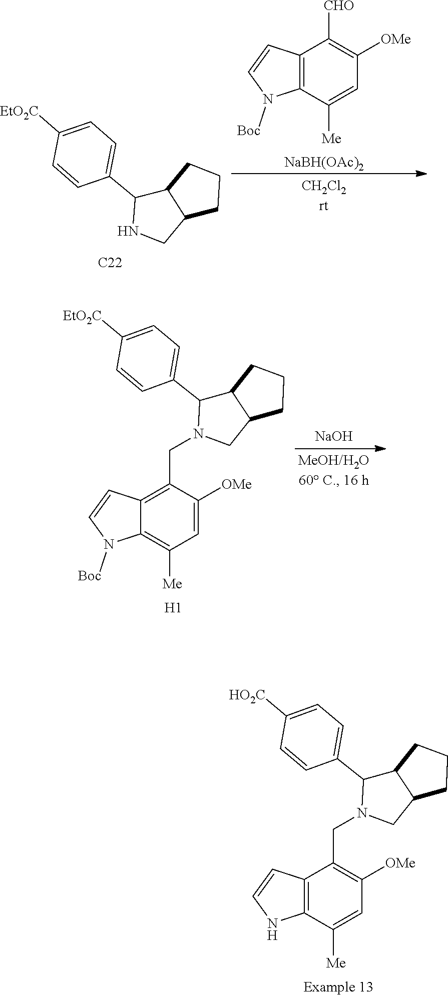

### Step 1: Synthesis of tert-butyl 4-((1-(4-(ethoxycarbonyl)phenyl)hexahydrocyclopenta[c]pyrrol-2(1H)-yl)methyl)-5-methoxy-7-methyl-1H-indole-1-carboxylate (H)

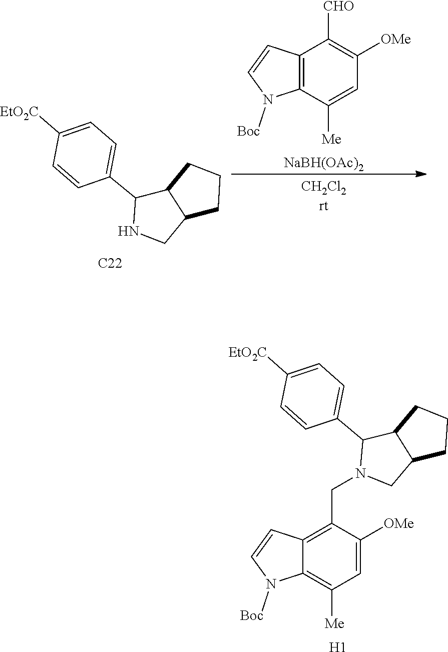

To a solution of 4-(octahydrocyclopenta[c]pyrrol-1-yl)benzoic acid (154 mg, 0.590 mmol) in THF (60 mL) was added tert-butyl 4-formyl-5-methoxy-7-methyl-1H-indole-1-carboxylate (170 mg, 0.590 mmol) and the mixture was stirred at room temperature for 3 h prior to the addition of NaBH(OAc)₃ (382 mg, 1.80 mmol). The resulting reaction mixture was stirred at room temperature for an additional 16 h. The reaction was quenched by the addition of aqueous NH₄Cl solution and extracted with ethyl acetate (3×30 ml). The combined organic layers were dried over anhydrous Na2SO₄ and concentrated in vacuo. The crude residue was purified by flash column chromatography (SiO₂, 4:1 petroleum ether/ethyl acetate) to afford pure desired product as a yellow solid (80 mg, 25%). LCMS (m/z): [M+H]⁺ calc'd for C₃₂H₄₁N₂O₅, 533.3; found, 533.

The following intermediate was synthesized using similar conditions as those described in Step 1, above, along with appropriate starting materials.

| Structure and Name | Intermediate | LCMS |
|---|---|---|
| 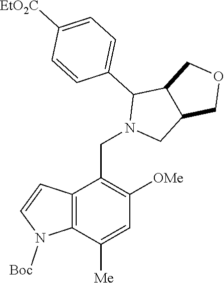 | H2 | LCMS (m/z): [M + H]⁺ calc'd for C₃₁H₃₉N₂O₆, 535.3; found, 535 |
| tert-butyl 4-((4-(4- | | |
| (ethoxycarbonyl)phenyl)tetrahydro-1H- | | |
| furo[3,4-c]pyrrol-5 (3H)-yl)methyl)-5- | | |
| methoxy-7-methyl-1H-indole-1- | | |
| carboxylate | | |
| 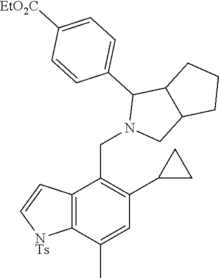 [SMILES: CCOC(=O)c1ccc(C2C3CCCC3CN2Cc2c(C3CC3)cc(C)c3c2ccn3S(=O)(=O)c2ccc(C)cc2)cc1] | H3 | LCMS (m/z): [M + H]⁺ calc'd for C₃₆H₄₁N₂O₄S, 597.3; found, 597.0 |
| ethyl 4-(2-((5-cyclopropyl-7-methyl-1- | | |
| tosyl-1H-indol-4- | | |
| yl)methyl)octahydrocyclopenta[c]pyrrol- | | |
| 1-yl)benzoate | | |

### Step 2: Synthesis of 4-(2-((5-methoxy-7-methyl-1H-indol-4-yl)methyl)octahydrocyclopenta[c]pyrrol-1-yl)benzoic acid (Example 13)

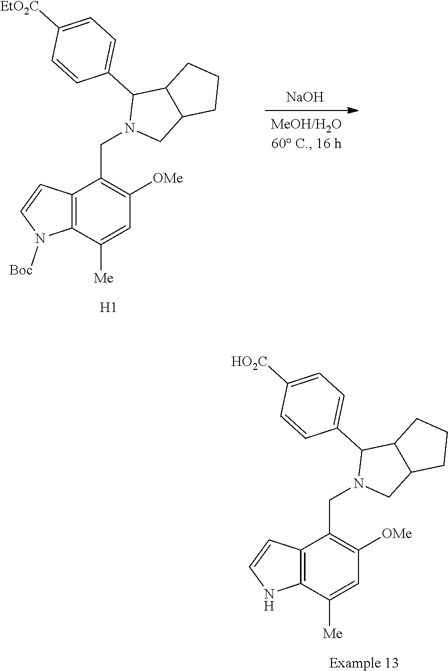

To a stirred solution of tert-butyl 4-((1-(4-(ethoxycarbonyl)phenyl)hexahydrocyclopenta[c]pyrrol-2(1H)-yl)methyl)-5-methoxy-7-methyl-1H-indole-1-carboxylate (80 mg, 0.15 mmol) in EtOH (3 mL) was added a solution of NaOH (24 mg, 0.60 mmol) in H₂O (0.3 mL). The mixture was stirred at 60° C. for 16 h, then cooled to rt, concentrated in vacuo, and the pH adjusted (pH ˜6.4-6.7) with a solution of citric acid (1 M, aqueous). The mixture was then concentrated under vacuum. The crude residue was purified by SFC (column: Gemini-C18 150×21.2 mm 5 μm; mobile phase A [H₂O (0.1% formic acid] and B [acetonitrile (0.1% trifluoroacetic acid)]; gradient B: 20-40% over 7 min) Example 13: 4-(2-((5-methoxy-7-methyl-1H-indol-4-yl)methyl)octahydrocyclopenta[c]pyrrol-1-yl)benzoic acid (34 mg, 65%) was obtained as a white solid. LCMS (m/z): [M+H]⁺ calc'd for C₂₅H₂₉N₂O₃, 405.2; found, 405.5. ¹H NMR (400 MHz, CD₃OD): δ 8.11-8.09 (d, J=8.0 Hz, 2H), 7.61 (d, J=8.0 Hz, 2H), 7.27 (d, J=3.2 Hz, 1H), 6.71 (s, 1H), 6.25 (d, J=3.2 Hz, 1H), 4.25 (m, 2H), 4.06 (br, 1H), 3.73 (m, 3H), 3.67 (m, 1H), 2.90 (m, 3H), 2.84 (s, 3H), 1.91 (m, 1H), 1.82 (m, 1H), 1.65 (m, 4H).

The two isomers of Example 13 were separated by chiral-SFC on a CHIRALPAK OJ-H 250×20 mm, 5 μm column. The mobile phase A [CO₂] and 35% B [Ethanol (0.2% NH₄OH)] at a flow rate of 12.5 mL/min and a column temperature of 39° C. The retention time of Isomer 1 is 3.64 min. The retention time of Isomer 2 is 4.51 min.

Isomer 1: 4-(2-((5-methoxy-7-methyl-1H-indol-4-yl)methyl)octahydrocyclopenta[c]pyrrol-1-yl)benzoic acid (11 mg). ¹H NMR (400 MHz, CD₃OD): δ 8.10-8.08 (d, J=8.0 Hz, 2H), 7.57-7.55 (d, J=8.0 Hz, 2H), 7.27 (d, J=2.8 Hz, 1H), 6.72 (s, 1H), 6.22-6.21 (d, J=2.8 Hz, 1H), 4.23 (m, 2H), 3.99 (br, 1H), 3.74 (m, 3H), 3.64 (m, 1H), 2.87 (mn, 3H), 2.48 (s, 3H), 1.92 (m, 1H), 1.82 (m, 1H), 1.66 (in, 4H).

Isomer 2: 4-(2-((5-methoxy-7-methyl-1H-indol-4-yl)methyl)octahydrocyclopenta[c]pyrrol-1-yl)benzoic acid (12 mg). EH NMR (400 MHz, CD₃oD): 6 8.10-8.09 (d, J 8.0 Hz, 2H), 7.57-7.55 (d, J 8.0 Hz, 2H), 7.27 (d, J 2.8 Hz, 1H), 6.72 (s, 1H), 6.22-6.21 (d, J=2.8 Hz, 1H), 4.22 (m, 2H), 4.02 (br, 1H), 3.74 (m, 3H), 3.64 (m, 1H), 2.87 (m, 3H), 2.48 (s, 3H), 1.91 (m, 1H), 1.81 (m, 1H), 1.66 (in, 4H).

The following examples were synthesized using the ester hydrolysis procedure described above using appropriate starting materials:

| | | Isomer Separation | |
|---|---|---|---|
| | | Method and | |
| | | Retention Time (if | |
| Name and Stucture | Ex. # | any) | LCMS + ¹H NMR |
| 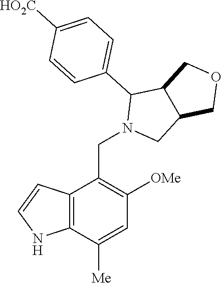 | Example 14 (Isomer 1) | Column: CHIRALPAK AD-3 100 × 4.6 mm I.D., 3 μm Mobile phase: 40% EtOH [1% NH_{3 (}7 M in MeOH)] in CO₂ Flow rate: 3 mL/min Column temperature: 40° C. Retention time = 0.851 min | LCMS (m/z): [M + H]⁺ calc'd for C₂₄H₂₇N₂O₄, 407.2; found, 407 ¹H NMR (400 MHz, CD₃OD): δ 8.10 (d, J = 8.4 Hz, 2H), 7.61 (d, J = 8.0 Hz, 2H), 7.27 (d, J = 3.2 Hz, 1H), 6.71 (s, 1H), 6.25 (d, J = 3.2 Hz, 1H), 4.21-4.15 (m, 2H), 4.02 (s, 1H), 3.93-3.88 (m, 2H), 3.74 (s, 3H), 3.67 (s, 1H), 3.58- 3.54 (m, 1H), 3.52-3.48 (m, 1H), 3.06 (s, 2H), 2.94 (s, 1H), 2.48 (s, 3H). |
| 4-((3aS,6aR)-5-((5-methoxy-7- | | | |
| methyl-1H-indol-4- | | | |
| yl)methyl)hexahydro-1H- | | | |
| furo[3,4-c]pyrrol-4-yl)benzoic | | | |
| acid | | | |
| 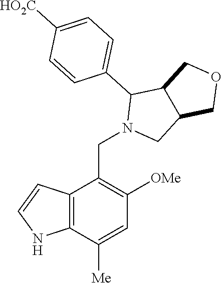 | Example 14 (Isomer 2) | Column: CHIRALPAK AD-3 100 × 4.6 mm I.D., 3 μm Mobile phase: 40% EtOH [1% NH_{3 (}7 M in MeOH)] in CO₂ Flow rate: 3 mL/min Column temperature: 40° C. Retention time = 2.146 min | LCMS (m/z): [M + H]⁺ calc'd for C₂₄H₂₇N₂O₄, 407.2; found, 407 ¹H NMR (400 MHz, CD₃OD): δ 8.11 (d, J = 8.0 Hz, 2H), 7.61 (d, J = 8.0 Hz, 2H), 7.27 (d, J = 3.2 Hz, 1H), 6.71 (s, 1H), 6.25 (d, J = 3.2 Hz, 1H), 4.21-4.16 (m, 2H), 4.02 (s, 1H), 3.93-3.88 (m, 2H), 3.74 (s, 3H), 3.70-3.66 (m, 1H), 3.58-3.54 (m, 1H), 3.52-3.48 (m, 1H), 3.07 (s, 2H), 2.95 (s, 1H), 2.48 (s, 3H). |
| 4-((3aS,6aR)-5-((5-methoxy-7- | | | |
| methyl-1H-indol-4- | | | |
| yl)methyl)hexahydro-1H- | | | |
| furo[3,4-c]pyrrol-4-yl)benzoic | | | |
| acid | | | |

### Synthesis of 4-(2-((5,7-dimethyl-1H-indol-4-yl)methyl)-2-azaspiro[3.5]nonan-1-yl)benzoic acid (Example 16)

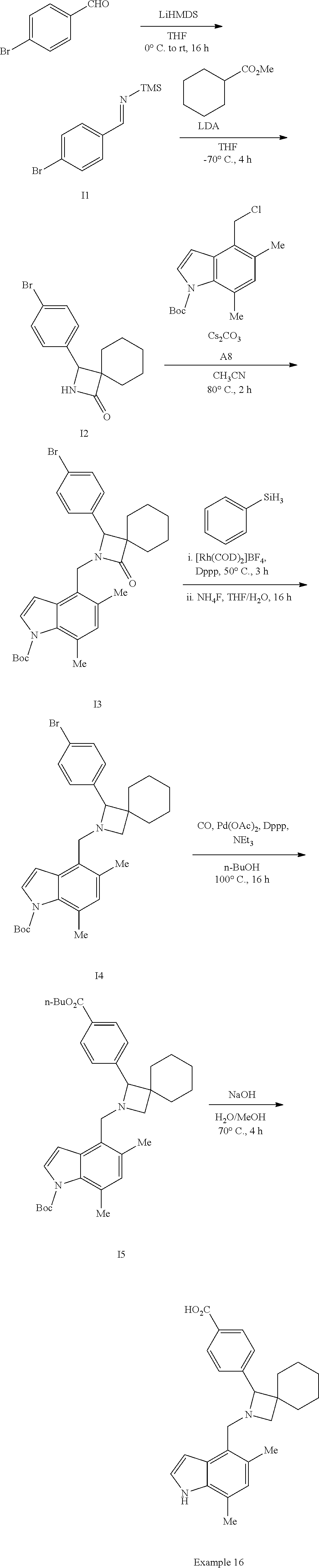

### Step 1: Synthesis of 1-(4-bromophenyl)-N-(trimethylsilyl)methanimine (I1)

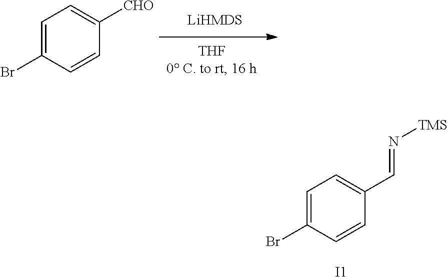

A solution of LiHMDS (1 M, 89.2 mL, 89.2 mmol) in THF (50 mL) was cooled to 0° C. and a solution of 4-bromobenzaldehyde (15 g, 81 mmol) in THF (50 mL) was added. The mixture was warmed to 20° C. and stirred for 16 h at that temp. The resulting solution was concentrated in vacuo prior to the addition of hexane. The resulting solids were filtered off and the filtrate concentrated. This process was repeated twice to give a pale yellow oil (21 g) which was taken to the next step without further purification.

### Step 2: Synthesis of 3-(4-bromophenyl)-2-azaspiro[3.5]nonan-1-one (I2)

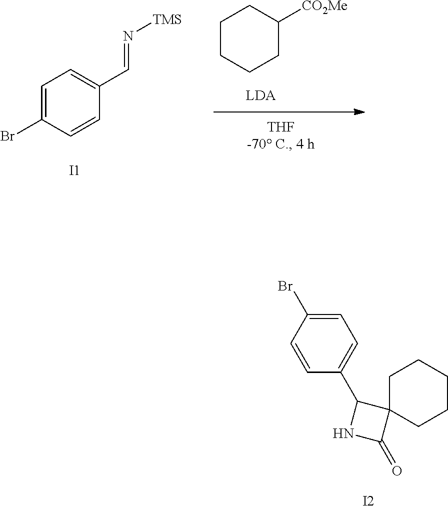

A solution of LDA (46.8 mL, 2 M) in THF (50 mL) was cooled to −78° C. under N₂ and a solution of methyl cyclohexanecarboxylate (13.3 g, 93.7 mmol) in THF (20 mL) was added. The mixture was stirred consecutively at −78° C. for 40 min, then at 20° C. for 10 min. The resulting mixture was re-cooled to −78° C. and a solution of 1-(4-bromophenyl)-N-(trimethylsilyl)methanimine (6.00 g, 23.4 mmol) in THF (50 mL) was added dropwise. The mixture was warmed to 20° C. and stirred for 15 h at that temp. The reaction was quenched with aqueous NH₄Cl and extracted with ethyl acetate (2×200 mL). The combined organic layers were dried over anhydrous Na₂SO₄, filtered, and concentrated in vacuo. The crude residue was purified by flash column chromatography (SiO₂, 4:1 petroleum ether/ethyl acetate) to afford pure desired product (3.4 g, 44%) as a yellow oil. LCMS (m/z): [M+H]⁺ calc'd for C₁₄H₁₇BrNO, 294.0; found, 293.9.

The following intermediates were synthesized using similar conditions as those described in step 2, above, along with appropriate starting materials.

| Structure and Name | Intermediate | LCMS |
|---|---|---|
| 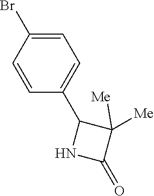 [SMILES: CC1(C)C(=O)NC1c1ccc(Br)cc1] | I6 | LCMS (m/z): [M + H]⁺ calc'd for C₁₁H₁₃BrNO, 254.0; found, 254 |
| 4-(4-bromophenyl)-3,3-dimethylazetidin-2-one | | |
| 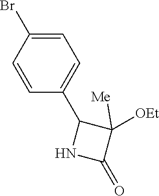 [SMILES: CCOC1(C)C(=O)NC1c1ccc(Br)cc1] | I7 | LCMS (m/z): [M + H]⁺ calc'd for C₁₂H₁₅BrNO₂, 284.0; found, 283.9 |
| 4-(4-bromophenyl)-3-ethoxy-3-methylazetidin-2- | | |
| one | | |
| 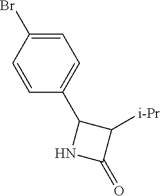 [SMILES: CC(C)C1C(=O)NC1c1ccc(Br)cc1] | I8 | LCMS (m/z): [M + H]⁺ calc'd for C₁₂H₁₅BrNO, 268.0; found, 268.1 |
| 4-(4-bromophenyl)-3-isopropylazetidin-2-one | | |

### Step 3: Synthesis of tert-butyl 4-((1-(4-bromophenyl)-3-oxo-2-azaspiro[3.5]nonan-2-yl)methyl)-5,7-dimethyl-1H-indole-1-carboxylate (I3)

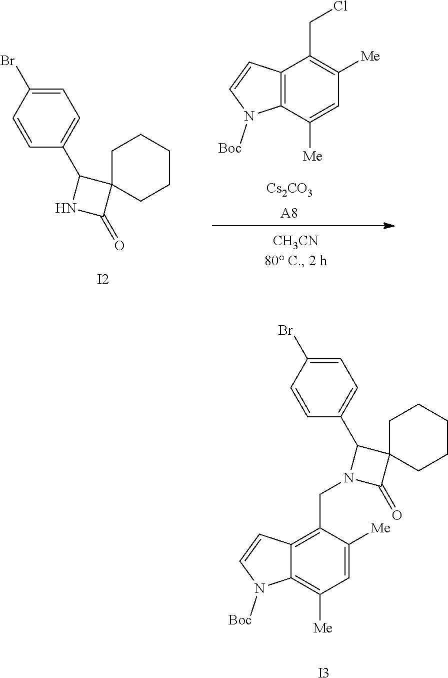

A solution of 3-(4-bromophenyl)-2-azaspiro[3.5]nonan-1-one (300 mg, 1.02 mmol), tert-butyl 4-(chloromethyl)-5,7-dimethyl-1H-indole-1-carboxylate (300 mg, 1.02 mmol) and Cs₂CO₃ (997 mg, 3.06 mmol) in MeCN (10 mL) was heated to 80° C. and stirred at that temp for 2 h. The resulting reaction mixture was concentrated and the residue purified by flash column chromatography (SiO₂, 20:1 petroleum ether/ethyl acetate) to afford pure desired product (320 mg, 53%) as white solid. LCMS (m/z): [M-Boc]⁺ calc'd for C₂₅H₂₇BrN₂O, 450.1; found, 450.7.

The following intermediates were synthesized using similar conditions as those described in step 3, above, along with appropriate starting materials.

| Structure and Name | Intermediate | LCMS |
|---|---|---|
| 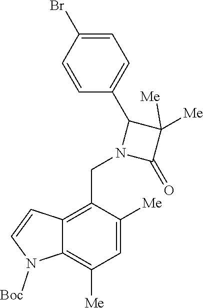 [SMILES: Cc1cc(C)c2c(ccn2C(=O)OC(C)(C)C)c1CN1C(=O)C(C)(C)C1c1ccc(Br)cc1] | I9 | LCMS (m/z): [M − Boc]⁺ calc'd for C₂₂H₂₃BrN₂O, 410.1; found, 410.9. |
| tert-butyl 4-((2-(4-bromophenyl)-3,3-dimethyl-4- | | |
| oxoazetidin-1-yl)methyl)-5,7-dimethyl-1H-indole-1- | | |
| carboxylate | | |
| 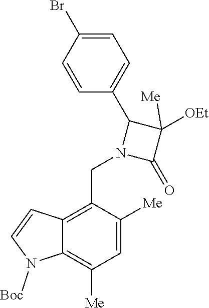 [SMILES: CCOC1(C)C(=O)N(Cc2c(C)cc(C)c3c2ccn3C(=O)OC(C)(C)C)C1c1ccc(Br)cc1] | I10 | LCMS (m/z): [M + H]⁺ calc'd for C₂₈H₃₃BrN₂NaO₄, 563.2; found, 563.0 |
| tert-butyl 4-((2-(4-bromophenyl)-3-ethoxy-3- | | |
| methyl-4-oxoazetidin-1-yl)methyl)-5,7-dimethyl- | | |
| 1H-indole-1-carboxylate | | |
| 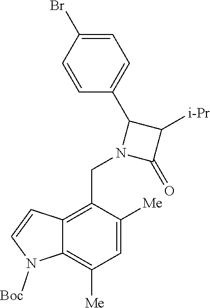 [SMILES: Cc1cc(C)c2c(ccn2C(=O)OC(C)(C)C)c1CN1C(=O)C(C(C)C)C1c1ccc(Br)cc1] | I11 | LCMS (m/z): [M + Na]⁺ calc'd for C₂₈H₃₃BrN₂NaO₃, 547.2; found, 546.7 |
| tert-butyl 4-((2-(4-bromophenyl)-3-isopropyl-4- | | |
| oxoazetidin-1-yl)methyl)-5,7-dimethyl-1H-indole-1- | | |
| carboxylate | | |

### Step 4: Synthesis of tert-butyl 4-((1-(4-bromophenyl)-2-azaspiro[3.5]nonan-2-yl)methyl)-5,7-dimethyl-1H-indole-1-carboxylate (I4)

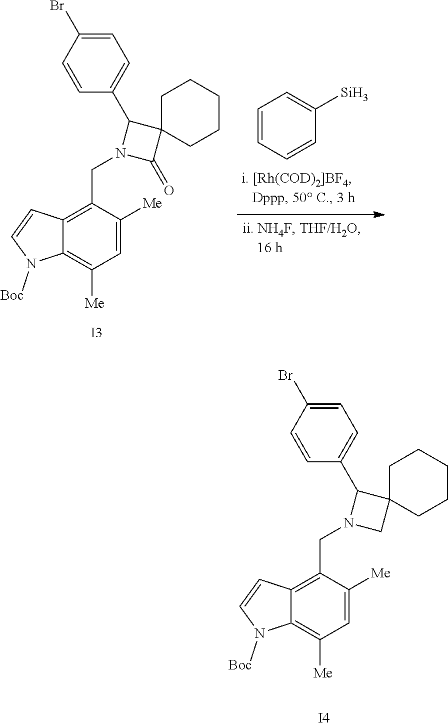

A solution of tert-butyl 4-((1-(4-bromophenyl)-3-oxo-2-azaspiro[3.5]nonan-2-yl)methyl)-5,7-dimethyl-1H-indole-1-carboxylate (220 mg, 0.400 mmol), phenylsilane (172 mg, 1.59 mmol), Dppp (33 mg, 0.080 mmol), and [Rh(COD)₂] BF₄ (16 mg, 0.040 mmol) in THF (1 mL) was stirred at 50° C. for 3 h. The reaction solution was cooled to 20° C., then aqueous NH₄F (0.1 mL) was added, and the mixture stirred at 20° C. for 20 h. The resulting mixture was concentrated and purified by flash column chromatography (SiO₂, 12:1 petroleum ether/ethyl acetate) to afford pure desired product (140 mg, 55%) as a colorless oil. LCMS (m/z): [M+H]⁺ calc'd for C₃₀H₃₈BrN₂O₂, 537.2; found, 537.0.

The following intermediates were synthesized using similar conditions as those described in step 4, above, along with appropriate starting materials.

| Structure and Name | Intermediate | LCMS |
|---|---|---|
| 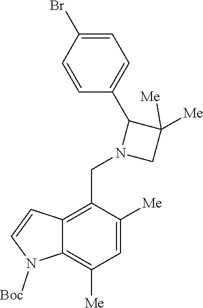 [SMILES: Cc1cc(C)c2c(ccn2C(=O)OC(C)(C)C)c1CN1CC(C)(C)C1c1ccc(Br)cc1] | I12 | LCMS (m/z): [M + H]⁺ calc'd for C₂₇H₃₄BrN₂O₂, 497.2; found, 496.9. |
| tert-butyl 4-((2-(4-bromophenyl)-3,3- | | |
| dimethylazetidin-1-yl)methyl)-5,7-dimethyl-1H- | | |
| indole-1-carboxylate | | |
| 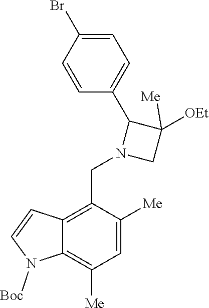 [SMILES: CCOC1(C)CN(Cc2c(C)cc(C)c3c2ccn3C(=O)OC(C)(C)C)C1c1ccc(Br)cc1] | I13 | LCMS (m/z): [M + H]⁺ calc'd for C₂₈H₃₆BrN₂O₃, 527.2; found, 527.0 |
| tert-butyl 4-((2-(4-bromophenyl)-3-ethoxy-3- | | |
| methylazetidin-1-yl)methyl)-5,7-dimethyl-1H- | | |
| indole-1-carboxylate | | |
| 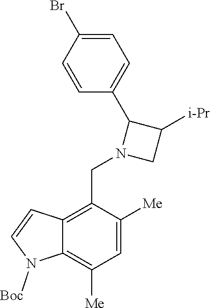 [SMILES: Cc1cc(C)c2c(ccn2C(=O)OC(C)(C)C)c1CN1CC(C(C)C)C1c1ccc(Br)cc1] | I14 | LCMS (m/z): [M + H]⁺ calc'd for C₂₈H₃₆BrN₂O₂, 511.2; found, 513 |
| tert-butyl 4-((2-(4-bromophenyl)-3- | | |
| isopropylazetidin-1-yl)methyl)-5,7-dimethyl-1H- | | |
| indole-1-carboxylate | | |

### Step 5: Synthesis of tert-butyl 4-((1-(4-(butoxycarbonyl)phenyl)-2-azaspiro[3.5]nonan-2-yl)methyl)-5,7-dimethyl-1H-indole-1-carboxylate (I5)

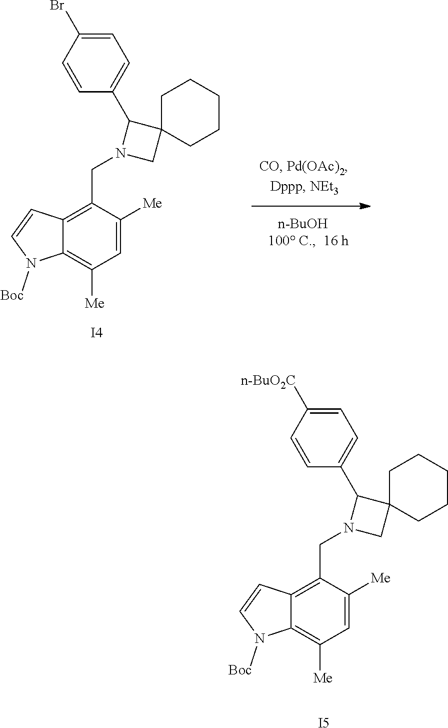

A solution of tert-butyl 4-((1-(4-bromophenyl)-2-azaspiro[3.5]nonan-2-yl)methyl)-5,7-dimethyl-1H-indole-1-carboxylate (140 mg, 0.260 mmol), Et₃N (1.32 g, 13.0 mmol), dppp (22 mg, 0.050 mmol), and Pd(OAc)₂ (5.6 mg, 0.026 mmol) in n-BuOH (10 mL) was stirred at 100° C. under a CO atmosphere for 16 h. The mixture was concentrated in vacuo and the residue was purified by flash column chromatography (SiO₂, 15:1 petroleum ether/ethyl acetate) to afford pure desired product (100 mg, 68%) as a yellow oil. LCMS (m/z): [M+H]⁺ calc'd for C₃₅H₄₇N2O₄, 559.4; found, 558.9.

The following intermediates were synthesized using similar conditions as those described in step 5, above, along with appropriate starting materials.

| Structure and Name | Intermediate | LCMS |
|---|---|---|
| 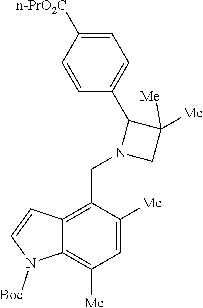 | I15 | LCMS (m/z): [M + H]⁺ calc'd for C₃₁H₄₁N₂O₄, 505.3; found, 504.8 |
| tert-butyl 4-((3,3-dimethyl-2-(4- | | |
| (propoxycarbonyl)phenyl)azetidin-1-yl)methyl)-5,7- | | |
| dimethyl-1H-indole-1-carboxylate | | |
| 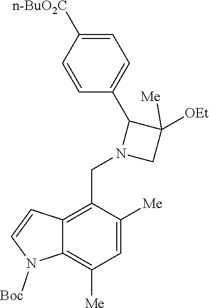 | I16 | LCMS (m/z): [M + H]⁺ calc'd for C₃₃H₄₅N₂O₅, 549.3; found, 549.1 |
| tert-butyl 4-((2-(4-(butoxycarbonyl)phenyl)-3- | | |
| ethoxy-3-methylazetidin-1-yl)methyl)-5,7-dimethyl- | | |
| 1H-indole-1-carboxylate | | |
| 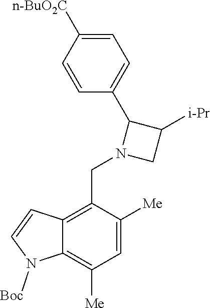 | I17 | LCMS (m/z): [M + H]⁺ calc'd for C₃₃H₄₅N₂O₄, 533.3; found, 532.9. |
| tert-butyl 4-((2-(4-(butoxycarbonyl)phenyl)-3- | | |
| isopropylazetidin-1-yl)methyl)-5,7-dimethyl-1H- | | |
| indole-1-carboxylate | | |

### Step 6: Synthesis of 4-(2-((5,7-dim ethyl-1H-indol-4-yl)methyl)-2-azas piro[3.5]nonan-1-yl)benzoic acid (Example 16)

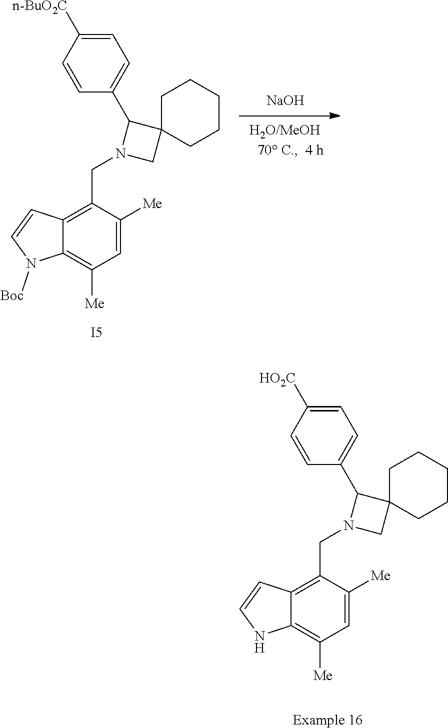

To a solution of tert-butyl 4-((1-(4-(butoxycarbonyl)phenyl)-2-azaspiro[3.5]nonan-2-yl)methyl)-5,7-dimethyl-1H-indole-1-carboxylate (100 mg, 0.180 mmol) in MeOH (5 mL) was added aqueous NaOH (3 M, 2 mL) and the mixture heated to 70° C. and stirred at that temp for 6 h before cooling to rt, and concentrating in vacuo. The crude residue was purified by prep-HPLC (column: Waters™ XBridge 2.1×50 mm 3.5 μm; mobile phase A [water (0.05% trifluoroacetic acid v/v)] and B [acetonitrile (0.05% trifluoroacetic acid)]; gradient B: 0-60% over 7 min).

Example 16: 4-(2-((5,7-dimethyl-1H-indol-4-yl)methyl)-2-azaspiro[3.5]nonan-1-yl)benzoic acid (50 mg, 69%) was obtained as a white solid. LCMS (m/z): [M+H]⁺ calc'd for C₂₆H₃₁N₂O₂, 403.2; found, 403.0. ¹H NMR (400 MHz, CD₃OD): δ 8.40 (br s, 0.6H), 7.95 (d, J=8.0 Hz, 2H), 7.37-7.22 (m, 3H), 6.79 (s, 1H), 6.55 (d, J=3.0 Hz, 1H), 4.90-4.88 (m, 1H), 4.67-4.47 (m, 2H), 3.85-3.66 (m, 2H), 2.44 (s, 3H), 2.40 (s, 3H), 1.85-1.70 (m, 2H), 1.65-1.54 (m, 2H), 1.54-1.45 (m, 1H), 1.38-1.20 (m, 3H), 1.16-1.04 (m, 1H), 1.02-0.89 (m, 1H).

Isomers of Example 16 were separated by SFC from 45 mg of material. Isomer 1 and Isomer 2 were purified on a CHIRALPAK AD-H column. The mobile phase A [CO₂] and B [i-PrOH (0.2% NH₄OH)] at a flow rate of 12.5 mL/min and a column temperature of 40.7° C. The retention time of Isomer 1 is 4.03 min. The retention time of Isomer 2 is 6.22 min.

Isomer 1: 4-(2-((5,7-dimethyl-1H-indol-4-yl)methyl)-2-azaspiro[3.5]nonan-1-yl)benzoic acid (15 mg, 37%) was obtained. LCMS (m/z): [M+H]⁺ calc'd for C₂₆H₃₁N₂O₂, 403.2; found, 403.1. ¹H NMR (400 MHz, CD₃OD): δ 7.93 (d, J=8.2 Hz, 2H), 7.35-7.23 (m, 3H), 6.78 (s, 1H), 6.54 (d, J=3.2 Hz, 1H), 4.73-4.61 (s, 1H), 4.38 (s, 2H), 3.61-3.41 (m, 2H), 2.44 (s, 3H), 2.41 (s, 3H), 1.84-1.74 (m, 1H), 1.73-1.65 (m, 1H), 1.64-1.54 (m, 2H), 1.52-1.44 (m, 1H), 1.32-1.20 (m, 3H), 1.15-1.04 (m, 1H), 0.96-0.88 (m, 1H).

Isomer 2: 4-(2-((5,7-dimethyl-1H-indol-4-yl)methyl)-2-azaspiro[3.5]nonan-1-yl)benzoic acid (16 mg, 39%) was obtained. LCMS (m/z): [M+H]⁺ calc'd for C₂₆H₃₁N₂O₂, 403.2; found, 403.0. ¹H NMR (400 MHz, CD₃OD): δ 7.93 (d, J=8.0 Hz, 2H), 7.33-7.25 (m, 3H), 6.78 (s, 1H), 6.54 (d, J=3.2 Hz, 1H), 4.79-4.65 (s, 1H), 4.42 (s, 2H), 3.65-3.49 (m, 2H), 2.44 (s, 3H), 2.41 (s, 3H), 1.83-1.75 (m, 1H), 1.74-1.66 (m, 1H), 1.64-1.54 (m, 2H), 1.53-1.45 (m, 1H), 1.31-1.18 (m, 3H), 1.15-1.06 (m, 1H), 0.97-0.89 (m, 1H).

The following examples were synthesized using the ester hydrolysis procedure described above using appropriate starting materials:

| | | Isomer Separation | |
|---|---|---|---|
| | | Method and | |
| | | Retention Time (if | |
| Name and Stucture | Ex. # | any) | LCMS + ¹H NMR |
| 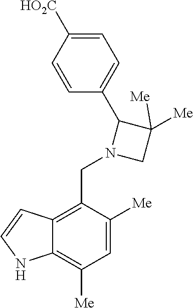 [SMILES: Cc1cc(C)c2[nH]ccc2c1CN1CC(C)(C)C1c1ccc(C(=O)O)cc1] | 17 | N/A | LCMS (m/z): [M + H]⁺ calc'd for C₂₃H₂₇N₂O₂, 363.2; found, 363.0 ¹H NMR (400 MHz, ): δ 8.46 (br s, 0.37 H), 7.91 (d, J = 8.0 Hz, 2H), 7.35-7.15 (m, 3H), 6.74 (s, 1H), 6.50 (d, J = 3.2 Hz, 1H), 4.69 (br s, 1H), 4.34 (s, 2H), 3.56- 3.44 (m, 1H), 3.41-3.31 (m, 1H), 2.40 (s, 3H), 2.38 (s, 3H), 1.21 (s, 3H), 0.86 (s, 3H). |
| 4-(1-((5,7-dimethyl-1H-indol-4- | | | |
| yl)methyl)-3,3-dimethylazetidin- | | | |
| 2-yl)benzoic acid | | | |
| 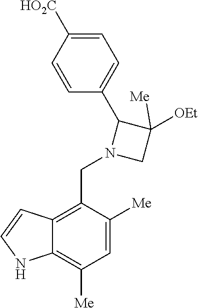 [SMILES: CCOC1(C)CN(Cc2c(C)cc(C)c3[nH]ccc23)C1c1ccc(C(=O)O)cc1] | 18 (Isomer 1) | Column: CHIRALPAK OD-H 250 × 20 mm, 5 μm Mobile phase: 30% EtOH (0.2% NH₄OH) in CO₂ Flow rate: 12.5 mL/min Column temperature: 40.3° C. Retention time = 5.75 min | LCMS (m/z): [M + H]⁺ calc'd for C₂₄H₂₉N₂O₃, 393.2; found, 393.1 ¹H NMR (400 MHz, CD₃OD): δ 7.94-7.88 (m, 2H), 7.27 (d, J = 8.2 Hz, 2H), 7.23 (d, J = 3.2 Hz, 1H), 6.73 (s, 1H), 6.50 (d, J = 3.2 Hz, 1H), 4.91-4.88 (m, 1H), 4.42 (s, 2H), 3.75-3.65 (m, 1H), 3.60-3.54 (m, 1H), 3.48-3.40 (m, 1H), 3.37-3.29 (m, 1H), 2.38 (d, J = 2.8 Hz, 6H), 1.15 (t, J = 7.0 Hz, 3H), 1.10 (s, 3H). |
| 4-(1-((5,7-dimethyl-1H-indol-4- | | | |
| yl)methyl)-3-ethoxy-3- | | | |
| methylazetidin-2-yl)benzoic acid | | | |
| 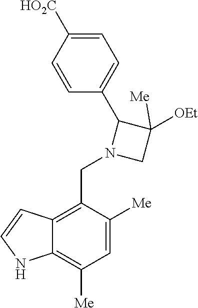 [SMILES: CCOC1(C)CN(Cc2c(C)cc(C)c3[nH]ccc23)C1c1ccc(C(=O)O)cc1] | 18 (Isomer 2) | Column: CHIRALPAK OD-H 250 × 20 mm, 5 μm Mobile phase: 30% EtOH (0.2% NH₄OH) in CO₂ Flow rate: 12.5 mL/min Column temperature: 40° C. Retention time = 6.35 min | LCMS (m/z): [M + H]⁺ calc'd for C₂₄H₂₉N₂O₃, 393.2; found, 392.9 ¹H NMR (400 MHz, CD₃OD): δ 7.91 (d, J = 8.4 Hz, 2H), 7.27 (d, J = 8.2 Hz, 2H), 7.22 (d, J = 3.2 Hz, 1H), 6.73 (s, 1H), 6.50 (d, J = 3.2 Hz, 1H), 4.91-4.88 (m, 1H), 4.42 (s, 2H), 3.74-3.64 (m, 1H), 3.61-3.52 (m, 1H), 3.48-3.40 (m, 1H), 3.37-3.30 (m, 1H), 2.42-2.35 (m, 6H), 1.15 (t, J = 7.0 Hz, 3H), 1.09 (s, 3H). |
| 4-(1-((5,7-dimethyl-1H-indol-4- | | | |
| yl)methyl)-3-ethoxy-3- | | | |
| methylazetidin-2-yl)benzoic acid | | | |
| 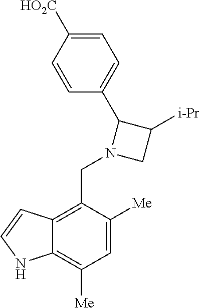 [SMILES: Cc1cc(C)c2[nH]ccc2c1CN1CC(C(C)C)C1c1ccc(C(=O)O)cc1] | 19 (Isomer 1) | Column: CHIRALPAK AD-H Mobile phase: 35% MeOH (0.2% NH₄OH) in CO₂ Flow rate: 12.5 mL/min Column temperature: 39.6° C. Retention time = 2.71 min | LCMS (m/z): [M + H]⁺ calc'd for C₂₄H₂₉N₂O₂, 377.2; found, 377.0 ¹H NMR (400 MHz, ): δ 7.89 (d, J = 8.1 Hz, 2H), 7.36 (d, J = 8.1 Hz, 2H), 7.23 (d, J = 3.1 Hz, 1H), 6.71 (s, 1H), 6.49 (d, J = 3.2 Hz, 1H), 5.20-5.03 (m, 1H), 4.38- 4.21 (m, 2H), 3.91-3.75 (m, 1H), 3.64-3.50 (m, 1H), 2.49- 2.42 (m, 1H), 2.38 (s, 3H), 2.33 (s, 3H), 2.01-1.89 (m, 1H), 0.76 (d, J = 6.5 Hz, 3H), 0.21 (d, J = 6.3 Hz, 3H). |
| 4-(1-((5,7-dimethyl-1H-indol-4- | | | |
| yl)methyl)-3-isopropylazetidin-2- | | | |
| yl)benzoic acid | | | |
| 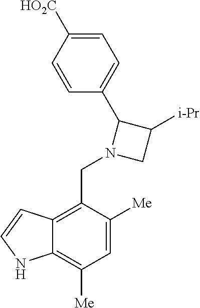 [SMILES: Cc1cc(C)c2[nH]ccc2c1CN1CC(C(C)C)C1c1ccc(C(=O)O)cc1] | 19 (Isomer 2) | Column: CHIRALPAK AD-H Mobile phase: 35% MeOH (0.2% NH₄OH) in CO₂ Flow rate: 12.5 mL/min Column temperature: 39.6° C. Retention time = 4.13 min | LCMS (m/z): [M + H]⁺ calc'd for C₂₄H₂₉N₂O₂, 377.2; found, 376.9 ¹H NMR (400 MHz, ): δ 7.93 (d, J = 7.8 Hz, 2H), 7.38 (d, J = 7.9 Hz, 2H), 7.27 (d, J = 3.1 Hz, 1H), 6.74 (s, 1H), 6.52 (d, J = 3.2 Hz, 1H), 5.25 (d, J = 7.9 Hz,1H), 4.49- 4.34 (m, 2H), 4.01-3.89 (m, 1H), 3.75-3.60 (m, 1H), 2.58- 2.45 (m, 1H), 2.40 (s, 3H), 2.36 (s, 3H), 2.06-1.94 (m, 1H), 0.79 (d, J = 6.5 Hz, 3H), 0.26-0.18 (m, 3H). |
| 4-(1-((5,7-dimethyl-1H-indol-4- | | | |
| yl)methyl)-3-isopropylazetidin-2- | | | |
| yl)benzoic acid | | | |

### Synthesis of 4-(5-(difluoromethyl)-2-((5-methoxy-7-methyl-1H-indol-4-yl)methyl)octahydrocyclopenta[c]pyrrol-1-yl)benzoic acid (Example 39)

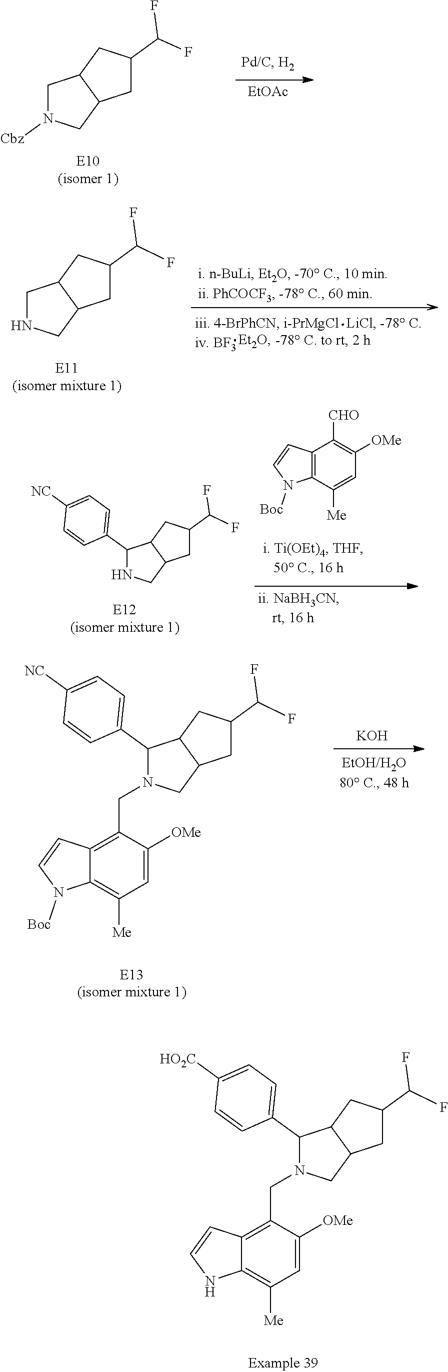

### Step 1: Synthesis of 5-(difluoromethyl)octahydrocyclopenta[c]pyrrole (E11)

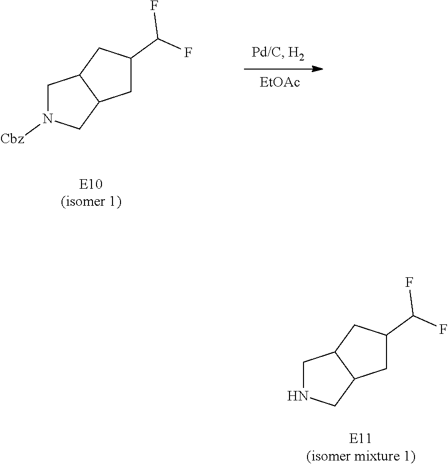 [SMILES: "EtOAc" ; O=C(OCc1ccccc1)N1CC2CC(C(F)F)CC2C1 ; "Pd/C, H2" ; FC(F)C1CC2CNCC2C1]

A mixture of E10 isomer 1 (11 g, 37.3 mmol) and Pd/C (1 g) in ethyl acetate (50 mL) was stirred at rt for 2 hours under H₂. The reaction mixture was filtered and concentrated to provide the product E11 isomer mixture 1 (4.5 g, 75% yield) as a yellow oil. LCMS (m/z): [M+H]+ calc'd for C₈H₁₄F₂N, 162.1; found, 162.1.

The following intermediates were synthesized using similar conditions as those described in step 1, above, along with appropriate starting materials.

| Structure and Name | Intermediate | LCMS |
|---|---|---|
| 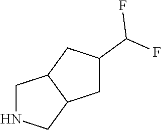 [SMILES: FC(F)C1CC2CNCC2C1] | E11 (Isomer mixture 2) | LCMS (m/z): [M + H]+ calc'd for C₈H₁₄F₂N, 162.1; found, 162.1 |
| 5-(difluoromethyl)octahydrocyclopenta[c]pyrrole | | |
| 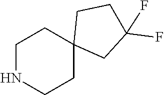 [SMILES: FC1(F)CCC2(CCNCC2)C1] | F21 (from F20) | LCMS (m/z): [M + H]+ calc'd for C₉H₁₆F₂N, 176; found, 176 |
| 2,2-difluoro-8-azaspiro[4.5]decane | | |
| 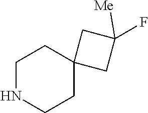 [SMILES: CC1(F)CC2(CCNCC2)C1] | F27 (from F26) | LCMS (m/z): [M + H]+ calc'd for C₉H₁₇FN, 158.1; found, 158.1 |
| 2-fluoro-2-methyl-7-azaspiro[3.5]nonane | | |

### Step 2: Synthesis of 4-(5-(difluoromethyl)octahydrocyclopenta[c]pyrrol-1-yl)benzonitrile

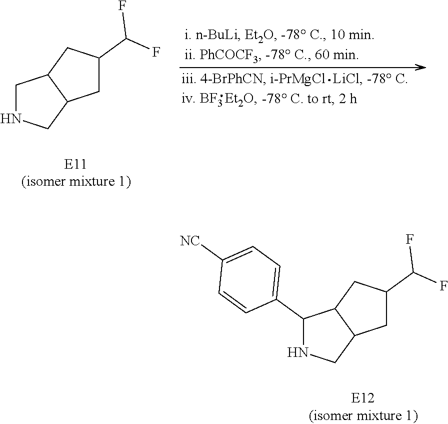 [SMILES: FC(F)C1CC2CNCC2C1 ; N#Cc1ccc(C2NCC3CC(C(F)F)CC32)cc1]

To a solution of i-PrMgCl•LiCl (8.1 mL, 10.5 mmol, 1.3 M) in THF cooled to 0° C. was slowly added 4-bromobenzonitrile (2.54 g, 14 mmol) in THF (10 mL) under an atmosphere of Argon, and the resulting solution was stirred at the same temperature for 2 h.

To a solution of E11 isomer mixture 1 (1.12 g, 7 mmol) in anhydrous ether (20 mL) cooled to −78° C. was slowly added n-BuLi (3.36 mL, 8.4 mmol, 2.5 M) under an atmosphere of Argon, and the resulting solution was stirred at the same temperature for 10 min. To this was then added a solution of PhCOCF3 (1.46 g, 8.4 mmol) in anhydrous ether (8 mL). The resulting mixture was stirred at −78° C. for 60 min, followed by the addition of the organometallic nucleophile reagent (prepared in front) in one portion, followed immediately by the addition of Boron trifluoride etherate (0.99 ml, 7 mmol). Subsequently, the reaction vessel was taken out of the low temperature bath and stirred at room temperature for 2 h. The reaction mixture was then cooled to 0° C. and quenched by the addition of methanol (2 mL). The reaction was diluted with 2M sodium hydroxide solution (50 mL), extracted with ethyl acetate (50 mL×3). The combined organic layers were washed with brine. The organic layer was dried over anhydrous Na₂SO₄. After filtration, the filtrate was concentrated in vacuo and the residue was purified by flash column chromatography (silica gel, 1-3% MeOH in CH₂Cl₂) to afford product E12 isomer mixture 1 (488 mg, 27% yield) as a colorless oil. LCMS (m/z): [M+H]⁺ calc'd for C₁₅H₁₇F₂N₂, 263.1; found, 263.3.

The following intermediates were synthesized using similar conditions as those described in step 2, above, along with appropriate starting materials.

| Structure and Name | Intermediate | LCMS |
|---|---|---|
| 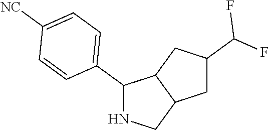 [SMILES: N#Cc1ccc(C2NCC3CC(C(F)F)CC32)cc1] | E12 (Isomer mixture 2) | LCMS (m/z): [M + H]+ calc'd for C₁₅H₁₇F₂N₂, 263.1; found, 263.2 |
| 4-(5-(difluoromethyl)octahydrocyclopenta[c]pyrrol- | | |
| 1-yl)benzonitrile | | |

### Step 3: Synthesis of tert-butyl 4-((1-(4-cyanophenyl)-5-(difluoromethyl)hexahydrocyclopenta[c]pyrrol-2(1H)-yl)methyl)-5-methoxy-7-methyl-11H-indole-1-carboxylate (E13)

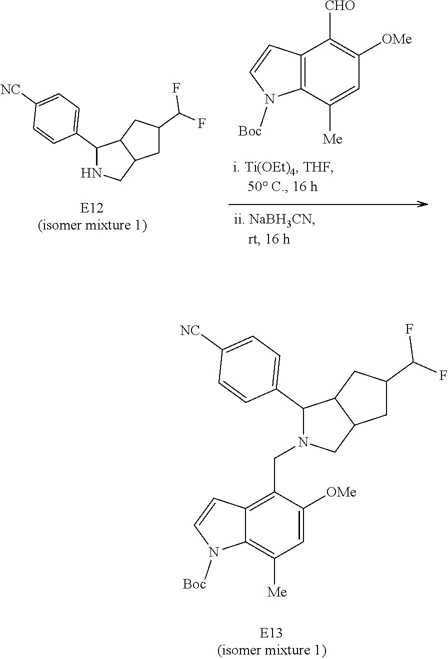 [SMILES: N#Cc1ccc(C2NCC3CC(C(F)F)CC32)cc1 ; COc1cc(C)c2c(ccn2C(=O)OC(C)(C)C)c1C=O ; COc1cc(C)c2c(ccn2C(=O)OC(C)(C)C)c1CN1CC2CC(C(F)F)CC2C1c1ccc(C#N)cc1]

To a solution of E12 isomer mixture 1 (430 mg, 1.64 mmol) and tert-butyl 4-formyl-5-methoxy-7-methyl-1H-indole-1-carboxylate (570 mg, 1.97 mmol) in THF (10 mL) was added Ti(EtO)₄ (748 mg, 3.28 mmol) at 0° C. and then the mixture stirred for 16 h at 50° C. To the reaction solution was added NaBH₃CN (516 mg, 8.2 mmol) at 0° C. The mixture was warmed to room temperature for 16 h. The mixture was quenched with 10 mL H₂O, diluted with EtOAc (50 mL×3), and washed with brine (10 mL). The organic layer was dried over anhydrous Na₂SO₄, filtered, and concentrated and purified by flash column chromatography (silica gel, 4:1 petroleum ether/ethyl acetate) to obtain pure E13 isomer mixture 1 (470 mg, 53% yield) as a white solid. LCMS (m/z): [M+H]⁺ calc'd for C₃₁H₃₆F₂N₃O₃, 536.3; found, 536.2.

The following intermediates were synthesized using similar conditions as those described in step 3, above, along with appropriate starting materials.

| Structure and Name | Intermediate | LCMS |
|---|---|---|
| 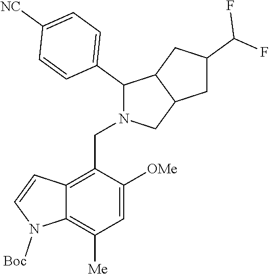 [SMILES: COc1cc(C)c2c(ccn2C(=O)OC(C)(C)C)c1CN1CC2CC(C(F)F)CC2C1c1ccc(C#N)cc1] | E13 (Isomer mixture 2) | LCMS (m/z): [M + H]+ calc'd for C₃₁H₃₆F₂N₃O₃, 536.3; found, 536.2 |
| tert-butyl 4-((1-(4-cyanophenyl)-5- | | |
| (difluoromethyl)hexahydrocyclopenta[c]pyrrol- | | |
| 2(1H)-yl)methyl)-5-methoxy-7-methyl-1H-indole-1- | | |
| carboxylate | | |

### Step 4: Synthesis of 4-(5-(difluoromethyl)-2-((5-methoxy-7-methyl-1H-indol-4-yl)methyl)octahydrocyclopenta[c]pyrrol-1-yl)benzoic acid (Example 39)

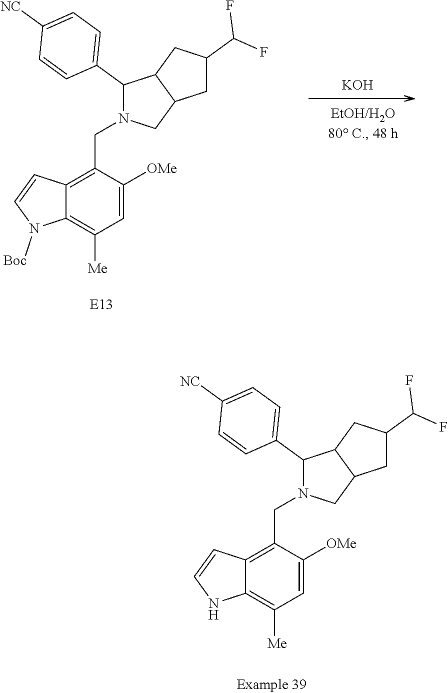 [SMILES: COc1cc(C)c2c(ccn2C(=O)OC(C)(C)C)c1CN1CC2CC(C(F)F)CC2C1c1ccc(C#N)cc1 ; "KOH" ; COc1cc(C)c2[nH]ccc2c1CN1CC2CC(C(F)F)CC2C1c1ccc(C#N)cc1]

To a solution of E13 (470 mg, 0.879 mmol) in EtOH (4 ml) was added a solution of KOH (984 mg, 17.58 mmol) in H₂O (0.8 ml). The mixture was stirred under N₂ atmosphere with balloon at 80° C. for 48 h. The reaction mixture was adjusted to pH=6.4-6.7 with a solution of citric acid (1 mol/L) and concentrated in vacuo. The residue was dissolved with MeOH and water, purified by Prep-HPLC (Waters SunFire 10 μm C18 column, 100 A, 250×19 mm. Solvent A was water/0.01% trifluoroacetic acid and solvent B was acetonitrile. The elution condition was a linear gradient increase of solvent B from 5% to 100% over 20 minutes at a flow rate of 30 mL/min) to give the product the racemate mixture of Example 39 (370 mg, 93% yield) as a white solid.

Example 39: 4-(5-(difluoromethyl)-2-((5-methoxy-7-methyl-1H-indol-4-yl)methyl)octahydrocyclopenta[c]pyrrol-1-yl)benzoic acid (370 mg, 93% yield) was obtained as a white solid. LCMS (m/z): [M+H]⁺ calc'd for C₂₆H₂₉F₂N₂O₃, 455.2; found, 455.1.

Isomers of Example 39 were separated by SFC from a solution of 370 mg of material dissolved in 50 mL MeOH. Isomer 1 and Isomer 2 were purified on a OZ 20*250 mm, 10 um (Daicel). The mobile phase C02/MeOH[0.2% NH3(7M in MeOH)]=55/45 at a flow rate of 100 g/min with back pressure 100 barand a column temperature of 35° C., cycle time 4 min., injection volume 2 mL, and detection wavelength 214 nm. The retention time of Isomer 1 is 1.69 min and Isomer 2 is 2.8 min.

Isomer 1: 4-(5-(difluoromethyl)-2-((5-methoxy-7-methyl-1H-indol-4-yl)methyl)octahydrocyclopenta[c]pyrrol-1-yl)benzoic acid (143.9 mg, 39% yield) as a white solid LCMS (m/z): [M+H]⁺ calc'd for C26H29F2N2O3, 455.2; found, 455.1. 1H NMR (400 MHz, MeOD) δ 8.10 (d, J=7.9 Hz, 2H), 7.56 (d, J=7.8 Hz, 2H), 7.29 (d, J=3.0 Hz, 1H), 6.72 (s, 1H), 6.24 (d, J=2.9 Hz, 1H), 5.89 (td, J=56.8, 4.6 Hz, 1H), 4.35 (s, 1H), 4.30 (s, 2H), 3.74 (s, 3H), 3.73-3.62 (m, 1H), 3.26 (d, J=10.3 Hz, 1H), 3.00 (d, J=37.8 Hz, 2H), 2.63 (s, 1H), 2.48 (s, 3H), 2.22-2.11 (m, 1H), 2.01 (d, J=7.6 Hz, 1H), 1.62 (dt, J=19.0, 12.3 Hz, 2H).

Isomer 2: 4-(5-(difluoromethyl)-2-((5-methoxy-7-methyl-1H-indol-4-yl)methyl)octahydrocyclopenta[c]pyrrol-1-yl)benzoic acid (155.7 mg, 42% yield) as a white solid LCMS (m/z): [M+H]⁺ calc'd for C26H29F2N2O3, 455.2; found, 455.1. 1H NMR (400 MHz, MeOD) δ 8.39 (s, 1H), 8.12 (d, J=7.9 Hz, 2H), 7.59 (d, J=7.9 Hz, 2H), 7.30 (d, J=2.9 Hz, 1H), 6.72 (s, 1H), 6.26 (d, J=2.8 Hz, 1H), 5.90 (td, J=56.6, 4.5 Hz, 1H), 4.44 (d, J=10.4 Hz, 1H), 4.35 (q, J=12.7 Hz, 2H), 3.74 (s, 3H), 3.73-3.66 (m, 1H), 3.34 (d, J=4.9 Hz, 1H), 3.02 (d, J=38.8 Hz, 2H), 2.64 (s, 1H), 2.48 (s, 3H), 2.23-2.11 (m, 1H), 2.01 (s, 1H), 1.64 (dt, J=19.3, 12.3 Hz, 2H).

The following examples were synthesized using the ester hydrolysis procedure described above using appropriate starting materials:

| | | Isomer Separation Method | |
|---|---|---|---|
| | | and Retention Time (if | |
| Name and Stucture | Ex. # | any) | LCMS + ¹H NMR |
| 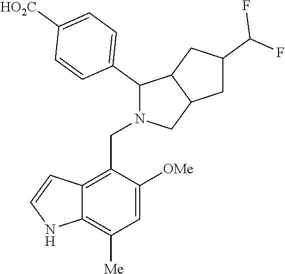 [SMILES: COc1cc(C)c2[nH]ccc2c1CN1CC2CC(C(F)F)CC2C1c1ccc(C(=O)O)cc1] | 39 (Isomer 3) | Column: OZ 20*250 mm, 10 um (Daicel) Column temperature: 35° C. Mobile phase: CO2/MeOH[0.2% NH₃(7M in MeOH)] = 55/45 Flow rate: 100 g/min Back pressure: 100 bar Detection wavelength: 214 nm Cycle time: 4 min Retention time = 1.48 min | LCMS (m/z): [M + H]+ calcd for C₂₆H₂₉F₂N₂O₃, 455.2; found, 455.1 ¹H NMR (400 MHz, MeOD) δ 8.11 (d, J = 8.0 Hz, 2H), 7.59 (d, J = 7.9 Hz, 2H), 7.29 (d, J = 3.0 Hz, 1H), 6.73 (s, 1H), 6.24 (d, J = 2.8 Hz, 1H), 5.88 (td, J = 56.6, 4.7 Hz, 1H), 4.25 (s, 2H), 4.13 (s, 1H), 3.74 (s, 3H), 3.68 (s, 1H), 2.99 (s, 3H), 2.87 (s, 1H), 2.49 (s, 3H), 1.83-1.73 (m, 3H), 1.69 (s, 1H). |
| 4-(5-(difluoromethyl)-2-((5- | | | |
| methoxy-7-methyl-1H-indol-4- | | | |
| yl)methyl)octahydrocyclopenta | | | |
| [c]pyrrol-1-yl)benzoic acid | | | |
| 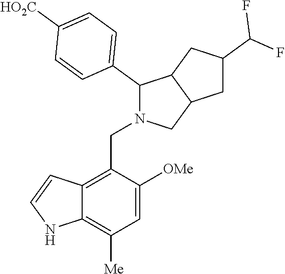 [SMILES: COc1cc(C)c2[nH]ccc2c1CN1CC2CC(C(F)F)CC2C1c1ccc(C(=O)O)cc1] | 39 (Isomer 4) | Column: OZ 20*250 mm, 10 um (Daicel) Column temperature: 35° C. Mobile phase: CO2/MeOH[0.2% NH₃(7M in MeOH)] = 55/45 Flow rate: 100 g/min Back pressure: 100 bar Detection wavelength: 214 nm Cycle time: 4 min Retention time = 2.25 min | LCMS (m/z): [M + H]+ calc'd for C₂₆H₂₉F₂N₂O₃, 455.2; found, 455.1 ¹H NMR (400 MHz, MeOD) δ 8.37 (s, 1H), 8.13 (d, J = 8.0 Hz, 2H), 7.61 (d, J = 8.0 Hz, 2H), 7.30 (d, J = 3.0 Hz, 1H), 6.73 (s, 1H), 6.26 (d, J = 2.7 Hz, 1H), 6.07-5.70 (m, 1H), 4.30 (s, 2H), 4.23 (s, 1H), 3.74 (s, 3H), 3.72-3.67 (m, 1H), 3.12 (d, J = 8.2 Hz, 1H), 3.01 (s, 2H), 2.90 (s, 1H), 2.49 (s, 3H), 1.79 (dd, J = 12.2, 7.1 Hz, 3H), 1.71 (s, 1H). |
| 4-(5-(difluoromethyl)-2-((5- | | | |
| methoxy-7-methyl-1H-indol-4- | | | |
| yl)methyl)octahydrocyclopenta | | | |
| [c]pyrrol-1-yl)benzoic acid | | | |
| 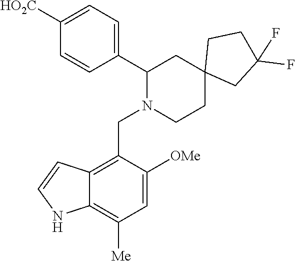 [SMILES: COc1cc(C)c2[nH]ccc2c1CN1CCC2(CCC(F)(F)C2)CC1c1ccc(C(=O)O)cc1] | 41 | | LCMS (m/z): [M + H]+ calc'd for C27H31F2N2O3, 469.2; found, 469.1 1H NMR (500 MHz, MeOD) δ 8.23 (d, J = 7.7 Hz, 2H), 7.74 (s, 2H), 7.34 (s, 1H), 6.77 (s, 1H), 6.37 (d, J = 2.8 Hz, 1H), 4.65- 4.62 (m, 1H), 4.37-4.33 (m, 1H), 4.25-4.21 (m, 1H), 3.76 (s, 3H), 3.48-3.42 (m, 2H), 2.51 (s, 3H), 2.44- 2.40 (m, 1H), 2.33-2.24 (m, 1H), 2.19-2.10 (m, 3H), 2.06-2.02 (m, 2H), 1.96-1.85 (m, 2H), 1.78- 1.74 (m, 1H). |
| 4-(2,2-difluoro-8-((5-methoxy- | | | |
| 7-methyl-1H-indol-4- | | | |
| yl)methyl)-8- | | | |
| azaspiro[4.5]decan-7-yl)benzoic | | | |
| acid | | | |
| 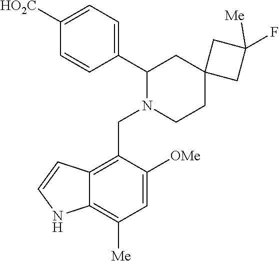 [SMILES: COc1cc(C)c2[nH]ccc2c1CN1CCC2(CC1c1ccc(C(=O)O)cc1)CC(C)(F)C2] | 42 (isomer 1) | Column: IC 20 x 250 mm, 10 μm(Daicel) Mobile phase: CO2/MEOH (0.2% Methanol ammonia) = 55/45 Flow rate: 100 g/min Column temperature: 35° C. Retention time = 2.422 min | LCMS (m/z): [M + H]+ calc'd for C₂₇H₃₁FN₂O₃, 451.2; found, 451.2 ¹H NMR (400 MHz, MeOD) δ 8.11 (d, J = 7.6 Hz, 2H), 7.59 (d, J = 7.2 Hz, 2H), 7.27 (d, J = 2.8 Hz, 1H), 6.73 (s, 1H), 6.31 (s, 1H), 4.16-4.22 (m, 1H), 3.92 (s, 1H), 3.74 (s, 3H), 3.12 (s, 1H), 2.48 (s, 3H), 2.30-2.37 (m, 2H), 2.14- 2.30 (m, 1H), 2.00- 2.10 (m, 2H), 1.93-1.96 (m, 3H), 1.58-1.61 (m, 1H), 1.37-1.46 (m, 3H) |
| 4-(2-fluoro-7-((5-methoxy-7- | | | |
| methyl-1H-indol-4-yl)methyl)- | | | |
| 2-methyl-7-azaspiro[3.5]nonan- | | | |
| 6-yl)benzoic acid | | | |
| 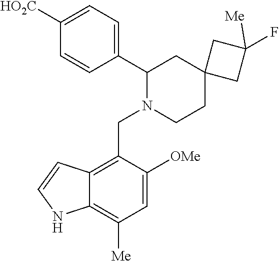 [SMILES: COc1cc(C)c2[nH]ccc2c1CN1CCC2(CC1c1ccc(C(=O)O)cc1)CC(C)(F)C2] | 42 (isomer 2) | Column: IC 20 x 250 mm, 10 μm(Daicel) Mobile phase: CO2/MEOH (0.2% Methanol ammonia) = 55/45 Flow rate: 100 g/min Column temperature: 35° C. Retention time = 3.185 min | LCMS (m/z): [M + H]⁺ calc'd for C₂₇H₃₁FN₂O₃, 451.2; found, 451.2 ¹H NMR (400MHz, MeOD) δ 8.09 (d, J = 8.0 Hz, 2H), 7.59 (d, J = 7.6 Hz, 2H), 7.25 (d, J = 3.2 Hz, 1H), 6.71 (s, 1H), 6.32 (s, 1H), 4.12 (s, 1H), 3.72-3.74 (m, 1H), 3.71 (s, 3H), 3.20-3.25 (m, 1H),2.85 (s, 1H), 2.47 (s, 3H), 2.35-2.40 (m, 1H), 2.24-2.28 (m, 1H), 2.09- 2.16 (m, 1H), 1.96-2.03 (m, 3H), 1.79-1.81 (m, 1H), 1.69-1.77 (m, 1H), 1.42-1.48 (m, 3H) |
| 4-(2-fluoro-7-((5-methoxy-7- | | | |
| methyl-1H-indol-4-yl)methyl)- | | | |
| 2-methyl-7-azaspiro[3.5]nonan- | | | |
| 6-yl)benzoic acid | | | |

### Synthesis of Compound 43

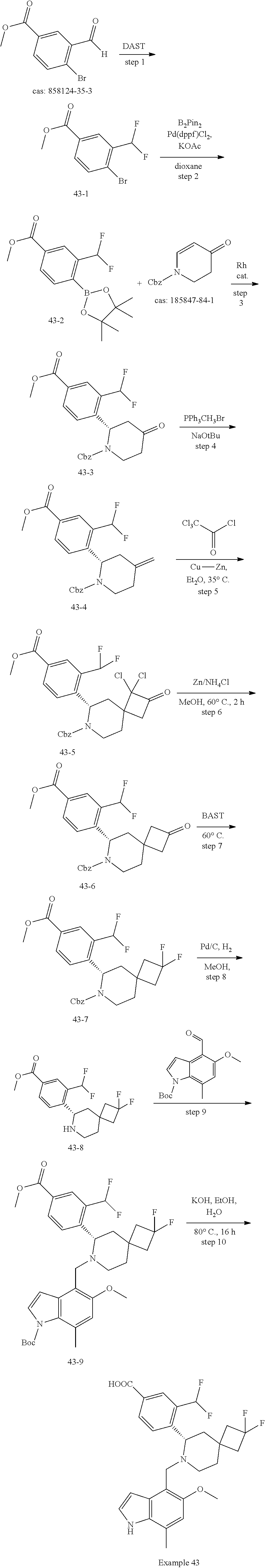

### Step 1

To a solution of methyl 4-bromo-3-formylbenzoate (10.2 g, 42.2 mmol) in dichloroethane (153 ml, 15 V) was added DAST (33.99 g, 210.9 mmol) at −80° C. under N₂ atmosphere with balloon and then warmed to room temperature for 4 h. The reaction mixture was quenched by NH₄Cl solution, concentrated under vacuum to afford product 43-1 as yellow oil (7.70 g, yeild 69%) LCMS (m/z): [M+H]⁺ calc'd for C₉H₈BrF₂O₂, 265.0; found, 265.0.

### Step 2

To a solution of 43-1 (7.70 g, 29.1 mmol) in dioxane (116 mL, 15 V) was added B₂Pin₂ (10.3 g, 40.7 mmol), Pd(dppf)Cl₂ CH₂Cl₂ (2.38 g, 2.91 mmol) and KOAc (8.28 g, 84.4 mmol) at room temperature under N₂ atmosphere with balloon and then warmed to 90° C. for 4 h and then filtered. The filtrate was concentrated in vacuum and the residue was purified by flash column chromatography (ethyl acetate: petroleum ether=10/1) on silica gel to afford product 43-2 as yellow oil (5.10 g, yield 56%). LCMS (m/z): [M+H]⁺ calc'd for C₁₅H₂₀BF₂O₄, 313.1; found, 313.0.

### Step 3

To a solution of 43-2 (5.06 g, 16.2 mmol, 1.5 equiv) and benzyl 4-oxo-3,4-dihydropyridine-1(2H)-carboxylate (2.50 g, 10.8 mmol, 1.0 equiv) in tert-Amyl alcohol (50 mL, 20 V), was added purified water (5 mL, 2 V), Rh(Acac)(C₂H₄)₂ (84 mg, 0.32 mmol) and (R, R )-Ph-PBE (164 mg, 0.324 mmol) at room temperature in the glove box. The mixture was stirred at 50° C. for 12 h and then filtered. The filtrate was concentrated in vacuum and the residue was purified by flash column chromatography (ethyl acetate: petroleum ether=4/1) on silica gel to afford product 43-3 as yellow oil (2.08 g, yield 46%, 100% ee). LCMS (m/z): [M+H]⁺ calc'd for C₂₂H₂₂F₂NO₅, 418.15; found, 418.19.

### Test Method: Column: CHIRALPAK IA 4.6*250 mm, 5 μm

Mobile phase: 60% hexane, 40% EtOH, 0.10% methanesulfonic acid
Flow rate: 1.0 mL/min
Column temperature: 30° C.
Retention time=7.9 min

The following intermediates were synthesized using similar conditions as those described in the steps, above, along with appropriate starting materials.

| Structure | Intermediate | LCMS | Chiral Separation conditions |
|---|---|---|---|
| 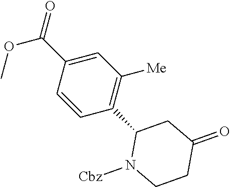 [SMILES: COC(=O)c1ccc([C@@H]2CC(=O)CCN2C(=O)OCc2ccccc2)c(C)c1] | 43-3i | LCMS (m/z): [M + H]⁺ calc'd for C₂₂H₂₄NO₅, 382.16; found, 382.24. | Column: CHIRALPAK IA 4.6*250 mm, 5 μm Mobile phase: 50% hexane, 50% EtOH, 0.1% methanesulfonic acid Flow rate: 1.0 mL/min Column temperature: 30° C. Retention time = 7.8 min |
| 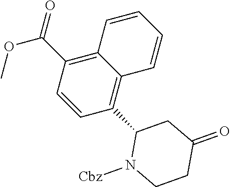 [SMILES: COC(=O)c1ccc([C@@H]2CC(=O)CCN2C(=O)OCc2ccccc2)c2ccccc12] | 43-3ii | LCMS (m/z): [M + H]⁺ calc'd for C₂₅H₂₃NO₅, 418.2; found, 418.24. | Column: CHIRALPAK IC 4.6*250 mm, 5 μm Mobile phase: 60% CO₂, 40% MeOH, 0.2% NH₄OH Flow rate: 2.5 mL/min Column temperature: 40.1° C. Retention time = 3.57 min |
| 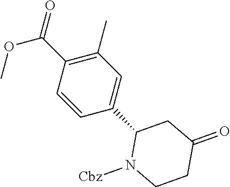 [SMILES: COC(=O)c1ccc([C@@H]2CC(=O)CCN2C(=O)OCc2ccccc2)cc1C] | 43-3iii | LCMS (m/z): [M + H]⁺ calc'd for C₂₂H₂₄NO₅, 382.2; found, 382.5. | Column: CHIRALPAK AD-H 4.6*250 mm, 5 μm Mobile phase: 60% CO₂, 40% EtOH, 0.2% NH₄OH Flow rate: 2.5 mL/min Column temperature: 40° C. Retention time = 2.8 min |
| 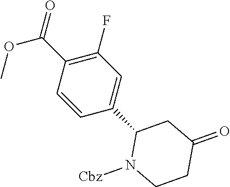 [SMILES: COC(=O)c1ccc([C@@H]2CC(=O)CCN2C(=O)OCc2ccccc2)cc1F] | 43-3iv | LCMS (m/z): [M + H]⁺ calc'd for C₂₁H₂₁FNO₅, 386.2; found, 386.1. | Column: CHIRALPAK AD-H 4.6*250 mm, 5 μm Mobile phase: 60% CO₂, 40% MeOH, 0.2% NH₄OH Flow rate: 2.5 mL/min Column temperature: 40° C. Retention time = 4.83 min |
| 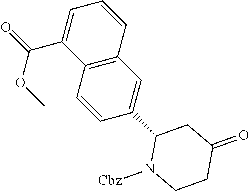 [SMILES: COC(=O)c1cccc2cc([C@@H]3CC(=O)CCN3C(=O)OCc3ccccc3)ccc12] | 43-3v | LCMS (m/z): [M + H]⁺ calc'd for C₂₅H₂₄NO₅, 418.2; found, 418.4. | Column: CHIRALPAK IC 4.6*250 mm, 5 μm Mobile phase: 60% CO₂, 40% MeOH, 0.2% NH₄OH Flow rate: 2.5 mL/min Column temperature: 40.1° C. Retention time = 6.02 min |

### Step 4

To a solution of PPh₃CH₃Br (2.668 g, 7.47 mmol, 1.5 equiv) in toluene (21.6 mL, 20 V), was added NaO^{t}Bu (684 mg, 7.12 mmol, 1.43 equiv) at room temperature under N₂ atmosphere with balloon. The mixture was stirred at room temperature for 2 h. To a solution of 43-3 (2.080 g 4.94 mmol, 1.0 equiv) in toluene (10.8 mL, 10 V), was added to the mixture at room temperature and then stirred for 1 h. The reaction mixture was quenched with 108 mL (50 V) saturated NH₄Cl solution and extracted with ethyl acetate (156 mL×2, 75 V), washed with brine (52 mL, 25 V), dried over Na₂SO4, concentrated under vacuum, and purified by flash column chromatography (ethyl acetate: petroleum ether= 1/16 to ⅛) on silica gel to get a colorless oil, 43-4 (1.16 g, yield 56%). LCMS (m/z): [M+H]⁺ calc'd for C₂₃H₂₄F₂NO₄, 416.2; found, 416.3.

### Step 5

To a suspension of Cu—Zn (3.5 g, 3% Cu) and 43-4 (1.0 g, 2.4 mmol, 1.0 equiv) in dioxane (70 mL, 70 V) was added trichloroacetyl chloride (4.4 g, 24 mmol, 10 equiv) at 20° C. over 30 min under a nitrogen atmosphere. The mixture was heated to 35° C. for 3 h. The reaction mixture was quenched by NH₄C1 solution. The solid was filtered off and the filtrate was extracted with ethyl acetate (30 mL×3, 30 V), washed with brine (40 mL, 40 V). The combined filtrate was dried over Na₂SO₄, concentrated under vacuum to give the oil crude product 43-5 (1.17 g) which was used in the next step without further purification. LCMS (m/z): [M+H]⁺ calc'd for C₂₅H₂₃Cl₂F₂NO₅, 526.1; found, 526.3.

### Step 6

To a solution of 43-5 (1.17 g, crude) in MeOH (50 mL) was added NH₄C1 (2.6 g, 48 mmol) and Zinc (1.6 g, 24 mmol) at rt. The reaction mixture was stirred at 60° C. for 2 h, and then filtered. The filtrate was concentrated in vacuum and the residue was purified by flash column chromatography (ethyl acetate: petroleum ether=4/1) on silica gel to afford product 43-6 as an oil (830 mg, two step yield 75%). LCMS (m/z): [M+H]⁺ calc'd for C₂₅H₂₆F₂NO₅, 458.2; found, 458.3.

### Step 7

43-6 (830 mg, 1.82 mmol) was dissolved in BAST (1.5 mL) at 0° C. The reaction mixture was stirred at 60° C. for 12 h. The reaction was cooled to room temperature and 15 mL ethyl acetate was added. The reaction mixture was poured into ice (10 g) very carefully. The residue was extracted with ethyl acetate (30 mL×3), washed with brine (30 mL), dried over Na₂SO₄. The filtrate was concentrated in vacuum and purified by flash column chromatography (ethyl acetate: petroleum ether=4/1) on silica gel to afford product 43-7 as a oil (569 mg, yield 65%). LCMS (m/z): [M+H]⁺ calc'd for C₂₅H₂₆F₄NO₄, 480.2; found, 480.1.

### Step 8

To a solution of 43-7 (569 mg, 1.19 mmol) in MeOH (17 mL) was added active carbon (569 mg). The reaction was heated to reflux for 0.5 h. Active carbon was filtered, and Pd/C (57 mg, Pd 10%) was added to the filtrate. The reaction mixture was stirred for 2 h at rt with a H₂ balloon. Pd/C was removed, and the filtrate was concentrated under vacuum to get the product 43-8 as an oil (328 mg, yield 80%). LCMS (m/z): [M+H]⁺ calc'd for C₁₇H₂₀F₄NO₂, 346.1; found, 346.2.

### Step 9

To a solution of 43-8 (330 mg, 0.95 mmol) in DCE (1.5 mL), was added aldehyde (316 mg, 1.09 mmol) and NaBH(OAc)₃ (632 mg, 2.98 mmol). The mixture was stirred at room temperature for 15 h. The reaction mixture was quenched with 1.0 mL of water then evaporated the solvent, concentrated to give the crude product. The crude product was purified by flash column chromatography (ethyl acetate: petroleum ether=10/1) on silica gel to afford product 43-9 as a white solid (270 mg, yield 46%). LCMS (m/z): [M+H]⁺ calc'd for C₃₃H₃₉F₄N₂O₅, 619.3; found, 619.2.

### Step 10

To a solution of 43-9 (270 mg, 0.44 mmol) in EtOH (8 mL) was add 30% o KOH solution (0.8 mL), The mixture was stirred at 80° C. for 4 h. The reaction mixture was adjusted to pH=6 and concentrated in vacuum to get a 120 mL of solution. The solution was purified by prep-HPLC. The preparation solution was concentrated to remove CH₃CN and extracted with ethyl acetate (20 mL×5), dried over Na₂SO₄, filtered. The filtrate was concentrated to dryness as a white solid to give Example 43 (32 mg, yield 15%). LCMS (m/z): [M+H]⁺ calc'd for C₂₇H₂₉F₄N₂O₃, 505.21; found, 505.29; ¹H NMR (400 MHz, CD₃OD): δ 8.34 (d, J=7.8 Hz, 1H), 8.26 (s, 1H), 7.95 (d, J=7.6 Hz, 1H), 7.3 3 (d, J=3.0 Hz, 1H), 7.17 (br, 1H), 7.04 (br, 1H), 6.77 (s, 1H), 6.46 (d, J=3.2 Hz, 1H), 4.56 (s, 1H), 4.19 (d, J=12.0 Hz, 1H), 4.05-3.87 (m, 1H), 3.79 (s, 3H), 3.50-3.39 (m, 1H), 3.27-3.05 (m, 1H), 2.80-2.55 (m, 2H), 2.52 (s, 3H), 2.42 (t, J=12.4 Hz, 2H), 2.25-1.95 (m, 3H), 1.92-1.80 (in, 1H).

The Example 43-77 were prepared as follows:

| | | Reference | | |
|---|---|---|---|---|
| Example | | synthetic | | |
| number | Structure | procedure | Starting materials | LCMS and HNMR |
| 44 | 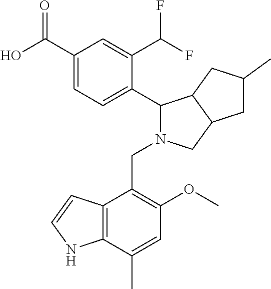 [SMILES: COc1cc(C)c2[nH]ccc2c1CN1CC2CC(C)CC2C1c1ccc(C(=O)O)cc1C(F)F] | Example 13 | CAS: 1263378-05-7 | 469 |
| 44 (Isomer 1) | 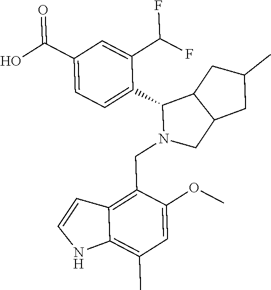 [SMILES: COc1cc(C)c2[nH]ccc2c1CN1CC2CC(C)CC2[C@H]1c1ccc(C(=O)O)cc1C(F)F] | Example 13 | CAS: 1263378-05-7 | 469 |
| 45 | 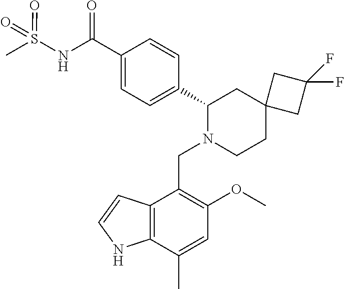 [SMILES: COc1cc(C)c2[nH]ccc2c1CN1CCC2(C[C@H]1c1ccc(C(=O)NS(C)(=O)=O)cc1)CC(F)(F)C2] | Example 33 | CAS: 2238811-87-3 | 532 |
| 46 | 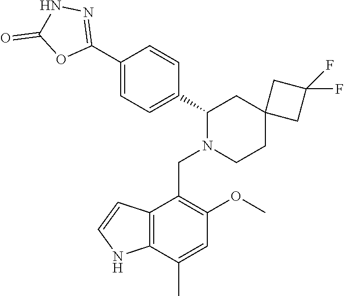 [SMILES: COc1cc(C)c2[nH]ccc2c1CN1CCC2(C[C@H]1c1ccc(-c3n[nH]c(=O)o3)cc1)CC(F)(F)C2] | Example 33 | CAS: 2238811-87-3 | 495 |
| 47 | 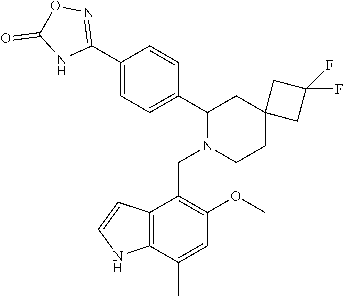 [SMILES: COc1cc(C)c2[nH]ccc2c1CN1CCC2(CC1c1ccc(-c3noc(=O)[nH]3)cc1)CC(F)(F)C2] | Example 33 | CAS: 2238811-87-3 | 495 |
| 47 (Isomer 1) | 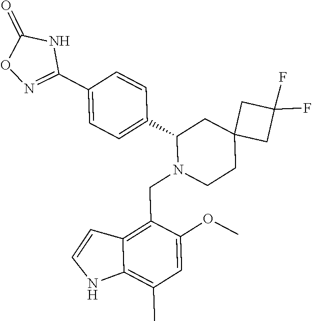 [SMILES: COc1cc(C)c2[nH]ccc2c1CN1CCC2(C[C@H]1c1ccc(-c3noc(=O)[nH]3)cc1)CC(F)(F)C2] | Example 33 | CAS: 2238811-87-3 | 495 |
| 48 | 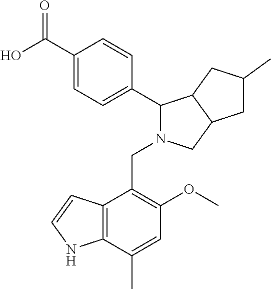 [SMILES: COc1cc(C)c2[nH]ccc2c1CN1CC2CC(C)CC2C1c1ccc(C(=O)O)cc1] | Example 13 | CAS: 1263378-05-7 | 419 |
| 49 | 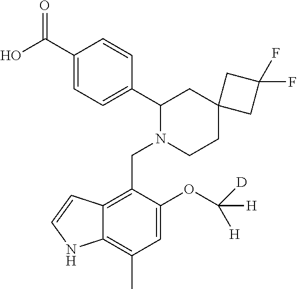 [SMILES: [2H]COc1cc(C)c2[nH]ccc2c1CN1CCC2(CC1c1ccc(C(=O)O)cc1)CC(F)(F)C2] | Example 33 | CAS: 2238811-87-3 | 456 |
| 50 | 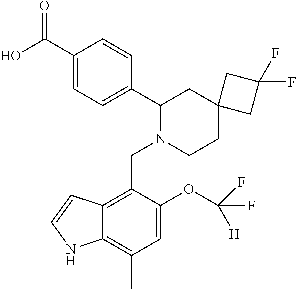 [SMILES: Cc1cc(OC(F)F)c(CN2CCC3(CC2c2ccc(C(=O)O)cc2)CC(F)(F)C3)c2cc[nH]c12] | Example 33 | CAS: 2238811-87-3 | 491 |
| 51 | 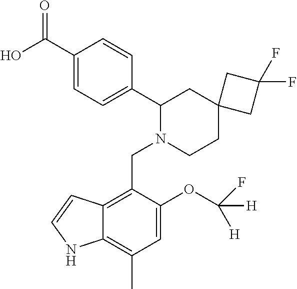 [SMILES: Cc1cc(OCF)c(CN2CCC3(CC2c2ccc(C(=O)O)cc2)CC(F)(F)C3)c2cc[nH]c12] | Example 33 | CAS: 2238811-87-3 | 473 |
| 52 | 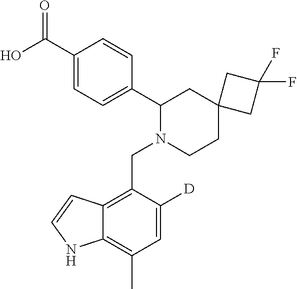 [SMILES: [2H]c1cc(C)c2[nH]ccc2c1CN1CCC2(CC1c1ccc(C(=O)O)cc1)CC(F)(F)C2] | Example 33 | CAS: 2238811-87-3 | 426 |
| 53 | 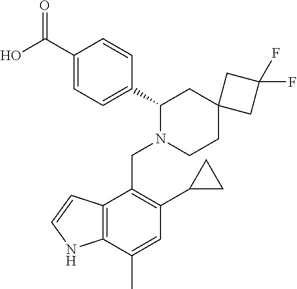 [SMILES: Cc1cc(C2CC2)c(CN2CCC3(C[C@H]2c2ccc(C(=O)O)cc2)CC(F)(F)C3)c2cc[nH]c12] | Example 33 | CAS: 2238811-87-3 | LCMS: m/z = 465.1 (M + 1, ESI+). ¹H NMR (400 MHz, MeOD) δ 8.31 (brs, 1H), 8.07 (d, J = 8.2 Hz, 2H), 7.62 (d, J = 8.0 Hz, 2H), 7.24 (d, J = 3.1 Hz, 1H), 6.59 (s, 1H), 6.40 (brs, 1H), 4.40 (d, J = 12.1 Hz, 1H), 4.15- 4.03 (m, 2H), 3.33-3.27 (m, 1H), 3.04-2.91 (m, 1H), 2.72- 2.53 (m, 2H), 2.43-2.33 (m, 4H), 2.27-2.17 (m, 1H), 2.08-1.66 (m, 5H), 0.78- 0.72 (m, 2H), 0.25 (brs, 1H), −0.00 (brs, 1H). |
| 54 | 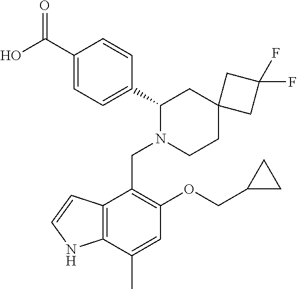 [SMILES: Cc1cc(OCC2CC2)c(CN2CCC3(C[C@H]2c2ccc(C(=O)O)cc2)CC(F)(F)C3)c2cc[nH]c12] | Example 33 | CAS: 2238811-87-3 | 495 |
| 55 | 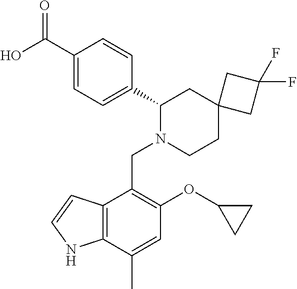 [SMILES: Cc1cc(OC2CC2)c(CN2CCC3(C[C@H]2c2ccc(C(=O)O)cc2)CC(F)(F)C3)c2cc[nH]c12] | Example 33 | CAS: 2238811-87-3 | 481 |
| 56 | 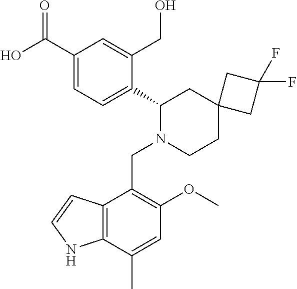 [SMILES: COc1cc(C)c2[nH]ccc2c1CN1CCC2(C[C@H]1c1ccc(C(=O)O)cc1CO)CC(F)(F)C2] | Example 36 | CAS: 2238811-87-3 | 484 |
| 57 | 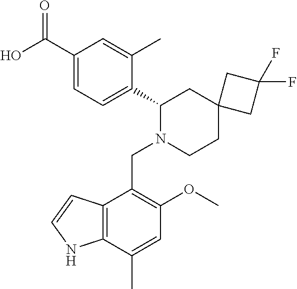 [SMILES: COc1cc(C)c2[nH]ccc2c1CN1CCC2(C[C@H]1c1ccc(C(=O)O)cc1C)CC(F)(F)C2] | Example 43 | CAS: 2238811-87-3 | LCMS (m/z): [M + H]⁺ calc'd for C₂₇H₃₁F₂N₂O₃, 469.23; found, 469.30.; ¹H NMR (400 MHz, CD₃OD): δ 8.05 (d, J = 6.9 Hz, 1H), 7.94 (s, 1H), 7.69 (d, J = 8.1 Hz, 1H), 7.33 (d, J = 3.1 Hz, 1H), 6.78 (s, 1H), 6.40 (d, J = 3.1 Hz, 1H), 4.55 (d, J = 11.3 Hz, 1H), 4.30 (d, J = 12.4 Hz, 1H), 4.15-4.05 (m, 1H), 3.79 (s, 3H), 3.54-3.42 (m, 1H), 3.32-3.24 (m, 1H), 2.80-2.65 (m, 2H), 2.56 (s, 3H), 2.52 (s, 3H), 2.50-2.40 (m, 2H), 2.26-2.12 (m, 1H), 2.11-2.00 (m, 2H), 1.96-1.85 (m, 1H). |
| 58 | 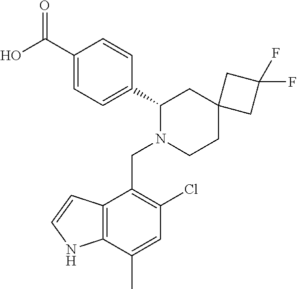 [SMILES: Cc1cc(Cl)c(CN2CCC3(C[C@H]2c2ccc(C(=O)O)cc2)CC(F)(F)C3)c2cc[nH]c12] | Example 33 | CAS: 2238811-87-3 | 459 |
| 59 | 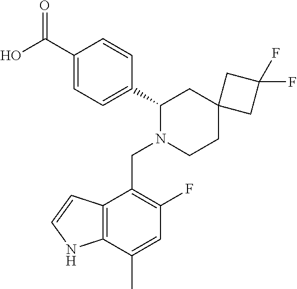 [SMILES: Cc1cc(F)c(CN2CCC3(C[C@H]2c2ccc(C(=O)O)cc2)CC(F)(F)C3)c2cc[nH]c12] | Example 33 | CAS: 2238811-87-3 | 443 |
| 60 | 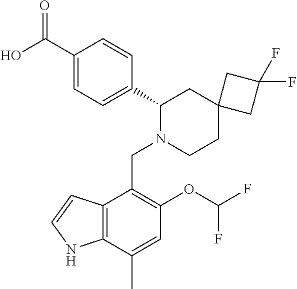 [SMILES: Cc1cc(OC(F)F)c(CN2CCC3(C[C@H]2c2ccc(C(=O)O)cc2)CC(F)(F)C3)c2cc[nH]c12] | Example 33 | CAS: 2238811-87-3 | 491 |
| 61 | 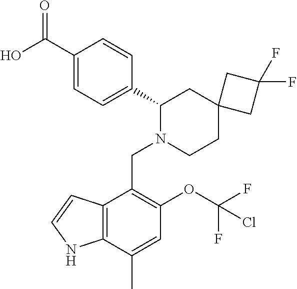 [SMILES: Cc1cc(OC(F)(F)Cl)c(CN2CCC3(C[C@H]2c2ccc(C(=O)O)cc2)CC(F)(F)C3)c2cc[nH]c12] | Example 33 | CAS: 2238811-87-3 | 525 |
| 62 | 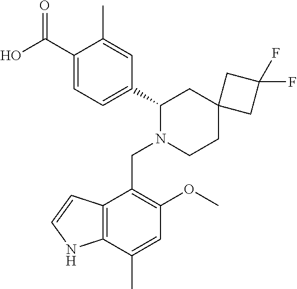 [SMILES: COc1cc(C)c2[nH]ccc2c1CN1CCC2(C[C@H]1c1ccc(C(=O)O)c(C)c1)CC(F)(F)C2] | Example 43 | CAS: 2238811-87-3 Column: CHIRALPAK whelk-ol 4.6*250 mm, 5 μm Mobile phase: 60% CO₂, 40% EtOH, 0.2% NH₄OH Flow rate: 2.5 mL/min Column temperature: 39.9° C. Retention time = 3.09 min | LCMS (m/z): [M + H]⁺ calc'd for C₂₇H₃₀F₂N₂O₃, 468.22; found, 469.2. ¹H NMR (400 MHz, CD³OD) δ 8.37 (s, 1H), 7.87 (d, J = 7.8 Hz, 1H), 7.48 (d, J = 11.6 Hz, 2H), 7.35 (d, J = 3.1 Hz, 1H), 6.78 (s, 1H), 6.33 (s, 1H), 4.47- 4.34 (m, 2H), 4.13 (d, J = 12.7 Hz, 1H), 3.78 (s, 3H), 3.49 (d, J = 12.3 Hz, 1H), 3.30- 3.22 (m, 1H), 2.82-2.62 (m, 5H), 2.54-2.42 (m, 5H), 2.32 (s, 1H), 2.14-2.02 (m, 2H), 1.92 (d, J = 13.7 Hz, 1H). |
| 63 | 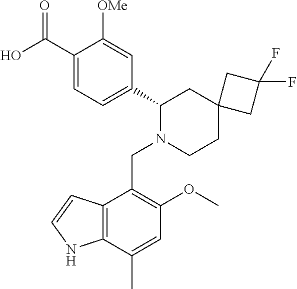 [SMILES: COc1cc([C@@H]2CC3(CCN2Cc2c(OC)cc(C)c4[nH]ccc24)CC(F)(F)C3)ccc1C(=O)O] | Example 36 | CAS: 2238811-87-3 | 485 |
| 64 | 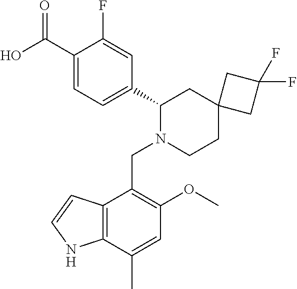 [SMILES: COc1cc(C)c2[nH]ccc2c1CN1CCC2(C[C@H]1c1ccc(C(=O)O)c(F)c1)CC(F)(F)C2] | Example 43 | CAS: 2238811-87-3 | LCMS (m/z): [M + H]⁺ calc'd for C₂₆H₂₇F₃N₂O₃, 473.21; found, 473.0. NMR (400 MHz, CD3OD) δ 8.38 (s, 1H), 7.88 (t, J = 7.6 Hz, 1H), 7.43 (t, J = 8.6 Hz, 2H), 7.33 (d, J = 3.1 Hz, 1H), 6.77 (s, 1H), 6.37 (d, J = 3.0 Hz, 1H), 4.41 (d, J = 11.5 Hz, 1H), 4.36 (d, J = 12.6 Hz, 1H), 4.07 (d, J = 12.7 Hz, 1H), 3.78 (s, 3H), 3.52-3.39 (m, 1H), 3.21-3.17 (m, 1H), 2.81- 2.59 (m, 2H), 2.51 (s, 3H), 2.45 (t, J = 12.8 Hz, 2H), 2.33- 2.19 (m, 1H), 2.14-1.98 (m, 2H), 1.93-1.84 (m, 1H). |
| 65 | 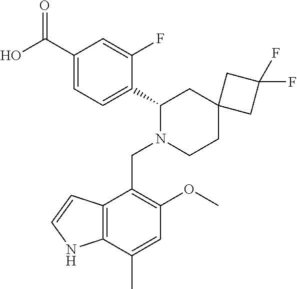 [SMILES: COc1cc(C)c2[nH]ccc2c1CN1CCC2(C[C@H]1c1ccc(C(=O)O)cc1F)CC(F)(F)C2] | Example 43 | CAS: 2238811-87-3 | 473 |
| 66 | 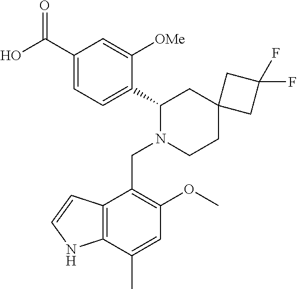 [SMILES: COc1cc(C(=O)O)ccc1[C@@H]1CC2(CCN1Cc1c(OC)cc(C)c3[nH]ccc13)CC(F)(F)C2] | Example 43 | CAS: 2238811-87-3 | 485 |
| 67 | 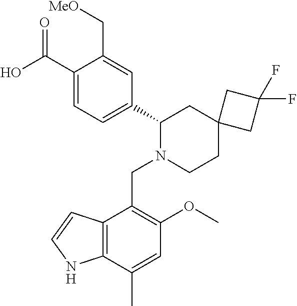 [SMILES: COCc1cc([C@@H]2CC3(CCN2Cc2c(OC)cc(C)c4[nH]ccc24)CC(F)(F)C3)ccc1C(=O)O] | Example 43 | CAS: 2238811-87-3 | 499 |
| 68 | 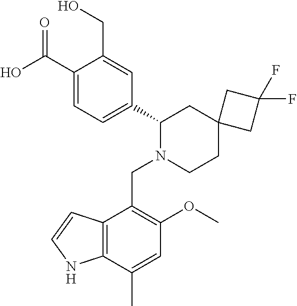 [SMILES: COc1cc(C)c2[nH]ccc2c1CN1CCC2(C[C@H]1c1ccc(C(=O)O)c(CO)c1)CC(F)(F)C2] | Example 43 | CAS: 2238811-87-3 | 485 |
| 69 | 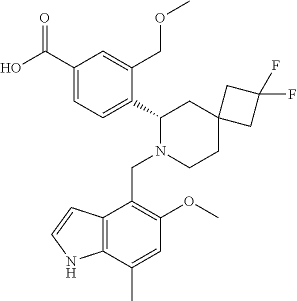 [SMILES: COCc1cc(C(=O)O)ccc1[C@@H]1CC2(CCN1Cc1c(OC)cc(C)c3[nH]ccc13)CC(F)(F)C2] | Example 43 | CAS: 2238811-87-3 | 499 |
| 70 | 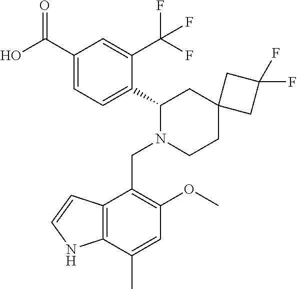 [SMILES: COc1cc(C)c2[nH]ccc2c1CN1CCC2(C[C@H]1c1ccc(C(=O)O)cc1C(F)(F)F)CC(F)(F)C2] | Example 43 | CAS: 2238811-87-3 | 523 |
| 71 | 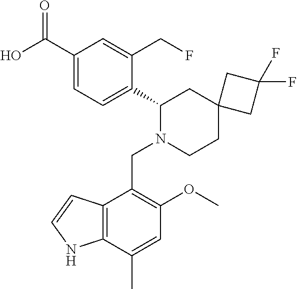 [SMILES: COc1cc(C)c2[nH]ccc2c1CN1CCC2(C[C@H]1c1ccc(C(=O)O)cc1CF)CC(F)(F)C2] | Example 36 | CAS: 2238811-87-3 | 487 |
| 72 | 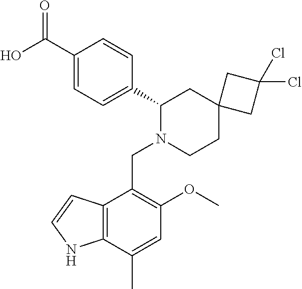 [SMILES: COc1cc(C)c2[nH]ccc2c1CN1CCC2(C[C@H]1c1ccc(C(=O)O)cc1)CC(Cl)(Cl)C2] | Example 33 | CAS: 2238811-87-3 | 486 |
| 73 | 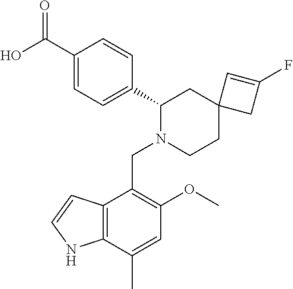 [SMILES: COc1cc(C)c2[nH]ccc2c1CN1CCC2(C=C(F)C2)C[C@H]1c1ccc(C(=O)O)cc1] | Example 33 | CAS: 2238811-87-3 | 435 |
| 74 | 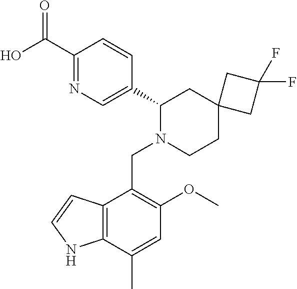 [SMILES: COc1cc(C)c2[nH]ccc2c1CN1CCC2(C[C@H]1c1ccc(C(=O)O)nc1)CC(F)(F)C2] | Example 43 | CAS: 2238811-87-3 | 456 |
| 75 | 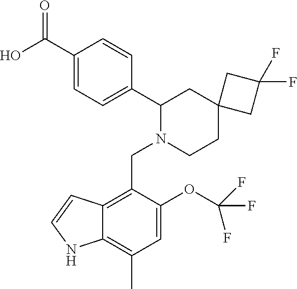 [SMILES: Cc1cc(OC(F)(F)F)c(CN2CCC3(CC2c2ccc(C(=O)O)cc2)CC(F)(F)C3)c2cc[nH]c12] | Example 33 | CAS: 2238811-87-3 | 509 |
| 76 | 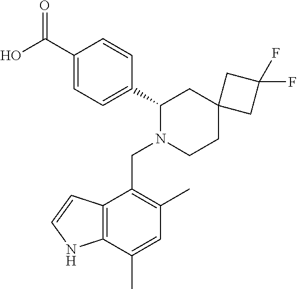 [SMILES: Cc1cc(C)c2[nH]ccc2c1CN1CCC2(C[C@H]1c1ccc(C(=O)O)cc1)CC(F)(F)C2] | Example 33 | CAS: 2238811-87-3 | 439 |
| 77 | 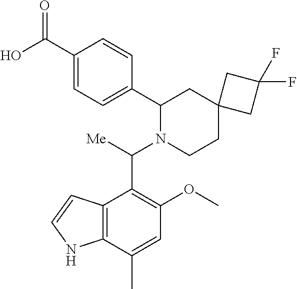 [SMILES: COc1cc(C)c2[nH]ccc2c1C(C)N1CCC2(CC1c1ccc(C(=O)O)cc1)CC(F)(F)C2] | Example 33 | CAS: 2238811-87-3 | 469 |
| 78 | 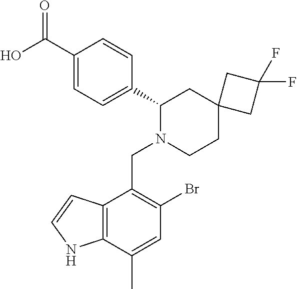 [SMILES: Cc1cc(Br)c(CN2CCC3(C[C@H]2c2ccc(C(=O)O)cc2)CC(F)(F)C3)c2cc[nH]c12] | Example 33 | CAS: 2238811-87-3 | 503 |
| 79 | | Example | CAS: 2238811-87-3 | 494 |
| | | 33 | | |
| 80 | 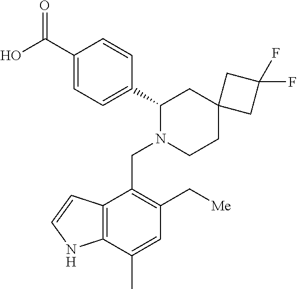 [SMILES: CCc1cc(C)c2[nH]ccc2c1CN1CCC2(C[C@H]1c1ccc(C(=O)O)cc1)CC(F)(F)C2] | Example 33 | CAS: 2238811-87-3 | LCMS (m/z): [M + H]⁺ calc'd for C₂₇H₃₀F₂N₂O₂, 453.24; found, 453.1. ¹H NMR (400 MHz, CD3OD) δ 8.34 (s, 1H), 8.11 (d, J = 8.1 Hz, 2H), 7.66 (d, J = 7.5 Hz, 2H), 7.23 (d, J = 3.1 Hz, 1H), 6.75 (s, 1H), 6.43 (d, J = 3.1 Hz, 1H), 4.02 (t, J = 12.3 Hz, 2H), 3.81 (d, J = 12.5 Hz, 1H), 3.16 (d, J = 10.6 Hz, 1H), 2.84- 2.52 (m, 4H), 2.44-2.34 (m, 6H), 2.18-2.22 (m, 1H), 2.01-1.74 (m, 3H), 0.87 (t, J = 7.2 Hz, 3H). |
| 81 | 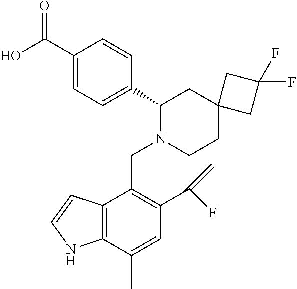 [SMILES: C=C(F)c1cc(C)c2[nH]ccc2c1CN1CCC2(C[C@H]1c1ccc(C(=O)O)cc1)CC(F)(F)C2] | Example 33 | CAS: 2238811-87-3 | LCMS (m/z): [M + H]⁺ calc'd for C₂₂H₂₄NO₅, 469.21; found, 469.1. ¹H NMR (400 MHz, CD3OD) δ 8.22 (d, J = 7.8 Hz, 2H), 7.73 (d, J = 7.3 Hz, 2H), 7.40 (d, 1H), 7.13 (s, 1H), 6.28 (s, 1H), 4.85-4.73 (dd, J = 3.3 Hz, 1H), 4.62 (dd, J = 12.7, 2.4 Hz, 1H), 4.54-4.45 (m, 2H), 3.51-3.41 (m, 1H), 2.80- 2.63 (m, 2H), 2.55-2.32 (m, 7H), 2.18-1.84 (m, 4H). |
| 82 | 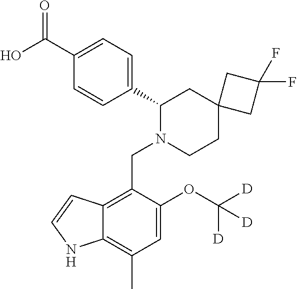 [SMILES: [2H]C([2H])([2H])Oc1cc(C)c2[nH]ccc2c1CN1CCC2(C[C@H]1c1ccc(C(=O)O)cc1)CC(F)(F)C2] | Example 43 | CAS: 2238811-87-3 | LCMS (m/z): [M + H]+ calc'd for C26H25D3F2N2O3, 458.23; found, 458.1. 1H NMR (400 MHz, MeOD) δ 8.40 (s, 0.49H), 8.16 (d, J = 8.0 Hz, 2H), 7.66 (d, J = 7.6 Hz, 2H), 7.31 (d, J = 2.8 Hz, 1H), 6.74 (s, 1H), 6.30 (d, J = 2.8 Hz, 1H), 4.43 (d, J = 10.8 Hz, 1H), 4.32 (d, J = 12.4 Hz, 1H), 4.08 (d, J = 12.8 Hz, 1H), 3.47 (d, J = 12.8 Hz, 1H), 3.24 (td, J = 13.2, 2.8 Hz, 1H), 2.80 2.62 (m, 2H), 2.49 (s, 3H), 2.43 (m, 2H), 2.36-2.26 (m, 1H), 2.12-2.01 (m, 2H), 1.90 (m, 1H). |
| 83 | 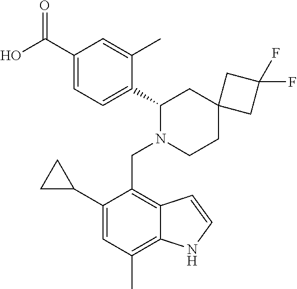 [SMILES: Cc1cc(C(=O)O)ccc1[C@@H]1CC2(CCN1Cc1c(C3CC3)cc(C)c3[nH]ccc13)CC(F)(F)C2] | Example 43 | CAS: 2238811-87-3 Column: CHIRALPAK AD-H 4.6*250 mm, 5 μm Mobile phase: 60% CO₂, 40% MeOH, 0.2% NH₄OH Flow rate: 2.5 mL/min Column temperature: 39.5° C. Retention time = 2.27 min | LCMS (m/z): [M + H]⁺ calc'd for C₂₆H₂₇F₃N₂O₃, 479.25; found, 479.0. ¹H NMR (400 MHz, MeOD) δ 8.40 (s, 0.3H), 8.00-7.87 (m, 2H), 7.67 (d, J = 8.1 Hz, 1H), 7.28 (d, J = 3.2 Hz, 1H), 6.62 (s, 1H), 6.51 (d, J = 3.1 Hz, 1H), 4.51-4.28 (m, 2H), 4.11 (d, J = 12.5 Hz, 1H), 3.37 (d, J = 12.4 Hz, 1H), 3.19-2.98 (m, 1H), 2.69-2.64 (m, 2H), 2.59 (s, 3H), 2.49-2.31 (m, 5H), 2.16 (t, J = 13.4 Hz, 1H), 2.00-1.89 (m, 2H), 1.88- 1.76 (m, 2H), 0.88-0.75 (m, 2H), 0.32-0.21 (m, 1H), 0.06-−0.05 (m, 1H) |
| 84 | 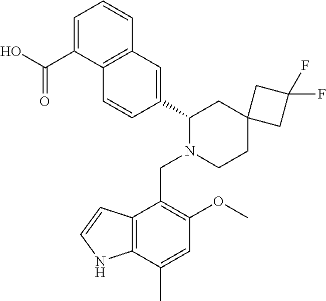 [SMILES: COc1cc(C)c2[nH]ccc2c1CN1CCC2(C[C@H]1c1ccc3c(C(=O)O)cccc3c1)CC(F)(F)C2] | Example 43 | CAS: 2238811-87-3 Column: CHIRALPAK OJ-H 4.6*250 mm, 5 μm Mobile phase: 60% CO₂, 40% MeOH, 0.2% NH₄OH Flow rate: 2.5 mL/min Column temperature: 40° C. Retention time = 2.72 min | LCMS (m/z): [M + H]⁺ calc'd for C₃₀H₃₀F₂N₂O₃, 505.58; found, 505.0. 1H NMR (400 MHz, MeOD) δ 8.89 (s, 1H), 8.15 (s, 1H), 7.98 (s, 2H), 7.70 (d, J = 30.6 Hz, 2H), 7.29 (s, 1H), 6.74 (s, 1H), 6.32 (s, 1H), 4.43 (d, J = 63.4 Hz, 2H), 4.11 (d, J = 10.6 Hz, 1H), 3.67 (s, 3H), 3.50 (s, 1H), 3.32 (s, 1H), 2.85-2.69 (m, 2H), 2.50 (s, 6H), 2.21- 2.02 (m, 2H), 1.94 (d, J = 14.3 Hz, 1H). |
| 85 | 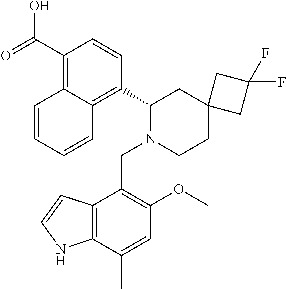 [SMILES: COc1cc(C)c2[nH]ccc2c1CN1CCC2(C[C@H]1c1ccc(C(=O)O)c3ccccc13)CC(F)(F)C2] | Example 43 | CAS: 2238811-87-3 Column: CHIRALPAK IC 4.6*250 mm, 5 μm Mobile phase: 60% CO₂, 40% MeOH, 0.2% NH₄OH Flow rate: 2.5 mL/min Column temperature: 40.1° C. Retention time = 3.28 min | LCMS (m/z): [M + H]⁺ calc'd for C₃₀H₃₀F₂N₂O₃, 504.22; found, 505.2. ¹H NMR (400 MHz, CD₃OD) δ 8.66 (d, J = 8.5 Hz, 1H), 8.34 (d, J = 8.5 Hz, 1H), 7.94 (d, J = 7.6 Hz, 1H), 7.88 (d, J = 7.6 Hz, 1H), 7.71 (t, J = 7.5 Hz, 1H), 7.67-7.60 (m, 1H), 7.30-7.21 (m, 1H), 6.71 (s, 1H), 6.29 (s, 1H), 5.33 (d, J = 12.1 Hz, 1H), 4.26 (d, J = 9.8 Hz, 1H), 4.11 (dd, J = 12.0, 3.6 Hz, 1H), 3.74 (s, 3H), 3.56- 3.40 (m, 2H), 2.79 (dd, J = 18.5, 10.4 Hz, 2H), 2.48- 2.29 (m, 6H), 2.17-2.04 (m, 2H), 1.94 (d, J = 14.9 Hz, 1H). |
| 86 | 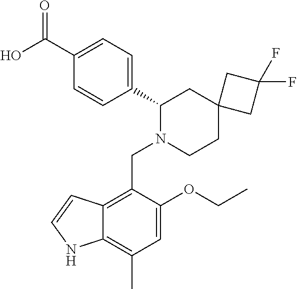 [SMILES: CCOc1cc(C)c2[nH]ccc2c1CN1CCC2(C[C@H]1c1ccc(C(=O)O)cc1)CC(F)(F)C2] | Example 43 | CAS: 2238811-87-3 Column: CHIRALPAK IC 4.6*250 mm, 5 μm Mobile phase: 60% CO₂, 40% MeOH, 0.2% NH₄OH Flow rate: 2.5 mL/min Column temperature: 39.9° C. Retention time = 3.28 min | LCMS (m/z): [M + H]+ calc'd for C27H30F2N2O3, 469.23; found, 468.9. 1H NMR (400 MHz, MeOD) δ 8.45 (s, 0.42H), 8.17 (d, J = 8.4 Hz, 2H), 7.69 (d, J = 7.6 Hz, 2H), 7.32 (d, J = 3.2 Hz, 1H), 6.78 (s, 1H), 6.30 (s, 1H), 4.41 (m, 2H), 4.16-4.07 (m, 2H), 4.05-3.95 (m, 1H), 3.51- 3.41 (m, 1H), 3.18 (m, 1H), 2.83-2.62 (m, 3H), 2.51 (s, 3H), 2.46 (m, 1H), 2.35 (s, 1H), 2.10 (m, 2H), 1.91 (m, 1H), 1.28 (t, J = 7.2 Hz, 3H). |

### BIOLOGICAL ASSAYS

### Biological Example 1. Factor B Binding Assay by TR-FRET

### Material and Reagents

1. Recombinant human Factor B catalytic domian (a.a. 470-764, C-terminal his-tagged, produced in-house)
2. 5× Kinase Buffer A (Thermo Fisher, CAT #PV3189)
3. LANCE Eu-W1024 Anti-6×His Antibody (PerkinElmer, CAT #AD0401)
4. Probe (TRFRET_tool 2, reported in WO 2015/009616)

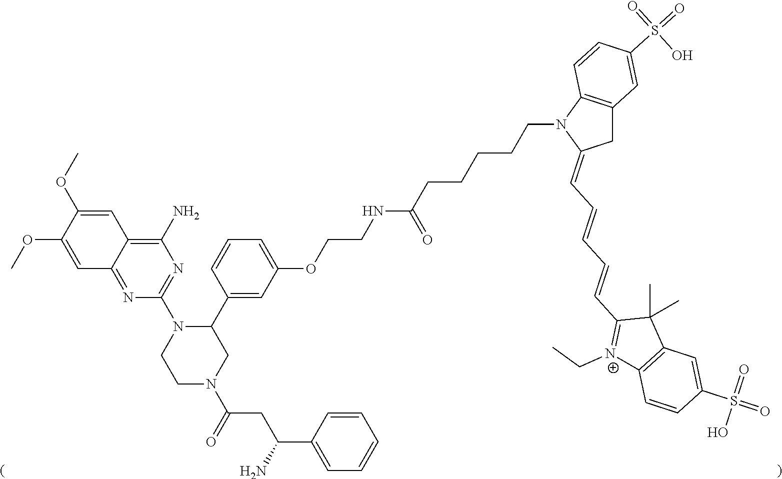 [SMILES: CC[N+]1=C(/C=C/C=C/C=C2\Cc3cc(S(=O)(=O)O)ccc3N2CCCCCC(=O)NCCOc2cccc(C3CN(C(=O)C[C@@H](N)c4ccccc4)CCN3c3nc(N)c4cc(OC)c(OC)cc4n3)c2)C(C)(C)c2cc(S(=O)(=O)O)ccc21]

5. DMSO (Thermo Fisher Scientific)
6. Compounds—10 mM stock in DMSO
7. Victor Nivo multimode plate reader (PerkinElmer)
8. OptiPlate-384, white opaque 384-well microplate (PerkinElmer, CAT #6007290)

### Experimental Procedure

Factor B binding affinity of each compound tested was determined using a time-resolved fluorescence resonance energy transfer (TR-FRET) technology. 10 nM recombinant his-tagged Factor B catalytic domain, varying concentrations of inhibitors, 4 nM LANCE Eu-W1024 Anti-6×His Antibody and 100 nM TR-FRET_tool2 tracer was incubated in 1× Kinase Buffer A for 1 h. Measurement was performed in a reaction volume of 15 μL by adding 5 μL of the test compound, 5 μL of Factor B/antibody mixture and 5 μL of tracer into white opaque 384-well assay plates. The TR-FRET signal was read on a plate reader with an excitation wavelength of 340 nm and detection wavelengths of 615 and 665 nm. Binding affinity was determined for each compound by measuring TR-FRET signal at various concentrations of compound and plotting the relative fluorescence Emission Ratio (665 nm/615 nm) against the inhibitor concentration to estimate the IC₅₀ from [Compound] vs Emission Ratio using the four parameters dose-response inhibition curve with a variable slope model in GraphPad Prism.

The binding affinity to recombinant Factor B catalytic domain of the compounds of the present invention was determined by the above assay, and IC₅₀ values (nM) are shown in the following Table 1.

TABLE 1
IC₅₀ values (nM) of the compounds in the
present invention against human Factor B.

| | | Factor B TR-FRET IC₅₀ |
|---|---|---|
| Example # | Isomer | (nM) |
| 1 | | 923.1 |
| 2 | 2 | 359.8 |
| 2 | 3 | 507.4 |
| 2 | 6 | 60.0 |
| 4 | | 172.7 |
| 6 | 2 | 142.3 |
| 7 | 1 | 312.7 |
| 7 | 2 | 157.2 |
| 8 | 1 | 7.0 |
| 8 | 2 | 245.3 |
| 9 | 2 | 40.6 |
| 10 | | 264.6 |
| 13 | 1 | 17.4 |
| 16 | | 304.6 |
| 21 | | 843.9 |
| 22 | | 45.0 |
| 23 | | 142.6 |
| 24 | 1 | 1411.0 |
| 24 | 2 | 729.2 |
| 25 | | 322.3 |
| 26 | 1 | 31.5 |
| 27 | 1 | 5.0 |
| 27 | 2 | 5.3 |
| 28 | 2 | 81.5 |
| 29 | 1 | 7.5 |
| 29 | 2 | 43.7 |
| 30 | | 131.9 |
| 32 | 1 | 26.3 |
| 34 | 1 | 7.7 |
| 34 | 2 | 179.7 |
| 36 | 2 | 16.6 |
| 36 | 3 | 125.4 |
| 37 | 1 | 321.6 |
| 37 | 3 | 222.1 |
| 37 | 4 | 359.4 |
| 38 | 4 | 4.8 |
| 39 | 1 | 2.6 |
| 39 | 2 | 4.1 |
| 39 | 3 | 7.3 |
| 40 | 1 | 1.8 |
| 40 | 2 | 3.0 |
| 41 | | 20.1 |
| 42 | 1 | 4 |
| 43 | | <2 |
| 57 | | <2 |
| 53 | | <2 |
| 47 | 1 | <2 |
| 64 | | <2 |
| 76 | 1 | <2 |
| 78 | 1 | 7.4 |
| 79 | 1 | 2.0 |
| 80 | | <2 |
| 81 | | 47.3 |
| 82 | | <2 |
| 62 | | <2 |
| 83 | | <2 |
| 84 | | 3.1 |
| 85 | | <2 |
| 86 | | 2.1 |

### Biological Example 2. Target Residence Time of Factor B Inhibitors Determined by Surface Plasmon Resonance (SPR)

### Material and Reagents

1. Recombinant human Factor B catalytic domain (a.a. 470-764, C-terminal his-tagged, produced in-house)
2. PBS-P+ Buffer 10× (Cytiva, CAT #28995084)
3. Series S Sensor Chip NTA (Cytiva, CAT #BR100532)
4. Amine Coupling Kit (Cytiva, CAT #BR100050)
5. DMSO (Millipore Sigma, CAT #34869-1 L)
6. Greiner 96 well plates, polypropylene (Sigma-Aldrich, CAT #M7310-100EA)
7. Microplate Foil, 96-well (Cytiva, CAT #28975816)
8. Biacore 8 k (Cytiva)

### Experimental Procedure

Biacore 8 k instrument was primed using 1× PBS-P+ buffer before docking a Cytiva NTA chip. Recombinant human Factor B catalytic domain were immobilized on a NTA chip to a level of about 5000 resonance units (RU) using 1× PBS-P+ buffer [20 mM phosphate buffer with 2.7 mM KCl, 137 mM NaCl, and 0.05% (v/v) Tween-20]. The protein ligand was further crosslinked to sensorchip surface by amine coupling kit. Immobilization and binding experiment were performed at room temperature.

After changing buffer to 1× PBS-P+ buffer with 2% (v/v) DMSO, a pre-run was performed for a period of at least 30 min at a flow rate of 30 μl/min to obtain a stable surface. The kinetic constants of the compounds were determined by single-cycle kinetics with six consecutive injections (or multi-cycle kinetics with eight consecutive injections) with an increasing compound concentration in ranges of 0.8-200 nM, 12.5-400 nM, 4.1-1,000 nM or 41-10,000 nM depending on the potency. Single-cycle kinetics experiments were performed with an association time of 60 s per concentration and a dissociation time of 300 s (or a dissociation time of 120 s for multi-cycle kinetics experiments). A flow rate of 30 μl/min was used. A blank run with the same conditions was performed before the compound was injected.

The SPR sensorgrams were analyzed with Biacore Insight Evaluation Software by using a method of double referencing. The resulting curve was fitted with a 1:1 binding model. Compounds that bound according to an induced fit model were fitted with a two-state reaction model. The kinetic constants (kₒₙ, k_{off}, K_{D}) of replicates were averaged. Binding half-life (t_{1/2}) for the 1:1 binding model and two-state reaction model was calculated from the dissociation constant k_{off} with the formula t_{1/2}=ln2/k_{off}. The target residence time (t_{1/2}) and the residence time (1/k_{off}) are shown in the following Table 2.

TABLE 2
Kinetic constants and target residence time of the compounds
in the present invention against human Factor B.

| Example # | Isomer | K_{D} (nM) | Residence time (s) | t_{1/2} (s) |
|---|---|---|---|---|
| Iptacopan | — | 9.93 | 100.1 | 69 |
| 27 | 1 | 6.25 | 191.4 | 132 |
| 27 | 2 | 4.25 | 308.9 | 213 |
| 29 | 1 | 7.03 | 236.4 | 163 |
| 47 | 1 | 0.53 | 4130 | 2862 |
| 53 | — | 0.38 | 6308.4 | 4372 |
| 57 | — | 0.17 | 6171.1 | 4277 |
| 43 | — | 0.77 | 4628.8 | 3208 |

Conclusion: Examples of present invention have significant binding affinity.

### Biological Example 3. Ocular Pharmacokinetic Studies in Rats

Three-month-old brown Norway rats were administered the Example compound via oral gavage as a suspension in 2 equiv 1 N HCl+30% PEG300+50% (20% Cremophor EL in water). Ocular tissues from both eyes and plasma were collected from rats per time point at 0.25, 0.5, 1, 6, and 24 h after administration. The ocular tissues collected were the retina and the posterior eye cup (RPE/choroid and posterior sclera). The tissues were diluted with phosphate buffered saline containing 10% acetonitrile and homogenized, centrifuged prior to analyses. The concentrations of the test article were measured in plasma and supernatants of ocular homogenates by HPLC-MS/MS in four individual retinas, four individual posterior eye cups, and two individual plasma samples at each time point. Chromatographic separation was carried out on Waters BEH C18 Column (2.1×50 mm, 1.7 μm) column (MAC-MOD Analytical, Chadds Ford, PA), using a gradient elution method with water and acetonitrile, both containing 0.025% formic acid—1 mM NH₄OAc. Mass spectrometric measurements in positive electrospray ionization were directed at quantifying the mass transition with [M+H]⁺ as the precursor ion on API6500, triple quadruple mass spectrometer (Sciex, Framingham, MA). The relevant pharmacokinetic parameters were estimated using noncompartmental methods using WinNonlin (Enterprise, version 8.2).

TABLE 3
The results of the PK studies are in rats (2 mpk PO)

| | | Retina | Plasma |
|---|---|---|---|
| | T½ (h) | AUC₀₋ₗₐₛₜ (nM · h) | AUC₀₋ₗₐₛₜ (nM · h) |
| Iptacopan | 5.3 | 141 | 13461 |
| Example 8 isomer 1 | 6.4 | 2226 | 10773 |
| Example 57 | 22.2 | 796.4 | 12,668 |

Conclusion: Compounds of present invention have better exposure in retina.

### Biological Example 4. In Vivo Assessment of Mouse AP Complement Functional Activity

Female C57BL/6 mice were administered with Example 57 formulation (20 mg/kg in 0.5% (w/v) methyl cellulose, 0.5% (v/v) Tween 80) by oral gavage 20 h before the end of study. To activate complement pathway, lipopolysaccharide (LPS) from Salmonella typhimurium (Sigma) was injected i.p. (2.5 mg/kg) 7.5 h prior to the end of the study. Control mice were given i.p. injection of saline solution and dosed with vehicle by oral gavage. Plasma samples were collected from mice at the end of the study. AP complement activation was assessed by measuring plasma C3 cleavage products C3b/iC3b/C3c with ELISA using rat anti-mouse C3b/iC3b/C3c monoclonal antibody (clone 2/11, Hycult biotech, 0.1ug/well) and goat anti-Rat IgG (whole molecule)-Peroxidase (Sigma) diluted in TBST (TBS/0.05% Tween20). The plasma C3b/iC3b/C3c are shown in the following table 4.

TABLE 4
Plasma C3b/iC3b/C3c after each treatment

| Treatment | Plasma C3b/iC3b/C3c (1 × 10\hat{ }6) |
|---|---|
| None | 168.00 |
| Saline | 157.75 |
| LPS | 370.50 **** |
| Example 57 | 210.25 ^{ns} |

Conclusion: The results show that Example 57 shows sustained inhibition in mouse in-vivo PD assay at 20 h. (*: p<0.05; **: p<0.01; ****: p<0.0001; ns: no significant difference)

### Biological Example 5. Ex Vivo Assessment of Plasma PD Inhibition

Male Sprague Dawley rats (n=3) were orally administered with vehicle (0.5% (w/v) methyl cellulose, 0.5% (v/v) Tween 80), compound Iptacopan or the Example compound formulation (in 0.5% (w/v) methyl cellulose, 0.5% (v/v) Tween 80) at 2 mg/kg. Serum samples from rats were collected at 0.25, 0.5, 1, 2, 4, 6, 8, and 24 h post dose and stored at −80° C. 96-well microtiter plates (Black Maxisorp, Invitrogen) are coated with 3 μg/ml LPS from strain Salmonella enteritidis for the alternative complement pathway (AP) ELISA (TLRGRADE, Enzo Life Sciences, in PBS/10 mM MgCl2) overnight at 4° C. The coated plates were washed with GVB buffer (Complement tech) containing 5 mM MgCl2 and 10 mM EGTA (classical and lectin pathways are blocked). Collected serum samples were diluted by addition of an equal volume of GVB buffer containing 10 mM MgCl2 and 20 mM EGTA. For a negative control, serum was diluted with GVB buffer containing 40 mM EDTA (blocking all complement pathways). Aliquots (50 ul) of the 50% serum samples were placed on the LPS-coated wells. The reaction plate was placed at 37° C. for 20 minutes (rat serum). The reaction was terminated by inverting the plate to empty wells and addition of blocking buffer (50 μL, SuperBlock™ T20 (TBS) Blocking Buffer, Thermo #37536). For detection of rat MAC deposition on LPS, anti-rat C5b-9 neoepitope detecting mAb 2A1 (HM3033-IA, Hycult Biotech, 0.1ug/well) and goat anti-mouse IgG (Fc specific)-Peroxidase (Sigma, #A2554) were used. The baseline (EDTA-treated serum) and the maximum signal (EGTA-treated serum from vehicle-treated mice) were used to generate percent inhibition values for each of the wells.

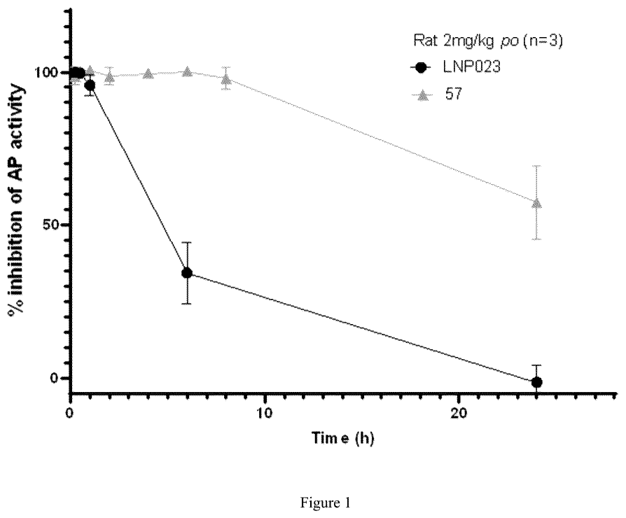

Rats (3 rats/group) were orally given compound Iptacopan or Example 57 (2 mg/kg), and then AP deposition inhibitory activity in 50% serum of compounds were assessed after 0.25, 0.5, 1, 2, 4, 6, 8, and 24 h of dosing. Each data point represents an average of AP activity in the rat serum in FIG. 1. The results show that Example 57 shows sustained inhibition in rat ex-vivo PD assay at 24 h.

TABLE 5
The results of the PD studies at 24 h in rats

| | Serum PD inhibition (%) at 24 h (2 mpk) |
|---|---|
| Iptacopan | −3 |
| Example 57 | 57.5 |

1-29. (canceled)

30. A compound of formula (I): 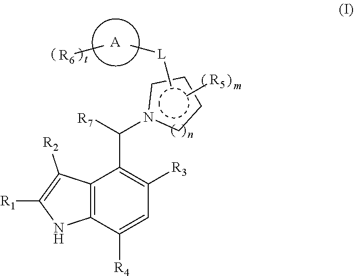
or tautomer, pharmaceutically acceptable salt thereof, wherein:
A is cycloalkyl, heterocyclyl, aryl or heteroaryl;
L is bond, (CRₐR_{b})ₚ or absent;
Rₐ and R_{b} are independently selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl and heteroaryl;
R₁ and R₂ are independently selected from the group consisting of hydrogen, deuterium, halogen, amino, cyano, hydroxy, alkyl, alkoxy, alkylthio, haloalkyl and hydroxyalkyl;
R₃ and R₄ are independently selected from the group consisting of hydrogen, deuterium, halogen, amino, cyano, hydroxy, alkyl, alkoxy, alkylthio, haloalkyl, haloalkenyl, hydroxyalkyl, deuterated alkoxy, haloalkoxy, cycloalkyl, heterocyclyl, aryl, heteroaryl, cycloalkylxoy, heterocyclylxoy, arylxoy and heteroarylxoy, optionally the hydroxy, alkyl, alkoxy, alkylthio, haloalkyl, hydroxyalkyl, deuterated alkoxy, haloalkoxy, cycloalkyl, heterocyclyl, aryl, heteroaryl, cycloalkylxoy, heterocyclylxoy, arylxoy and heteroarylxoy substituted with one or more substituents selected from deuterium, halogen, amino, cyano, hydroxy, alkyl, alkoxy, alkylthio, haloalkyl, hydroxyalkyl, cycloalkyl, heterocyclyl, aryl and heteroaryl;
R₅ is independently selected from the group consisting of hydrogen, deuterium, halogen, amino, cyano, hydroxy, alkyl, alkenyl, alkynyl, alkoxy, alkylthio, haloalkyl, hydroxyalkyl, cycloalkyl, heterocyclyl, aryl and heteroaryl, optionally the amino, alkyl, alkoxy, alkylthio, haloalkyl, hydroxyalkyl, cycloalkyl, heterocyclyl, aryl and heteroaryl substituted with one or more substituents selected from deuterium, halogen, amino, cyano, hydroxy, alkyl, alkoxy, alkylthio, haloalkyl and hydroxyalkyl;
or, two of R₅ together with the C atom to which they are attached form cycloalkyl or heterocyclyl, optionally the cycloalkyl or heterocyclyl substituted with one or more substituents selected from deuterium, halogen, amino, cyano, hydroxy, alkyl, alkoxy, alkylalkoxy, alkoxyalkyl, alkylthio, haloalkyl and hydroxyalkyl;
R₆ is selected from the group consisting of hydrogen, deuterium, halogen, amino, cyano, hydroxy, alkyl, alkoxy, alkylthio, haloalkyl, hydroxyalkyl, cycloalkyl, heterocyclyl, aryl and heteroaryl, —(CH₂)ᵣOR₈, —(CH₂)ᵣC(O)R₈, —S(O)NHalkyl, —SO₂alkyl, —C(O)NHSO₂alkyl and —SO₂NHC(O)alkyl;
or, R₆ together with the C atom in 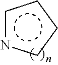
to form cycloalkyl or heterocyclyl, optionally the cycloalkyl or heterocyclyl substituted with one or more substituents selected from deuterium, halogen, amino, cyano, hydroxy, alkyl, alkoxy, alkylalkoxy, alkoxyalkyl, alkylthio, haloalkyl and hydroxyalkyl;
R₇ is selected from the group consisting of hydrogen, deuterium, halogen, amino, cyano, hydroxy, alkyl, alkoxy, alkylthio, haloalkyl and hydroxyalkyl;
R₈ is selected from the group consisting of hydrogen, deuterium, halogen, amino, cyano, hydroxy, alkyl, alkoxy, alkylthio, haloalkyl and hydroxyalkyl;
p is 1, 2 or 3;
r is 0, 1, 2 or 3;
t is 1, 2 or 3;
m is 1, 2 or 3; and
n is 0, 1, 2 or 3;
provided that if
R₁ and R₂ is hydrogen, R₃ is cyclopropyl or methoxy, R₄ is methyl, L is bond, R₆ is —COOH or —COOCH₃, R₇ is hydrogen or trifluoromethyl, A is phenyl, and n is 1, 2 or 3, R₅ is not hydrogen or 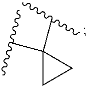 [SMILES: *C1(*)CC1]
R₁ and R₂ is hydrogen, R₄ is methyl, L is bond, R₇ is hydrogen, A is phenyl, pyridine or thiazolyl, m is 1, and n is 2, R₅ is not hydrogen, amino, hydroxy, methyl, ethyl, methoxy, ethyoxyl, propoxy, methylol, ethoxyl, cyanomethyl and methylamino;
R₁ and R₂ is hydrogen, R₄ is methyl, L is bond, R₇ is hydrogen, A is phenyl, m is 2 or 3, and n is 2, R₅ is not hydrogen or methyl.

31. The compound of claim 30, or tautomer, pharmaceutically acceptable salt thereof, wherein A is C₆₋₁₀ aryl or 5-10 membered heteroaryl;
preferably, A is phenyl, benzocycloalkyl, or 5-8 membered heteroaryl containing 1, 2 or 3 of N heteroatoms
more preferably, A is

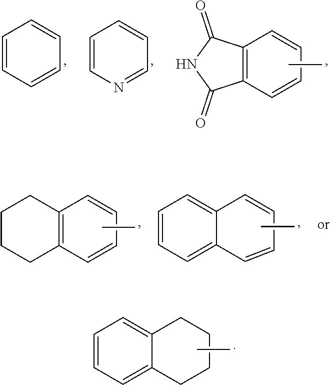

32. The compound of claim 30, or tautomer, pharmaceutically acceptable salt thereof, wherein L is bond, CH₂ or absent; or, R₁ and R₂ are hydrogen;
or, R₃ and R₄ are independently selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ haloalkenyl C₁₋₆ hydroxyalkyl, deuterated C₁₋₆ alkoxy, C₁₋₆ haloalkoxy, C₃₋₆ cycloalkyl, 4-10 membered heterocyclyl, C₆₋₁₀ aryl, 5-10 membered heteroaryl, C₃₋₆ cycloalkyloxy, 4-10 membered heterocyclyloxy, C₆₋₁₀ aryloxy and 5-10 membered heteroaryloxy, optionally the C₁₋₆ alkyl, C₁₋₆ alkoxy, C₁₋₆ alkylthio, C₁₋₆ haloalkyl, C₁₋₆ hydroxyalkyl, deuterated C₁₋₆ alkoxy, C₁₋₆ haloalkoxy substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₆ cycloalkyl, 4-10 membered heterocyclyl, C₆₋₁₀ aryl and 5-10 membered heteroaryl;
preferably, R₃ and R₄ are independently selected from the group consisting of deuterium, halogen, C₁₋₃ alkyl, C₁₋₃ alkoxy, deuterated C₁₋₃ alkoxy, C₁₋₃ haloalkoxy, C₃₋₆ cycloalkyl and C₃₋₆ cycloalkyloxy, optionally the C₁₋₃ alkyl, C₁₋₃ alkoxy, deuterated C₁₋₃ alkoxy, C₁₋₃ haloalkoxy substituted with one or more substituents selected from C₃₋₆ cycloalkyl, 4-6 membered heterocyclyl, C₆₋₁₀ aryl and 5-10 membered heteroaryl;
or, R₆ is selected from the group consisting of hydrogen, deuterium, halogen, amino, cyano, hydroxy, C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, —(CH₂)ᵣC₁₋₆ alkoxy, —(CH₂)ᵣC(O)OH, —S(O)NHC₁₋₆ alkyl, —SO₂C₁₋₆ alkyl, —C(O)NHSO₂C₁₋₆ alkyl and —SO₂NHC(O)C₁₋₆ alkyl; preferably, R₆ is —F, -OMe, —CH₂OH, —CH₂OCH₃, —CH₂F, —CF₂H, —CF₃, —COOH, —C(O)NHSO₂CH₃, —S(O)NHCH₃, or 5-6membered heterocyclyl containing 1-3 of heteroatom selected from N, O and S, or 5-6 membered heteroaryl containing 1-3 of heteroatom selected from N, O and S; or, R₅ is independently selected from the group consisting of hydrogen, deuterium, halogen, amino, cyano, hydroxy, C₁₋₆ alkyl, C₂₋₆ alkenyl, C₂₋₆ alkynyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl, optionally the C₁₋₆ alkyl, C₁₋₆ alkoxy, C₁₋₆ alkylthio, C₁₋₆ haloalkyl, C₁₋₆ hydroxyalkyl, C₃₋₈ cycloalkyl, 4-10 membered heterocyclyl, C₅₋₁₀ aryl and 5-10 membered heteroaryl substituted with one or more substituents selected from deuterium, halogen, amino, cyano, hydroxy, C₁₋₆ alkyl, C₁₋₆ alkoxy, C₁₋₆ alkylthio, C₁₋₆ haloalkyl and C₁₋₆ hydroxyalkyl;
or, two of R₅ together with the C atom to which they are attached form C₃₋₆ cycloalkyl or 4-6 membered heterocyclyl containing 1, 2 or 3 heteroatoms selected from N, O or S, optionally substituted with one or more substituents selected from deuterium, halogen, amino, cyano, hydroxy, C₁₋₆ alkyl, C₁₋₆ alkoxy, C₁₋₆ alkylC₁₋₆ alkoxy, C₁₋₆ alkoxyC₁₋₆ alkyl, C₁₋₆ alkylthio, C₁₋₆ haloalkyl and C₁₋₆ hydroxyalkyl; or R₇ is hydrogen or C₁₋₃ alkyl.

33. The compound of claim 30, or tautomer, pharmaceutically acceptable salt thereof, wherein the compound is of formula (II-a)-(II-e): 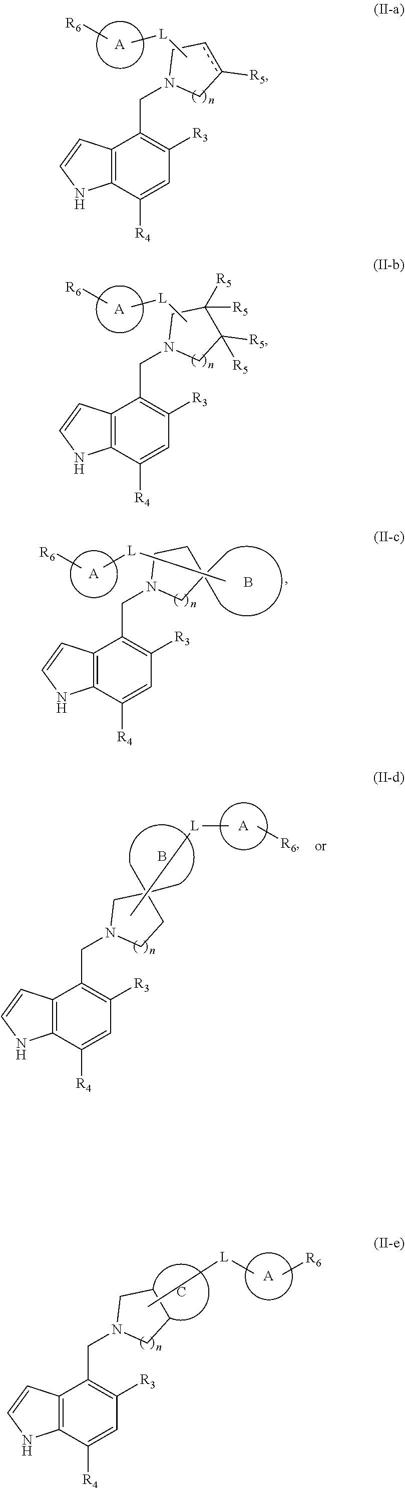
wherein, [symbol]
is single or double bond;
R₅ is s independently selected from the group consisting of hydrogen, deuterium, halogen, amino, cyano, hydroxy, C₁₋₃ alkyl, C₁₋₃ alkoxy, C₁₋₃ alkylthio, C₁₋₃ haloalkyl, C₁₋₃ hydroxyalkyl, C₃₋₆ cycloalkyl, 4-6 membered heterocyclyl containing 1, 2 or 3 heteroatoms selected from N, O or S, C₅₋₁₀ aryl and 5-6 membered heteroaryl containing 1, 2 or 3 heteroatoms selected from N, O or S, optionally the C₁₋₃ alkyl, C₁₋₃ alkoxy, C₁₋₃ alkylthio, C₁₋₃ haloalkyl, C₁₋₃ hydroxyalkyl, C₃₋₆ cycloalkyl, 4-6 membered heterocyclyl, C₅₋₁₀ aryl and 5-6 membered heteroaryl substituted with one or more substituents selected from deuterium, halogen, amino, cyano, hydroxy, C₁₋₃ alkyl, C₁₋₃ alkoxy, C₁₋₃ alkylthio, C₁₋₃ haloalkyl and C₁₋₃ hydroxyalkyl; 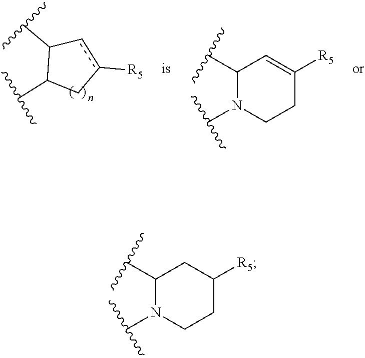
B is 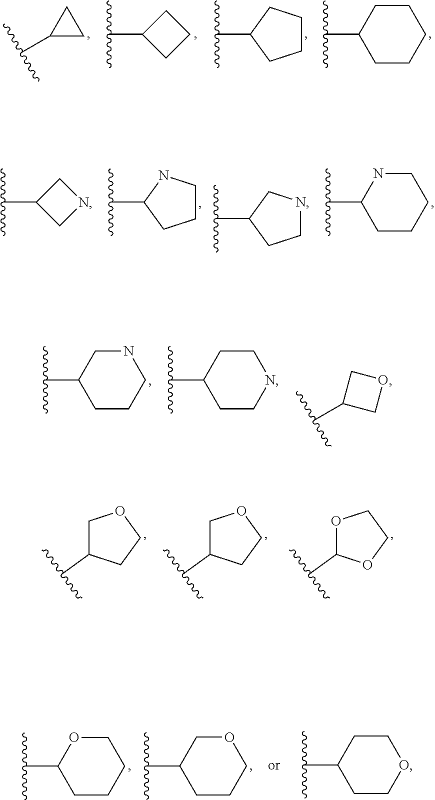
optionally substituted with one or more substituents selected from deuterium, halogen, amino, cyano, hydroxy, C₁₋₃ alkyl, C₂₄ alkenyl, C₂₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl; and
C is 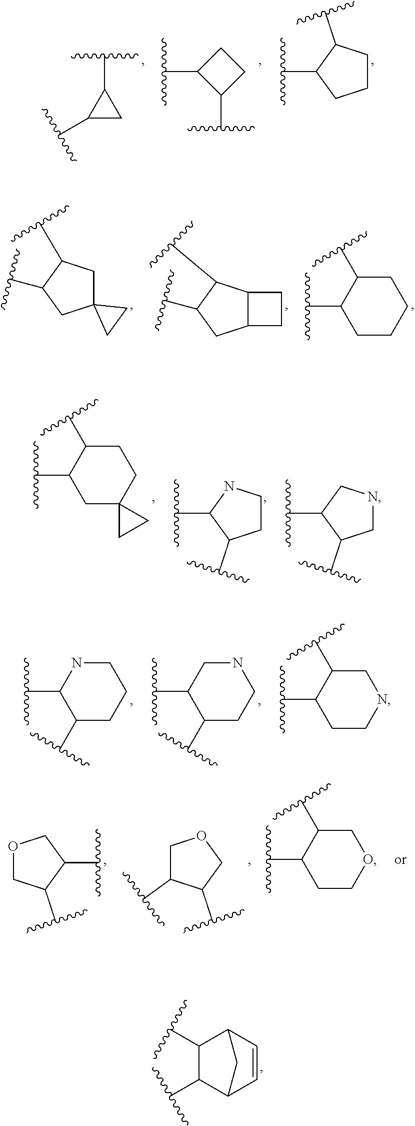
optionally substituted with one or more substituents selected from deuterium, halogen, amino, cyano, hydroxy, C₁₋₃ alkyl, C₂₄ alkenyl, C₂₄ alkynyl, C₁₋₃ alkoxy, C₁₋₃ alkylC₁₋₃ alkoxy, C₁₋₃ alkoxyCl₁₋₃ alkyl, C₁₋₃alkylthio, C₁₋₃ haloalkyl and C₁₋₃ hydroxyalkyl.

34. The compound of claim 33, or tautomer, pharmaceutically acceptable salt thereof, wherein the compound is of formula (III-a)-(III-e): 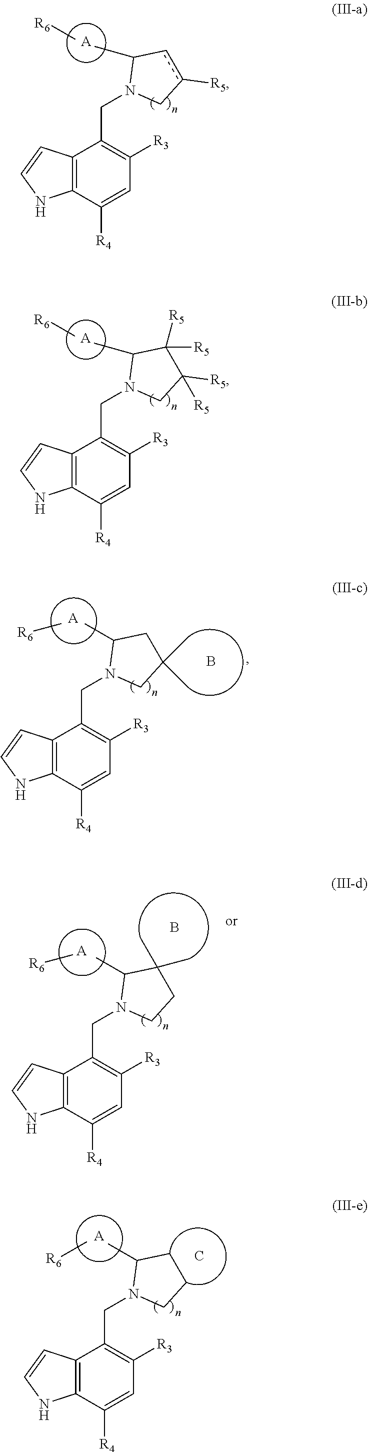
wherein, 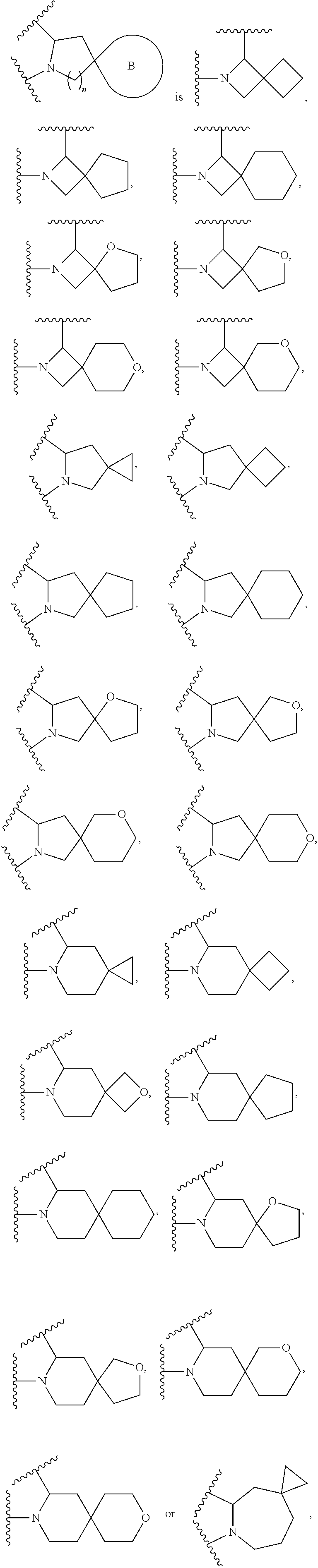
or optionally substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxy alkyl; 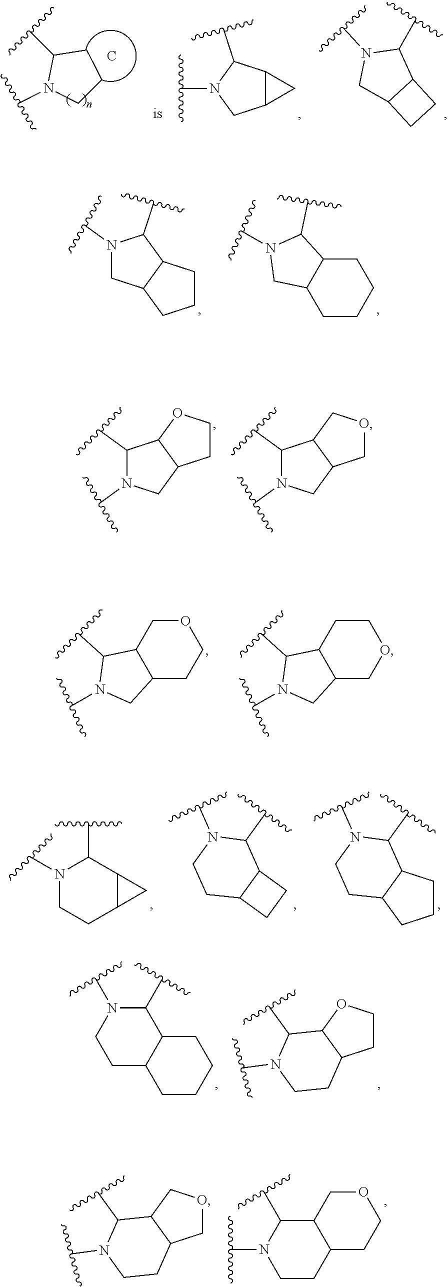 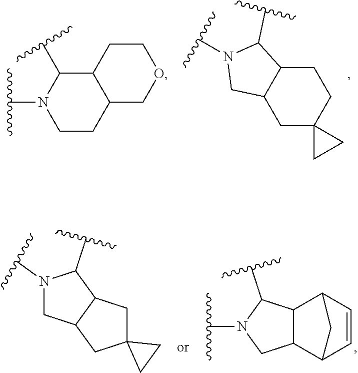 [SMILES: *C1C2CCOCC2CCN1* ; *C1C2CCC3(CC3)CC2CN1* ; *C1C2CC3(CC3)CC2CN1* ; *C1C2C3C=CC(C3)C2CN1*]
optionally substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl.

35. The compound of claim 34, or tautomer, pharmaceutically acceptable salt thereof, wherein, 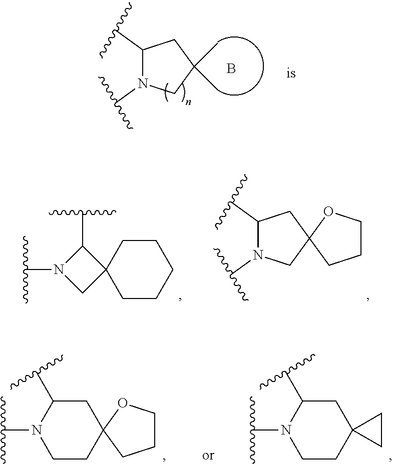
optionally substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃; and 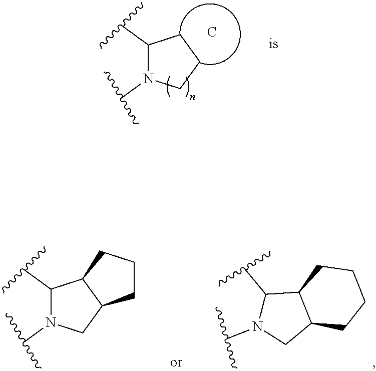
optionally substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyC₁₋₃ alkyl, C₁₋₃alkylthio, C₁₋₃ haloalkyl and C₁₋₃ hydroxyalkyl.

36. The compound of claim 34, or tautomer, pharmaceutically acceptable salt thereof, wherein,
A is 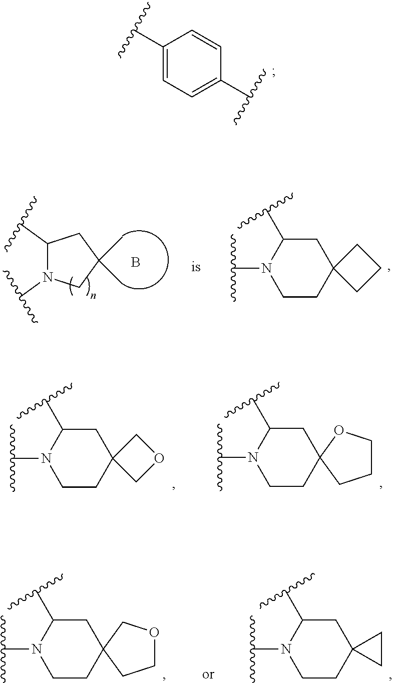
optionally substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylC₁₋₃ alkoxy, C₁₋₃ alkoxyCl₁₋₃ alkyl, C₁₋₃alkylthio, C₁₋₃ haloalkyl and C₁₋₃ hydroxyalkyl; 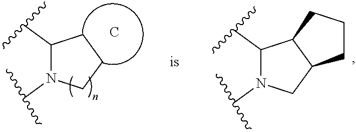
optionally substituted with one or more substituents selected from deuterium, halogen, amino, cyano, hydroxy, C₁₋₃ alkyl, C₂₋₄ alkenyl, C₂₋₄ alkynyl, C₁₋₃ alkoxy, C₁₋₃ alkylCl₁₋₃ alkoxy, C₁₋₃ alkoxyCl₁₋₃ alkyl, C₁₋₃alkylthio, C₁₋₃ haloalkyl and C₁₋₃ hydroxyalkyl;
each of R₃ and R₄ is independently selected from deuterium, halogen, C₁₋₃ alkyl, C₁₋₃ alkoxy, C₃₋₆ cycloalkyl, C₃₋₆ cycloalkyloxy, deuterated C₁₋₃ alkoxy, C₁₋₃ haloalkoxy and C₃₋₆ cycloalkylC₁₋₃ alkoxy;
R₅ is hydrogen, halogen, cyano, C₁₋₃ alkyl, C₂₋₄ alkenyl, C₂₋₄ alkynyl, C₁₋₃ alkoxy, C₁₋₃ haloalkyl, C₁₋₃ alkylC₁₋₃ alkoxy, C₁₋₃ alkoxyCl₁₋₃ alkyl, C₃₋₆ cycloalkyl, 5 membered heteroaryl containing 1 or 2 ring heteroatoms independently selected from N or O; and
R₆ is —COOH or —S(O)NHCH₃.

37. The compound of claim 30, or tautomer, pharmaceutically acceptable salt thereof, wherein the compound is of formula (IV): 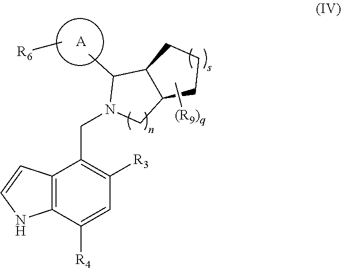
R₉ is hydrogen, halogen, amino, cyano, hydroxy, C₁₋₃ alkyl, C₂₋₄ alkenyl, C₂₋₄ alkynyl, C₁-3 alkoxy, C₁₋₃alkylthio, C₁₋₃ haloalkyl and C₁₋₃ hydroxyalkyl, optionally substituted with one or more substituents selected from halogen, amino, hydroxy, C₁₋₃ alkyl, C₁₋₃ alkoxy, C₁₋₃ alkylamino, C₃₋₆ cycloalkyl and 5-6 membered heterocyclyl containing 1 or 2 ring heteroatoms independently selected from N or 0;
or, two of R₉ together with the C atom to which they are attached from C₃₋₆ cycloalkyl;
optionally substituted with one or more substituents selected from deuterium, halogen, amino, cyano, hydroxy, C₁₋₃ alkyl, C₂₋₄ alkenyl, C₂₋₄ alkynyl, C₁₋₃ alkylamino, C₁₋₃ alkoxy, C₁₋₃ alkylCl₁₋₃ alkoxy, C₁₋₃ alkoxyCl₁₋₃ alkyl, C₁₋₃alkylthio, C₁₋₃ haloalkyl and C₁₋₃ hydroxyalkyl;
n is 1 or 2;
q is 1, 2 or 3, and
s is 0, 1 or 2.

38. The compound of claim 30, or tautomer, pharmaceutically acceptable salt thereof, wherein the compound is of formula (V-a)-(V-c): 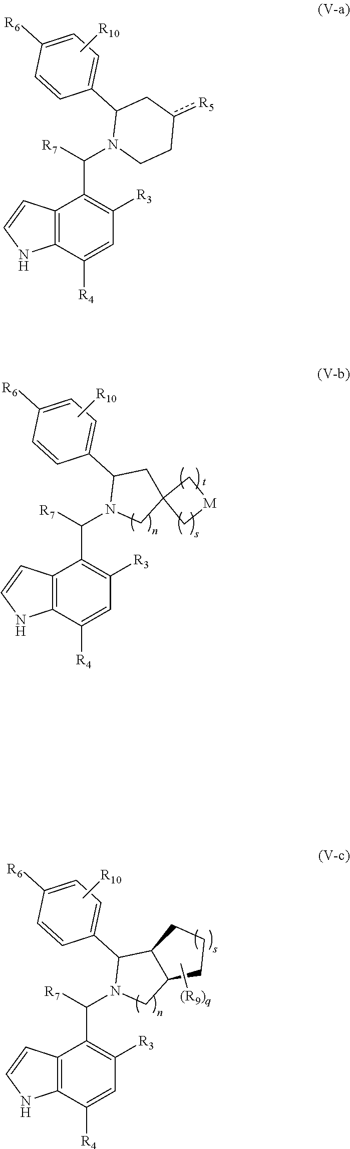
M is O or CR_{c}R_{d};
R_{c} and R_{d} are independently selected from hydrogen, halogen or C₁₋₃ alkyl;
R₃ and R₄ are independently selected from C₁₋₃ alkyl, C₁₋₃ alkoxy or C₃₋₆ cycloalkyl;
R₅ is hydrogen, halogen, C₁₋₃ alkyl or C₁₋₃ haloalkyl;
R₆ is —COOH, —C(O)NHSO₂CH₃ or —S(O)NHCH₃;
R₇ is hydrogen, C₁₋₃ alkyl or C₁₋₃ hydroxyalkyl;
R₉ is hydrogen, halogen, C₁₋₃ alkyl or C₁₋₃ haloalkyl;
or, two of R₉ together with the C atom to which they are attached form C₃₋₆ cycloalkyl;
R₁₀ is hydrogen, C₁₋₃ alkyl or C₁₋₃ haloalkyl;
n is 1 or 2;
q is 1, 2 or 3,
s is 0, 1 or 2, and
tis 1 or 2.

39. The compound of claim 30, or tautomer, pharmaceutically acceptable salt thereof, wherein the compound is of formula (VI): 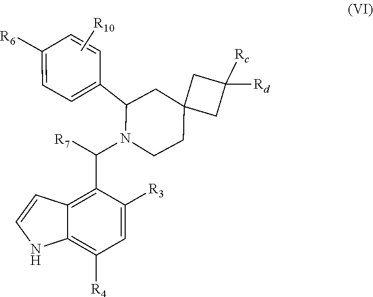
R₃ and R₄ are independently selected from deuterium, halogen, C₁₋₃ alkyl, C₁₋₃ alkoxy, C₃₋₆ cycloalkyl, C₃₋₆ cycloalkyloxy, deuterated C₁₋₃ alkoxy, C₁₋₃ haloalkoxy and C₃₋₆ cycloalkylC₁₋₃ alkoxy;
R₆ is —COOH, —C(O)NHSO₂CH₃, —S(O)NHCH₃, 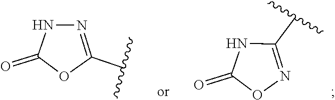 [SMILES: *c1n[nH]c(=O)o1 ; *c1noc(=O)[nH]1]
R₇ is hydrogen, C₁₋₃ alkyl or C₁₋₃ hydroxyalkyl;
R₁₀ is hydrogen, C₁₋₃ alkyl or C₁₋₃ haloalkyl;
each of R_{c} and R_{d} is independently selected from hydrogen, halogen, C₁₋₃ alkyl and C₁₋₃ haloalkyl.

40. The compound of claim 39, or tautomer, pharmaceutically acceptable salt thereof, wherein the compound is of formula (VI-a):

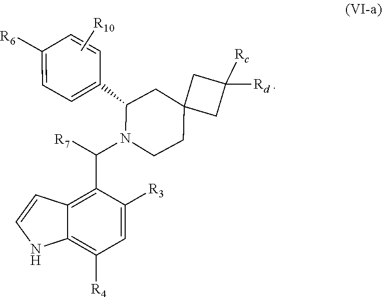

41. The compound of claim 40, or tautomer, pharmaceutically acceptable salt thereof, wherein:
R₃ and R₄ are independently selected from deuterium, halogen, C₁₋₃ alkyl, C₁₋₃ alkoxy, C₃₋₆ cycloalkyl;
R₆ is —COOH, —C(O)NHSO₂CH₃, —S(O)NHCH₃;
R₇ is hydrogen, C₁₋₃ alkyl or C₁₋₃ hydroxyalkyl;
R₁₀ is hydrogen, C₁₋₃ alkyl or C₁₋₃ haloalkyl;
each of R_{c} and R_{d} is independently selected from hydrogen, halogen, C₁₋₃ alkyl and C₁₋₃ haloalkyl.

42. The compound of claim 40, or tautomer, pharmaceutically acceptable salt thereof, wherein the compound is of formula (VI-b): 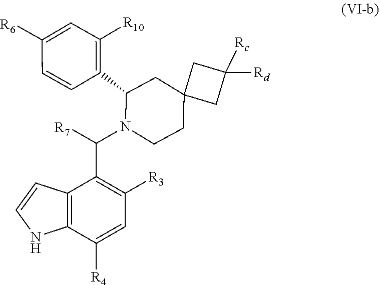
R₃ and R₄ are independently selected from deuterium, halogen, C₁₋₃ alkyl, C₁₋₃ alkoxy, cyclopropyl, cyclobutyl;
R₆ is —COOH, —C(O)NHSO₂CH₃, —S(O)NHCH₃;
R₇ is hydrogen, C₁₋₃ alkyl or C₁₋₃ hydroxyalkyl;
R₁₀ is hydrogen, C₁₋₃ alkyl or C₁₋₃ haloalkyl, wherein the haloalkyl group contains at least two halogen atoms selected from F;
each of R_{c} and R_{d} is independently selected from hydrogen, halogen, C₁₋₃ alkyl and C₁₋₃ haloalkyl.

43. The compound of claim 30, or tautomer, pharmaceutically acceptable salt thereof, wherein the compound selected from the following structure:

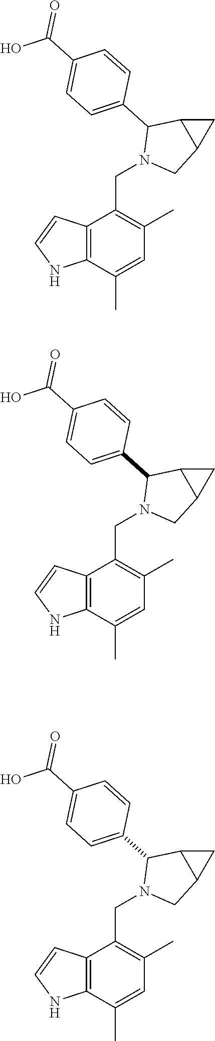 [SMILES: Cc1cc(C)c2[nH]ccc2c1CN1CC2CC2C1c1ccc(C(=O)O)cc1 ; Cc1cc(C)c2[nH]ccc2c1CN1CC2CC2[C@@H]1c1ccc(C(=O)O)cc1 ; Cc1cc(C)c2[nH]ccc2c1CN1CC2CC2[C@H]1c1ccc(C(=O)O)cc1]

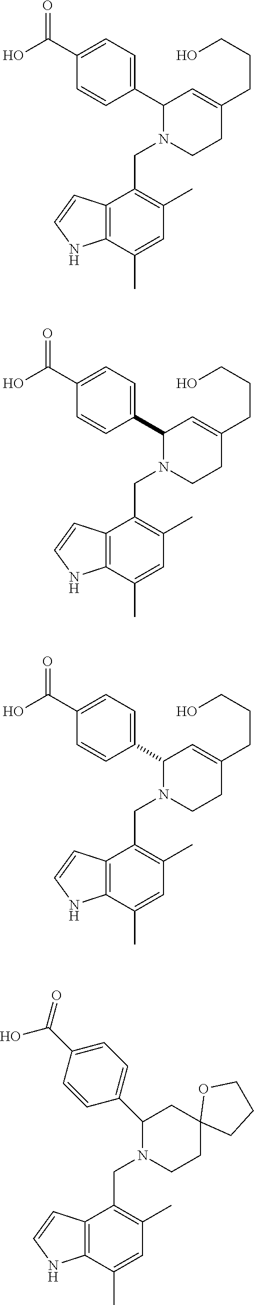 [SMILES: Cc1cc(C)c2[nH]ccc2c1CN1CCC(CCCO)=CC1c1ccc(C(=O)O)cc1 ; Cc1cc(C)c2[nH]ccc2c1CN1CCC(CCCO)=C[C@@H]1c1ccc(C(=O)O)cc1 ; Cc1cc(C)c2[nH]ccc2c1CN1CCC(CCCO)=C[C@H]1c1ccc(C(=O)O)cc1 ; Cc1cc(C)c2[nH]ccc2c1CN1CCC2(CCCO2)CC1c1ccc(C(=O)O)cc1]

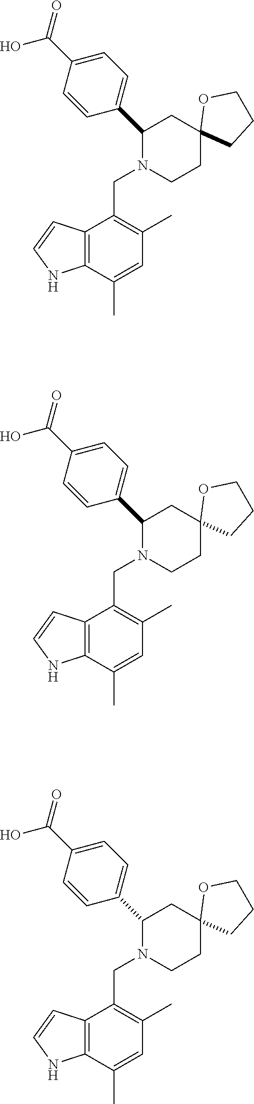 [SMILES: Cc1cc(C)c2[nH]ccc2c1CN1CC[C@]2(CCCO2)C[C@@H]1c1ccc(C(=O)O)cc1 ; Cc1cc(C)c2[nH]ccc2c1CN1CC[C@@]2(CCCO2)C[C@@H]1c1ccc(C(=O)O)cc1 ; Cc1cc(C)c2[nH]ccc2c1CN1CC[C@@]2(CCCO2)C[C@H]1c1ccc(C(=O)O)cc1]

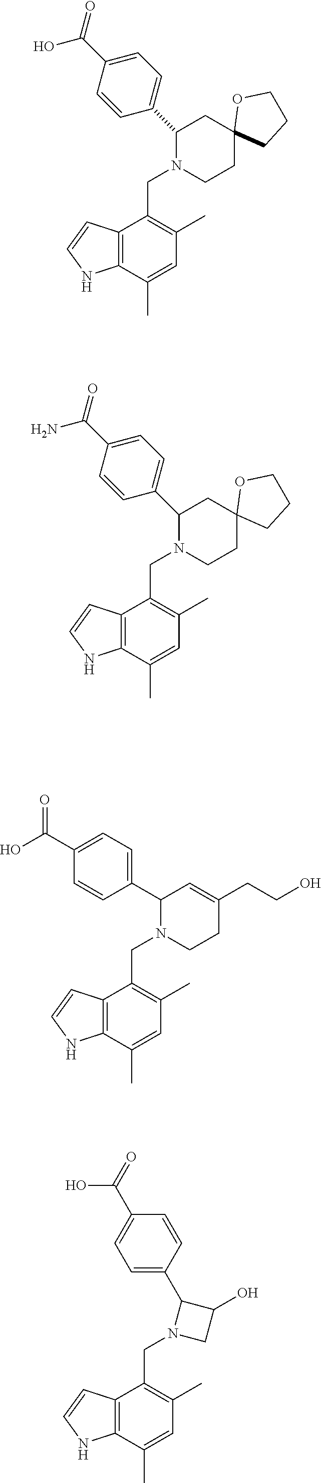 [SMILES: Cc1cc(C)c2[nH]ccc2c1CN1CC[C@]2(CCCO2)C[C@H]1c1ccc(C(=O)O)cc1 ; Cc1cc(C)c2[nH]ccc2c1CN1CCC2(CCCO2)CC1c1ccc(C(N)=O)cc1 ; Cc1cc(C)c2[nH]ccc2c1CN1CCC(CCO)=CC1c1ccc(C(=O)O)cc1 ; Cc1cc(C)c2[nH]ccc2c1CN1CC(O)C1c1ccc(C(=O)O)cc1]

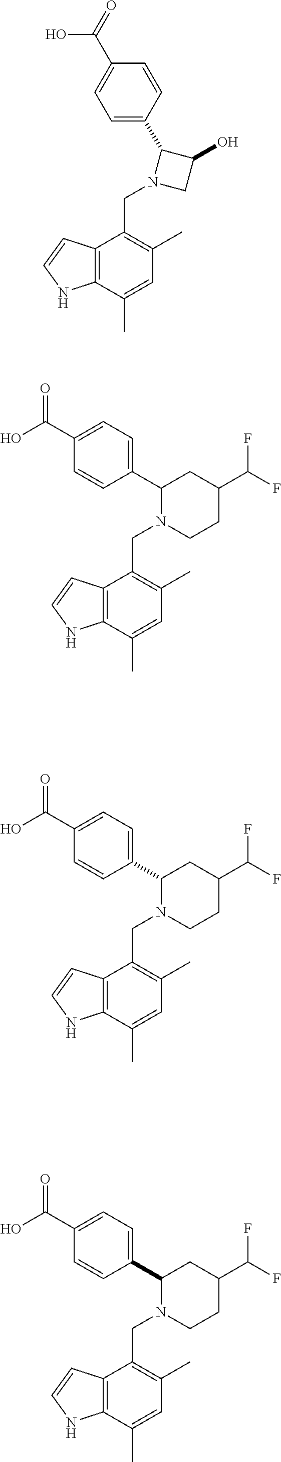 [SMILES: Cc1cc(C)c2[nH]ccc2c1CN1C[C@H](O)[C@H]1c1ccc(C(=O)O)cc1 ; Cc1cc(C)c2[nH]ccc2c1CN1CCC(C(F)F)CC1c1ccc(C(=O)O)cc1 ; Cc1cc(C)c2[nH]ccc2c1CN1CCC(C(F)F)C[C@H]1c1ccc(C(=O)O)cc1 ; Cc1cc(C)c2[nH]ccc2c1CN1CCC(C(F)F)C[C@@H]1c1ccc(C(=O)O)cc1]

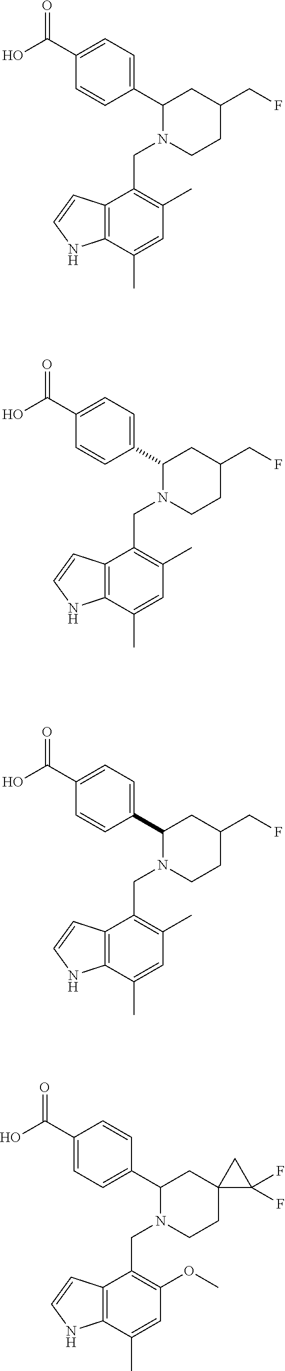 [SMILES: Cc1cc(C)c2[nH]ccc2c1CN1CCC(CF)CC1c1ccc(C(=O)O)cc1 ; Cc1cc(C)c2[nH]ccc2c1CN1CCC(CF)C[C@H]1c1ccc(C(=O)O)cc1 ; Cc1cc(C)c2[nH]ccc2c1CN1CCC(CF)C[C@@H]1c1ccc(C(=O)O)cc1 ; COc1cc(C)c2[nH]ccc2c1CN1CCC2(CC1c1ccc(C(=O)O)cc1)CC2(F)F]

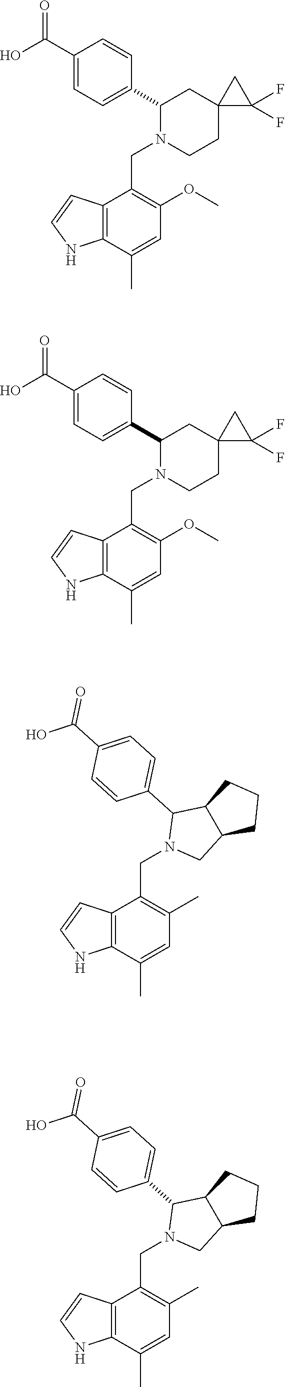 [SMILES: COc1cc(C)c2[nH]ccc2c1CN1CCC2(C[C@H]1c1ccc(C(=O)O)cc1)CC2(F)F ; COc1cc(C)c2[nH]ccc2c1CN1CCC2(C[C@@H]1c1ccc(C(=O)O)cc1)CC2(F)F ; Cc1cc(C)c2[nH]ccc2c1CN1C[C@@H]2CCC[C@@H]2C1c1ccc(C(=O)O)cc1 ; Cc1cc(C)c2[nH]ccc2c1CN1C[C@@H]2CCC[C@@H]2[C@H]1c1ccc(C(=O)O)cc1]

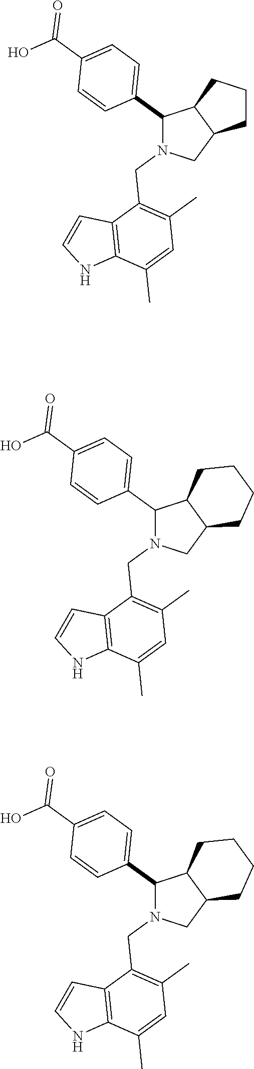 [SMILES: Cc1cc(C)c2[nH]ccc2c1CN1C[C@@H]2CCC[C@@H]2[C@@H]1c1ccc(C(=O)O)cc1 ; Cc1cc(C)c2[nH]ccc2c1CN1C[C@@H]2CCCC[C@@H]2C1c1ccc(C(=O)O)cc1 ; Cc1cc(C)c2[nH]ccc2c1CN1C[C@@H]2CCCC[C@@H]2[C@@H]1c1ccc(C(=O)O)cc1]

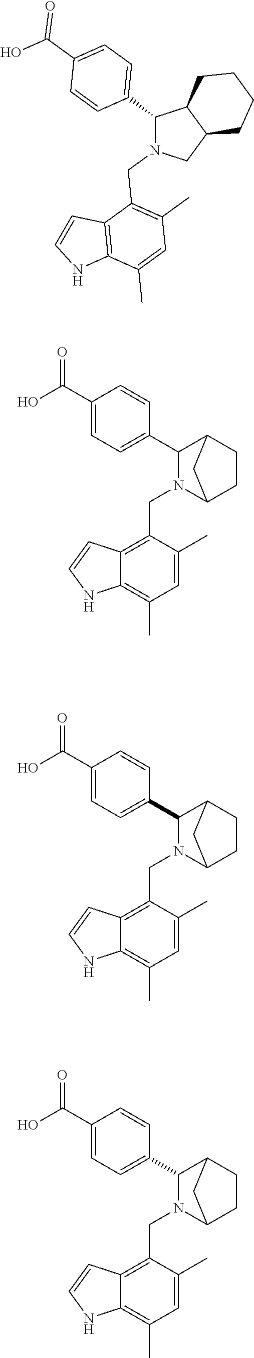 [SMILES: Cc1cc(C)c2[nH]ccc2c1CN1C[C@@H]2CCCC[C@@H]2[C@H]1c1ccc(C(=O)O)cc1 ; Cc1cc(C)c2[nH]ccc2c1CN1C2CCC(C2)C1c1ccc(C(=O)O)cc1 ; Cc1cc(C)c2[nH]ccc2c1CN1C2CCC(C2)[C@@H]1c1ccc(C(=O)O)cc1 ; Cc1cc(C)c2[nH]ccc2c1CN1C2CCC(C2)[C@H]1c1ccc(C(=O)O)cc1]

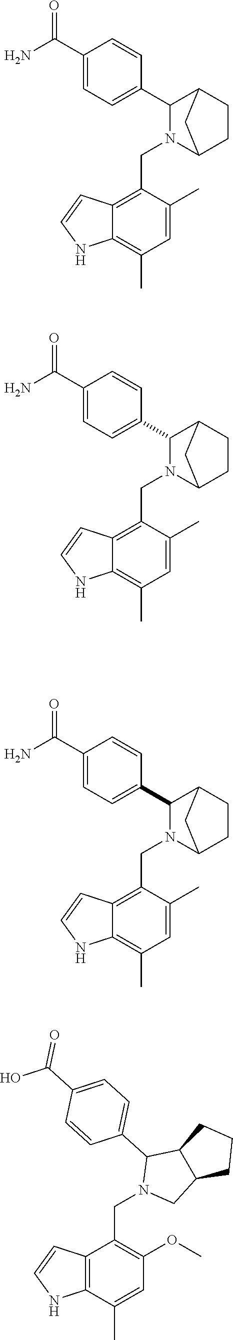 [SMILES: Cc1cc(C)c2[nH]ccc2c1CN1C2CCC(C2)C1c1ccc(C(N)=O)cc1 ; Cc1cc(C)c2[nH]ccc2c1CN1C2CCC(C2)[C@H]1c1ccc(C(N)=O)cc1 ; Cc1cc(C)c2[nH]ccc2c1CN1C2CCC(C2)[C@@H]1c1ccc(C(N)=O)cc1 ; COc1cc(C)c2[nH]ccc2c1CN1C[C@@H]2CCC[C@@H]2C1c1ccc(C(=O)O)cc1]

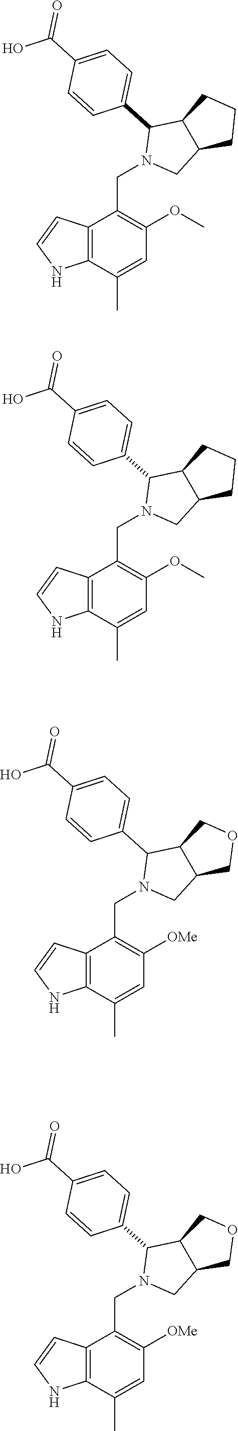 [SMILES: COc1cc(C)c2[nH]ccc2c1CN1C[C@@H]2CCC[C@@H]2[C@@H]1c1ccc(C(=O)O)cc1 ; COc1cc(C)c2[nH]ccc2c1CN1C[C@@H]2CCC[C@@H]2[C@H]1c1ccc(C(=O)O)cc1 ; COc1cc(C)c2[nH]ccc2c1CN1C[C@@H]2COC[C@@H]2C1c1ccc(C(=O)O)cc1 ; COc1cc(C)c2[nH]ccc2c1CN1C[C@@H]2COC[C@@H]2[C@H]1c1ccc(C(=O)O)cc1]

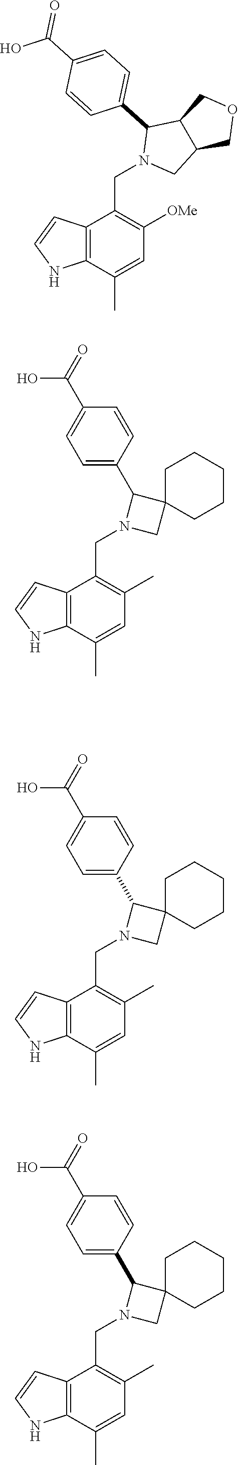

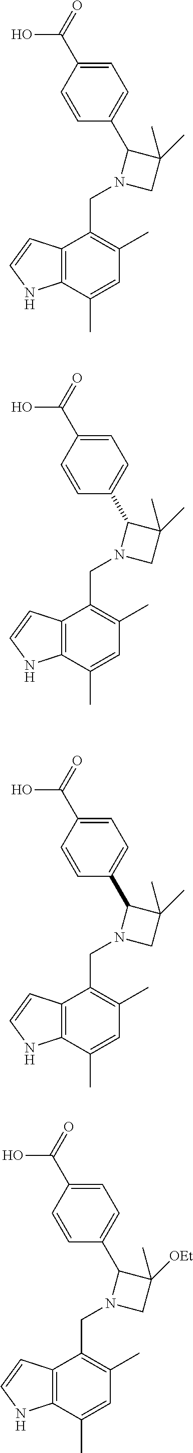

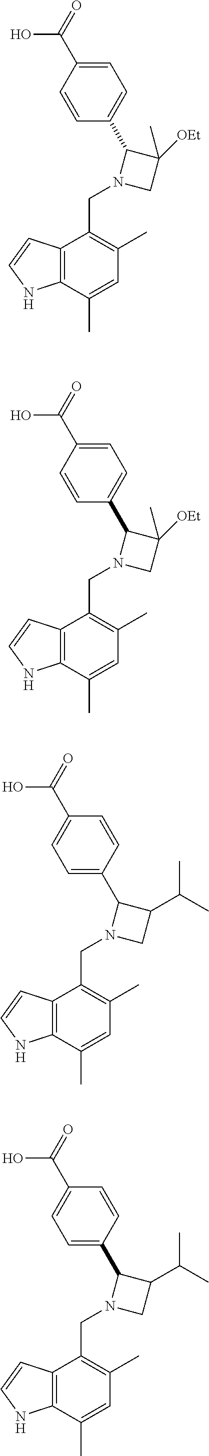

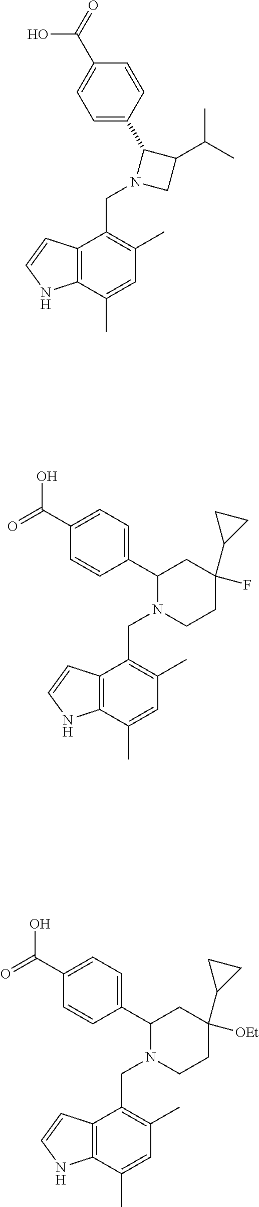 [SMILES: Cc1cc(C)c2[nH]ccc2c1CN1CC(C(C)C)[C@H]1c1ccc(C(=O)O)cc1 ; Cc1cc(C)c2[nH]ccc2c1CN1CCC(F)(C2CC2)CC1c1ccc(C(=O)O)cc1 ; CCOC1(C2CC2)CCN(Cc2c(C)cc(C)c3[nH]ccc23)C(c2ccc(C(=O)O)cc2)C1]

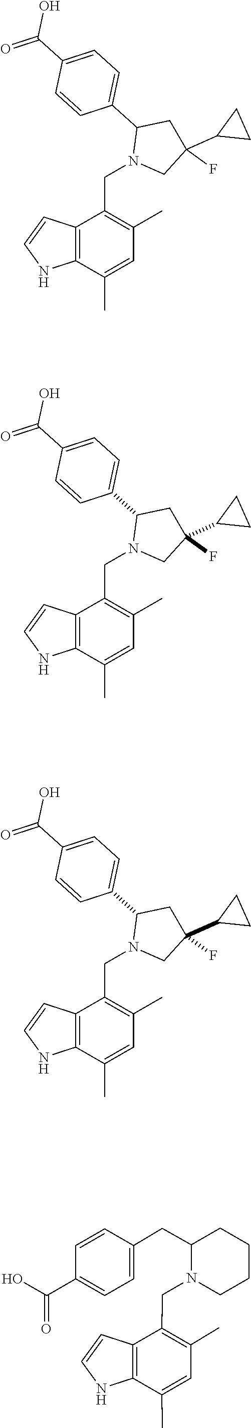

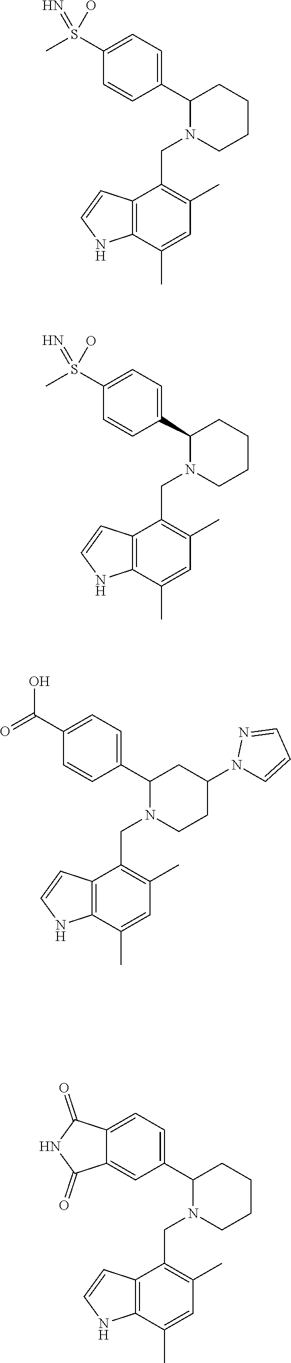

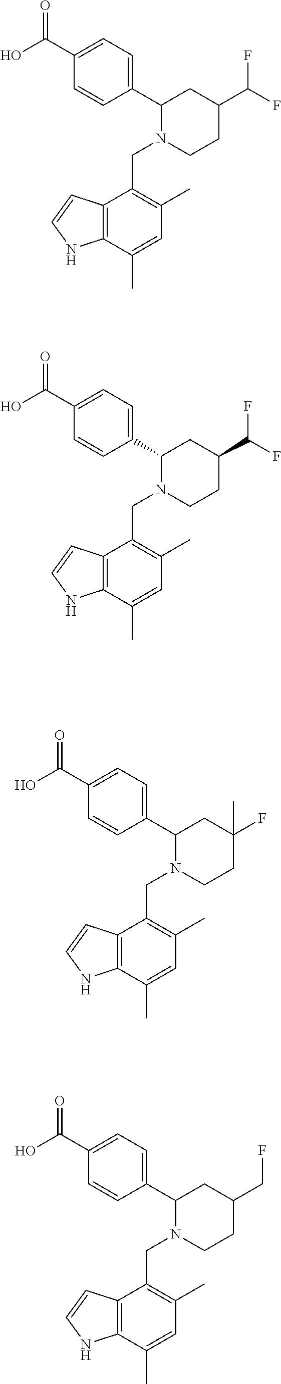 [SMILES: Cc1cc(C)c2[nH]ccc2c1CN1CCC(C(F)F)CC1c1ccc(C(=O)O)cc1 ; Cc1cc(C)c2[nH]ccc2c1CN1CC[C@H](C(F)F)C[C@H]1c1ccc(C(=O)O)cc1 ; Cc1cc(C)c2[nH]ccc2c1CN1CCC(C)(F)CC1c1ccc(C(=O)O)cc1 ; Cc1cc(C)c2[nH]ccc2c1CN1CCC(CF)CC1c1ccc(C(=O)O)cc1]

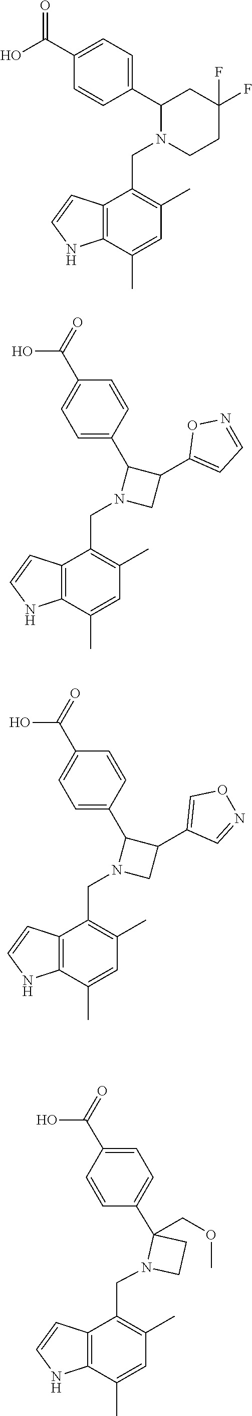 [SMILES: Cc1cc(C)c2[nH]ccc2c1CN1CCC(F)(F)CC1c1ccc(C(=O)O)cc1 ; Cc1cc(C)c2[nH]ccc2c1CN1CC(c2ccno2)C1c1ccc(C(=O)O)cc1 ; Cc1cc(C)c2[nH]ccc2c1CN1CC(c2cnoc2)C1c1ccc(C(=O)O)cc1 ; COCC1(c2ccc(C(=O)O)cc2)CCN1Cc1c(C)cc(C)c2[nH]ccc12]

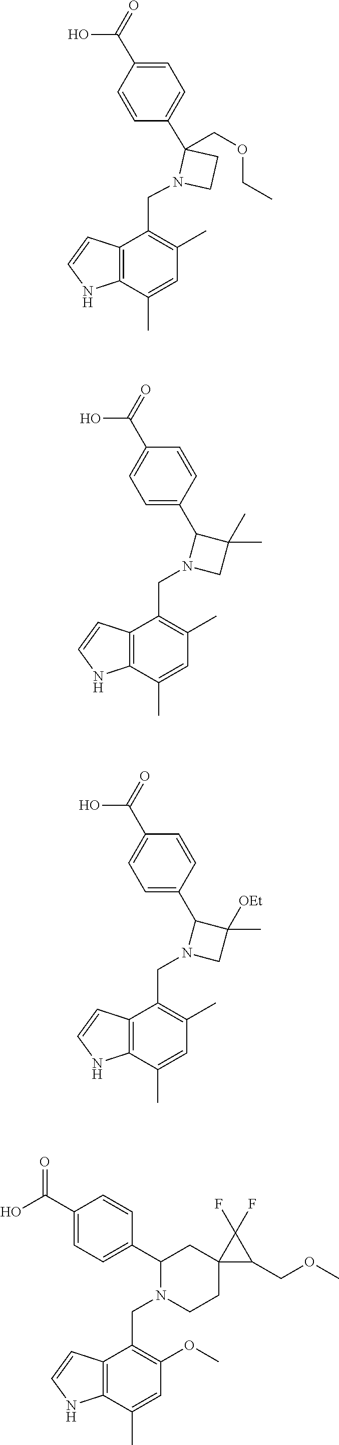 [SMILES: CCOCC1(c2ccc(C(=O)O)cc2)CCN1Cc1c(C)cc(C)c2[nH]ccc12 ; Cc1cc(C)c2[nH]ccc2c1CN1CC(C)(C)C1c1ccc(C(=O)O)cc1 ; CCOC1(C)CN(Cc2c(C)cc(C)c3[nH]ccc23)C1c1ccc(C(=O)O)cc1 ; COCC1C(F)(F)C12CCN(Cc1c(OC)cc(C)c3[nH]ccc13)C(c1ccc(C(=O)O)cc1)C2]

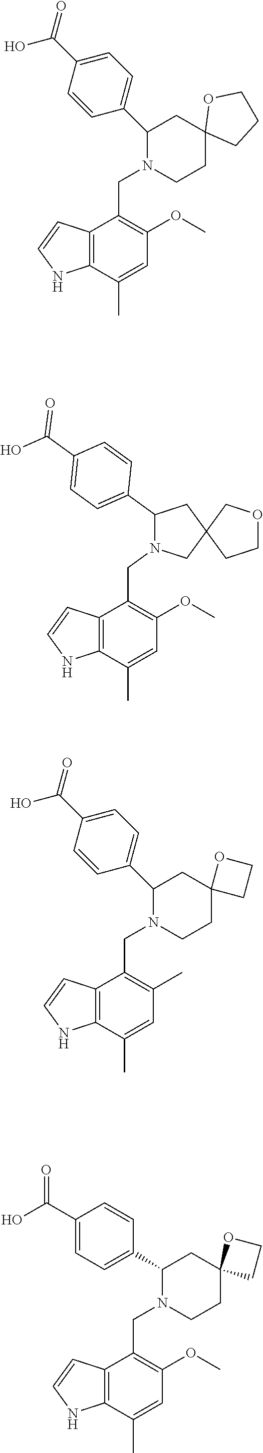 [SMILES: COc1cc(C)c2[nH]ccc2c1CN1CCC2(CCCO2)CC1c1ccc(C(=O)O)cc1 ; COc1cc(C)c2[nH]ccc2c1CN1CC2(CCOC2)CC1c1ccc(C(=O)O)cc1 ; Cc1cc(C)c2[nH]ccc2c1CN1CCC2(CCO2)CC1c1ccc(C(=O)O)cc1 ; COc1cc(C)c2[nH]ccc2c1CN1CC[C@@]2(CCO2)C[C@H]1c1ccc(C(=O)O)cc1]

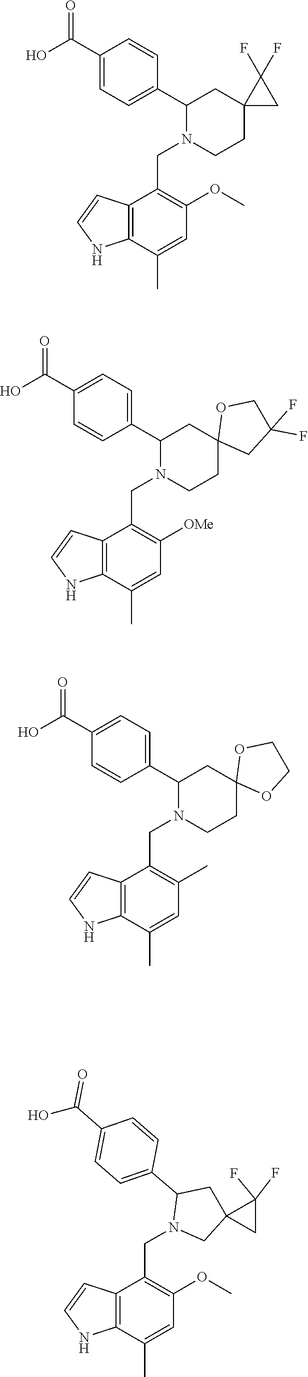 [SMILES: COc1cc(C)c2[nH]ccc2c1CN1CCC2(CC1c1ccc(C(=O)O)cc1)CC2(F)F ; COc1cc(C)c2[nH]ccc2c1CN1CCC2(CC1c1ccc(C(=O)O)cc1)CC(F)(F)CO2 ; Cc1cc(C)c2[nH]ccc2c1CN1CCC2(CC1c1ccc(C(=O)O)cc1)OCCO2 ; COc1cc(C)c2[nH]ccc2c1CN1CC2(CC1c1ccc(C(=O)O)cc1)CC2(F)F]

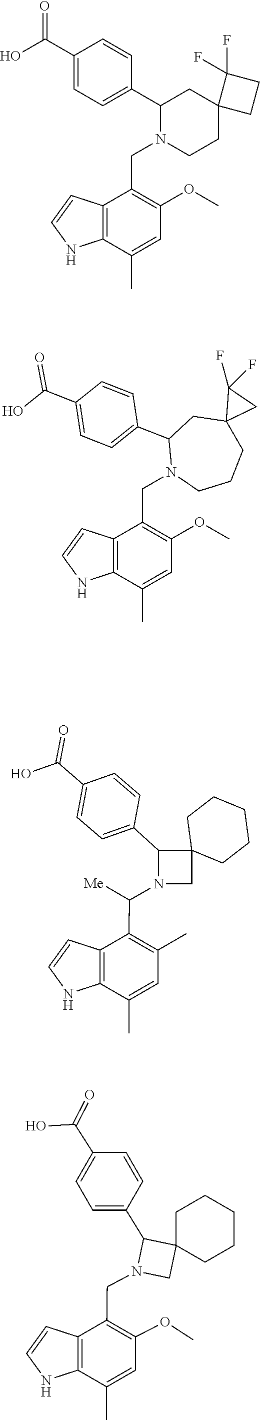 [SMILES: COc1cc(C)c2[nH]ccc2c1CN1CCC2(CCC2(F)F)CC1c1ccc(C(=O)O)cc1 ; COc1cc(C)c2[nH]ccc2c1CN1CCCC2(CC1c1ccc(C(=O)O)cc1)CC2(F)F ; Cc1cc(C)c2[nH]ccc2c1C(C)N1CC2(CCCCC2)C1c1ccc(C(=O)O)cc1 ; COc1cc(C)c2[nH]ccc2c1CN1CC2(CCCCC2)C1c1ccc(C(=O)O)cc1]

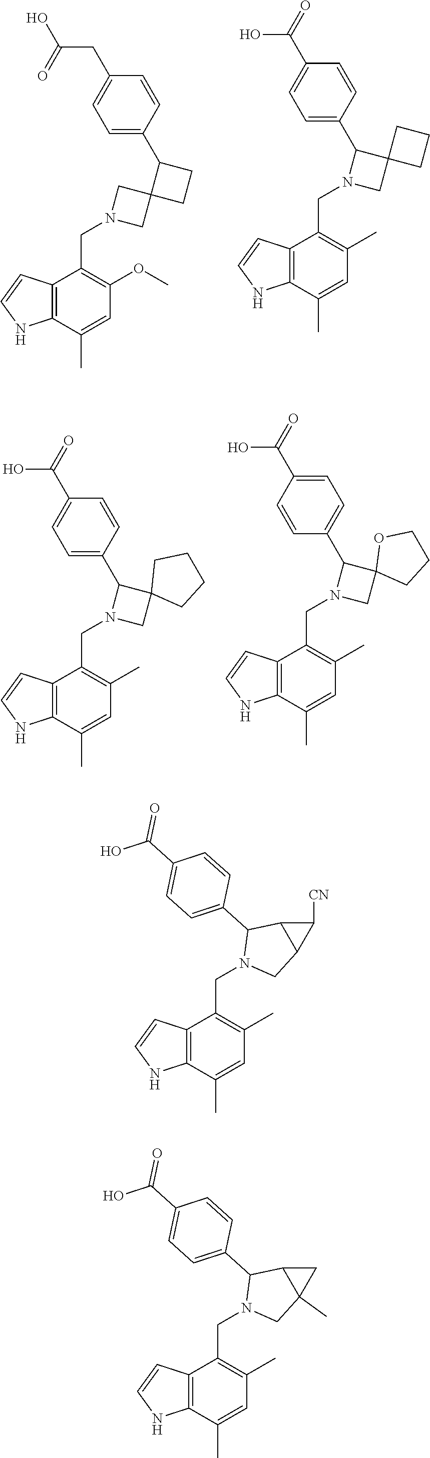 [SMILES: COc1cc(C)c2[nH]ccc2c1CN1CC2(CCC2c2ccc(CC(=O)O)cc2)C1 ; Cc1cc(C)c2[nH]ccc2c1CN1CC2(CCC2)C1c1ccc(C(=O)O)cc1 ; Cc1cc(C)c2[nH]ccc2c1CN1CC2(CCCC2)C1c1ccc(C(=O)O)cc1 ; Cc1cc(C)c2[nH]ccc2c1CN1CC2(CCCO2)C1c1ccc(C(=O)O)cc1 ; Cc1cc(C)c2[nH]ccc2c1CN1CC2C(C#N)C2C1c1ccc(C(=O)O)cc1 ; Cc1cc(C)c2[nH]ccc2c1CN1CC2(C)CC2C1c1ccc(C(=O)O)cc1]

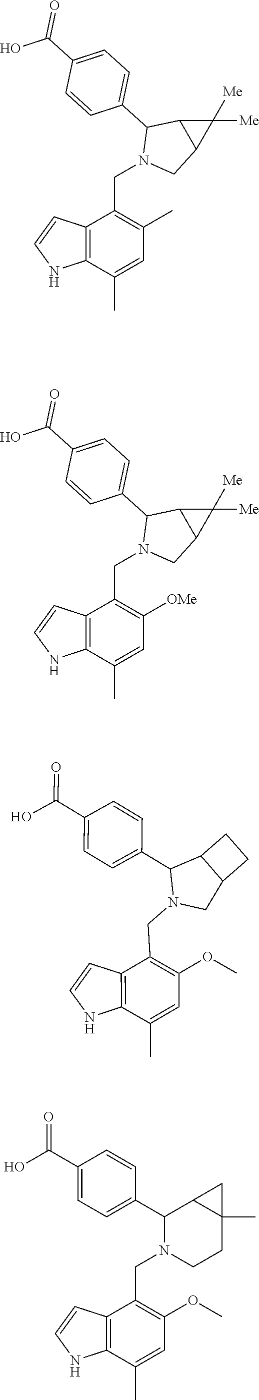 [SMILES: Cc1cc(C)c2[nH]ccc2c1CN1CC2C(C1c1ccc(C(=O)O)cc1)C2(C)C ; COc1cc(C)c2[nH]ccc2c1CN1CC2C(C1c1ccc(C(=O)O)cc1)C2(C)C ; COc1cc(C)c2[nH]ccc2c1CN1CC2CCC2C1c1ccc(C(=O)O)cc1 ; COc1cc(C)c2[nH]ccc2c1CN1CCC2(C)CC2C1c1ccc(C(=O)O)cc1]

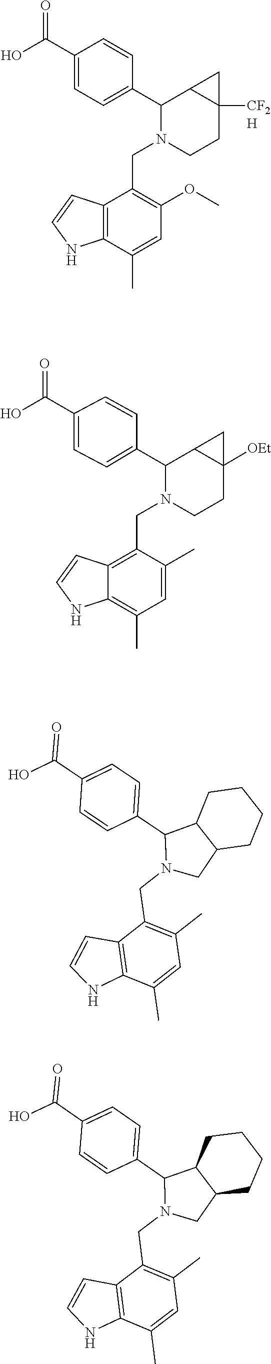

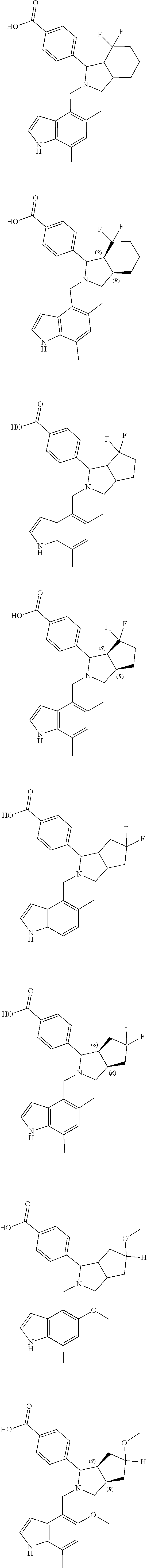

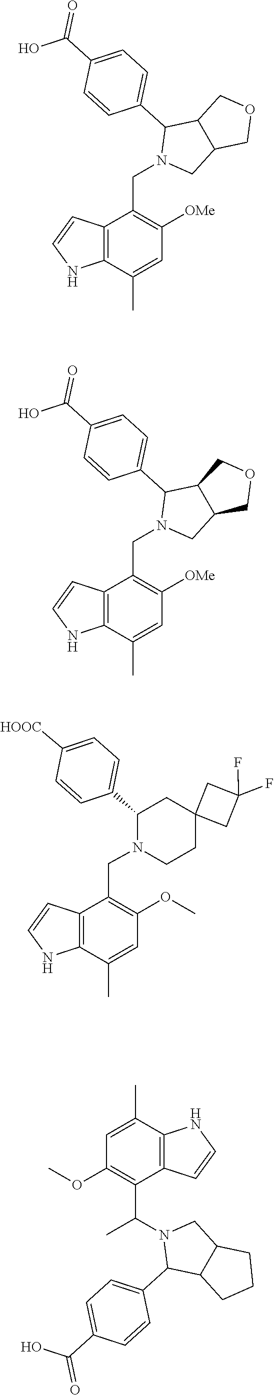 [SMILES: COc1cc(C)c2[nH]ccc2c1CN1CC2COCC2C1c1ccc(C(=O)O)cc1 ; COc1cc(C)c2[nH]ccc2c1CN1C[C@@H]2COC[C@@H]2C1c1ccc(C(=O)O)cc1 ; COc1cc(C)c2[nH]ccc2c1CN1CCC2(C[C@H]1c1ccc(C(=O)O)cc1)CC(F)(F)C2 ; COc1cc(C)c2[nH]ccc2c1C(C)N1CC2CCCC2C1c1ccc(C(=O)O)cc1]

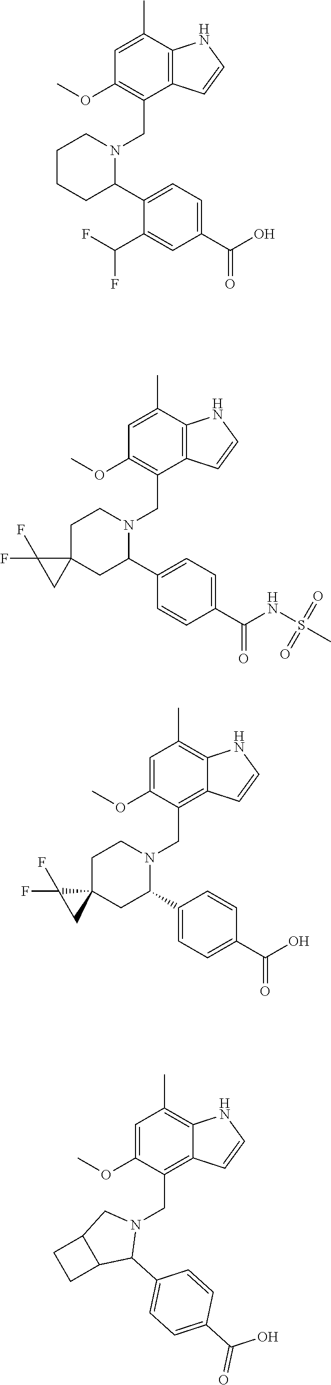 [SMILES: COc1cc(C)c2[nH]ccc2c1CN1CCCCC1c1ccc(C(=O)O)cc1C(F)F ; COc1cc(C)c2[nH]ccc2c1CN1CCC2(CC1c1ccc(C(=O)NS(C)(=O)=O)cc1)CC2(F)F ; COc1cc(C)c2[nH]ccc2c1CN1CC[C@@]2(C[C@H]1c1ccc(C(=O)O)cc1)CC2(F)F ; COc1cc(C)c2[nH]ccc2c1CN1CC2CCC2C1c1ccc(C(=O)O)cc1]

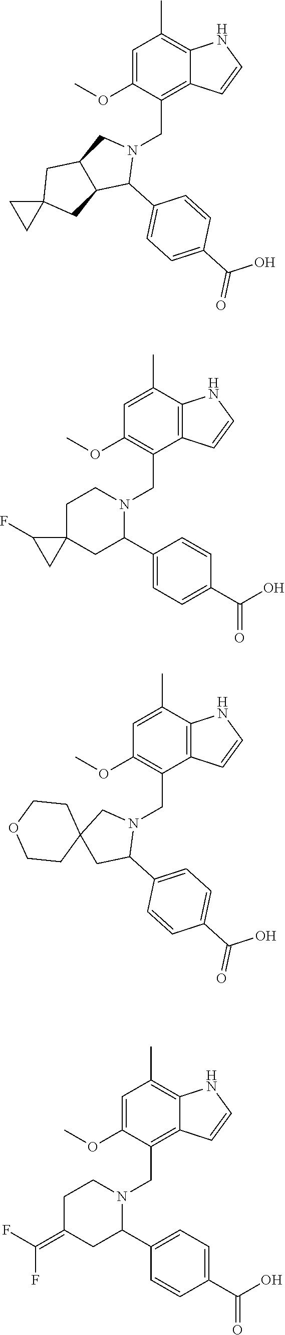

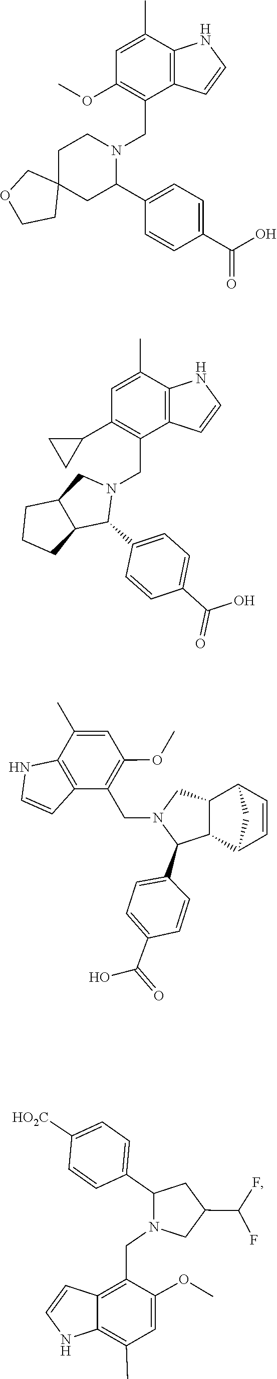 [SMILES: COc1cc(C)c2[nH]ccc2c1CN1CCC2(CCOC2)CC1c1ccc(C(=O)O)cc1 ; Cc1cc(C2CC2)c(CN2C[C@@H]3CCC[C@@H]3[C@H]2c2ccc(C(=O)O)cc2)c2cc[nH]c12 ; COc1cc(C)c2[nH]ccc2c1CN1C[C@H]2[C@H]([C@@H]3C=C[C@H]2C3)[C@H]1c1ccc(C(=O)O)cc1 ; COc1cc(C)c2[nH]ccc2c1CN1CC(C(F)F)CC1c1ccc(C(=O)O)cc1]

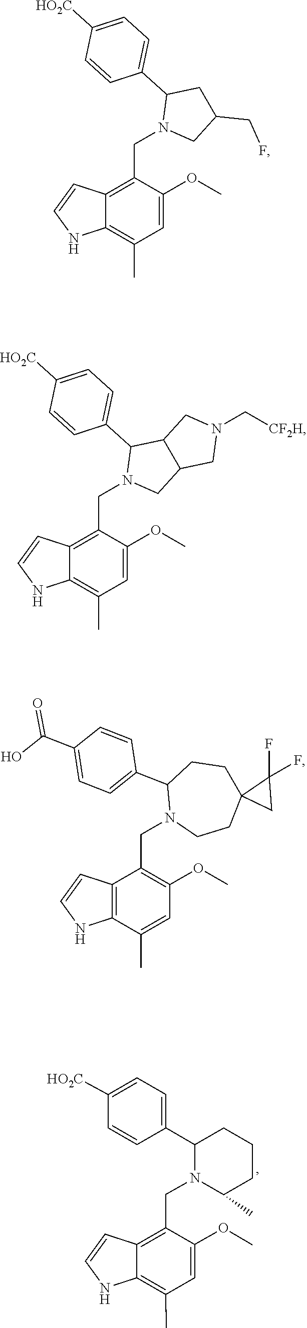 [SMILES: COc1cc(C)c2[nH]ccc2c1CN1CC(CF)CC1c1ccc(C(=O)O)cc1 ; COc1cc(C)c2[nH]ccc2c1CN1CC2CN(CC(F)F)CC2C1c1ccc(C(=O)O)cc1 ; COc1cc(C)c2[nH]ccc2c1CN1CCC2(CCC1c1ccc(C(=O)O)cc1)CC2(F)F ; COc1cc(C)c2[nH]ccc2c1CN1C(c2ccc(C(=O)O)cc2)CCC[C@@H]1C]

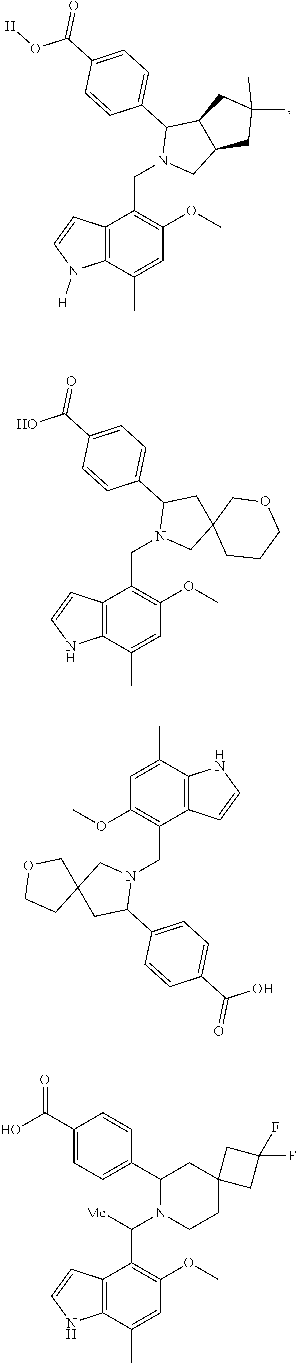 [SMILES: COc1cc(C)c2[nH]ccc2c1CN1C[C@@H]2CC(C)(C)C[C@@H]2C1c1ccc(C(=O)O)cc1 ; COc1cc(C)c2[nH]ccc2c1CN1CC2(CCCOC2)CC1c1ccc(C(=O)O)cc1 ; COc1cc(C)c2[nH]ccc2c1CN1CC2(CCOC2)CC1c1ccc(C(=O)O)cc1 ; COc1cc(C)c2[nH]ccc2c1C(C)N1CCC2(CC1c1ccc(C(=O)O)cc1)CC(F)(F)C2]

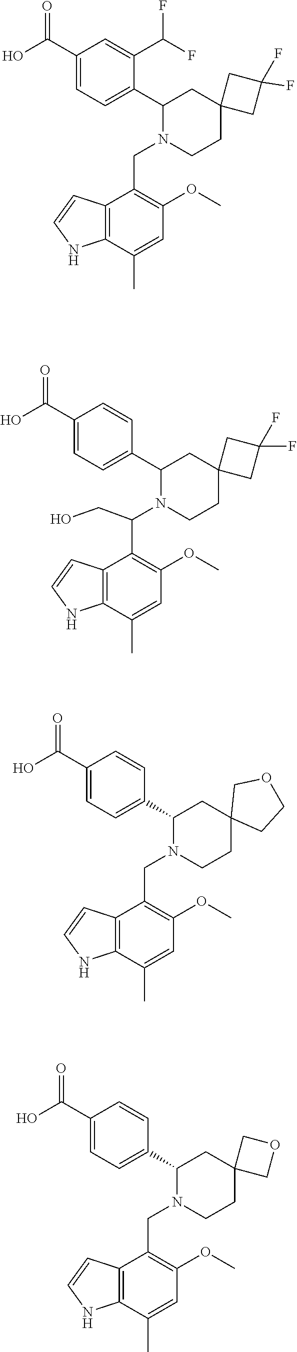 [SMILES: COc1cc(C)c2[nH]ccc2c1CN1CCC2(CC1c1ccc(C(=O)O)cc1C(F)F)CC(F)(F)C2 ; COc1cc(C)c2[nH]ccc2c1C(CO)N1CCC2(CC1c1ccc(C(=O)O)cc1)CC(F)(F)C2 ; COc1cc(C)c2[nH]ccc2c1CN1CCC2(CCOC2)C[C@H]1c1ccc(C(=O)O)cc1 ; COc1cc(C)c2[nH]ccc2c1CN1CCC2(COC2)C[C@H]1c1ccc(C(=O)O)cc1]

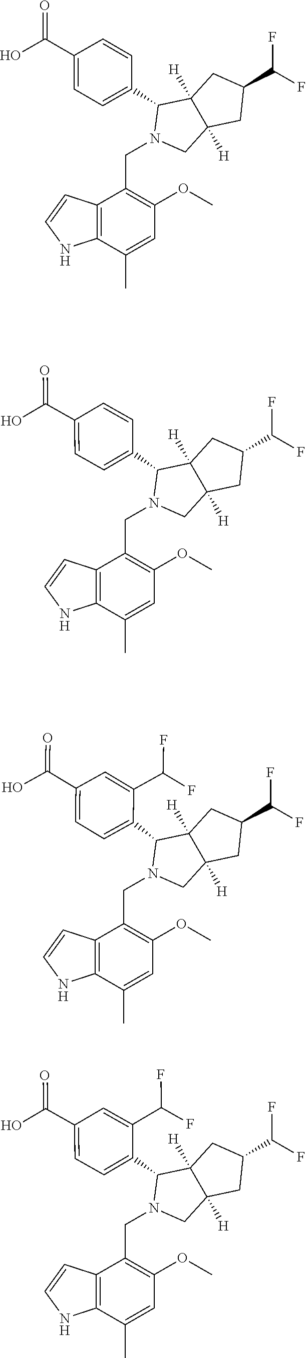 [SMILES: COc1cc(C)c2[nH]ccc2c1CN1C[C@@H]2C[C@H](C(F)F)C[C@@H]2[C@H]1c1ccc(C(=O)O)cc1 ; COc1cc(C)c2[nH]ccc2c1CN1C[C@@H]2C[C@@H](C(F)F)C[C@@H]2[C@H]1c1ccc(C(=O)O)cc1 ; COc1cc(C)c2[nH]ccc2c1CN1C[C@@H]2C[C@H](C(F)F)C[C@@H]2[C@H]1c1ccc(C(=O)O)cc1C(F)F ; COc1cc(C)c2[nH]ccc2c1CN1C[C@@H]2C[C@@H](C(F)F)C[C@@H]2[C@H]1c1ccc(C(=O)O)cc1C(F)F]

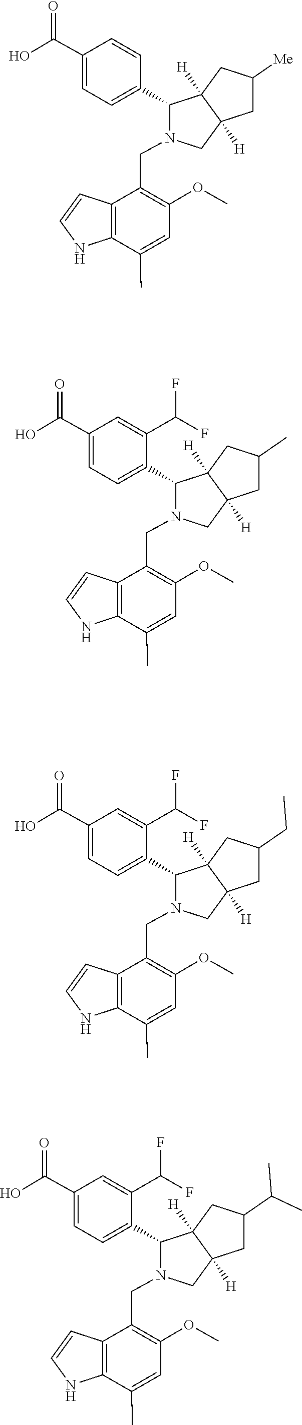 [SMILES: COc1cc(C)c2[nH]ccc2c1CN1C[C@@H]2CC(C)C[C@@H]2[C@H]1c1ccc(C(=O)O)cc1 ; COc1cc(C)c2[nH]ccc2c1CN1C[C@@H]2CC(C)C[C@@H]2[C@H]1c1ccc(C(=O)O)cc1C(F)F ; CCC1C[C@H]2CN(Cc3c(OC)cc(C)c4[nH]ccc34)[C@H](c3ccc(C(=O)O)cc3C(F)F)[C@H]2C1 ; COc1cc(C)c2[nH]ccc2c1CN1C[C@@H]2CC(C(C)C)C[C@@H]2[C@H]1c1ccc(C(=O)O)cc1C(F)F]

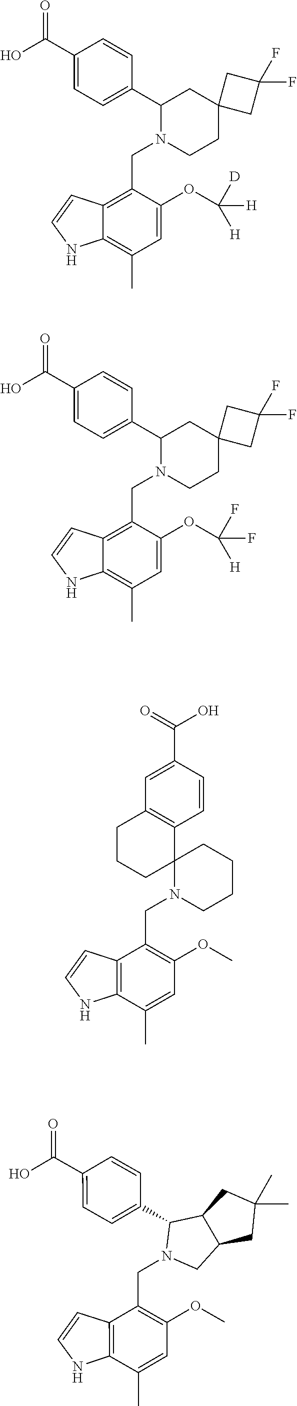 [SMILES: [2H]COc1cc(C)c2[nH]ccc2c1CN1CCC2(CC1c1ccc(C(=O)O)cc1)CC(F)(F)C2 ; Cc1cc(OC(F)F)c(CN2CCC3(CC2c2ccc(C(=O)O)cc2)CC(F)(F)C3)c2cc[nH]c12 ; COc1cc(C)c2[nH]ccc2c1CN1CCCCC12CCCc1cc(C(=O)O)ccc12 ; COc1cc(C)c2[nH]ccc2c1CN1C[C@@H]2CC(C)(C)C[C@@H]2[C@H]1c1ccc(C(=O)O)cc1]

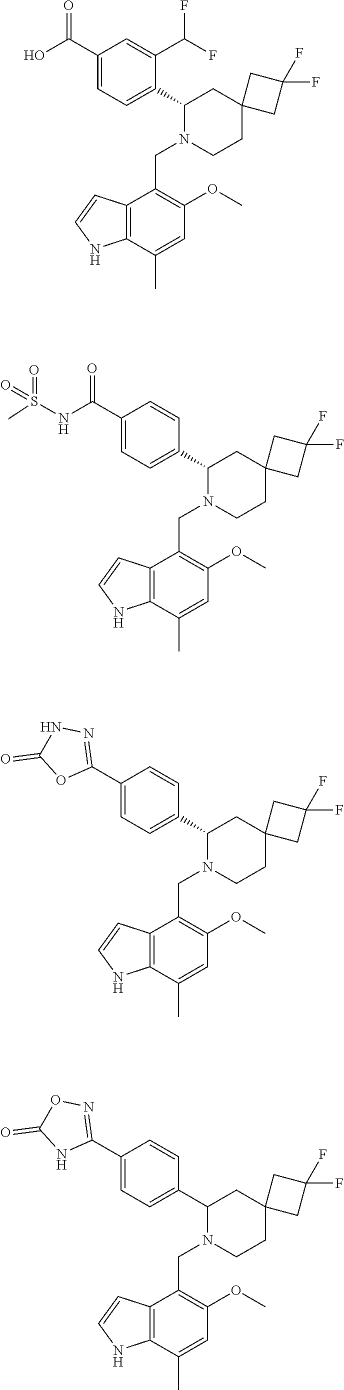 [SMILES: COc1cc(C)c2[nH]ccc2c1CN1CCC2(C[C@H]1c1ccc(C(=O)O)cc1C(F)F)CC(F)(F)C2 ; COc1cc(C)c2[nH]ccc2c1CN1CCC2(C[C@H]1c1ccc(C(=O)NS(C)(=O)=O)cc1)CC(F)(F)C2 ; COc1cc(C)c2[nH]ccc2c1CN1CCC2(C[C@H]1c1ccc(-c3n[nH]c(=O)o3)cc1)CC(F)(F)C2 ; COc1cc(C)c2[nH]ccc2c1CN1CCC2(CC1c1ccc(-c3noc(=O)[nH]3)cc1)CC(F)(F)C2]

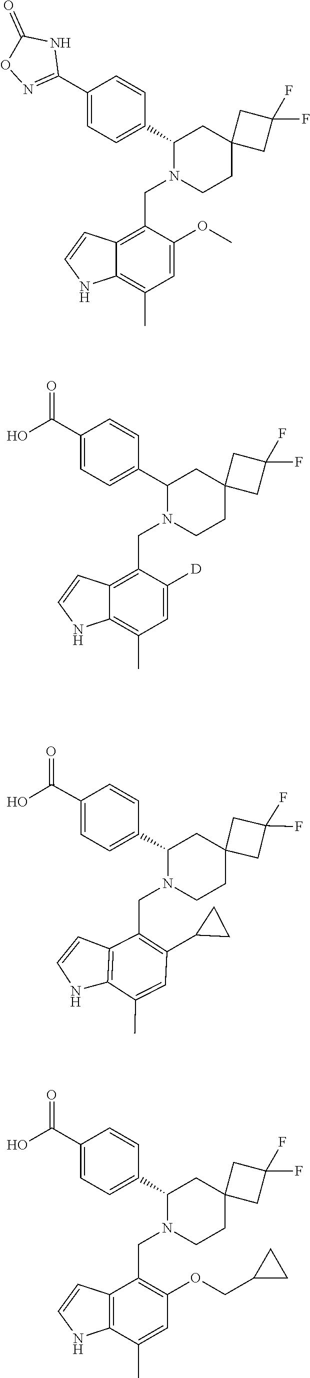 [SMILES: COc1cc(C)c2[nH]ccc2c1CN1CCC2(C[C@H]1c1ccc(-c3noc(=O)[nH]3)cc1)CC(F)(F)C2 ; [2H]c1cc(C)c2[nH]ccc2c1CN1CCC2(CC1c1ccc(C(=O)O)cc1)CC(F)(F)C2 ; Cc1cc(C2CC2)c(CN2CCC3(C[C@H]2c2ccc(C(=O)O)cc2)CC(F)(F)C3)c2cc[nH]c12 ; Cc1cc(OCC2CC2)c(CN2CCC3(C[C@H]2c2ccc(C(=O)O)cc2)CC(F)(F)C3)c2cc[nH]c12]

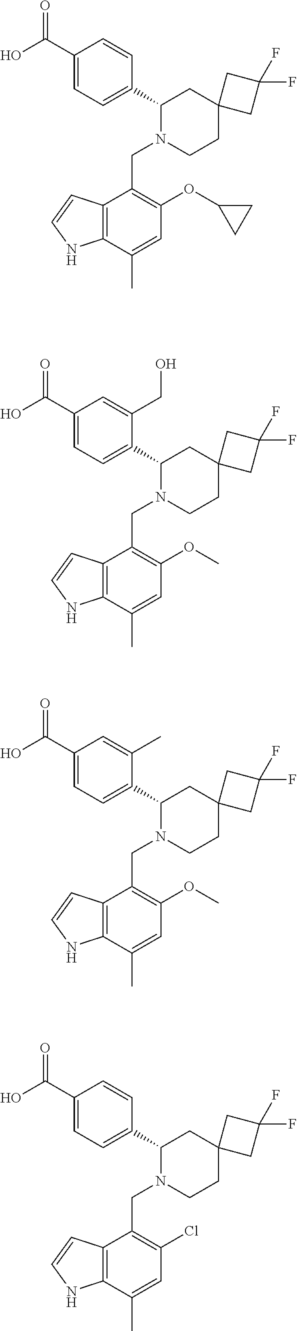 [SMILES: Cc1cc(OC2CC2)c(CN2CCC3(C[C@H]2c2ccc(C(=O)O)cc2)CC(F)(F)C3)c2cc[nH]c12 ; COc1cc(C)c2[nH]ccc2c1CN1CCC2(C[C@H]1c1ccc(C(=O)O)cc1CO)CC(F)(F)C2 ; COc1cc(C)c2[nH]ccc2c1CN1CCC2(C[C@H]1c1ccc(C(=O)O)cc1C)CC(F)(F)C2 ; Cc1cc(Cl)c(CN2CCC3(C[C@H]2c2ccc(C(=O)O)cc2)CC(F)(F)C3)c2cc[nH]c12]

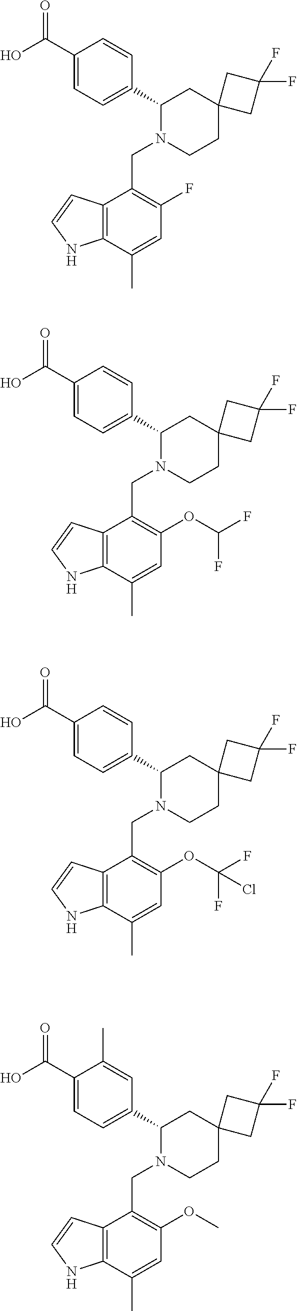 [SMILES: Cc1cc(F)c(CN2CCC3(C[C@H]2c2ccc(C(=O)O)cc2)CC(F)(F)C3)c2cc[nH]c12 ; Cc1cc(OC(F)F)c(CN2CCC3(C[C@H]2c2ccc(C(=O)O)cc2)CC(F)(F)C3)c2cc[nH]c12 ; Cc1cc(OC(F)(F)Cl)c(CN2CCC3(C[C@H]2c2ccc(C(=O)O)cc2)CC(F)(F)C3)c2cc[nH]c12 ; COc1cc(C)c2[nH]ccc2c1CN1CCC2(C[C@H]1c1ccc(C(=O)O)c(C)c1)CC(F)(F)C2]

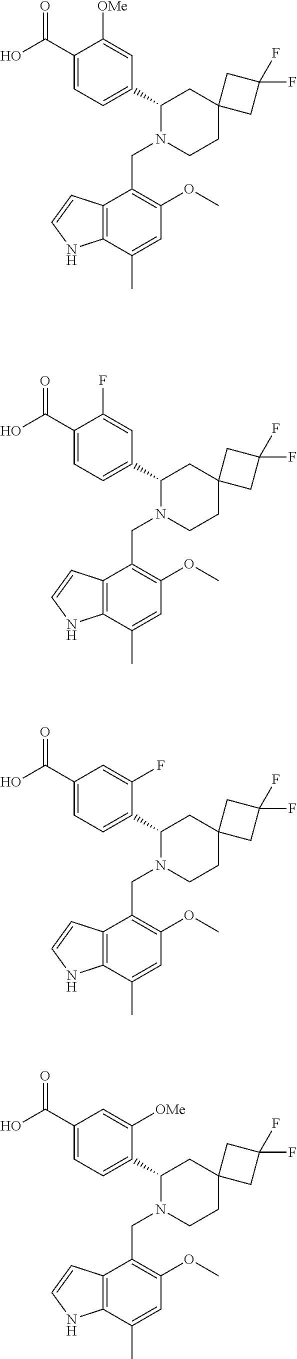 [SMILES: COc1cc([C@@H]2CC3(CCN2Cc2c(OC)cc(C)c4[nH]ccc24)CC(F)(F)C3)ccc1C(=O)O ; COc1cc(C)c2[nH]ccc2c1CN1CCC2(C[C@H]1c1ccc(C(=O)O)c(F)c1)CC(F)(F)C2 ; COc1cc(C)c2[nH]ccc2c1CN1CCC2(C[C@H]1c1ccc(C(=O)O)cc1F)CC(F)(F)C2 ; COc1cc(C(=O)O)ccc1[C@@H]1CC2(CCN1Cc1c(OC)cc(C)c3[nH]ccc13)CC(F)(F)C2]

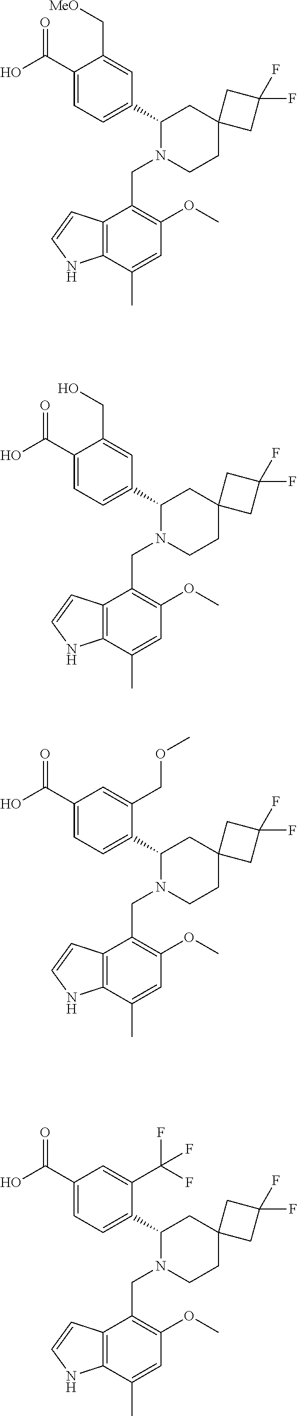 [SMILES: COCc1cc([C@@H]2CC3(CCN2Cc2c(OC)cc(C)c4[nH]ccc24)CC(F)(F)C3)ccc1C(=O)O ; COc1cc(C)c2[nH]ccc2c1CN1CCC2(C[C@H]1c1ccc(C(=O)O)c(CO)c1)CC(F)(F)C2 ; COCc1cc(C(=O)O)ccc1[C@@H]1CC2(CCN1Cc1c(OC)cc(C)c3[nH]ccc13)CC(F)(F)C2 ; COc1cc(C)c2[nH]ccc2c1CN1CCC2(C[C@H]1c1ccc(C(=O)O)cc1C(F)(F)F)CC(F)(F)C2]

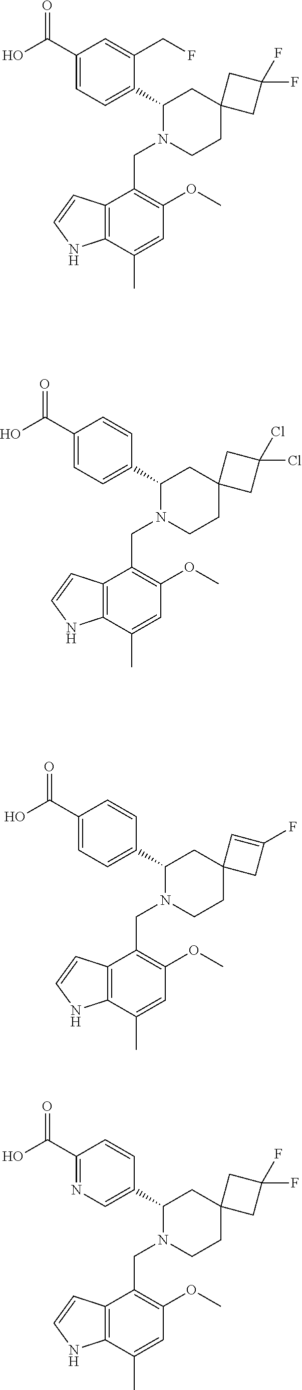 [SMILES: COc1cc(C)c2[nH]ccc2c1CN1CCC2(C[C@H]1c1ccc(C(=O)O)cc1CF)CC(F)(F)C2 ; COc1cc(C)c2[nH]ccc2c1CN1CCC2(C[C@H]1c1ccc(C(=O)O)cc1)CC(Cl)(Cl)C2 ; COc1cc(C)c2[nH]ccc2c1CN1CCC2(C=C(F)C2)C[C@H]1c1ccc(C(=O)O)cc1 ; COc1cc(C)c2[nH]ccc2c1CN1CCC2(C[C@H]1c1ccc(C(=O)O)nc1)CC(F)(F)C2]

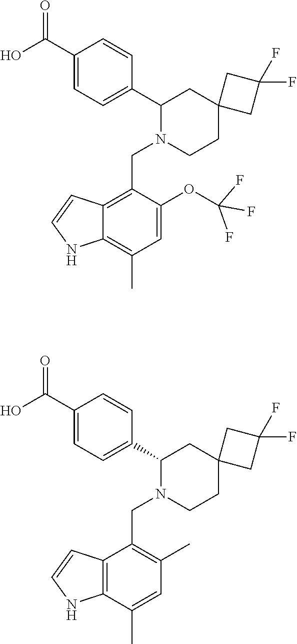 [SMILES: Cc1cc(OC(F)(F)F)c(CN2CCC3(CC2c2ccc(C(=O)O)cc2)CC(F)(F)C3)c2cc[nH]c12 ; Cc1cc(C)c2[nH]ccc2c1CN1CCC2(C[C@H]1c1ccc(C(=O)O)cc1)CC(F)(F)C2]

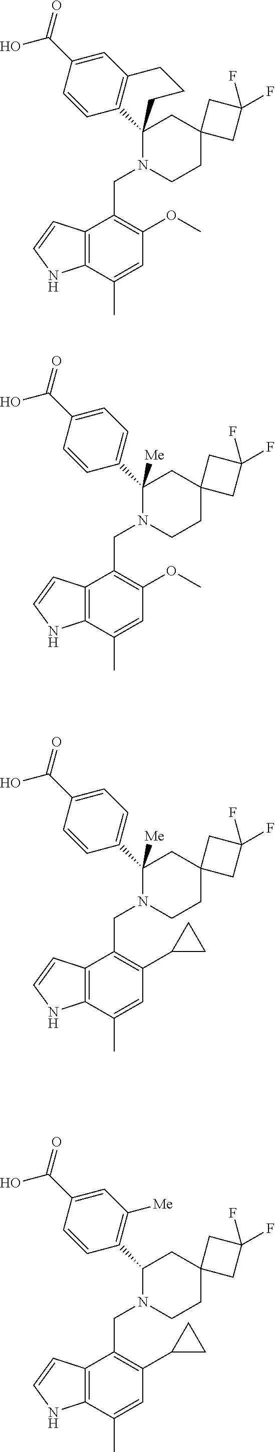

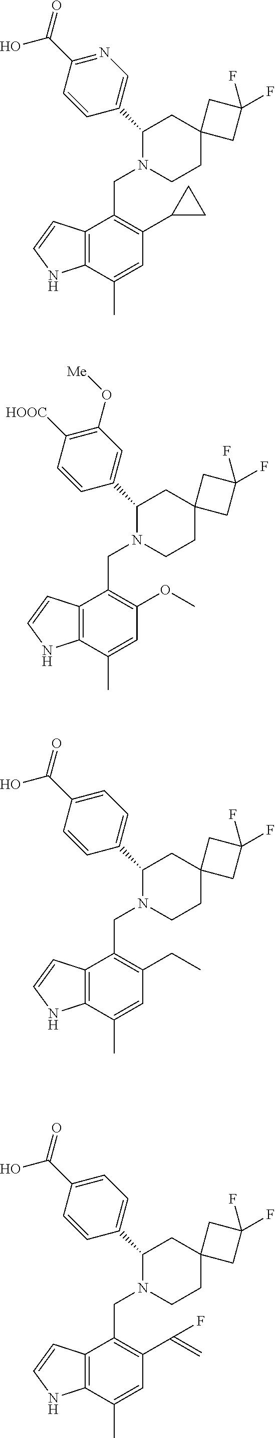 [SMILES: Cc1cc(C2CC2)c(CN2CCC3(C[C@H]2c2ccc(C(=O)O)nc2)CC(F)(F)C3)c2cc[nH]c12 ; COc1cc([C@@H]2CC3(CCN2Cc2c(OC)cc(C)c4[nH]ccc24)CC(F)(F)C3)ccc1C(=O)O ; CCc1cc(C)c2[nH]ccc2c1CN1CCC2(C[C@H]1c1ccc(C(=O)O)cc1)CC(F)(F)C2 ; C=C(F)c1cc(C)c2[nH]ccc2c1CN1CCC2(C[C@H]1c1ccc(C(=O)O)cc1)CC(F)(F)C2]

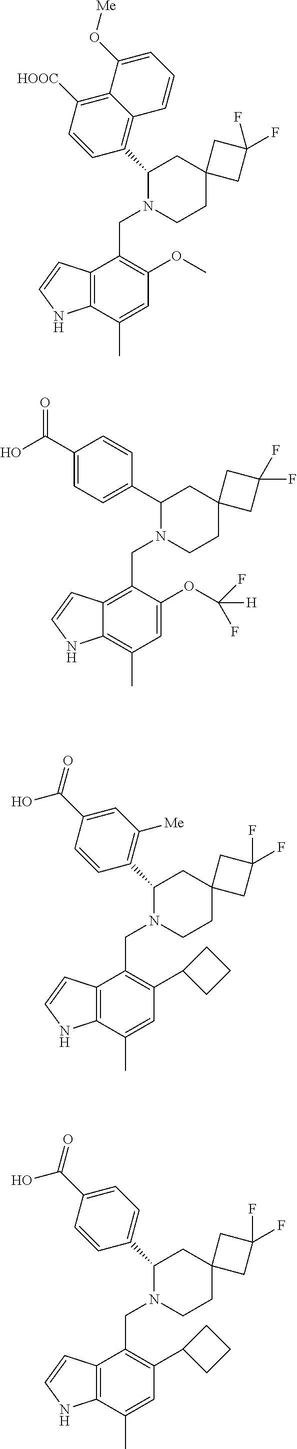 [SMILES: COc1cc(C)c2[nH]ccc2c1CN1CCC2(C[C@H]1c1ccc(C(=O)O)c3c(OC)cccc13)CC(F)(F)C2 ; Cc1cc(OC(F)F)c(CN2CCC3(CC2c2ccc(C(=O)O)cc2)CC(F)(F)C3)c2cc[nH]c12 ; Cc1cc(C(=O)O)ccc1[C@@H]1CC2(CCN1Cc1c(C3CCC3)cc(C)c3[nH]ccc13)CC(F)(F)C2 ; Cc1cc(C2CCC2)c(CN2CCC3(C[C@H]2c2ccc(C(=O)O)cc2)CC(F)(F)C3)c2cc[nH]c12]

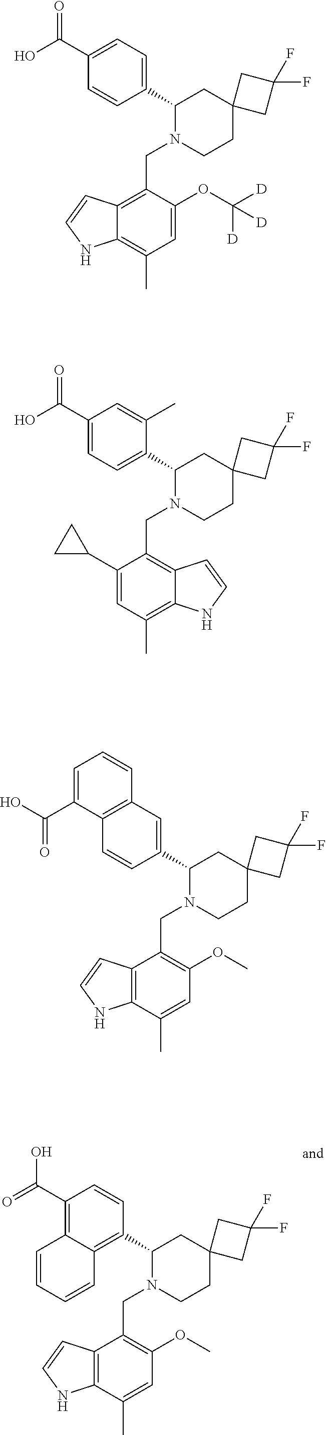 [SMILES: [2H]C([2H])([2H])Oc1cc(C)c2[nH]ccc2c1CN1CCC2(C[C@H]1c1ccc(C(=O)O)cc1)CC(F)(F)C2 ; Cc1cc(C(=O)O)ccc1[C@@H]1CC2(CCN1Cc1c(C3CC3)cc(C)c3[nH]ccc13)CC(F)(F)C2 ; COc1cc(C)c2[nH]ccc2c1CN1CCC2(C[C@H]1c1ccc3c(C(=O)O)cccc3c1)CC(F)(F)C2 ; COc1cc(C)c2[nH]ccc2c1CN1CCC2(C[C@H]1c1ccc(C(=O)O)c3ccccc13)CC(F)(F)C2]

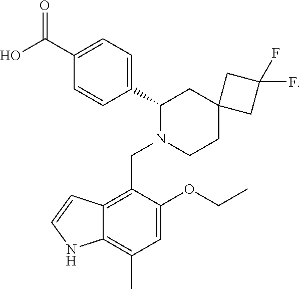 [SMILES: CCOc1cc(C)c2[nH]ccc2c1CN1CCC2(C[C@H]1c1ccc(C(=O)O)cc1)CC(F)(F)C2]

44. A pharmaceutical composition comprising a therapeutically effective amount of the compound of claim 30, or tautomer, pharmaceutically acceptable salt thereof, and a pharmaceutically acceptable carrier or excipient.

45. A pharmaceutical composition of claim 44, wherein, the amount of the compound, tautomer, cis- or trans-isomer, mesomer, racemate, enantiomer, diastereomer, or mixture thereof, or pharmaceutically acceptable salts thereof, is about 0.1%˜95% by weight of free base; preferably, is about 0.5%˜85% by weight of free base;
more preferably, is about 1%˜60% by weight of free base;
more preferably, is about 10%˜50% by weight of free base;
more preferably, is about 15-40% by weight of free base;
more preferably, is about 20-30% by weight of free base;
more preferably, is about 20-25% by weight of free base.

46. A pharmaceutical composition of claim 44, wherein, the pharmaceutical composition is in the form of tablet, capsule, liquid or injection; or the pharmaceutical composition is in an immediate release dosage or sustained release dosage; or, the pharmaceutical composition comprises at least one pharmaceutically acceptable excipient, carrier, or vehicle selected from the group consisting of: fillers, disintegrants, glidants, lubricants or diluents; or, the unit dosage of the compound, tautomer, cis- or trans-isomer, mesomer, racemate, enantiomer, diastereomer, or mixture thereof, or pharmaceutically acceptable salts thereof, is about 1-1000 mg by weight of free base;
preferably, is about 1-500 mg by weight of free base;
more preferably, is about 3-300 mg by weight of free base;
more preferably, is about 5-200 mg by weight of free base;
more preferably, is 1 mg, 2 mg, 3 mg, 5 mg, 10 mg, 20 mg, 40 mg, 50 mg, 60 mg, 80 mg, 100 mg, 200 mg, 300 mg, 400 mg or 500 mg by weight of free base.

47. A method of modulating complement alternative pathway activity in a subject, wherein the method comprises administering to the subject a therapeutically effective amount of the compound according to claim 30.

48. A method of treating a disorder or a disease in a subject mediated by complement activation, in particular mediated by activation of the complement alternative pathway, wherein the method comprises administering to the subject a therapeutically effective amount of the compound according to claim 30.

49. The method of claim 48, in which the disease or disorder is selected from the group consisting of: age-related macular degeneration, geographic atrophy, diabetic retinopathy, uveitis, retinitis pigmentosa, macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, post-operative inflammation, retinal vein occlusion, neurological disorders, multiple sclerosis, stroke, Guillain Barre Syndrome, traumatic brain injury, Parkinson's disease, disorders of inappropriate or undesirable complement activation, hemodialysis complications, hyperacute allograft rejection, xenograft rejection, interleukin-2 induced toxicity during IL-2 therapy, inflammatory disorders, inflammation of autoimmune diseases, Crohn's disease, adult respiratory distress syndrome, myocarditis, post-ischemic reperfusion conditions, myocardial infarction, balloon angioplasty, post-pump syndrome in cardiopulmonary bypass or renal bypass, atherosclerosis, hemodialysis, renal ischemia, mesenteric artery reperfusion after aortic reconstruction, infectious disease or sepsis, immune complex disorders and autoimmune diseases, rheumatoid arthritis, systemic lupus erythematosus, SLE nephritis, proliferative nephritis, liver fibrosis, hemolytic anemia, myasthenia gravis, tissue regeneration, neural regeneration, dyspnea, hemoptysis, ARDS, asthma, chronic obstructive pulmonary disease, emphysema, pulmonary embolisms and infarcts, pneumonia, fibrogenic dust diseases, pulmonary fibrosis, asthma, allergy, bronchoconstriction, hypersensitivity pneumonitis, parasitic diseases, Goodpasture's Syndrome, pulmonary vasculitis, Pauci-immune vasculitis, immune complex-associated inflammation, antiphospholipid syndrome, membrane nephropathy, paroxysmal sleep hemoglobinurine, IgA nephropathy, glomerulonephritis and obesity.
